SEQUENCE LISTING (26 sequences)

SEQ ID NO: 1 - DNA, 3282 bp, Homo sapiens gamma-aminobutyric acid (GABA) A receptor, pi (GABRP)
  CDS (157)..(1476)
gggacagggctgaggatgaggagaaccctggggacccagaagaccgtgccttgcccggaagtcctgcctgtaggcctgaaggacttgccctaacagagcctcaacaactacctggtgattcctacttcagccccttggtgtgagcagcttctcaacatgaactacagcctccacttggccttcgtgtgtctgagtctcttcactgagaggatgtgcatccaggggagtcagttcaacgtcgaggtcggcagaagtgacaagctttccctgcctggctttgagaacctcacagcaggatataacaaatttctcaggcccaattttggtggagaacccgtacagatagcgctgactctggacattgcaagtatctctagcatttcagagagtaacatggactacacagccaccatatacctccgacagcgctggatggaccagcggctggtgtttgaaggcaacaagagcttcactctggatgcccgcctcgtggagttcctctgggtgccagatacttacattgtggagtccaagaagtccttcctccatgaagtcactgtgggaaacaggctcatccgcctcttctccaatggcacggtcctgtatgccctcagaatcacgacaactgttgcatgtaacatggatctgtctaaataccccatggacacacagacatgcaagttgcagctggaaagctggggctatgatggaaatgatgtggagttcacctggctgagagggaacgactctgtgcgtggactggaacacctgcggcttgctcagtacaccatagagcggtatttcaccttagtcaccagatcgcagcaggagacaggaaattacactagattggtcttacagtttgagcttcggaggaatgttctgtatttcattttggaaacctacgttccttccactttcctggtggtgttgtcctgggtttcattttggatctctctcgattcagtccctgcaagaacctgcattggagtgacgaccgtgttatcaatgaccacactgatgatcgggtcccgcacttctcttcccaacaccaactgcttcatcaaggccatcgatgtgtacctggggatctgctttagctttgtgtttggggccttgctagaatatgcagttgctcactacagttccttacagcagatggcagccaaagatagggggacaacaaaggaagtagaagaagtcagtattactaatatcatcaacagctccatctccagctttaaacggaagatcagctttgccagcattgaaatttccagcgacaacgttgactacagtgacttgacaatgaaaaccagcgacaagttcaagtttgtcttccgagaaaagatgggcaggattgttgattatttcacaattcaaaaccccagtaatgttgatcactattccaaactactgtttcctttgatttttatgctagccaatgtattttactgggcatactacatgtatttttgagtcaatgttaaatttcttgcatgccataggtcttcaacaggacaagataatgatgtaaatggtattttaggccaagtgtgcacccacatccaatggtgctacaagtgactgaaataatatttgagtctttctgctcaaagaatgaagctccaaccattgttctaagctgtgtagaagtcctagcattataggatcttgtaatagaaacatcagtccattcctctttcatcttaatcaaggacattcccatggagcccaagattacaaatgtactcagggctgtttattcggtggctccctggtttgcatttacctcatataaagaatgggaaggagaccattgggtaaccctcaagtgtcagaagttgtttctaaagtaactatacatgttttttactaaatctctgcagtgcttataaaatacattgttgcctatttagggagtaacattttctagtttttgtttctggttaaaatgaaatatgggcttatgtcaattcattggaagtcaatgcactaactcaataccaagatgagtttttaaataatgaatattatttaataccacaacagaattatccccaatttccaataagtcctatcattgaaaattcaaatataagtgaagaaaaaattagtagatcaacaatctaaacaaatccctcggttctaagatacaatggattccccatactggaaggactctgaggctttattcccccactatgcatatcttatcattttattattatacacacatccatcctaaactatactaaagcccttttcccatgcatggatggaaatggaagatttttttgtaacttgttctagaagtcttaatatgggctgttgccatgaaggcttgcagaattgagtccattttctagctgcctttattcacatagtgatggggtactaaaagtactgggttgactcagagagtcgctgtcattctgtcattgctgctactctaacactgagcaacactctcccagtggcagatcccctgtatcattccaagaggagcattcatccctttgctctaatgatcaggaatgatgcttattagaaaacaaactgcttgacccaggaacaagtggcttagcttaagtaaacttggctttgctcagatccctgatccttccagctggtctgctctgagtggcttatcccgcatgagcaggagcgtgctggccctgagtactgaactttctgagtaacaatgagacacgttacagaacctatgttcaggttgcgggtgagctgccctctccaaatccagccagagatgcacattcctcggccagtctcagccaacagtaccaaaagtgatttttgagtgtgccagggtaaaggcttccagttcagcctcagttattttagacaatctcgccatctttaatttcttagcttcctgttctaataaatgcacggctttacctttcctgtcagaaataaaccaaggctctaaaagatgatttcccttctgtaactccctagagccacaggttctcattccttttcccattatacttctcacaattcagtttctatgagtttgatcacctgatttttttaacaaaatatttctaacgggaatgggtgggagtgctggtgaaaagagatgaaatgtggttgtatgagccaatcatatttgtgattttttaaaaaaagtttaaaaggaaatatctgttctgaaaccccacttaagcattgtttttatataaaaacaatgataaagatgtgaactgtgaaataaatataccatattagctacccaccaaaaaaaaaaaaaaaaaa

SEQ ID NO: 2 - protein, 440 aa, Homo sapiens gamma-aminobutyric acid (GABA) A receptor, pi (GABRP)
MNYSLHLAFVCLSLFTERMCIQGSQFNVEVGRSDKLSLPGFENLTAGYNKFLRPNFGGEPVQIALTLDIASISSISESNMDYTATIYLRQRWMDQRLVFEGNKSFTLDARLVEFLWVPDTYIVESKKSFLHEVTVGNRLIRLFSNGTVLYALRITTTVACNMDLSKYPMDTQTCKLQLESWGYDGNDVEFTWLRGNDSVRGLEHLRLAQYTIERYFTLVTRSQQETGNYTRLVLQFELRRNVLYFILETYVPSTFLVVLSWVSFWISLDSVPARTCIGVTTVLSMTTLMIGSRTSLPNTNCFIKAIDVYLGICFSFVFGALLEYAVAHYSSLQQMAAKDRGTTKEVEEVSITNIINSSISSFKRKISFASIEISSDNVDYSDLTMKTSDKFKFVFREKMGRIVDYFTIQNPSNVDHYSKLLFPLIFMLANVFYWAYYMYF

SEQ ID NO: 3 - DNA, 5540 bp, Homo sapiens tumor endothelial marker 8 precursor (TEM8)
  CDS (144)..(1835)
aattgcttccggggagttgcgagggagcgagggggaataaaggacccgcgaggaagggcccgcggatggcgcgtccctgagggtcgtggcgagttcgcggagcgtgggaaggagcggaccctgctctccccgggctgcgggccatggccacggcggagcggagagccctcggcatcggcttccagtggctctctttggccactctggtgctcatctgcgccgggcaagggggacgcagggaggatgggggtccagcctgctacggcggatttgacctgtacttcattttggacaaatcaggaagtgtgctgcaccactggaatgaaatctattactttgtggaacagttggctcacaaattcatcagcccacagttgagaatgtcctttattgttttctccacccgaggaacaaccttaatgaaactgacagaagacagagaacaaatccgtcaaggcctagaagaactccagaaagttctgccaggaggagacacttacatgcatgaaggatttgaaagggccagtgagcagatttattatgaaaacagacaagggtacaggacagccagcgtcatcattgctttgactgatggagaactccatgaagatctctttttctattcagagagggaggctaataggtctcgagatcttggtgcaattgtttactgtgttggtgtgaaagatttcaatgagacacagctggcccggattgcggacagtaaggatcatgtgtttcccgtgaatgacggctttcaggctctgcaaggcatcatccactcaattttgaagaagtcctgcatcgaaattctagcagctgaaccatccaccatatgtgcaggagagtcatttcaagttgtcgtgagaggaaacggcttccgacatgcccgcaacgtggacagggtcctctgcagcttcaagatcaatgactcggtcacactcaatgagaagcccttttctgtggaagatacttatttactgtgtccagcgcctatcttaaaagaagttggcatgaaagctgcactccaggtcagcatgaacgatggcctctcttttatctccagttctgtcatcatcaccaccacacactgttctgacggttccatcctggccatcgccctgctgatcctgttcctgctcctagccctggctctcctctggtggttctggcccctctgctgcactgtgattatcaaggaggtccctccaccccctgccgaggagagtgaggaagaagatgatgatggtctgcctaagaaaaagtggccaacggtagacgcctcttattatggtgggagaggcgttggaggcattaaaagaatggaggttcgttggggagaaaagggctccacagaagaaggtgctaagttggaaaaggcaaagaatgcaagagtcaagatgccggagcaggaatatgaattccctgagccgcgaaatctcaacaacaatatgcgtcggccttcttccccccggaagtggtactctccaatcaagggaaaactcgatgccttgtgggtcctactgaggaaaggatatgatcgtgtgtctgtgatgcgtccacagccaggagacacggggcgctgcatcaacttcaccagggtcaagaacaaccagccagccaagtacccactcaacaacgcctaccacacctcctcgccgcctcctgcccccatctacactcccccacctcctgcgccccactgccctcccccgccccccagcgcccctacccctcccatcccgtccccaccttccacccttccccctcctccccaggctccacctcccaacagggcacctcctccctcccgccctcctccaaggccttctgtctagagcccaaagttcctgctctgggctctctcagaaacttcaggagatgttagaacaagtctttccagttagagaagaggagtggtgataaagcccactgaccttcacacattctaaaaattggttggcaatgccagtataccaacaatcatgatcagctgaaagaaacagatattttaaattgccagaaaacaaatgatgaggcaactacagtcagatttatagccagccatctatcacctctagaaggttccagagacagtgaaactgcaagatgctctcaacaggattatgtctcatggagaccagtaagaaaatcatttatctgaaggtgaaatgcagagttggataagaaatacattgctgggtttctaaaatgctgccttcctgcctctactccacctccatccctggactttggacccttggcctaggagcctaaggaccttcacccctgtgcaccacccaagaaagaggaaaactttgcctacaactttggaaatgctggggtccctggtgtggtaagaaactcaacatcagacgggtatgcagaaggatgttcttctgggatttgcaggtacataaaaaatgtatggcatcttttccttgcaaattcttccagtttccaagtgagaaggggagcaggtgtttactgatggaaaaggtatgttgctatgttgatgtgtaagtgaaatcagttgtgtgcaatagacaggggcgtattcatgggagcatcagccagtttctaaaacccacaggccatcagcagctagaggtggctggctttggccagacatggaccctaaatcaacagacaatggcattgtcgaagagcaacctgttaatgaatcatgttaaaaatcaaggtttggcttcagtttaaatcacttgaggtatgaagtttatcctgttttccagagataaacataagttgatcttcccaaaataccatcattaggacctatcacacaatatcactagttttttttgtttgtttgttttttgttttttttcttggtaaagccatgcaccacagacttctgggcagagctgagagacaatggtcctgacataataaggatctttgattaacccccataaggcatgtgtgtgtatacaaatatacttctctttggcttttcgacatagaacctcagctgttaaccaaggggaaatacatcagatctgcaacacagaaatgctctgcctgaaatttccaccatgcctaggactcaccccatttatccaggtctttctggatctgtttaatcaataagccctataatcacttgctaaacactgggcttcatcacccagggataaaaacagagatcattgtcttggacctcctgcatcagcctattcaaaattatctctctctctagctttccacaaatcctaaaattcctgtcccaagccacccaaattctcagatcttttctggaacaaggcagaatataaaataaatatacatttagtggcttgggctatggtctccaaagatccttcaaaaatacatcaagccagcttcattcactcactttacttagaacagagatataagggcctgggatgcatttattttatcaataccaatttttgtggccatggcagacattgctaatcaatcacagcactatttcctattaagcccactgatttcttcacaatccttctcaaattacaattccaaagagccgccactcaacagtcagatgaacccaacagtcagatgagagaaatgaaccctacttgctatctctatcttagaaagcaaaaacaaacaggagtttccagggagaatgggaaagccagggggcataaaaggtacagtcaggggaaaatagatctaggcagagtgccttagtcagggaccacgggcgctgaatctgcagtgccaacaccaaactgacacatctccaggtgtacctccaaccctagccttctcccacagctgcctacaacagagtctcccagccttctcagagagctaaaaccagaaatttccagactcatgaaagcaaccccccagcctctccccaaccctgccgcattgtctaatttttagaacactaggcttcttctttcatgtagttcctcataagcaggggccagaatatctcagccacctgcagtgacattgctggacccctgaaaaccattccataggagaatgggttccccaggctcacagtgtagagacattgagcccatcacaactgttttgactgctggcagtctaaaacagtccacccaccccatggcactgccgcgtgattcccgcggccattcagaagttcaagccgagatgctgacgttgctgagcaacgagatggtgagcatcagtgcaaatgcaccattcagcacatcagtcatatgcccagtgcagttacaagatgttgtttcggcaaagcattttgatggaatagggaactgcaaatgtatgatgattttgaaaaggctcagcaggatttgttcttaaaccgactcagtgtgtcatccccggttatttagaattacagttaagaaggagaaacttctataagactgtatgaacaaggtgatatcttcatagtgggctattacaggcaggaaaatgttttaactggtttacaaaatccatcaatacttgtgtcattccctgtaaaaggcaggagacatgtgattatgatcaggaaactgcacaaaattattgttttcagcccccgtgttattgtccttttgaactgttttttttttattaaagccaaatttgtgttgtatatattcgtattccatgtgttagatggaagcatttcctatccagtgtgaataaaaagaacagttgtagtaaattattataaagccgatgatatttcatggcaggttattctaccaagctgtgcttgttggtttttcccatgactgtattgcttttataaatgtacaaatagttactgaaatgacgagacccttgtttgcacagcattaataagaaccttgataagaaccatattctgttgacagccagctcacagtttcttgcctgaagcttggtgcaccctccagtgagacacaagatctctcttttaccaaagttgagaacagagctggtggattaattaatagtcttcgatatctggccatgggtaacctcattgtaactatcatcagaatgggcagagatgatcttgaagtgtcacatacactaaagtccaaacactatgtcagatgggggtaaaatccattaaagaacaggaaaaaataattataagatgataagcaaatgtttcagcccaatgtcaacccagttaaaaaaaaaattaatgctgtgtaaaatggttgaattagtttgcaaactatataaagacatatgcagtaaaaagtctgttaatgcacatcctgtgggaatggagtgttctaaccaattgccttttcttgttatctgagctctcctatattatcatactcagataaccaaattaaaagaattagaatatgatttttaatacacttaacattaaactcttctaactttcttctttctgtgataattcagaagatagttatggatcttcaatgcctctgagtcattgttataaaaaatcagttatcactataccatgctataggagactgggcaaaacctgtacaatgacaaccctggaagttgctttttttaaaaaaataataaatttcttaaatcaaaaaaaaaaaaaaaaaaaa

SEQ ID NO: 4 - protein, 564 aa, Homo sapiens tumor endothelial marker 8 precursor (TEM8)
MATAERRALGIGFQWLSLATLVLICAGQGGRREDGGPACYGGFDLYFILDKSGSVLHHWNEIYYFVEQLAHKFISPQLRMSFIVFSTRGTTLMKLTEDREQIRQGLEELQKVLPGGDTYMHEGFERASEQIYYENRQGYRTASVIIALTDGELHEDLFFYSEREANRSRDLGAIVYCVGVKDFNETQLARIADSKDHVFPVNDGFQALQGIIHSILKKSCIEILAAEPSTICAGESFQVVVRGNGFRHARNVDRVLCSFKINDSVTLNEKPFSVEDTYLLCPAPILKEVGMKAALQVSMNDGLSFISSSVIITTTHCSDGSILAIALLILFLLLALALLWWFWPLCCTVIIKEVPPPPAEESEEEDDDGLPKKKWPTVDASYYGGRGVGGIKRMEVRWGEKGSTEEGAKLEKAKNARVKMPEQEYEFPEPRNLNNNMRRPSSPRKWYSPIKGKLDALWVLLRKGYDRVSVMRPQPGDTGRCINFTRVKNNQPAKYPLNNAYHTSSPPPAPIYTPPPPAPHCPPPPPSAPTPPIPSPPSTLPPPPQAPPPNRAPPPSRPPPRPSV

SEQ ID NO: 5 - DNA, 3654 bp, Homo sapiens cadherin 11, type 2, OB-cadherin (osteoblast) (CDH11)
  CDS (435)..(2822)
agatgccgcgggggccgctcgcagccgccgctgacttgtgaatgggaccgggactggggccgggactgacaccgcagcgcttgccctgcgccagggactggcggctcggaggttgcgtccaccctcaagggccccagaaatcactgtgttttcagctcagcggccctgtgacattccttcgtgttgtcatttgttgagtgaccaatcagatgggtggagtgtgttacagaaattggcagcaagtatccaatgggtgaagaagaagctaactggggacgtgggcagccctgacgtgatgagctcaaccagcagagacattccatcccaagagaggtctgcgtgacgcgtccgggaggccaccctcagcaagaccaccgtacagttggtggaaggggtgacagctgcattctcctgtgcctaccacgtaaccaaaaatgaaggagaactactgtttacaagccgccctggtgtgcctgggcatgctgtgccacagccatgcctttgccccagagcggcgggggcacctgcggccctccttccatgggcaccatgagaagggcaaggaggggcaggtgctacagcgctccaagcgtggctgggtctggaaccagttcttcgtgatagaggagtacaccgggcctgaccccgtgcttgtgggcaggcttcattcagatattgactctggtgatgggaacattaaatacattctctcaggggaaggagctggaaccatttttgtgattgatgacaaatcagggaacattcatgccaccaagacgttggatcgagaagagagagcccagtacacgttgatggctcaggcggtggacagggacaccaatcggccactggagccaccgtcggaattcattgtcaaggtccaggacattaatgacaaccctccggagttcctgcacgagacctatcatgccaacgtgcctgagaggtccaatgtgggaacgtcagtaatccaggtgacagcttcagatgcagatgaccccacttatggaaatagcgccaagttagtgtacagtatcctcgaaggacaaccctatttttcggtggaagcacagacaggtatcatcagaacagccctacccaacatggacagggaggccaaggaggagtaccacgtggtgatccaggccaaggacatgggtggacatatgggcggactctcagggacaaccaaagtgacgatcacactgaccgatgtcaatgacaacccaccaaagtttccgcagagcgtataccagatgtctgtgtcagaagcagccgtccctggggaggaagtaggaagagtgaaagctaaagatccagacattggagaaaatggcttagtcacatacaatattgttgatggagatggtatggaatcgtttgaaatcacaacggactatgaaacacaggagggggtgataaagctgaaaaagcctgtagattttgaaaccaaaagagcctatagcttgaaggtagaggcagccaacgtgcacatcgacccgaagtttatcagcaatggccctttcaaggacactgtgaccgtcaagatctcagtagaagatgctgatgagccccctatgttcttggccccaagttacatccacgaagtccaagaaaatgcagctgctggcaccgtggttgggagagtgcatgccaaagaccctgatgctgccaacagcccgataaggtattccatcgatcgtcacactgacctcgacagatttttcactattaatccagaggatggttttattaaaactacaaaacctctggatagagaggaaacagcctggctcaacatcactgtctttgcagcagaaatccacaatcggcatcaggaagccaaagtcccagtggccattagggtccttgatgtcaacgataatgctcccaagtttgctgccccttatgaaggtttcatctgtgagagtgatcagaccaagccactttccaaccagccaattgttacaattagtgcagatgacaaggatgacacggccaatggaccaagatttatcttcagcctaccccctgaaatcattcacaatccaaatttcacagtcagagacaaccgagataacacagcaggcgtgtacgcccggcgtggagggttcagtcggcagaagcaggacttgtaccttctgcccatagtgatcagcgatggcggcatcccgcccatgagtagcaccaacaccctcaccatcaaagtctgcgggtgcgacgtgaacggggcactgctctcctgcaacgcagaggcctacattctgaacgccggcctgagcacaggcgccctgatcgccatcctcgcctgcatcgtcattctcctggtcattgtagtattgtttgtgaccctgagaaggcaaaagaaagaaccactcattgtctttgaggaagaagatgtccgtgagaacatcattacttatgatgatgaagggggtggggaagaagacacagaagcctttgatattgccaccctccagaatcctgatggtatcaatggatttatcccccgcaaagacatcaaacctgagtatcagtacatgcctagacctgggctccggccagcgcccaacagcgtggatgtcgatgacttcatcaacacgagaatacaggaggcagacaatgaccccacggctcctccttatgactccattcaaatctacggttatgaaggcaggggctcagtggccgggtccctgagctccctagagtcggccaccacagattcagacttggactatgattatctacagaactggggacctcgttttaagaaactagcagatttgtatggttccaaagacacttttgatgacgattcttaacaataacgatacaaatttggccttaagaactgtgtctggcgttctcaagaatctagaagatgtgtaaacaggtatttttttaaatcaaggaaaggctcatttaaaacaggcaaagttttacagagaggatacatttaataaaactgcgaggacatcaaagtggtaaatactgtgaaataccttttctcacaaaaaggcaaatattgaagttgtttatcaacttcgctagaaaaaaaaaacacttggcatacaaaatatttaagtgaaggagaagtctaacgctgaactgacaatgaagggaaattgtttatgtgttatgaacatccaagtctttcttcttttttaagttgtcaaagaagcttccacaaaattagaaaggacaacagttctgagctgtaatttcgccttaaactctggacactctatatgtagtgcatttttaaacttgaaatatataatattcagccagcttaaacccatacaatgtatgtacaatacaatgtacaattatgtctcttgagcatcaatcttgttactgctgattcttgtaaatctttttgcttctactttcatcttaaactaatacgtgccagatataactgtcttgtttcagtgagagacgccctatttctatgtcatttttaatgtatctatttgtacaattttaaagttcttattttagtatacgtataaatatcagtattctgacatgtaagaaaatgttacggcatcacacttatattttatgaacattgtactgttgctttaatatgagcttcaatataagaagcaatctttgaaataaaaaaagatttttttttaaaaaaaa

SEQ ID NO: 6 - protein, 796 aa, Homo sapiens cadherin 11, type 2, OB-cadherin (osteoblast) (CDH11)
MKENYCLQAALVCLGMLCHSHAFAPERRGHLRPSFHGHHEKGKEGQVLQRSKRGWVWNQFFVIEEYTGPDPVLVGRLHSDIDSGDGNIKYILSGEGAGTIFVIDDKSGNIHATKTLDREERAQYTLMAQAVDRDTNRPLEPPSEFIVKVQDINDNPPEFLHETYHANVPERSNVGTSVIQVTASDADDPTYGNSAKLVYSILEGQPYFSVEAQTGIIRTALPNMDREAKEEYHVVIQAKDMGGHMGGLSGTTKVTITLTDVNDNPPKFPQSVYQMSVSEAAVPGEEVGRVKAKDPDIGENGLVTYNIVDGDGMESFEITTDYETQEGVIKLKKPVDFETKRAYSLKVEAANVHIDPKFISNGPFKDTVTVKISVEDADEPPMFLAPSYIHEVQENAAAGTVVGRVHAKDPDAANSPIRYSIDRHTDLDRFFTINPEDGFIKTTKPLDREETAWLNITVFAAEIHNRHQEAKVPVAIRVLDVNDNAPKFAAPYEGFICESDQTKPLSNQPIVTISADDKDDTANGPRFIFSLPPEIIHNPNFTVRDNRDNTAGVYARRGGFSRQKQDLYLLPIVISDGGIPPMSSTNTLTIKVCGCDVNGALLSCNAEAYILNAGLSTGALIAILACIVILLVIVVLFVTLRRQKKEPLIVFEEEDVRENIITYDDEGGGEEDTEAFDIATLQNPDGINGFIPRKDIKPEYQYMPRPGLRPAPNSVDVDDFINTRIQEADNDPTAPPYDSIQIYGYEGRGSVAGSLSSLESATTDSDLDYDYLQNWGPRFKKLADLYGSKDTFDDDS

SEQ ID NO: 7 - DNA, 2079 bp, Homo sapiens type II membrane serine protease (TMPRSS4)
  CDS (251)..(1519)
gagaggcagcagcttgttcagcggacaaggatgctgggcgtgagggaccaaggcctgccctgcactcgggcctcctccagccagtgctgaccagggacttctgacctgctggccagccaggacctgtgtggggaggccctcctgctgccttggggtgacaatctcagctccaggctacagggagaccgggaggatcacagagccagcatggtacaggatcctgacagtgatcaacctctgaacagcctcgatgtcaaacccctgcgcaaaccccgtatccccatggagaccttcagaaagtgtggggatccccatcatcatagcactactgagcctggcgagtatcatcattgtggttgtcctcatcaaggtgattctggataaatactacttcctctgcgggcagcctctccacttcatcccgaggaagcagctgtgtgacggagagctggactgtcccttgggggaggacgaggagcactgtgtcaagagcttccccgaagggcctgcagtggcagtccgcctctccaaggaccgatccacactgcaggtgctggactcggccacagggaactggttctctgcctgtttcgacaacttcacagaagctctcgctgagacagcctgtaggcagatgggctacagcagcaaacccactttcagagctgtggagattggcccagaccaggatctggatgttgttgaaatcacagaaaacagccaggagcttcgcatgcggaactcaagtgggccctgtctctcaggctccctggtctccctgcactgtcttgcctgtgggaagagcctgaagaccccccgtgtggtgggtggggaggaggcctctgtggattcttggccttggcaggtcagcatccagtacgacaaacagcacgtctgtggagggagcatcctggacccccactgggtcctcacggcagcccactgcttcaggaaacataccgatgtgttcaactggaaggtgcgggcaggctcagacaaactgggcagcttcccatccctggctgtggccaagatcatcatcattgaattcaaccccatgtaccccaaagacaatgacatcgccctcatgaagctgcagttcccactcactttctcaggcacagtcaggcccatctgtctgcccttctttgatgaggagctcactccagccaccccactctggatcattggatggggctttacgaagcagaatggagggaagatgtctgacatactgctgcaggcgtcagtccaggtcattgacagcacacggtgcaatgcagacgatgcgtaccagggggaagtcaccgagaagatgatgtgtgcaggcatcccggaagggggtgtggacacctgccagggtgacagtggtgggcccctgatgtaccaatctgaccagtggcatgtggtgggcatcgttagctggggctatggctgcgggggcccgagcaccccaggagtatacaccaaggtctcagcctatctcaactggatctacaatgtctggaaggctgagctgtaatgctgctgcccctttgcagtgctgggagccgcttccttcctgccctgcccacctggggatcccccaaagtcagacacagagcaagagtccccttgggtacacccctctgcccacagcctcagcatttcttggagcagcaaagggcctcaattcctgtaagagaccctcgcagcccagaggcgcccagaggaagtcagcagccctagctcggccacacttggtgctcccagcatcccagggagagacacagcccactgaacaaggtctcaggggtattgctaagccaagaaggaactttcccacactactgaatggaagcaggctgtcttgtaaaagcccagatcactgtgggctggagaggagaaggaaagggtctgcgccagccctgtccgtcttcacccatccccaagcctactagagcaagaaaccagttgtaatataaaatgcactgccctactgttggtatgactaccgttacctactgttgtcattgttattacagctatggccactattattaaagagctgtgtaacatcaaaaaaaaaaaaaaaaaaaa

SEQ ID NO: 8 - protein, 423 aa, Homo sapiens type II membrane serine protease (TMPRSS4)
MSNPCANPVSPWRPSESVGIPIIIALLSLASIIIVVVLIKVILDKYYFLCGQPLHFIPRKQLCDGELDCPLGEDEEHCVKSFPEGPAVAVRLSKDRSTLQVLDSATGNWFSACFDNFTEALAETACRQMGYSSKPTFRAVEIGPDQDLDVVEITENSQELRMRNSSGPCLSGSLVSLHCLACGKSLKTPRVVGGEEASVDSWPWQVSIQYDKQHVCGGSILDPHWVLTAAHCFRKHTDVFNWKVRAGSDKLGSFPSLAVAKIIIIEFNPMYPKDNDIALMKLQFPLTFSGTVRPICLPFFDEELTPATPLWIIGWGFTKQNGGKMSDILLQASVQVIDSTRCNADDAYQGEVTEKMMCAGIPEGGVDTCQGDSGGPLMYQSDQWHVVGIVSWGYGCGGPSTPGVYTKVSAYLNWIYNVWKAEL

SEQ ID NO: 9 - DNA, 2456 bp, Homo sapiens retinoic acid induced 3 (RAI3)
  CDS (254)..(1324)
ataacagcatgaagtgccgtggaactggaataggcgtgtcctctccctcgaccctccccctccttgtccctctgctcacccctcgctcgttccctccctccggcgagggccgcctttataacaactgctcagagtgcgagggcgggatagctgtccaaggtctcccccagcactgaggagctcgcctgctgccctcttgcgcgcgggaagcagcaccaagttcacggccaacgccttggcactagggtccagaatggctacaacagtccctgatggttgccgcaatggcctgaaatccaagtactacagactttgtgataaggctgaagcttggggcatcgtcctagaaacggtggccacagccggggttgtgacctcggtggccttcatgctcactctcccgatcctcgtctgcaaggtgcaggactccaacaggcgaaaaatgctgcctactcagtttctcttcctcctgggtgtgttgggcatctttggcctcaccttcgccttcatcatcggactggacgggagcacagggcccacacgcttcttcctctttgggatcctcttttccatctgcttctcctgcctgctggctcatgctgtcagtctgaccaagctcgtccgggggaggaagcccctttccctgttggtgattctgggtctggccgtgggcttcagcctagtccaggatgttatcgctattgaatatattgtcctgaccatgaataggaccaacgtcaatgtcttttctgagctttccgctcctcgtcgcaatgaagactttgtcctcctgctcacctacgtcctcttcttgatggcgctgaccttcctcatgtcctccttcaccttctgtggttccttcacgggctggaagagacatggggcccacatctacctcacgatgctcctctccattgccatctgggtggcctggatcaccctgctcatgcttcctgactttgaccgcaggtgggatgacaccatcctcagctccgccttggctgccaatggctgggtgttcctgttggcttatgttagtcccgagttttggctgctcacaaagcaacgaaaccccatggattatcctgttgaggatgctttctgtaaacctcaactcgtgaagaagagctatggtgtggagaacagagcctactctcaagaggaaatcactcaaggttttgaagagacaggggacacgctctatgccccctattccacacattttcagctgcagaaccagcctccccaaaaggaattctccatcccacgggcccacgcttggccgagcccttacaaagactatgaagtaaagaaagagggcagctaactctgtcctgaagagtgggacaaatgcagccgggcggcagatctagcgggagctcaaagggatgtgggcgaaatcttgagtcttctgagaaaactgtacaagacactacgggaacagtttgcctccctcccagcctcaaccacaattcttccatgctggggctgatgtgggctagtaagactccagttcttagaggcgctgtagtatttttttttttttgtctcatcctttggatacttcttttaagtgggagtctcaggcaactcaagtttagacccttactctttttgtttgttttttgaaacaggatcttgctctgtcacccaggcttgagtgcagtggtgcgatcacagcccagtgcagcctcgaccacctgtgctcaagcaatcctcccatctccatctcccaaagtgctgggatgacaggcgtgagccacagctcccagcctaggcccttaatcttgctgttattttccatggactaaaggtctggtcatctgagctcacgctggctcacacagctctaggggcctgctcctctaactcacagtgggttttgtgaggctctgtggcccagagcagacctgcatatctgagcaaaaatagcaaaagcctctctcagcccactggcctgaatctacactggaagccaacttgctggcacccccgctccccaacccttcttgcctgggtaggagaggctaaagatcaccctaaatttactcatctctctagtgctgcctcacattgggcctcagcagctccccagcaccaattcacaggtcacccctctcttcttgcactgtccccaaacttgctgtcaattccgagatctaatctccccctacgctctgccaggaattctttcagacctcactagcacaagcccggttgctccttgtcaggagaatttgtagatcattctcacttcaaattcctggggctgatacttctctcatcttgcaccccaacctctgtaaatagatttaccgcatttacggctgcattctgtaagtgggcatggtctcctaatggaggagtgttcattgtataataagttattcacctgagtatgcaataaagatgtggtggccactctttcatggtggtggcagcaaaaaaaaaaaaaa

SEQ ID NO: 10 - protein, 357 aa, Homo sapiens retinoic acid induced 3 (RAI3)
MATTVPDGCRNGLKSKYYRLCDKAEAWGIVLETVATAGVVTSVAFMLTLPILVCKVQDSNRRKMLPTQFLFLLGVLGIFGLTFAFIIGLDGSTGPTRFFLFGILFSICFSCLLAHAVSLTKLVRGRKPLSLLVILGLAVGFSLVQDVIAIEYIVLTMNRTNVNVFSELSAPRRNEDFVLLLTYVLFLMALTFLMSSFTFCGSFTGWKRHGAHIYLTMLLSIAIWVAWITLLMLPDFDRRWDDTILSSALAANGWVFLLAYVSPEFWLLTKQRNPMDYPVEDAFCKPQLVKKSYGVENRAYSQEEITQGFEETGDTLYAPYSTHFQLQNQPPQKEFSIPRAHAWPSPYKDYEVKKEGS

SEQ ID NO: 11 - DNA, 1609 bp, Homo sapiens homeo box B2 (HOXB2)
  CDS (121)..(1188)
atctccccctcccaaaatcgctccattacataaatcggggggggtgcaggagggggggtcccttccgatcctccctcctgacgccccccccagcagccccctcccccaccattgaaagccatgaattttgaatttgagagggagattgggtttataaacagccagccgtcgctcgccgagtgtctgacttccttccccgctgtcttggagacatttcaaacttcatcaatcaaggagtcgacattaattcctcctcctcctcctttcgagcaaaccttccccagcctccagcccggcgcctccacccttcagagacccaggagccaaaagcgagccgaagatgggcctgctctgccgccgccaccgccgccgccactccccgctgcccccccggcccccgagttcccttggatgaaagagaagaaatccgccaagaaacccagccaatccgccacgtctccttctccggccgcctccgccgttccggcctccggggtcggatcgcctgcagatggcctgggactgccggaggctggtggcggcggggcgcgcaggctgcgcacggcttacaccaacacgcagctgctggaactggagaaggaattccactttaataagtacctgtgccggccacgccgcgtcgagatcgcggccttgctggacctcaccgaaaggcaggtcaaagtctggtttcagaaccggcgcatgaagcacaagcggcagacgcagcaccgagagccgccggatggggagcctgcctgcccgggagccctggaggacatctgcgaccctgccgaggaacccgcggccagcccgggcggcccctccgcctcgcgggcggcgtgggaagcctgctgtcacccgccggaggtggtgccgggggccttaagcgcggacccccggcctttagccgttcgcttagagggcgcaggcgcgtcgagtcccggctgcgcgctgcgcggggccggcgggctggagcccgggccattgccagaagacgtcttctcggggcgccaggattcacctttccttcccgacctcaacttcttcgcggccgactcctgtctccagctatccggaggcctctcccctagcctacagggttctctcgacagcccggtccctttttccgaggaagagctggattttttcaccagtacgctctgtgccatcgacctgcagtttccctaacctgtttcctcctcccggtcctttcgacccccgcgctccttggccgtctactggaaaaatcgagcctctcccaccctcagtcgcatagacttatgtgttttgctaaaattcaggtattactgaattagcgtttaatccacttcctttcttcttcttctaaaatattgggcactcggttatcttttaaaattcacacagaaaaattccgtttggtagactccttccaatgaaatctcaggaataattaaactctagggggactttcttaaaaataactagagggacctattttcctcttttttatgttttagactgtagattatttattaaaattctttaataataggaaaaggggaaagtatttattgtacattattttcatagattaaataaatgtctttataataccaaaaaaaaa

SEQ ID NO: 12 - protein, 356 aa, Homo sapiens homeo box B2 (HOXB2)
MNFEFEREIGFINSQPSLAECLTSFPAVLETFQTSSIKESTLIPPPPPFEQTFPSLQPGASTLQRPRSQKRAEDGPALPPPPPPPLPAAPPAPEFPWMKEKKSAKKPSQSATSPSPAASAVPASGVGSPADGLGLPEAGGGGARRLRTAYTNTQLLELEKEFHFNKYLCRPRRVEIAALLDLTERQVKVWFQNRRMKHKRQTQHREPPDGEPACPGALEDICDPAEEPAASPGGPSASRAAWEACCHPPEVVPGALSADPRPLAVRLEGAGASSPGCALRGAGGLEPGPLPEDVFSGRQDSPFLPDLNFFAADSCLQLSGGLSPSLQGSLDSPVPFSEEELDFFTSTLCAIDLQFP

SEQ ID NO: 13 - DNA, 439 bp, Homo sapiens S100 calcium binding protein P (S100P)
  CDS (22)..(309)
ggtgggtctgaatctagcaccatgacggaactagagacagccatgggcatgatcatagacgtcttttcccgatattcgggcagcgagggcagcacgcagaccctgaccaagggggagctcaaggtgctgatggagaaggagctaccaggcttcctgcagagtggaaaagacaaggatgccgtggataaattgctcaaggacctggacgccaatggagatgcccaggtggacttcagtgagttcatcgtgttcgtggctgcaatcacgtctgcctgtcacaagtactttgagaaggcaggactcaaatgatgccctggagatgtcacagattcctgcagagccatggtcccaggcttcccaaaagtgtttgttggcaattattcccctaggctgagcctgctcatgtacctctgattaataaatgcttatgaaaaaaaaa

SEQ ID NO: 14 - protein, 95 aa, Homo sapiens S100 calcium binding protein P (S100P)
MTELETAMGMIIDVFSRYSGSEGSTQTLTKGELKVLMEKELPGFLQSGKDKDAVDKLLKDLDANGDAQVDFSEFIVFVAAITSACHKYFEKAGLK

SEQ ID NO: 15 - DNA, 428 bp, Homo sapiens S100 calcium binding protein A2 (S100A2)
  CDS (41)..(334)
ctgggtctgtctctgccacctggtctgccacagatccatgatgtgcagttctctggagcaggcgctggctgtgctggtcactaccttccacaagtactcctgccaagagggcgacaagttcaagctgagtaagggggaaatgaaggaacttctgcacaaggagctgcccagctttgtgggggagaaagtggatgaggaggggctgaagaagctgatgggcagcctggatgagaacagtgaccagcaggtggacttccaggagtatgctgttttcctggcactcatcactgtcatgtgcaatgacttcttccagggctgcccagaccgaccctgaagcagaactcttgacttcctgccatggatctcttgggcccaggactgttgatgcctttgagttttgtattcaataaactttttttgtctgttga

SEQ ID NO: 16 - protein, 97 aa, Homo sapiens S100 calcium binding protein A2 (S100A2)
MCSSLEQALAVLVTTFHKYSCQEGDKFKLSKGEMKELLHKELPSFVGEKVDEEGLKKLMGSLDENSDQQVDFQEYAVFLALITVMCNDFFQGCPDRP

SEQ ID NO: 17 - DNA, 470 bp, Homo sapiens S100 calcium binding protein A6 (calcyclin) (S100A6)
  CDS (103)..(375)
gggaccgctataaggccagtcggactgcgacatagcccatcccctcgaccgctcgcgtcgcatttggccgcctccctaccgctccaagcccagccctcagccatggcatgccccctggatcaggccattggcctcctcgtggccatcttccacaagtactccggcagggagggtgacaagcacaccctgagcaagaaggagctgaaggagctgatccagaaggagctcaccattggctcgaagctgcaggatgctgaaattgcaaggctgatggaagacttggaccggaacaaggaccaggaggtgaacttccaggagtatgtcaccttcctgggggccttggctttgatctacaatgaagccctcaagggctgaaaataaatagggaagatggagacacctctgggggtcctctctgagtcaaatccagtggtgggtaattgtacaataaattttttttggtcaaattt

SEQ ID NO: 18 - protein, 90 aa, Homo sapiens S100 calcium binding protein A6 (calcyclin) (S100A6)
MACPLDQAIGLLVAIFHKYSGREGDKHTLSKKELKELIQKELTIGSKLQDAEIARLMEDLDRNKDQEVNFQEYVTFLGALALIYNEALKG

SEQ ID NO: 19 - DNA, 595 bp, Homo sapiens S100 calcium binding protein A11 (S100A11)
  CDS (121)..(438)
gggcaaggctgggccgggaagggcgtgggttgaggagaggctccagacccgcacgccgcgcgcacagagctctcagcgccgctcccagccacagcctcccgcgcctcgctcagctccaacatggcaaaaatctccagccctacagagactgagcggtgcatcgagtccctgattgctgtcttccagaagtatgctggaaaggatggttataactacactctctccaagacagagttcctaagcttcatgaatacagaactagctgccttcacaaagaaccagaaggaccctggtgtccttgaccgcatgatgaagaaactggacaccaacagtgatggtcagctagatttctcagaatttcttaatctgattggtggcctagctatggcttgccatgactccttcctcaaggctgtcccttcccagaagcggacctgaggaccccttggccctggccttcaaacccaccccctttccttccagcctttctgtcatcatctccacagcccacccatcccctgagcacactaaccacctcatgcaggccccacctgccaatagtaataaagcaatgtcacttttttaaaacatgaaa

SEQ ID NO: 20 - protein, 105 aa, Homo sapiens S100 calcium binding protein A11 (S100A11)
MAKISSPTETERCIESLIAVFQKYAGKDGYNYTLSKTEFLSFMNTELAAFTKNQKDPGVLDRMMKKLDTNSDGQLDFSEFLNLIGGLAMACHDSFLKAVPSQKRT

SEQ ID NO: 21 - DNA, 649 bp, Homo sapiens S100 calcium binding protein A10 (S100A10)
  CDS (112)..(405)
agaatacactcacaagccactccgctgctcgcctctccgccccgcgtccagctcgcccagctcgcccagcgtccgccgcgcctcgccaaggcttcaacggaccacaccaaaatgccatctcaaatggaacacgccatggaaaccatgatgtttacatttcacaaattcgctggggataaaggctacttaacaaaggaggacctgagagtactcatggaaaaggagttccctggatttttggaaaatcaaaaagaccctctggctgtggacaaaataatgaaggacctggaccagtgtagagatggcaaagtgggcttccagagcttcttttccctaattgcgggcctcaccattgcatgcaatgactattttgtagtacacatgaagcagaagggaaagaagtaggcagaaatgagcagttcgctcctccctgataagagttgtccaaagggtcgcttaaggaatctgccccacagcttcccccatagaaggatttcatgagcagatcaggacacttagcaaatgtaaaaataaaatctaactctcatttgacaagcagagaaagaaaagttaaataccagataagcttttgatttttgtattgtttgcatccccttgccctcaataaataaagttcttttttagttcc

SEQ ID NO: 22 - protein, 97 aa, Homo sapiens S100 calcium binding protein A10 (S100A10)
MPSQMEHAMETMMFTFHKFAGDKGYLTKEDLRVLMEKEFPGFLENQKDPLAVDKIMKDLDQCRDGKVGFQSFFSLIAGLTIACNDYFVVHMKQKGKK

SEQ ID NO: 23 - DNA, 512 bp, Homo sapiens S100 calcium binding protein A4 (S100A4)
  CDS (70)..(375)
attcttcccctctctacaaccctctctcctcagcgcttcttctttcttggtttgatcctgactgctgtcatggcgtgccctctggagaaggccctggatgtgatggtgtccaccttccacaagtactcgggcaaagagggtgacaagttcaagctcaacaagtcagaactaaaggagctgctgacccgggagctgcccagcttcttggggaaaaggacagatgaagctgctttccagaagctgatgagcaacttggacagcaacagggacaacgaggtggacttccaagagtactgtgtcttcctgtcctgcatcgccatgatgtgtaacgaattctttgaaggcttcccagataagcagcccaggaagaaatgaaaactcctctgatgtggttggggggtctgccagctggggccctccctgtcgccagtgggcacttttttttttccaccctggctccttcagacacgtgcttgatgctgagcaagttcaataaagattcttggaagttt

SEQ ID NO: 24 - protein, 101 aa, Homo sapiens S100 calcium binding protein A4 (S100A4)
MACPLEKALDVMVSTFHKYSGKEGDKFKLNKSELKELLTRELPSFLGKRTDEAAFQKLMSNLDSNRDNEVDFQEYCVFLSCIAMMCNEFFEGFPDKQPRKK

SEQ ID NO: 25 - DNA, 309 bp, Homo sapiens S100 calcium binding protein A14 (calgizzarin) (S100A14)
  CDS (1)..(309)
atggcaaaaatctccagccctacagagactgagcggtgcattgagtccctgattgctgttttccagaagtatgctggaaaggatggttacaaccgcaatctctccaagacggagttcctaagcttcatgaatacagagctggctgcctttacaaagaaccagaaggaccccggtgtccttgaccacatgaagaaactggatgtcagcagtgatgggcagttagatttcccaaaatttcttaatctgattggtggcctagctgtggcttgccatgactccttcctcaaggctgtcccctcccagaagtag

SEQ ID NO: 26 - protein, 102 aa, Homo sapiens S100 calcium binding protein A14 (calgizzarin) (S100A14)
MAKISSPTETERCIESLIAVFQKYAGKDGYNRNLSKTEFLSFMNTELAAFTKNQKDPGVLDHMKKLDVSSDGQLDFPKFLNLIGGLAVACHDSFLKAVPSQK

(19) United States
(12) Patent Application Publication
(10) Pub. No.: US 2009/0233286 A1
(43) Pub. Date: Sep. 17, 2009

(54) Methods of diagnosis and prognosis of pancreatic cancer

(75) Inventors: Davendra Segara, New South Wales (AU); Andrew Biankin, New South Wales (AU); Robert Sutherland, New South Wales (AU); Susan Henshall, New South Wales (AU)

Correspondence Address: COOPER & DUNHAM, LLP, 30 Rockefeller Plaza, 20th Floor, NEW YORK, NY, 10112, US

(73) Assignee: Garvan Institute of Medical Research

(21) Appl. No.: 12/288,676

(22) Filed: Oct. 22, 2008

Related U.S. Application Data
(62) Division of application No. 10/548,460, filed on Apr. 14, 2006, now abandoned.

(30) Foreign Application Priority Data
Feb. 18, 2003 (AU) 2003900747

(51) Int. Cl. C12Q 1/68

(52) U.S. Cl. 435/6

(57) ABSTRACT

Disclosed herein are methods of diagnosing pancreatic cancer in a subject. Also provided are methods of monitoring the efficacy of a therapeutic treatment of pancreatic cancer as well as methods of determining the likelihood that a subject having pancreatic cancer will survive, and methods of determining the suitability of a subject having pancreatic cancer for surgical resection therapy.

This application is a divisional application of U.S. Ser. No. 10/548,460, which is a §371 National Stage of PCT International Application No. PCT/AU2004/000194, filed Feb. 18, 2004, claiming priority of Australian Provisional Application No. 2003900747, filed Feb. 18, 2003, the contents of all of which are hereby incorporated by reference.

### FIELD OF THE INVENTION

The present invention relates to the identification of nucleic acid and protein expression profiles and nucleic acids, products, and antibodies thereto that are involved in pancreatic cancer; and to the use of such expression profiles and compositions in the diagnosis, prognosis and therapy of pancreatic cancer. More particularly, this invention relates to novel genes that are expressed at elevated or reduced levels in malignant tissues and uses therefor in the diagnosis of cancer or malignant tumors in human subjects. This invention also relates to the use of nucleic acid or antibody probes to specifically detect pancreatic cancer cells, wherein over-expression or reduced expression of nucleic acids hybridizing to the probes is highly associated with the occurrence and/or recurrence of an pancreatic tumor, and/or the likelihood of patient survival. The diagnostic and prognostic test of the present invention is particularly useful for the early detection of pancreatic cancer or metastases thereof, or other cancers, and for monitoring the progress of disease, such as, for example, during remission or following surgery or chemotherapy. The present invention is also directed to methods of therapy wherein the activity of a protein encoded by a diagnostic/prognostic gene described herein is modulated.

### BACKGROUND OF THE INVENTION

1. General

As used herein the term “derived from” shall be taken to indicate that a specified integer are obtained from a particular source albeit not necessarily directly from that source.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The embodiments of the invention described herein with respect to any single embodiment shall be taken to apply mutatis mutandis to any other embodiment of the invention described herein.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be limited in scope by the specific examples described herein. Functionally equivalent products, compositions and methods are clearly within the scope of the invention, as described herein.

The present invention is performed without undue experimentation using, unless otherwise indicated, conventional techniques of molecular biology, microbiology, virology, recombining DNA technology, peptide synthesis in solution, solid phase peptide synthesis, and immunology. Such procedures are described, for example, in the following texts that are incorporated herein by reference:
1. Sambrook, Fritsch & Maniatis, Molecular Cloning: A Laboratory Manual, Cold Spring Harbor Laboratories, New York, Second Edition (1989), whole of Vols I, II, and III;
2. DNA Cloning: A Practical Approach, Vols. I and II (D. N. Glover, ed., 1985), IRL Press, Oxford, whole of text;
3. Oligonucleotide Synthesis: A Practical Approach (M. J. Gait, ed., 1984) IRL Press, Oxford, whole of text, and particularly the papers therein by Gait, pp 1-22; Atkinson et al., pp 35-81; Sproat et al., pp 83-115; and Wu et al., pp 135-151;
4. Nucleic Acid Hybridization: A Practical Approach (B. D. Hames & S. J. Higgins, eds., 1985) IRL Press, Oxford, whole of text;
5. Perbal, B., A Practical Guide to Molecular Cloning (1984);
6. Wünsch, E., ed. (1974) Synthese von Peptiden in Houben-Weyls Metoden der Organischen Chemie (Müler, E., ed.), vol. 15, 4th edn., Parts 1 and 2, Thieme, Stuttgart.
7. Handbook of Experimental Immunology, Vols. I-IV (D. M. Weir and C. C. Blackwell, eds., 1986, Blackwell Scientific Publications).

This specification contains nucleotide and amino acid sequence information prepared using Patentin Version 3.1, presented herein after the claims. Each nucleotide sequence is identified in the sequence listing by the numeric indicator <210> followed by the sequence identifier (e.g. <210>1, <210>2, <210>3, etc). The length and type of sequence (DNA, protein (PRT), etc), and source organism for each nucleotide sequence, are indicated by information provided in the numeric indicator fields <211>, <212> and <213>, respectively. Nucleotide sequences referred to in the specification are defined by the term “SEQ ID NO:”, followed by the sequence identifier (eg. SEQ ID NO: 1 refers to the sequence in the sequence listing designated as <400>1).

The designation of nucleotide residues referred to herein are those recommended by the IUPAC-IUB Biochemical Nomenclature Commission, wherein A represents Adenine, C represents Cytosine, G represents Guanine, T represents thymine, Y represents a pyrimidine residue, R represents a purine residue, M represents Adenine or Cytosine, K represents Guanine or Thymine, S represents Guanine or Cytosine, W represents Adenine or Thymine, H represents a nucleotide other than Guanine, B represents a nucleotide other than Adenine, V represents a nucleotide other than Thymine, D represents a nucleotide other than Cytosine and N represents any nucleotide residue.

2. Description of the Related Art

Cancer is a multi-factorial disease and major cause of morbidity in humans and other animals, and deaths resulting from cancer in humans are increasing and expected to surpass deaths from heart disease in future. Carcinomas of the lung, prostate, breast, colon and pancreas are major contributing factors to total cancer death in humans. For example, prostate cancer is the fourth most prevalent cancer and the second leading cause of cancer death in males. With few exceptions, metastatic disease from carcinoma is fatal. Even if patients survive their primary cancers, recurrence or metastases are common.

It is widely recognized that simple and rapid tests for solid cancers or tumors have considerable clinical potential. Not only can such tests be used for the early diagnosis of cancer but they also allow the detection of tumor recurrence following surgery and chemotherapy. A number of cancer-specific blood tests have been developed which depend upon the detection of tumor-specific antigens in the circulation (Catalona, W. J., et al., 1991, “Measurement of prostate-specific antigen in serum as a screening test for prostate cancer”, N. Engl. J. Med. 324, 1156-1161; Barrenetxea, G., et al., 1998, “Use of serum tumor markers for the diagnosis and follow-up of breast cancer”, Oncology, 55, 447-449; Cairns, P., and Sidreansky, D., 1999, “Molecular methods for the diagnosis of cancer”. Biochim. Biophys. Acta. 1423, C 11-C 18).

The incidence of pancreatic cancer parallels closely its mortality, with the vast majority of subjects with this fatal disease dying within a year of their diagnosis (World Cancer Report, ed. P. Kleihues and B. W. Stewart. 2003, Geneva: World Health Organisation & International Agency for Research on Cancer). Pancreatic cancer presents commonly as a clinically advanced disease with few treatment options. At the time of diagnosis 85% of tumours have extended beyond the pancreas (Warshaw and Fernandez-del Castillo, N Engl J Med, 326(7), 455-465, 1992).

Earlier detection of pancreatic cancer may improve prognosis, yet at present there are no adequate means of detecting tumours at an early stage. There is a need to understand the molecular pathology of pancreatic cancer to facilitate the development of a greater understanding of tumour development, the identification of prognostic indicators of outcome and targets for novel treatment and prevention strategies (Urrutia and DiMagno, Gastroenterol., 110(1), 306-310, 1996).

In 2000, 572 cases of pancreatic cancer were reported in NSW, Australia, giving an incidence rate of approximately 9 cases for every 100,000 of general population. Whilst pancreatic cancer is the eleventh-most frequently diagnosed cancer in Australia, it is the fifth-highest cause of cancer-related death by organ site behind lung, colorectal, breast and prostate cancers (Cancer in Australia 1998: Incidence and mortality data for 1998, Australian Institute for Health and Welfare (AIHW)).

The incidence of pancreatic cancer in other developed countries is similar to that in Australia. Overall it is slightly higher in men as compared to women (Ferlay et al., GLOBOCAN 2000: Cancer Incidence, Mortality and Prevalence Worldwide, 2001, IARC Press: Lyon). The incidence in developing countries is much lower. The World Cancer Report recently published by the World Health Organisation, identified pancreatic cancer as the 14^{th} most common cause of cancer worldwide, with approximately 216,000 new cases diagnosed each year (World Cancer Report, ed. P. Kleihues and B. W. Stewart. 2003, Geneva: World Health Organisation & International Agency for Research on Cancer).

Whilst mortality rates of most human cancers have shown significant improvement over the last 30 years, pancreatic cancer remains an exception to this trend. The mortality rate for pancreatic cancer closely parallels its incidence and is among the worst of all human cancers, with less than 5% of subjects surviving the illness to 5 years in the United States (Sohn et al, J Gastro Surg., 4(6), 567-579, 2000; Geer and Brennan, Am J Surg, 165(1), 68-73, 1993).

### Risk Factors

It is estimated that 3 to 10% of pancreatic cancers are likely to be caused by inherited factors (Hruban et al, Pancreatic Cancer, in The Genetic Basis of Human Cancer, B. Vogelstein and K. W. Kinzler, Editors. 1996, McGraw-Hill: London. pp. 603-613). One retrospective study showed that the relative risk of developing pancreatic caner is 5.3 times the risk of the general population if a close relative has the disease and 1.9 times the risk for those who have a relative with any type of cancer (Falk et al. Am J Epidemiol 128(2), 324-336, 1988). Subjects with three or more affected family members have a 57-fold increase in risk (Tersmette et al., Clin Cancer Res 7(3), 738-744, 2001). No genetic marker for high risk families has yet been identified. Several familial cancer syndromes are also associated with pancreatic cancer (Table 1), but these only account for approximately 20% of families with a predisposition to pancreatic cancer and less than 2% of overall incidence of the disease (Jaffee et al, Cancer Cell 2(1), 25-28, 2002). Mutations of tumour suppressor genes such as BRCA1 and BRCA2 or DNA mismatch repair genes such as MSH1 and MLH1 are more likely to be passed in the germ-line than to be acquired in the pancreatic cancer cell (Hruban et al., Ann Oncol. 10(Suppl 4), 69-73, 1999).

TABLE 1
Genetic disorders and germ-line genetic alterations
associated with familial pancreatic cancer.

| | | Increased risk of |
|---|---|---|
| Disorder | Gene (location) | pancreatic cancer |
| Hereditary pancreatitis | PRSS1 (7q35) | ×50 |
| Hereditary nonpolyposis | MSH2, MLH1 | unspecified |
| colorectal cancer | | |
| lynch variant II | | |
| Hereditary breast and | BRCA2 (13q12-q13) | ×3.5-20 |
| ovarian cancer | | |
| Familial atypical multiple | p16INK4a (9q21) | ×12-20 |
| mole melanoma | | |
| syndrome (FAMMM) | | |
| Peutz-Jeghers syndrome | STK11/LKB1 (19q13) | ×130 |

Source: Jaffee et al., Cancer Cell. 2, 25-28 (2002).

Smoking, age, chronic pancreatitis and diabetes are the most significant risk factors for pancreatic cancer identified to date, which raises the question of whether pancreatic cancer might be considered a preventable disease (Gapstur and Gann, JAMA 286(8), 967-968, 2001). The relative risk of smokers developing pancreatic cancer is 1.5 times that of non-smokers and the risk is related to the amount smoked (Gold and Goldin, Surg. Oncol. Clin. Nth Am. 7(1), 67-91, 1998), with males who smoke over 40 cigarettes a day increasing their risk tenfold (Fuchs et al., Arch Intern Med. 156(19), 2255-2260, 1996). Approximately 25% of pancreatic cancer cases are attributable to smoking, with increasing evidence of a relationship between smoking and activating mutations of the K-ras oncogene (Hruban et al., Am J. Pathol. 143(2), 545-554, 1993). There is strong evidence based on molecular and epidemiological studies that smoking is a primary risk factor in pancreatic cancer.

The risk of pancreatic cancer increases significantly with advancing age, peaking between 60 and 80 years. Pancreatic cancer rarely occurs in subjects younger than 40 years (World Cancer Report, ed. P. Kleihues and B. W. Stewart. 2003, Geneva: World Health Organisation & International Agency for Research on Cancer). The role of chronic pancreatitis in the development of pancreatic cancer is less clear. Case control studies have demonstrated a positive association between chronic pancreatitis and pancreatic cancer but others have found no significant association (Karlson et al., Gastroenterol 113(2), 587-592, 1997). One retrospective study found that chronic pancreatitis identified within 10 years of the diagnosis of pancreatic cancer conferred a 5.7-15 times increased risk of pancreatic cancer (Lowenfels et al., N Engl J. Med. 328(20), 1433-1437, 1993), suggesting a possible common aetiological factor for these conditions rather than causal relationship. Furthermore, pancreatic cancer is often complicated by duct obstruction by the tumour mass with subsequent development of the histological changes of chronic pancreatitis within the pancreas.

The higher incidence of pancreatic cancer in developed countries suggests that the Western-style diet, high in animal fat and protein may confer risk. Meta-analysis identified obesity as being weakly associated with increased risk (de Gonzalez et al., Br J Cancer 89(3), 519-523, 2003), while methods of food preparation have been implicated (Ghadirian et al., Cancer Epidemiol Biomarkers Prev, 4(8), 895-899, 1995). The evidence for coffee, tea, cereals and other specific food groups is not conclusive (Ahlgren, Sem. Oncol. 23(2), 241-250, 1996), and moderate alcohol consumption appears not to be a significant risk factor (Silverman et al., Cancer Res. 55(21), 4899-4905, 1995). Diabetes mellitus is associated with pancreatic cancer and is often diagnosed at the time of pancreatic cancer diagnosis (Gapstur et al. JAMA 283(19), 2552-2558, 2000). Diabetes is also a predisposing factor to pancreatic cancer according to meta-analysis (Everhart and Wright, JAMA 273(20), 1605-1609, 1995). In summary, whilst various risk factors have been identified as conferring some predisposition to the development of the disease, there remains much to be understood of the aetiology of pancreatic cancer.

### Pathology

Tumours of the pancreas display a spectrum of pathologies from the benign to the malignant. By far the most common of pancreatic exocrine tumours is ductal adenocarcinoma which accounts for 85% of cases and the majority of these (60%) arise in the head of the pancreas (Kloppel et al., Histological Typing of Tumours of the Exocrine Pancreas. 1996, Geneva: World Health Organisation). The cell of origin in pancreatic cancer is controversial. The morphology of pancreatic cancer cells hold similarities with pancreatic ductal cells, suggesting that it is the cell of origin. Emerging evidence of developmental plasticity in the exocrine pancreas, however, suggests that the cell of origin may be a pluripotent stem cell termed the centrilobular cell whose differentiation is mediated by the Notch signalling pathway (Miyamoto et al., Cancer Cell, 2003. 3(6), 565-576, 2003). The centrilobular cells are found at the junction of the acinus and the duct and are thought to proliferate to become the precursor lesions.

Histological grading of pancreatic adenocarcinoma into three categories considers glandular differentiation, mucin production, number of mitoses and amount of nuclear atypia (Kloppel et al., Histological Typing of Tumours of the Exocrine Pancreas. 1996, Geneva: World Health Organisation). The majority of pancreatic cancers are moderately differentiated and histological grade has been reported to independently predict prognosis (Luftges et al., J. Pathol. 191(2), 154-161, 2000).

Identification of morphological changes in precursor lesions have contributed to the development of a progression model in pancreatic cancer (Hruban et al., Clin Cancer Res. 6(8), 2969-2972, 2000), which follows the hyperplasia, in-situ, invasive carcinoma multistep sequence of human cancer (Vogelstein and Kinzler, Trends in Genetics 9(4), 138-141, 1993). The change from normal ductal cuboidal epithelium to the low columnar epithelium of cancer is evidenced by progression through a series of intermediate lesions termed ‘pancreatic intraepithelial neoplasia’ (PanIN) (Hruban et al., Am. J. Surg. Pathol. 25(5), 579-586, 2001; Biankin et al., Pathol. 35(1), 14-24, 2003). The characterisation of a progression model has enabled further research into pancreatic tumorigenesis. In particular it allows investigators to study the early molecular changes associated in the transformation of normal ducts to precursor PanIN lesions and through to invasive cancer.

### Detection and Management

Pancreatic cancer presents commonly as a clinically advanced disease with few treatment options. Surgery provides the only chance of cure. Despite controversies in the diagnosis and management of pancreatic cancer, variations in investigations and treatment alternatives to surgery have produced little benefit to the overall survival (Engelken et al., Eur. J. Surg. One. 29(4), 368-373, 2003). The greatest improvements in pancreatic cancer mortality are likely to have come from improvements in perioperative care provided by specialist centres.

Common symptoms of pancreatic cancer are non-specific and include pain, jaundice, anorexia, early satiety and weight-loss (Warshaw and Fernandez-del Castillo, N Engl J Med, 326(7), 455-465, 1992). Pain presents as the most frequent symptom of pancreatic malignancy and is present in 80% of subjects with non-resectable cancer (DiMagno et al., Gastroenterol. 117(6), 1464-1484, 1999). At the time of diagnosis over 85% of tumours have extended beyond the pancreas (Warshaw and Fernandez-del Castillo, N Engl J Med, 326(7), 455-465, 1992). Computer tomography (CT) scanning is the recommended primary investigation of suspected pancreatic carcinoma, and is useful in the assessment for resectability and staging (Gloor et al., Cancer 79(9), 1780-1786, 1999). The staging system used in this study is that of the International Union Against Cancer (UICC) (Table 2). Endoscopic ultrasound is a specialised investigation which may give a better assessment of local invasion compared to CT and provides an opportunity for fine needle aspiration biopsy (FNAB) as part of the procedure (Wiersema, Pancreatol. 1(6), 625-632, 2001; Burris et al., J. Clin. Oncol. 15(6), 2403-2413, 1997). If no evidence of local or distant spread of cancer exists, then the subject may be treated by resection of their cancer by pancreaticoduodenectomy (Whipple's procedure). Subjects with clinical or radiological evidence of invasive disease are inoperable and usually undergo a percutaneous FNAB to confirm diagnosis prior to palliative treatment (Gloor et al., Cancer 79(9), 1780-1786, 1999).

Adjuvant chemotherapy and radiotherapy make only minor differences to outcome in pancreatic cancer and their role is controversial (Burris et al., J. Clin. Oncol. 15(6), 2403-2413, 1997). Recent trials reported that combined chemoradiotherapy was detrimental and that treatment with 5-fluorouracil alone showed a small survival advantage (Neoptolemos et al., Lancet 358(9293), 1576-1585, 2001). Gemcitabine improves survival marginally in subjects with advanced disease and offers better quality of life in the palliative setting (Burris et al., J. Clin. Oncol. 15(6), 2403-2413, 1997).

A tumour marker that is both highly sensitive and specific to the diagnosis of pancreatic cancer is yet to be found. In a review of tumour markers, levels of CA 19-9 were found to have the greatest sensitivity (70%) and specificity (87%) to pancreatic malignancy (Ebert et al., J. Cancer Res. & Clin. Oncol. 127(7), 449-454, 2001). However, use of CA 19-9 in the diagnosis of pancreatic cancer varies, and is not recommended by the American Gastroenterological Association guidelines for diagnosis of pancreatic cancer (DiMagno et al., Gastroenterol. 117(6), 1464-1484, 1999). CA 19-9 may be of more clinical utility in the follow-up of subjects after treatment rather than as a diagnostic tool (Lamerz, Ann. Oncol. 10(Suppl 4), 145-149, 1999).

### Prognostic Factors

Overall prognosis for pancreatic cancer is very poor, with less than 5% of subjects surviving 5 years after diagnosis (Sohn et al., J Gastro Surg., 4(6), 567-579, 2000; Geer and Brennan, Am J Surg, 165(1), 68-73, 1993). There are few substantial series reporting significant prognostic markers in pancreatic cancer. In subjects who undergo operative resection, positive surgical margins, lymph node involvement and large tumour size are poor prognostic factors (Geer and Brennan, Am J Surg, 165(1), 68-73, 1993). Other parameters such as DNA ploidy and perineural invasion have been investigated, and the results are suggestive of an association with outcome but are not conclusive (Biankin et al. J Clin Oncol, 20(23), 4531-4542, 2002). In addition, the preoperative assessment of criteria such as tumour size and lymph node involvement is difficult. Novel molecular markers in pancreatic cancer have the potential to give greater accuracy in predicting prognosis and treatment response, and serve to guide subject and clinician in treatment decisions.

TABLE 2
International Union Against Cancer (UICC)
classification for pancreatic cancer.
T: Primary Tumour

| | |
|---|---|
| TX | Primary Tumour cannot be assessed |
| T0 | No evidence of primary tumour |
| T1 | Tumour limited to the pancreas |
| T1a | Tumour 2 cm or less in greatest dimension |
| T1b | Tumour more than 2 cm in greatest dimension |
| T2 | Tumour extends directly into any of duodenum, bile duct, |
| | or peripancreatic tissue |
| T3 | Tumour extends directly into any of the following: stomach, |
| | spleen, colon, adjacent large vessels |

N: Regional lymph nodes

| | |
|---|---|
| NX | Regional lymph nodes cannot be assessed |
| N0 | No regional lymph node metastasis |
| N1 | Regional lymph node metastasis |

M: Distant metastasis

| | |
|---|---|
| MX | Presence of distant metastasis cannot be assessed |
| M0 | No regional lymph node metastasis |
| M1 | Distant metastasis |

Stage Grouping

| | | | |
|---|---|---|---|
| Stage I | T1 | N0 | M0 |
| | T2 | N0 | M0 |
| Stage II | T3 | N0 | M0 |
| Stage III | Any T | N1 | M0 |
| Stage IV | Any T | Any N | M1 |

### SUMMARY OF THE INVENTION

In work leading up to the present invention, the inventors sought to identify nucleic acid markers that were diagnostic of pancreatic cancers generally, or diagnostic of pancreatic cancer by virtue of their modulated expression in cancer tissues derived from a patient cohort compared to their expression in healthy or non-cancerous cells and tissues.

As exemplified herein, the inventors identified a number of genes whose expression is altered (up-regulated or down-regulated) in individuals with pancreatic cancer compared to healthy individuals., eg., subjects who do not have pancreatic cancer. The particular genes are identified in Table 3 (up-regulated genes) and Table 4 (down-regulated genes). The list of genes and proteins exemplified herein by Tables 3 and 4 were identified by a statistical analysis as outlined in the examples which gave a P-value, eg., by comparison of expression to the expression of that gene in normal pancreas.

Analysis of the diagnostic genes set forth in Tables 3 and 4 indicates that many of those genes fall into discrete classes, based upon their functionalities, wherein those classes are selected from the group consisting of:

(i) genes encoding membrane proteins (Table 5);
(ii) genes encoding extracellular proteins (Table 6);
(iii) genes encoding proteins of the TGF-β signalling pathway (Table 7);
(iv) genes encoding WNT signalling pathway proteins (Table 8);
(v) genes encoding proteins of nucleotide metabolism (Table 9);
(vi) genes encoding proteins involved in smooth muscle contraction (Table 10);
(vii) genes encoding mitochondrial proteins (Table 11);
(viii) genes encoding collagens, proteins of collagen synthesis or fibrillins (Table 12);
(ix) genes encoding inflammatory response pathway proteins (Table 13);
(x) genes encoding endoplasmic reticulum (ER) proteins (Table 14);
(xi) genes encoding apoptotic proteins (Table 15);
(xii) genes encoding G1/S phase cell cycle control proteins (Table 16);
(xiii) genes encoding matrix metalloproteinases (Table 17);
(xiv) genes encoding proteins involved in retinoic acid signal transduction (Table 18);
(xv) genes encoding calcium channel proteins (Table 19);
(xvi) genes encoding cathepsin proteins (Table 20);
(xvii) genes encoding viral oncoprotein homologs (Table 21);
(xviii) genes encoding S100 calcium binding proteins (Table 22);
(xix) genes encoding homeobox proteins (Table 23);
(xx) genes encoding zinc finger proteins (Table 24); and
(xxi) genes encoding heat shock proteins (Table 25).

As will be known to the skilled artisan, the GenBANK Accession Nos. set forth in any one of Tables 3-25 provide access to publicly available nucleotide and amino acid sequence data for any one or more genes used in the presently-disclosed diagnostic/prognostic assays or other processes/methods disclosed herein. Accordingly, each of the nucleotide and amino acid sequences contained in the GenBank database or database of the National Center for Biotechnology Information (NCBI) of the U.S. National Library of Medicine, 8600 Rockville Pike, Bethesda, Md. 20894, USA, under an Accession No. referred to in any one of Tables 3-25 is incorporated herein by reference, specifically the nucleic acid and amino acid sequences of each of Genbank accession numbers NM_{—}005980.1 (SEQ ID NO:13 and SEQ ID NO:14, respectively), NM_{—}005978.2 (SEQ ID NO:15 and SEQ ID NO:16, respectively), NM_{—}014624.2 (SEQ ID NO:17 and SEQ ID NO:18, respectively), NM_{—}005620.1 (SEQ ID NO:19 and SEQ ID NO:20, respectively), NM_{—}002966.1 (SEQ ID NO:21 and SEQ ID NO:22, respectively), NM_{—}002961.2 (SEQ ID NO:23 and SEQ ID NO:24, respectively) and NM_{—}021039.1 (SEQ ID NO:25 and SEQ ID NO:26, respectively). Sequences of the diagnostic.prognostic markers referred to herein are also available in other databases, e.g., European Molecular Biology Laboratory (EMBL) and DNA Database of Japan (DDBJ).

One aspect of the present invention relates to nucleic acid-based assays for diagnosing a pancreatic cancer in a human or animal subject.

Accordingly, one embodiment provides a method of detecting a pancreatic cancer-associated transcript in a biological sample, the method comprising contacting the biological sample with a polynucleotide that selectively hybridizes to a sequence at least 80% identical to a sequence as shown in any one of Tables 3 to 25. Preferably the percentage identity to a nucleotide sequence disclosed in any one of Tables 3 to 25 is at least about 85% or 90% or 95%, and still more preferably at least about 98% or 99%.

Alternatively, or in addition, the present invention provides a method of detecting a pancreatic cancer-associated transcript in a biological sample, the method comprising contacting the biological sample with a polynucleotide that selectively hybridizes to a sequence at least 80% identical to a sequence having the GenBank Accession No. AF 279145. Preferably the percentage identity to a sequence having the GenBank Accession No. AF 279145 is at least about 85% or 90% or 95%, and still more preferably at least about 98% or 99%. For the purposes of nomenclature, the sequence set forth in GenBank Accession No. AF 279145 relates to the homo sapiens tumor endothelial marker TEM8, the nucleotide sequence of which is also set forth herein as SEQ ID NO: 3. The amino acid sequence encoded by the TEM8 gene is provided herein as SEQ ID NO: 4.

In a preferred embodiment, the present invention relates to the use of nucleic acid selected from the group consisting of gamma-aminobutyric acid (GABA) A receptor, pi (GABRP)-encoding nucleic acid exemplified herein by SEQ ID NO: 1; tumor endothelial marker 8 precursor (TEM8)-encoding nucleic acid exemplified herein by SEQ ID NO: 3; cadherin 11, type 2 (CDH11)-encoding nucleic acid exemplified herein by SEQ ID NO: 5; type II membrane serine protease (TMPRSS4)-encoding nucleic acid exemplified herein by SEQ ID NO: 7; retinoic acid induced 3 (RAI3) gene exemplified herein by SEQ ID NO: 9; and homeo box B2 (HOXB2)-encoding nucleic acid exemplified herein by SEQ ID NO: 11. In accordance with this embodiment, the present invention clearly encompasses a method of detecting a pancreatic cancer-associated transcript in a biological sample, the method comprising contacting the biological sample with a polynucleotide that selectively hybridizes to a sequence at least 80% identical to a sequence selected from the group consisting of SEQ ID Nos: 1, 3, 5, 7, 9, and 11. Preferably the percentage identity to any one of SEQ ID Nos: 1, 3, 5, 7, 9, or 11 is at least about 85% or 90% or 95%, and still more preferably at least about 98% or 99%.

In a more preferred embodiment, the present invention provides a method of diagnosing a pancreatic cancer in a human or animal subject being tested said method comprising contacting a biological sample from said subject being tested with a nucleic acid probe for a time and under conditions sufficient for hybridization to occur and then detecting the hybridization wherein an enhanced level of hybridization of the probe for the subject being tested compared to the hybridization obtained for a control subject not having pancreatic cancer indicates that the subject being tested has a pancreatic cancer, and wherein said nucleic acid probe comprises a sequence selected from the group consisting of:
(i) a sequence comprising at least about 20 contiguous nucleotides from a sequence selected from the group consisting of SEQ ID NOs: 1, 3, 5, 7, 9 and 11;
(ii) a sequence that hybridizes under at least low stringency hybridization conditions to at least about 20 contiguous nucleotides from a sequence selected from the group consisting of SEQ ID NOs: 1, 3, 5, 7, 9 and 11;
(iii) a sequence that is at least about 80% identical to a sequence selected from the group consisting of SEQ ID NOs: 1, 3, 5, 7, 9 and 11;
(iv) a sequence that encodes an amino acid sequence selected from the group consisting of SEQ ID NOs: 2, 4, 6, 8, 10 and 12; and
(v) a sequence that is complementary to any one of the sequences set forth in (i) or (ii) or (iii) or (iv).

In a particularly preferred embodiment, the diagnostic/prognostic assay of the present invention depends upon the use of a HOX B2-encoding nucleic acid probe. In accordance with this preferred embodiment, the present invention provides a method of diagnosing a pancreatic cancer in a human or animal subject being tested said method comprising contacting a biological sample from said subject being tested with a nucleic acid probe for a time and under conditions sufficient for hybridization to occur and then detecting the hybridization wherein an enhanced level of hybridization of the probe for the subject being tested compared to the hybridization obtained for a control subject not having pancreatic cancer indicates that the subject being tested has a pancreatic cancer, and wherein said nucleic acid probe comprises a sequence selected from the group consisting of:
(i) a sequence comprising at least about 20 contiguous nucleotides from SEQ ID NO: 11;
(ii) a sequence that hybridizes under at least low stringency hybridization conditions to at least about 20 contiguous nucleotides from SEQ ID NO: 11;
(iii) a sequence that is at least about 80% identical to SEQ ID NO: 11;
(iv) a sequence that encodes the amino acid sequence set forth in SEQ ID NO: 12; and
(v) a sequence that is complementary to any one of the sequences set forth in (i) or (ii) or (iii) or (iv).

In a preferred embodiment, the present invention provides a method of diagnosing a pancreatic cancer in a human or animal subject being tested said method comprising contacting a biological sample from said subject being tested with a nucleic acid probe for a time and under conditions sufficient for hybridization to occur and then detecting the hybridization wherein a modified level of hybridization of the probe for the subject being tested compared to the hybridization obtained for a control subject not having pancreatic cancer indicates that the subject being tested has a pancreatic cancer, and wherein said nucleic acid probe comprises a sequence selected from the group consisting of:

(i) a sequence comprising at least about 20 contiguous nucleotides from nucleic acid encoding a membrane protein and having an Accession Number selected from the group consisting of: NM_{—}004363.1, NM_{—}003979.2, NM_{—}004696.1, NM_{—}002888.1, BC005008.1, NM_{—}005672.1, S59049.1, AI631159_RC, NM_{—}004476.1, NM_{—}000227.1, NM_{—}000593.2, NM_{—}013451.1, NM_{—}002888.1, AL162079.1, NM_{—}001945.1, M85289.1, BG170541, NM_{—}002510.1, AV713720, NM_{—}003272.1, NM_{—}004334.1, AI741056_RC, U07139.1, AI356412_RC, AL161958.1, NM_{—}006670.1, NM_{—}003641.1, AF000425.1, NM_{—}012329.1, AW151360_RC, NM_{—}012449.1, NM_{—}003507.1, M81635.1, NM_{—}003332.1, BC000961.2, NM_{—}003174.2, NM_{—}001663.2, NM_{—}001904.1, M76446.1, NM_{—}002231.2, U45448.1, NM_{—}001502.1, NM_{—}001169.1 and NM_{—}016295.1;
(ii) a sequence that hybridizes under at least low stringency hybridization conditions to at least about 20 contiguous nucleotides from nucleic acid encoding a membrane protein and having an Accession Number selected from the group consisting of: NM_{—}004363.1, NM_{—}003979.2, NM_{—}004696.1, NM_{—}002888.1, BC005008.1, NM_{—}005672.1, S59049.1, AI631159_RC, NM_{—}004476.1, NM_{—}000227.1, NM_{—}000593.2, NM_{—}013451.1, NM_{—}002888.1, AL162079.1, NM_{—}001945.1, M85289.1, BG170541, NM_{—}002510.1, AV713720, NM_{—}003272.1, NM_{—}004334.1, AI741056_RC, U07139.1, AI356412_RC, AL161958.1, NM_{—}006670.1, NM_{—}003641.1, AF000425.1, NM_{—}012329.1, AW151360_RC, NM_{—}012449.1, NM_{—}003507.1, M81635.1, NM_{—}003332.1, BC000961.2, NM_{—}003174.2, NM_{—}001663.2, NM_{—}001904.1, M76446.1, NM_{—}002231.2, U45448.1, NM_{—}001502.1, NM_{—}001169.1 and NM_{—}016295.1;
(iii) a sequence that encodes a membrane protein having an Accession Number selected from the group consisting of: NM_{—}004363.1, NM_{—}003979.2, NM_{—}004696.1, NM_{—}002888.1, BC005008.1, NM_{—}005672.1, S59049.1, AI631159_RC, NM_{—}004476.1, NM_{—}000227.1, NM_{—}000593.2, NM_{—}013451.1, NM_{—}002888.1, AL162079.1, NM_{—}001945.1, M85289.1, BG170541, NM_{—}002510.1, AV713720, NM_{—}003272.1, NM_{—}004334.1, AI741056_RC, U07139.1, AI356412_RC, AL161958.1, NM_{—}006670.1, NM_{—}003641.1, AF000425.1, NM_{—}012329.1, AW151360-RC, NM_{—}012449.1, NM_{—}003507.1, M81635.1, NM_{—}003332.1, BC000961.2, NM_{—}003174.2, NM_{—}001663.2, NM_{—}001904.1, M76446.1, NM_{—}002231.2, U45448.1, NM_{—}001502.1, NM_{—}001169.1 and NM_{—}016295.1; and
(iv) a sequence that is complementary to any one of the sequences set forth in (i) or (ii) or (iii).

Preferably, the membrane protein is selected from the group consisting of selected from the group consisting of a type II membrane serine protease (TMPRSS4) exemplified herein by SEQ ID NO: 8, a homolog of a type II membrane serine protease (TMPRSS4) exemplified herein by SEQ ID NO: 8, a polypeptide encoded by a retinoic acid induced 3 (RAI3) gene as exemplified herein by SEQ ID NO: 10 and a homolog of a polypeptide encoded by a retinoic acid induced 3 (RAI3) gene as exemplified herein by SEQ ID NO: 10. In accordance with this preferred embodiment, the present invention provides a method of diagnosing a pancreatic cancer in a human or animal subject being tested said method comprising contacting a biological sample from said subject being tested with a nucleic acid probe for a time and under conditions sufficient for hybridization to occur and then detecting the hybridization wherein an enhanced level of hybridization of the probe for the subject being tested compared to the hybridization obtained for a control subject not having pancreatic cancer indicates that the subject being tested has a pancreatic cancer, and wherein said nucleic acid probe comprises a sequence selected from the group consisting of:
(i) a sequence comprising at least about 20 contiguous nucleotides from a sequence selected from the group consisting of SEQ ID NO: 7 and SEQ ID NO: 9;
(ii) a sequence that hybridizes under at least low stringency hybridization conditions to at least about 20 contiguous nucleotides from a sequence selected from the group consisting of SEQ ID NO: 7 and SEQ ID NO: 9;
(iii) a sequence that is at least about 80% identical to a sequence selected from the group consisting of SEQ ID NO: 7 and SEQ ID NO: 9;
(iv) a sequence that encodes an amino acid sequence selected from the group consisting of SEQ ID NO: 8 and SEQ ID NO: 10; and
(v) a sequence that is complementary to any one of the sequences set forth in (i) or (ii) or (iii) or (iv).

In an alternative preferred embodiment, the present invention provides a method of diagnosing a pancreatic cancer in a human or animal subject being tested said method comprising contacting a biological sample from said subject being tested with a nucleic acid probe for a time and under conditions sufficient for hybridization to occur and then detecting the hybridization wherein a modified level of hybridization of the probe for the subject being tested compared to the hybridization obtained for a control subject not having pancreatic cancer indicates that the subject being tested has a pancreatic cancer, and wherein said nucleic acid probe comprises a sequence selected from the group consisting of:

(i) a sequence comprising at least about 20 contiguous nucleotides from nucleic acid encoding an extracellular protein and having an Accession Number selected from the group consisting of: NM_{—}004591.1, M13436.1, M31159.1, NM_{—}005940.2, X02761.1, BF590263_RC, BF218922, NM_{—}000095.1, NM_{—}000584.1, BC002710.1, AF154054.1, NM_{—}003247.1, NM_{—}002160.1, NM_{—}006533.1, NM_{—}002546.1, NM_{—}013372.1, NM_{—}004385.1, NM_{—}003118.1, NM_{—}003014.2, NM_{—}001945.1, M85289.1, NM_{—}000138.1, NM_{—}005567.2, NM_{—}002090.1, NM_{—}013253.1, NM_{—}012445.1, NM_{—}002933.1, BF508685_RC and NM_{—}006229.1;
(ii) a sequence that hybridizes under at least low stringency hybridization conditions to at least about 20 contiguous nucleotides from nucleic acid encoding an extracellular protein and having an Accession Number selected from the group consisting of: NM_{—}004591.1, M13436.1, M31159.1, NM_{—}005940.2, X02761.1, BF590263_RC, BF218922, NM_{—}000095.1, NM_{—}000584.1, BC002710.1, AF154054.1, NM_{—}003247.1, NM_{—}002160.1, NM_{—}006533.1, NM_{—}002546.1, NM_{—}013372.1, NM_{—}004385.1, NM_{—}003118.1, NM_{—}003014.2, NM_{—}001945.1, M85289.1, NM_{—}000138.1, NM_{—}005567.2, NM_{—}002090.1, NM_{—}013253.1, NM_{—}012445.1, NM_{—}002933.1, BF508685_RC and NM_{—}006229.1;
(iii) a sequence that encodes an extracellular protein having an Accession Number selected from the group consisting of: NM_{—}004591.1, M13436.1, M31159.1, NM_{—}005940.2, X02761.1, BF590263_RC, BF218922, NM_{—}000095.1, NM_{—}000584.1, BC002710.1, AF154054.1, NM_{—}003247.1, NM_{—}002160.1, NM_{—}006533.1, NM_{—}002546.1, NM_{—}013372.1, NM_{—}004385.1, NM_{—}003118.1, NM_{—}003014.2, NM_{—}001945.1, M85289.1, NM_{—}000138.1, NM_{—}005567.2, NM_{—}002090.1, NM_{—}013253.1, NM_{—}012445.1, NM_{—}002933.1, BF508685_RC and NM_{—}006229.1; and
(iv) a sequence that is complementary to any one of the sequences set forth in (i) or (ii) or (iii).

In an alternative preferred embodiment, the present invention provides a method of diagnosing a pancreatic cancer in a human or animal subject being tested said method comprising contacting a biological sample from said subject being tested with a nucleic acid probe for a time and under conditions sufficient for hybridization to occur and then detecting the hybridization wherein an enhanced level of hybridization of the probe for the subject being tested compared to the hybridization obtained for a control subject not having pancreatic cancer indicates that the subject being tested has a pancreatic cancer, and wherein said nucleic acid probe comprises a sequence selected from the group consisting of:

(i) a sequence comprising at least about 20 contiguous nucleotides from nucleic acid encoding a protein of the TGF-β signalling pathway and having an Accession Number selected from the group consisting of: M13436.1, AF288571.1, BC002704.1, U44378.1 and NM_{—}001904.1;
(ii) a sequence that hybridizes under at least low stringency hybridization conditions to at least about 20 contiguous nucleotides from nucleic acid encoding a protein of the TGF-β signalling pathway and having an Accession Number selected from the group consisting of: M13436.1, AF288571.1, BC002704.1, U44378.1 and NM_{—}001904.1;
(iii) a sequence that encodes a protein of the TGF-β signalling pathway having an Accession Number selected from the group consisting of: M13436.1, AF288571.1, BC002704.1, U44378.1 and NM_{—}001904.1; and
(iv) a sequence that is complementary to any one of the sequences set forth in (i) or (ii) or (iii).

In an alternative preferred embodiment, the present invention provides a method of diagnosing a pancreatic cancer in a human or animal subject being tested said method comprising contacting a biological sample from said subject being tested with a nucleic acid probe for a time and under conditions sufficient for hybridization to occur and then detecting the hybridization wherein a modified level of hybridization of the probe for the subject being tested compared to the hybridization obtained for a control subject not having pancreatic cancer indicates that the subject being tested has a pancreatic cancer, and wherein said nucleic acid probe comprises a sequence selected from the group consisting of:

(i) a sequence comprising at least about 20 contiguous nucleotides from nucleic acid encoding a WNT signalling pathway protein and having an Accession Number selected from the group consisting of: NM_{—}003014.2, AF311912.1, AF143679.1, NM_{—}013253.1, L37882.1, NM_{—}003882.1, U91903.1, NM_{—}003507.1, NM_{—}030775.1, NM_{—}001904.1 and NM_{—}013266.1;
(ii) a sequence that hybridizes under at least low stringency hybridization conditions to at least about 20 contiguous nucleotides from nucleic acid encoding a WNT signalling pathway protein and having an Accession Number selected from the group consisting of: NM_{—}003014.2, AF311912.1, AF143679.1, NM_{—}013253.1, L37882.1, NM_{—}003882.1, U91903.1, NM_{—}003507.1, NM_{—}030775.1, NM_{—}001904.1 and NM_{—}013266.1;
(iii) a sequence that encodes a WNT signalling pathway protein having an Accession Number selected from the group consisting of: NM_{—}003014.2, AF311912.1, AF143679.1, NM_{—}013253.1, L37882.1, NM_{—}003882.1, U91903.1, NM_{—}003507.1, NM_{—}030775.1, NM_{—}001904.1 and NM_{—}013266.1; and
(iv) a sequence that is complementary to any one of the sequences set forth in (i) or (ii) or (iii).

In an alternative preferred embodiment, the present invention provides a method of diagnosing a pancreatic cancer in a human or animal subject being tested said method comprising contacting a biological sample from said subject being tested with a nucleic acid probe for a time and under conditions sufficient for hybridization to occur and then detecting the hybridization wherein an enhanced level of hybridization of the probe for the subject being tested compared to the hybridization obtained for a control subject not having pancreatic cancer indicates that the subject being tested has a pancreatic cancer, and wherein said nucleic acid probe comprises a sequence selected from the group consisting of:

(i) a sequence comprising at least about 20 contiguous nucleotides from nucleic acid encoding a protein of nucleotide metabolism and having an Accession Number selected from the group consisting of: BE971383 and NM_{—}002970.1;
(ii) a sequence that hybridizes under at least low stringency hybridization conditions to at least about 20 contiguous nucleotides from nucleic acid encoding a protein of nucleotide metabolism and having an Accession Number selected from the group consisting of: BE971383 and NM_{—}002970.1;
(iii) a sequence that encodes a protein of nucleotide metabolism having an Accession Number selected from the group consisting of: BE971383 and NM_{—}002970.1; and
(iv) a sequence that is complementary to any one of the sequences set forth in (i) or (ii) or (iii).

In an alternative preferred embodiment, the present invention provides a method of diagnosing a pancreatic cancer in a human or animal subject being tested said method comprising contacting a biological sample from said subject being tested with a nucleic acid probe for a time and under conditions sufficient for hybridization to occur and then detecting the hybridization wherein an enhanced level of hybridization of the probe for the subject being tested compared to the hybridization obtained for a control subject not having pancreatic cancer indicates that the subject being tested has a pancreatic cancer, and wherein said nucleic acid probe comprises a sequence selected from the group consisting of:

(i) a sequence comprising at least about 20 contiguous nucleotides from nucleic acid encoding a protein involved in smooth muscle contraction and having an Accession Number selected from the group consisting of: NM_{—}005965.1, NM_{—}006097.1, NM_{—}001613.1 and AI082078_RC;
(ii) a sequence that hybridizes under at least low stringency hybridization conditions to at least about 20 contiguous nucleotides from nucleic acid encoding a protein involved in smooth muscle contraction and having an Accession Number selected from the group consisting of: NM_{—}005965.1, NM_{—}006097.1, NM_{—}001613.1 and AI082078_RC;
(iii) a sequence that encodes a protein involved in smooth muscle contraction having an Accession Number selected from the group consisting of: NM_{—}005965.1, NM_{—}006097.1, NM_{—}001613.1 and AI082078_RC; and
(iv) a sequence that is complementary to any one of the sequences set forth in (i) or (ii) or (iii).

In an alternative preferred embodiment, the present invention provides a method of diagnosing a pancreatic cancer in a human or animal subject being tested said method comprising contacting a biological sample from said subject being tested with a nucleic acid probe for a time and under conditions sufficient for hybridization to occur and then detecting the hybridization wherein a modified level of hybridization of the probe for the subject being tested compared to the hybridization obtained for a control subject not having pancreatic cancer indicates that the subject being tested has a pancreatic cancer, and wherein said nucleic acid probe comprises a sequence selected from the group consisting of:

(i) a sequence comprising at least about 20 contiguous nucleotides from nucleic acid encoding a mitochondrial protein and having an Accession Number selected from the group consisting of: NM_{—}000104.2, NM_{—}002064.1, NM_{—}000784.1, NM_{—}003359.1, R92925_RC, NM_{—}004294.1, T67741_RC and NM_{—}001914.1;
(ii) a sequence that hybridizes under at least low stringency hybridization conditions to at least about 20 contiguous nucleotides from nucleic acid encoding a mitochondrial protein and having an Accession Number selected from the group consisting of: NM_{—}000104.2, NM_{—}002064.1, NM_{—}000784.1, NM_{—}003359.1, R92925_RC, NM_{—}004294.1, T67741_RC and NM_{—}001914.1;
(iii) a sequence that encodes a mitochondrial protein having an Accession Number selected from the group consisting of: NM_{—}000104.2, NM_{—}002064.1, NM_{—}000784.1, NM_{—}003359.1, R92925_RC, NM_{—}004294.1, T67741_RC and NM_{—}001914.1; and
(iv) a sequence that is complementary to any one of the sequences set forth in (i) or (ii) or (iii).

In an alternative preferred embodiment, the present invention provides a method of diagnosing a pancreatic cancer in a human or animal subject being tested said method comprising contacting a biological sample from said subject being tested with a nucleic acid probe for a time and under conditions sufficient for hybridization to occur and then detecting the hybridization wherein an enhanced level of hybridization of the probe for the subject being tested compared to the hybridization obtained for a control subject not having pancreatic cancer indicates that the subject being tested has a pancreatic cancer, and wherein said nucleic acid probe comprises a sequence selected from the group consisting of:

(i) a sequence comprising at least about 20 contiguous nucleotides from nucleic acid encoding a collagen, a protein of collagen synthesis or a fibrillin and having an Accession Number selected from the group consisting of: NM_{—}002593.2, NM_{—}001854.1, AL575735_RC, AI983428_RC, NM_{—}000138.1, X05610.1, NM_{—}000089.1, AI743621_RC and AU144167_RC;
(ii) a sequence that hybridizes under at least low stringency hybridization conditions to at least about 20 contiguous nucleotides from nucleic acid encoding a collagen, a protein of collagen synthesis or a fibrillin and having an Accession Number selected from the group consisting of: NM_{—}002593.2, NM_{—}001854.1, AL575735_RC, AI983428_RC, NM_{—}000138.1, X05610.1, NM_{—}000089.1, AI743621_RC and AU144167_RC;
(iii) a sequence that encodes a collagen, a protein of collagen synthesis or a fibrillin having an Accession Number selected from the group consisting of: NM_{—}002593.2, NM_{—}001854.1, AL575735_RC, AI983428_RC, NM_{—}000138.1, X05610.1, NM_{—}000089.1, AI743621_RC and AU144167_RC; and
(iv) a sequence that is complementary to any one of the sequences set forth in (i) or (ii) or (iii).

In an alternative preferred embodiment, the present invention provides a method of diagnosing a pancreatic cancer in a human or animal subject being tested said method comprising contacting a biological sample from said subject being tested with a nucleic acid probe for a time and under conditions sufficient for hybridization to occur and then detecting the hybridization wherein an enhanced level of hybridization of the probe for the subject being tested compared to the hybridization obtained for a control subject not having pancreatic cancer indicates that the subject being tested has a pancreatic cancer, and wherein said nucleic acid probe comprises a sequence selected from the group consisting of:

(i) a sequence comprising at least about 20 contiguous nucleotides from nucleic acid encoding an inflammatory response pathway protein and having an Accession Number selected from the group consisting of: NM_{—}000089.1, BC005858.1, X02761.1, AK026737.1, NM_{—}005562.1, AI743621_RC and AU144167_RC;
(ii) a sequence that hybridizes under at least low stringency hybridization conditions to at least about 20 contiguous nucleotides from nucleic acid encoding an inflammatory response pathway protein and having an Accession Number selected from the group consisting of: NM_{—}000089.1, BC005858.1, X02761.1, AK026737.1, NM_{—}005562.1, AI743621_RC and AU144167_RC;
(iii) a sequence that encodes an inflammatory response pathway protein having an Accession Number selected from the group consisting of: NM_{—}000089.1, BC005858.1, X02761.1, AK026737.1, NM_{—}005562.1, AI743621_RC and AU144167_RC; and
(iv) a sequence that is complementary to any one of the sequences set forth in (i) or (ii) or (iii).

In an alternative preferred embodiment, the present invention provides a method of diagnosing a pancreatic cancer in a human or animal subject being tested said method comprising contacting a biological sample from said subject being tested with a nucleic acid probe for a time and under conditions sufficient for hybridization to occur and then detecting the hybridization wherein an enhanced level of hybridization of the probe for the subject being tested compared to the hybridization obtained for a control subject not having pancreatic cancer indicates that the subject being tested has a pancreatic cancer, and wherein said nucleic acid probe comprises a sequence selected from the group consisting of:

(i) a sequence comprising at least about 20 contiguous nucleotides from nucleic acid encoding an endoplasmic reticulum (ER) protein and having an Accession Number selected from the group consisting of: NM_{—}004353.1, AV691323, BC000961.2, NM_{—}000961.1 and AI753659_RC;
(ii) a sequence that hybridizes under at least low stringency hybridization conditions. to at least about 20 contiguous nucleotides from nucleic acid encoding an endoplasmic reticulum (ER) protein and having an Accession Number selected from the group consisting of: NM_{—}004353.1, AV691323, BC000961.2, NM_{—}000961.1 and AI753659_RC;
(iii) a sequence that encodes an endoplasmic reticulum (ER) protein having an Accession Number selected from the group consisting of: NM_{—}004353.1, AV691323, BC000961.2, NM_{—}000961.1 and AI753659_RC; and
(iv) a sequence that is complementary to any one of the sequences set forth in (i) or (ii) or (iii).

In an alternative preferred embodiment, the present invention provides a method of diagnosing a pancreatic cancer in a human or animal subject being tested said method comprising contacting a biological sample from said subject being tested with a nucleic acid probe for a time and under conditions sufficient for hybridization to occur and then detecting the hybridization wherein a modified level of hybridization of the probe for the subject being tested compared to the hybridization obtained for a control subject not having pancreatic cancer indicates that the subject being tested has a pancreatic cancer, and wherein said nucleic acid probe comprises a sequence selected from the group consisting of:

(i) a sequence comprising at least about 20 contiguous nucleotides from nucleic acid encoding an apoptotic protein and having an Accession Number selected from the group consisting of: NM_{—}000546.2 and AF201370.1;
(ii) a sequence that hybridizes under at least low stringency hybridization conditions to at least about 20 contiguous nucleotides from nucleic acid encoding an apoptotic protein and having an Accession Number selected from the group consisting of: NM_{—}000546.2 and AF201370.1;
(iii) a sequence that encodes an apoptotic protein having an Accession Number selected from the group consisting of: NM_{—}000546.2 and AF201370.1; and
(iv) a sequence that is complementary to any one of the sequences set forth in (i) or (ii) or (iii).

In an alternative preferred embodiment, the present invention provides a method of diagnosing a pancreatic cancer in a human or animal subject being tested said method comprising contacting a biological sample from said subject being tested with a nucleic acid probe for a time and under conditions sufficient for hybridization to occur and then detecting the hybridization wherein a modified level of hybridization of the probe for the subject being tested compared to the hybridization obtained for a control subject not having pancreatic cancer indicates that the subject being tested has a pancreatic cancer, and wherein said nucleic acid probe comprises a sequence selected from the group consisting of:

(i) a sequence comprising at least about 20 contiguous nucleotides from nucleic acid encoding a G1/S phase cell cycle control protein and having an Accession Number selected from the group consisting of: NM_{—}001237.1, NM_{—}000546.2, NM_{—}003674.1, BE407516, R78668_RC, NM_{—}000077.1, BC000076.1 and NM_{—}000389.1;
(ii) a sequence that hybridizes under at least low stringency hybridization conditions to at least about 20 contiguous nucleotides from nucleic acid encoding a G1/S phase cell cycle control protein and having an Accession Number selected from the group consisting of: NM_{—}001237.1, NM_{—}000546.2, NM_{—}003674.1, BE407516, R78668_RC, NM_{—}000077.1, BC000076.1 and NM_{—}000389.1;
(iii) a sequence that encodes a G1/S phase cell cycle control protein having an Accession Number selected from the group consisting of: NM_{—}001237.1, NM_{—}000546.2, NM_{—}003674.1, BE407516, R78668_RC, NM_{—}000077.1, BC000076.1 and NM_{—}000389.1; and
(iv) a sequence that is complementary to any one of the sequences set forth in (i) or (ii) or (iii).

In an alternative preferred embodiment, the present invention provides a method of diagnosing a pancreatic cancer in a human or animal subject being tested said method comprising contacting a biological sample from said subject being tested with a nucleic acid probe for a time and under conditions sufficient for hybridization to occur and then detecting the hybridization wherein a modified level of hybridization of the probe for the subject being tested compared to the hybridization obtained for a control subject not having pancreatic cancer indicates that the subject being tested has a pancreatic cancer, and wherein said nucleic acid probe comprises a sequence selected from the group consisting of:

(i) a sequence comprising at least about 20 contiguous nucleotides from nucleic acid encoding a matrix metalloproteinase and having an Accession Number selected from the group consisting of: NM_{—}005940.2, NM_{—}004995.2, NM_{—}003254.1, NM_{—}004530.1, AF219624.1 and W45551_RC;
(ii) a sequence that hybridizes under at least low stringency hybridization conditions to at least about 20 contiguous nucleotides from nucleic acid encoding a matrix metalloproteinase and having an Accession Number selected from the group consisting of: NM_{—}005940.2, NM_{—}004995.2, NM_{—}003254.1, NM_{—}004530.1, AF219624.1 and W45551_RC;
(iii) a sequence that encodes a matrix metalloproteinase polypeptide having an Accession Number selected from the group consisting of: NM_{—}005940.2, NM_{—}004995.2, NM_{—}003254.1, NM_{—}004530.1, AF219624.1 and W45551_RC; and
(iv) a sequence that is complementary to any one of the sequences set forth in (i) or (ii) or (iii).

In an alternative preferred embodiment, the present invention provides a method of diagnosing a pancreatic cancer in a human or animal subject being tested said method comprising contacting a biological sample from said subject being tested with a nucleic acid probe for a time and under conditions sufficient for hybridization to occur and then detecting the hybridization wherein a modified level of hybridization of the probe for the subject being tested compared to the hybridization obtained for a control subject not having pancreatic cancer indicates that the subject being tested has a pancreatic cancer, and wherein said nucleic acid probe comprises a sequence selected from the group consisting of:

(i) a sequence comprising at least about 20 contiguous nucleotides from nucleic acid encoding a retinoic acid signal transduction or retinoic acid pathway protein and having an Accession Number selected from the group consisting of: NM_{—}003979.2, NM_{—}002888.1, NM_{—}002888.1, NM_{—}005771.1, NM_{—}012420.1, AI806984_RC and BC000069.1;
(ii) a sequence that hybridizes under at least low stringency hybridization conditions to at least about 20 contiguous nucleotides from nucleic acid encoding a retinoic acid signal transduction or retinoic acid pathway protein and having an Accession Number selected from the group consisting of: NM_{—}003979.2, NM_{—}002888.1, NM_{—}002888.1, NM_{—}005771.1, NM_{—}012420.1, AI806984_RC and BC000069.1;
(iii) a sequence that encodes a retinoic acid signal transduction or retinoic acid pathway protein having an Accession Number selected from the group consisting of: NM_{—}003979.2, NM_{—}002888.1, NM_{—}002888.1, NM_{—}005771.1, NM_{—}012420.1, AI806984_RC and BC000069.1; and
(iv) a sequence that is complementary to any one of the sequences set forth in (i) or (ii) or (iii).

Preferably, the retinoic acid signal transduction or retinoic acid pathway protein is a polypeptide encoded by a retinoic acid induced 3 (RAI3) gene as exemplified herein by SEQ ID NO: 10 or a homolog thereof. In accordance with this preferred embodiment, the present invention provides a method of diagnosing a pancreatic cancer in a human or animal subject being tested said method comprising contacting a biological sample from said subject being tested with a nucleic acid probe for a time and under conditions sufficient for hybridization to occur and then detecting the hybridization wherein an enhanced level of hybridization of the probe for the subject being tested compared to the hybridization obtained for a control subject not having pancreatic cancer indicates that the subject being tested has a pancreatic cancer, and wherein said nucleic acid probe comprises a sequence selected from the group consisting of:
(i) a sequence comprising at least about 20 contiguous nucleotides from the sequence set firth in SEQ ID NO: 9;
(ii) a sequence that hybridizes under at least low stringency hybridization conditions to at least about 20 contiguous nucleotides from the sequence set forth in SEQ ID NO: 9;
(iii) a sequence that is at least about 80% identical to the sequence set forth in SEQ ID NO: 9;
(iv) a sequence that encodes the amino acid sequence set forth in SEQ ID NO: 10; and
(v) a sequence that is complementary to any one of the sequences set forth in (i) or (ii) or (iii) or (iv).

In an alternative preferred embodiment, the present invention provides a method of diagnosing a pancreatic cancer in a human or animal subject being tested said method comprising contacting a biological sample from said subject being tested with a nucleic acid probe for a time and under conditions sufficient for hybridization to occur and then detecting the hybridization wherein a modified level of hybridization of the probe for the subject being tested compared to the hybridization obtained for a control subject not having pancreatic cancer indicates that the subject being tested has a pancreatic cancer, and wherein said nucleic acid probe comprises a sequence selected from the group consisting of:

(i) a sequence comprising at least about 20 contiguous nucleotides from nucleic acid encoding a calcium channel protein and having an Accession Number selected from the group consisting of: U07139.1 and NM_{—}005183.1;
(ii) a sequence that hybridizes under at least low stringency hybridization conditions to at least about 20 contiguous nucleotides from nucleic acid encoding a calcium channel protein and having an Accession Number selected from the group consisting of: U07139.1 and NM_{—}005183.1;
(iii) a sequence that encodes a calcium channel protein having an Accession Number selected from the group consisting of: U07139.1 and NM_{—}005183.1; and
(iv) a sequence that is complementary to any one of the sequences set forth in (i) or (ii) or (iii).

In an alternative preferred embodiment, the present invention provides a method of diagnosing a pancreatic cancer in a human or animal subject being tested said method comprising contacting a biological sample from said subject being tested with a nucleic acid probe for a time and under conditions sufficient for hybridization to occur and then detecting the hybridization wherein a modified level of hybridization of the probe for the subject being tested compared to the hybridization obtained for a control subject not having pancreatic cancer indicates that the subject being tested has a pancreatic cancer, and wherein said nucleic acid probe comprises a sequence selected from the group consisting of:

(i) a sequence comprising at least about 20 contiguous nucleotides from nucleic acid encoding a cathepsin polypeptide and having an Accession Number selected from the group consisting of: NM_{—}001910.1, NM_{—}000396.1, W47179_RC, AI246687_RC, AK024855.1, NM_{—}003793.2 and NM_{—}001335.1;
(ii) a sequence that hybridizes under at least low stringency hybridization conditions to at least about 20 contiguous nucleotides from nucleic acid encoding a cathepsin polypeptide and having an Accession Number selected from the group consisting of: NM_{—}001910.1, NM_{—}000396.1, W47179_RC, AI246687_RC, AK024855.1, NM_{—}003793.2 and NM_{—}001335.1;
(iii) a sequence that encodes a cathepsin polypeptide having an Accession Number selected from the group consisting of: NM_{—}001910.1, NM_{—}000396.1, W47179_RC, AI246687_RC, AK024855.1, NM_{—}003793.2 and NM_{—}001335.1; and
(iv) a sequence that is complementary to any one of the sequences set forth in (i) or (ii) or (iii).

In an alternative preferred embodiment, the present invention provides a method of diagnosing a pancreatic cancer in a human or animal subject being tested said method comprising contacting a biological sample from said subject being tested with a nucleic acid probe for a time and under conditions sufficient for hybridization to occur and then detecting the hybridization wherein a modified level of hybridization of the probe for the subject being tested compared to the hybridization obtained for a control subject not having pancreatic cancer indicates that the subject being tested has a pancreatic cancer, and wherein said nucleic acid probe comprises a sequence selected from the group consisting of:

(i) a sequence comprising at least about 20 contiguous nucleotides from nucleic acid encoding a viral oncoprotein homolog and having an Accession Number selected from the group consisting of: NM_{—}005564.1, AI760277_RC, AW592266_RC, M927480_RC, AI356412_RC, NM_{—}005402.1, NM_{—}005402.1, NM_{—}002908.1, NM_{—}002467.1, M19720, NM_{—}002466.1 and NM_{—}000104.2;
(ii) a sequence that hybridizes under at least low stringency hybridization conditions to at least about 20 contiguous nucleotides from nucleic acid encoding a viral oncoprotein homolog and having an Accession Number selected from the group consisting of: NM_{—}005564.1, AI760277_RC, AW592266_RC, AA927480_RC, AI356412_RC, NM_{—}005402.1, NM_{—}005402.1, NM_{—}002908.1, NM_{—}002467.1, M19720, NM_{—}002466.1 and NM_{—}000104.2;
(iii) a sequence that encodes a viral oncoprotein homolog having an Accession Number selected from the group consisting of: NM_{—}005564.1, AI760277_RC, AW592266_RC, AA927480_RC, AI356412_RC, NM_{—}005402.1, NM_{—}005402.1, NM_{—}002908.1, NM_{—}002467.1, M19720, NM_{—}002466.1 and NM_{—}000104.2; and
(iv) a sequence that is complementary to any one of the sequences set forth in (i) or (ii) or (iii).

In an alternative preferred embodiment, the present invention provides a method of diagnosing a pancreatic cancer in a human or animal subject being tested said method comprising contacting a biological sample from said subject being tested with a nucleic acid probe for a time and under conditions sufficient for hybridization to occur and then detecting the hybridization wherein an enhanced level of hybridization of the probe for the subject being tested compared to the hybridization obtained for a control subject not having pancreatic cancer indicates that the subject being tested has a pancreatic cancer, and wherein said nucleic acid probe comprises a sequence selected from the group consisting of:

(i) a sequence comprising at least about 20 contiguous nucleotides from nucleic acid encoding an S100 calcium binding protein and having an Accession Number selected from the group consisting of: NM_{—}005980.1, NM_{—}005978.2, NM_{—}014624.2, NM_{—}005620.1, NM_{—}002966.1, NM_{—}002961.2 and NM_{—}021039.1;
(ii) a sequence that hybridizes under at least low stringency hybridization conditions to at least about 20 contiguous nucleotides from nucleic acid encoding an S100 calcium binding protein and having an Accession Number selected from the group consisting of: NM_{—}005980.1, NM_{—}005978.2, NM_{—}014624.2, NM_{—}005620.1, NM_{—}002966.1, NM_{—}002961.2 and NM_{—}021039.1;
(iii) a sequence that encodes an S100 calcium binding protein having an Accession Number selected from the group consisting of: NM_{—}005980.1, NM_{—}005978.2, NM_{—}014624.2, NM_{—}005620.1, NM_{—}002966.1, NM_{—}002961.2 and NM_{—}021039.1; and
(iv) a sequence that is complementary to any one of the sequences set forth in (i) or (ii) or (iii).

In an alternative preferred embodiment, the present invention provides a method of diagnosing a pancreatic cancer in a human or animal subject being tested said method comprising contacting a biological sample from said subject being tested with a nucleic acid probe for a time and under conditions sufficient for hybridization to occur and then detecting the hybridization wherein a modified level of hybridization of the probe for the subject being tested compared to the hybridization obtained for a control subject not having pancreatic cancer indicates that the subject being tested has a pancreatic cancer, and wherein said nucleic acid probe comprises a sequence selected from the group consisting of:

(i) a sequence comprising at least about 20 contiguous nucleotides from nucleic acid encoding a homeobox protein and having an Accession Number selected from the group consisting of: NM_{—}018952.1, NM_{—}002145.1, AK000445.1, S49765.1 and NM_{—}002144.1;
(ii) a sequence that hybridizes under at least low stringency hybridization conditions to at least about 20 contiguous nucleotides from nucleic acid encoding a homeobox protein and having an Accession Number selected from the group consisting of: NM_{—}018952.1, NM_{—}002145.1, AK000445.1, S49765.1 and NM_{—}002144.1;
(iii) a sequence that encodes a homeobox protein having an Accession Number selected from the group consisting of: NM_{—}018952.1, NM_{—}002145.1, AK000445.1, S49765.1 and NM_{—}002144.1; and
(iv) a sequence that is complementary to any one of the sequences set forth in (i) or (ii) or (iii).

Preferably, the homeobox protein is a homeo box B2 (HOXB2)-encoding nucleic acid exemplified herein by SEQ ID NO: 11 or a homolog thereof. In accordance with this preferred embodiment, the present invention provides a method of diagnosing a pancreatic cancer in a human or animal subject being tested said method comprising contacting a biological sample from said subject being tested with a nucleic acid probe for a time and under conditions sufficient for hybridization to occur and then detecting the hybridization wherein an enhanced level of hybridization of the probe for the subject being tested compared to the hybridization obtained for a control subject not having pancreatic cancer indicates that the subject being tested has a pancreatic cancer, and wherein said nucleic acid probe comprises a sequence selected from the group consisting of:
(i) a sequence comprising at least about 20 contiguous nucleotides from the sequence set firth in SEQ ID NO: 11;
(ii) a sequence that hybridizes under at least low stringency hybridization conditions to at least about 20 contiguous nucleotides from the sequence set forth in SEQ ID NO: 11;
(iii) a sequence that is at least about 80% identical to the sequence set forth in SEQ ID NO: 11;
(iv) a sequence that encodes the amino acid sequence set forth in SEQ ID NO: 12; and
(v) a sequence that is complementary to any one of the sequences set forth in (i) or (ii) or (iii) or (iv).

In an alternative preferred embodiment, the present invention provides a method of diagnosing a pancreatic cancer in a human or animal subject being tested said method comprising contacting a biological sample from said subject being tested with a nucleic acid probe for a time and under conditions sufficient for hybridization to occur and then detecting the hybridization wherein a modified level of hybridization of the probe for the subject being tested compared to the hybridization obtained for a control subject not having pancreatic cancer indicates that the subject being tested has a pancreatic cancer, and wherein said nucleic acid probe comprises a sequence selected from the group consisting of:

(i) a sequence comprising at least about 20 contiguous nucleotides from nucleic acid encoding a zinc finger protein and having an Accession Number selected from the group consisting of: AL567808_RC, NM_{—}006299.1, NM_{—}007150.1, AU150728_RC, NM_{—}003428.1, NM_{—}020657.1, AA121673_RC, NM_{—}006526.1, NM_{—}015871.1, AI493587_RC, NM_{—}006006.1 and NM_{—}006963.1;
(ii) a sequence that hybridizes under at least low stringency hybridization conditions to at least about 20 contiguous nucleotides from nucleic acid encoding a zinc finger protein and having an Accession Number selected from the group consisting of: AL567808_RC, NM_{—}006299.1, NM_{—}007150.1, AU150728_RC, NM_{—}003428.1, NM_{—}020657.1, AA121673_RC, NM_{—}006526.1, NM_{—}015871.1, AI493587_RC, NM_{—}006006.1 and NM_{—}006963.1;
(iii) a sequence that encodes a zinc finger protein having an Accession Number selected from the group consisting of: AL567808_RC, NM_{—}006299.1, NM_{—}007150.1, AU150728_RC, NM_{—}003428.1, NM_{—}020657.1, AA121673_RC, NM_{—}006526.1, NM_{—}015871.1, AI493587_RC, NM_{—}006006.1 and NM_{—}006963.1; and
(iv) a sequence that is complementary to any one of the sequences set forth in (i) or (ii) or (iii).

In an alternative preferred embodiment, the present invention provides a method of diagnosing a pancreatic cancer in a human or animal subject being tested said method comprising contacting a biological sample from said subject being tested with a nucleic acid probe for a time and under conditions sufficient for hybridization to occur and then detecting the hybridization wherein a modified level of hybridization of the probe for the subject being tested compared to the hybridization obtained for a control subject not having pancreatic cancer indicates that the subject being tested has a pancreatic cancer, and wherein said nucleic acid probe comprises a sequence selected from the group consisting of:

(i) a sequence comprising at least about 20 contiguous nucleotides from nucleic acid encoding a heat shock protein and having an Accession Number selected from the group consisting of: NM_{—}004353.1, NM_{—}005346.2, NM_{—}005345.3, R01140_RC, BG403660, BE256479, AB034951.1, NM_{—}016292.1 and AI1393937;
(ii) a sequence that hybridizes under at least low stringency hybridization conditions to at least about 20 contiguous nucleotides from nucleic acid encoding a heat shock protein and having an Accession Number selected from the group consisting of: NM_{—}004353.1, NM_{—}005346.2, NM_{—}005345.3, R01140_RC, BG403660, BE256479, AB034951.1, NM_{—}016292.1 and AI393937;
(iii) a sequence that encodes a heat shock protein having an Accession Number selected from the group consisting of: NM_{—}004353.1, NM_{—}005346.2, NM_{—}005345.3, R01140_RC, BG403660, BE256479, AB034951.1, NM_{—}016292.1 and AI393937; and
(iv) a sequence that is complementary to any one of the sequences set forth in (i) or (ii) or (iii).

As used herein, the term “modified level” includes an enhanced, increased or elevated level of an integer being assayed, or alternatively, a reduced or decreased level of an integer being assayed.

In one embodiment an elevated, enhanced or increased level of expression of the nucleic acid is detected. In an alternative preferred embodiment, a reduced level of a diagnostic marker is indicative of pancreatic cancer.

Those skilled in the art will be aware that as a carcinoma progresses, metastases occur in organs and tissues outside the site of the primary tumor. For example, in the case of pancreatic cancer, metastases may appear in a tissue selected from the group consisting of omentum, abdominal fluid, lymph nodes, lung, liver, brain, and bone. Accordingly, the term “pancreatic cancer” as used herein shall be taken to include an early or developed tumor of the pancreas and optionally, any metastases outside the pancreas that occurs in a subject having a primary tumor of the pancreas.

Preferably, the pancreatic cancer that is diagnosed according to the present invention is an a carcinoma, an adenocarcinoma, and more preferably an epithelial carcinoma, pancreatic exocrine tumour, such as, for example, a ductal adenocarcinoma, or a pancreatic intraepithelial neoplasia.

The present invention encompasses diagnostic/prognostic assays at any stage of disease progression. As used herein, the term “diagnosis”, and variants thereof, such as, but not limited to “diagnose”, “diagnosed” or “diagnosing” shall not be limited to a primary diagnosis of a clinical state, however should be taken to include any primary diagnosis or prognosis of a clinical state. For example, the “diagnostic assay” formats described herein are equally relevant to assessing the remission of a patient, or monitoring disease recurrence, or tumor recurrence, such as following surgery or chemotherapy, or determining the appearance of metastases of a primary tumor. All such uses of the assays described herein are encompassed by the present invention.

Both classical hybridization and amplification formats, and combinations thereof, are encompassed by the invention. In one embodiment, the hybridization comprises performing a nucleic acid hybridization reaction between a labeled probe and a second nucleic acid in the biological sample from the subject being tested, and detecting the label. In another embodiment, the hybridization comprising performing a nucleic acid amplification reaction eg., polymerase chain reaction (PCR), wherein the probe consists of a nucleic acid primer and nucleic acid copies of the nucleic acid in the biological sample are amplified. As will be known to the skilled artisan, amplification may proceed classical nucleic acid hybridization detection systems, to enhance specificity of detection, particularly in the case of less abundant mRNA species in the sample.

In one embodiment, the sample is preferably prepared on a solid matrix e.g., a histology slide or nucleic acid chip or tissue chip. Alternatively, the sample can be solubilized e.g., to produce an extract for hybridization.

Preferably, the subject method further comprises obtaining the sample from a subject. Preferably, the sample has been obtained previously from a subject.

A further aspect of the present invention relates to protein-based or antigen-based or antibody-based methods for diagnosing a pancreatic cancer in a human or other mammal.

Accordingly, in one embodiment, the present invention provides a method of detecting a pancreatic cancer-associated polypeptide in a biological sample the method comprising contacting the biological sample with an antibody that binds specifically to a pancreatic cancer-associated polypeptide in the biological sample, the polypeptide being encoded by a polynucleotide that selectively hybridizes to a sequence at least 80% identical to a sequence as shown in Table 3 or Table 4.

Preferably the percentage identity to a sequence disclosed in any one of Tables 3 or 4 is at least about 85% or 90% or 95%, and still more preferably at least about 98% or 99%.

In a preferred embodiment, the present invention provides a method of diagnosing a pancreatic cancer in a human or animal subject being tested said method comprising contacting a biological sample from said subject being tested with an antibody for a time and under conditions sufficient for an antigen-antibody complex to form and then detecting the complex wherein a modified level of the antigen-antibody complex for the subject being tested compared to the amount of the antigen-antibody complex formed for a control subject not having pancreatic cancer indicates that the subject being tested has a pancreatic cancer, and wherein said antibody binds to a polypeptide comprising an amino acid sequence comprising at least about 10 contiguous amino acid residues of a sequence having at least about 80% identity to a sequence set forth in Table 3 or 4.

Alternatively, or in addition, the present invention provides a method of diagnosing a pancreatic cancer in a human or animal subject being tested said method comprising contacting a biological sample from said subject being tested with an antibody for a time and under conditions sufficient for an antigen-antibody complex to form and then detecting the complex wherein a modified level of the antigen-antibody complex for the subject being tested compared to the amount of the antigen-antibody complex formed for a control subject not having pancreatic cancer indicates that the subject being tested has a pancreatic cancer, and wherein said antibody binds to a polypeptide comprising an amino acid sequence comprising at least about 10 contiguous amino acid residues of a sequence having at least about 80% identity to a sequence having the GenBank Accession No. AF 279145. For the purposes of nomenclature, the amino acid sequence set forth in GenBank Accession No. AF 279145 relates to the homo sapiens tumor endothelial marker TEM8 (i.e., SEQ ID NO: 4).

In a preferred embodiment, the present invention provides a method of diagnosing a pancreatic cancer in a human or animal subject being tested said method comprising contacting a biological sample from said subject being tested with an antibody for a time and under conditions sufficient for an antigen-antibody complex to form and then detecting the complex wherein an enhanced level of the antigen-antibody complex for the subject being tested compared to the amount of the antigen-antibody complex formed for a control subject not having pancreatic cancer indicates that the subject being tested has a pancreatic cancer, and wherein said antibody binds to a polypeptide comprising an amino acid sequence comprising at least about 10 contiguous amino acid residues of a sequence having at least about 80% identity to a sequence selected from the group consisting of SEQ ID NOs: 2, 6, 8, 10 and 12.

In a particularly preferred embodiment, the diagnostic/prognostic assay of the present invention depends upon the use of antibodies that specifically binds to a HOX B2 protein. In accordance with this preferred embodiment, the present invention provides a method of diagnosing a pancreatic cancer in a human or animal subject being tested said method comprising contacting a biological sample from said subject being tested with an antibody for a time and under conditions sufficient for an antigen-antibody complex to form and then detecting the complex wherein an enhanced level of the antigen-antibody complex for the subject being tested compared to the amount of the antigen-antibody complex formed for a control subject not having pancreatic cancer indicates that the subject being tested has a pancreatic cancer, and wherein said antibody binds to a polypeptide comprising an amino acid sequence comprising at least about 10 contiguous amino acid residues of a sequence having at least about 80% identity to the sequence set forth in SEQ ID NO: 12.

In a preferred embodiment, the present invention provides a method of diagnosing a pancreatic cancer in a human or animal subject being tested said method comprising contacting a biological sample from said subject being tested with an antibody for a time and under conditions sufficient for an antigen-antibody complex to form and then detecting the complex wherein a modified level of the antigen-antibody complex for the subject being tested compared to the amount of the antigen-antibody complex formed for a control subject not having pancreatic cancer indicates that the subject being tested has a pancreatic cancer, and wherein said antibody binds to a membrane protein comprising an amino acid sequence having an Accession Number selected from the group consisting of: NM_{—}004363.1, NM_{—}003979.2, NM_{—}004696.1, NM_{—}002888.1, BC005008.1, NM_{—}005672.1, S59049.1, AI631159_RC, NM_{—}004476.1, NM_{—}000227.1, NM_{—}000593.2, NM_{—}013451.1, NM_{—}002888.1, AL162079.1, NM_{—}001945.1, M85289.1, BG170541, NM_{—}002510.1, AV713720, NM_{—}003272.1, NM_{—}004334.1, AI741056_RC, U07139.1, AI356412_RC, AL161958.1, NM_{—}006670.1, NM_{—}003641.1, AF000425.1, NM_{—}012329.1, AW151360_RC, NM_{—}012449.1, NM_{—}003507.1, M81635.1, NM_{—}003332.1, BC000961.2, NM_{—}003174.2, NM_{—}001663.2, NM_{—}001904.1, M76446.1, NM_{—}002231.2, U45448.1, NM_{—}001502.1, NM_{—}001169.1 and NM_{—}016295.1.

Preferably, the amount of the antigen-antibody complex formed is enhanced in the subject sample relative to the control sample, and the membrane protein is selected from the group consisting of selected from the group consisting of a type II membrane serine protease (TMPRSS4) exemplified herein by SEQ ID NO: 8, a homolog of a type II membrane serine protease (TMPRSS4) exemplified herein by SEQ ID NO: 8, a polypeptide encoded by a retinoic acid induced 3 (RAI3) gene as exemplified herein by SEQ ID NO: 10 and a homolog of a polypeptide encoded by a retinoic acid induced 3 (RAI3) gene as exemplified herein by SEQ ID NO: 10.

In an alternative preferred embodiment, the present invention provides a method of diagnosing a pancreatic cancer in a human or animal subject being tested said method comprising contacting a biological sample from said subject being tested with an antibody for a time and under conditions sufficient for an antigen-antibody complex to form and then detecting the complex wherein a modified level of the antigen-antibody complex for the subject being tested compared to the amount of the antigen-antibody complex formed for a control subject not having pancreatic cancer indicates that the subject being tested has a pancreatic cancer, and wherein said antibody binds to an extracellular protein comprising an amino acid sequence having an Accession Number selected from the group consisting of: NM_{—}004591.1, M13436.1, M31159.1, NM_{—}005940.2, X02761.1, BF590263_RC, BF218922, NM_{—}000095.1, NM_{—}000584.1, BC002710.1, AF154054.1, NM_{—}003247.1, NM_{—}002160.1, NM_{—}006533.1, NM_{—}002546.1, NM_{—}013372.1, NM_{—}004385.1, NM_{—}003118.1, NM_{—}003014.2, NM_{—}001945.1, M85289.1, NM_{—}000138.1, NM_{—}005567.2, NM_{—}002090.1, NM_{—}013253.1, NM_{—}012445.1, NM_{—}002933.1, BF508685_RC and NM_{—}006229.1. Antigen-based diagnostic/prognostic assays, including multiplex assays or multi-analyte tests, of levels of extracellular proteins (and/or secreted proteins) are particularly amenable to detection in bodily fluids such as, for example, urine, ascites, whole blood, serum, peripheral blood mononuclear cells (PBMC) or a buffy coat fraction. Accordingly, such assay targets are particularly preferred for non-invasive diagnostic or prognostic assays, in addition to being useful for the immunohistochemical approaches by which membrane-localized proteins, intracellular proteins, organellar proteins or nuclear proteins are assayed.

In an alternative preferred embodiment, the present invention provides a method of diagnosing a pancreatic cancer in a human or animal subject being tested said method comprising contacting a biological sample from said subject being tested with an antibody for a time and under conditions sufficient for an antigen-antibody complex to form and then detecting the complex wherein an enhanced level of the antigen-antibody complex for the subject being tested compared to the amount of the antigen-antibody complex formed for a control subject not having pancreatic cancer indicates that the subject being tested has a pancreatic cancer, and wherein said antibody binds to a protein of the TGF-β signalling pathway having an Accession Number selected from the group consisting of: M13436.1, AF288571.1, BC002704.1, U44378.1 and NM_{—}001904.1.

In an alternative preferred embodiment, the present invention provides a method of diagnosing a pancreatic cancer in a human or animal subject being tested said method comprising contacting a biological sample from said subject being tested with an antibody for a time and under conditions sufficient for an antigen-antibody complex to form and then detecting the complex wherein a modified level of the antigen-antibody complex for the subject being tested compared to the amount of the antigen-antibody complex formed for a control subject not having pancreatic cancer indicates that the subject being tested has a pancreatic cancer, and wherein said antibody binds to a WNT signalling pathway protein having an Accession Number selected from the group consisting of: NM_{—}003014.2, AF311912.1, AF143679.1, NM_{—}013253.1, L37882.1, NM_{—}003882.1, U91903.1, NM_{—}003507.1, NM_{—}030775.1, NM_{—}001904.1 and NM_{—}013266.1.

In an alternative preferred embodiment, the present invention provides a method of diagnosing a pancreatic cancer in a human or animal subject being tested said method comprising contacting a biological sample from said subject being tested with an antibody for a time and under conditions sufficient for an antigen-antibody complex to form and then detecting the complex wherein an enhanced level of the antigen-antibody complex for the subject being tested compared to the amount of the antigen-antibody complex formed for a control subject not having pancreatic cancer indicates that the subject being tested has a pancreatic cancer, and wherein said antibody binds to a protein of nucleotide metabolism having an Accession Number selected from the group consisting of: BE971383 and NM_{—}002970.1.

In an alternative preferred embodiment, the present invention provides a method of diagnosing a pancreatic cancer in a human or animal subject being tested said method comprising contacting a biological sample from said subject being tested with an antibody for a time and under conditions sufficient for an antigen-antibody complex to form and then detecting the complex wherein an enhanced level of the antigen-antibody complex for the subject being tested compared to the amount of the antigen-antibody complex formed for a control subject not having pancreatic cancer indicates that the subject being tested has a pancreatic cancer, and wherein said antibody binds to a protein involved in smooth muscle contraction having an Accession Number selected from the group consisting of: NM_{—}005965.1, NM_{—}006097.1, NM_{—}001613.1 and AI082078_RC.

In an alternative preferred embodiment, the present invention provides a method of diagnosing a pancreatic cancer in a human or animal subject being tested said method comprising contacting a biological sample from said subject being tested with an antibody for a time and under conditions sufficient for an antigen-antibody complex to form and then detecting the complex wherein a modified level of the antigen-antibody complex for the subject being tested compared to the amount of the antigen-antibody complex formed for a control subject not having pancreatic cancer indicates that the subject being tested has a pancreatic cancer, and wherein said antibody binds to a mitochondrial protein having an Accession Number selected from the group consisting of: NM_{—}000104.2, NM_{—}002064.1, NM_{—}000784.1, NM_{—}003359.1, R92925_RC, NM_{—}004294.1, T67741_RC and NM_{—}001914.1.

In an alternative preferred embodiment, the present invention provides a method of diagnosing a pancreatic cancer in a human or animal subject being tested said method comprising contacting a biological sample from said subject being tested with an antibody for a time and under conditions sufficient for an antigen-antibody complex to form and then detecting the complex wherein an enhanced level of the antigen-antibody complex for the subject being tested compared to the amount of the antigen-antibody complex formed for a control subject not having pancreatic cancer indicates that the subject being tested has a pancreatic cancer, and wherein said antibody binds to a collagen, a protein of collagen synthesis or a fibrillin having an Accession Number selected from the group consisting of: NM_{—}002593.2, NM_{—}001854.1, AL575735_RC, AI983428_RC, NM_{—}000138.1, X05610.1, NM_{—}000089.1, AI743621_RC and AU144167_RC.

In an alternative preferred embodiment, the present invention provides a method of diagnosing a pancreatic cancer in a human or animal subject being tested said method comprising contacting a biological sample from said subject being tested with an antibody for a time and under conditions sufficient for an antigen-antibody complex to form and then detecting the complex wherein an enhanced level of the antigen-antibody complex for the subject being tested compared to the amount of the antigen-antibody complex formed for a control subject not having pancreatic cancer indicates that the subject being tested has a pancreatic cancer, and wherein said antibody binds to an inflammatory response pathway protein having an Accession Number selected from the group consisting of: NM_{—}000089.1, BC005858.1, X02761.1, AK026737.1, NM_{—}005562.1, AI743621_RC and AU144167_RC.

In an alternative preferred embodiment, the present invention provides a method of diagnosing a pancreatic cancer in a human or animal subject being tested said method comprising contacting a biological sample from said subject being tested with an antibody for a time and under conditions sufficient for an antigen-antibody complex to form and then detecting the complex wherein an enhanced level of the antigen-antibody complex for the subject being tested compared to the amount of the antigen-antibody complex formed for a control subject not having pancreatic cancer indicates that the subject being tested has a pancreatic cancer, and wherein said antibody binds to an endoplasmic reticulum (ER) protein having an Accession Number selected from the group consisting of: NM_{—}004353.1, AV691323, BC000961.2, NM_{—}000961.1 and AI753659_RC.

In an alternative preferred embodiment, the present invention provides a method of diagnosing a pancreatic cancer in a human or animal subject being tested said method comprising contacting a biological sample from said subject being tested with an antibody for a time and under conditions sufficient for an antigen-antibody complex to form and then detecting the complex wherein a modified level of the antigen-antibody complex for the subject being tested compared to the amount of the antigen-antibody complex formed for a control subject not having pancreatic cancer indicates that the subject being tested has a pancreatic cancer, and wherein said antibody binds to an apoptotic protein having an Accession Number selected from the group consisting of: NM_{—}000546.2 and AF201370.1.

In an alternative preferred embodiment, the present invention provides a method of diagnosing a pancreatic cancer in a human or animal subject being tested said method comprising contacting a biological sample from said subject being tested with an antibody for a time and under conditions sufficient for an antigen-antibody complex to form and then detecting the complex wherein a modified level of the antigen-antibody complex for the subject being tested compared to the amount of the antigen-antibody complex formed for a control subject not having pancreatic cancer indicates that the subject being tested has a pancreatic cancer, and wherein said antibody binds to a G1/S phase cell cycle control protein having an Accession Number selected from the group consisting of: NM_{—}001237.1, NM_{—}000546.2, NM_{—}003674.1, BE407516, R78668_RC, NM_{—}000077.1, BC000076.1 and NM_{—}000389.1.

In an alternative preferred embodiment, the present invention provides a method of diagnosing a pancreatic cancer in a human or animal subject being tested said method comprising contacting a biological sample from said subject being tested with an antibody for a time and under conditions sufficient for an antigen-antibody complex to form and then detecting the complex wherein a modified level of the antigen-antibody complex for the subject being tested compared to the amount of the antigen-antibody complex formed for a control subject not having pancreatic cancer indicates that the subject being tested has a pancreatic cancer, and wherein said antibody binds to a matrix metalloproteinase polypeptide having an Accession Number selected from the group consisting of: NM_{—}005940.2, NM_{—}004995.2, NM_{—}003254.1, NM_{—}004530.1, AF219624.1 and W45551_RC.

In an alternative preferred embodiment, the present invention provides a method of diagnosing a pancreatic cancer in a human or animal subject being tested said method comprising contacting a biological sample from said subject being tested with an antibody for a time and under conditions sufficient for an antigen-antibody complex to form and then detecting the complex wherein a modified level of the antigen-antibody complex for the subject being tested compared to the amount of the antigen-antibody complex formed for a control subject not having pancreatic cancer indicates that the subject being tested has a pancreatic cancer, and wherein said antibody binds to a retinoic acid signal transduction or retinoic acid pathway protein having an Accession Number selected from the group consisting of: NM_{—}003979.2, NM_{—}002888.1, NM_{—}002888.1, NM_{—}005771.1, NM_{—}012420.1, AI806984_RC and BC000069.1.

Preferably, the amount of the antigen-antibody complex for the subject being tested is enhanced compared to the amount of the antigen-antibody complex formed for a control subject not having pancreatic cancer and the retinoic acid signal transduction or retinoic acid pathway protein is a polypeptide encoded by a retinoic acid induced 3 (RAI3) gene as exemplified herein by SEQ ID NO: 10 or a homolog thereof.

In an alternative preferred embodiment, the present invention provides a method of diagnosing a pancreatic cancer in a human or animal subject being tested said method comprising contacting a biological sample from said subject being tested with an antibody for a time and under conditions sufficient for an antigen-antibody complex to form and then detecting the complex wherein a modified level of the antigen-antibody complex for the subject being tested compared to the amount of the antigen-antibody complex formed for a control subject not having pancreatic cancer indicates that the subject being tested has a pancreatic cancer, and wherein said antibody binds to a calcium channel protein having an Accession Number selected from the group consisting of: U07139.1 and NM_{—}005183.1.

In an alternative preferred embodiment, the present invention provides a method of diagnosing a pancreatic cancer in a human or animal subject being tested said method comprising contacting a biological sample from said subject being tested with an antibody for a time and under conditions sufficient for an antigen-antibody complex to form and then detecting the complex wherein a modified level of the antigen-antibody complex for the subject being tested compared to the amount of the antigen-antibody complex formed for a control subject not having pancreatic cancer indicates that the subject being tested has a pancreatic cancer, and wherein said antibody binds to a cathepsin polypeptide having an Accession Number selected from the group consisting of: NM_{—}001910.1, NM_{—}000396.1, W47179_RC, AI246687_RC, AK024855.1, NM_{—}003793.2 and NM_{—}001335.1.

In an alternative preferred embodiment, the present invention provides a method of diagnosing a pancreatic cancer in a human or animal subject being tested said method comprising contacting a biological sample from said subject being tested with an antibody for a time and under conditions sufficient for an antigen-antibody complex to form and then detecting the complex wherein a modified level of the antigen-antibody complex for the subject being tested compared to the amount of the antigen-antibody complex formed for a control subject not having pancreatic cancer indicates that the subject being tested has a pancreatic cancer, and wherein said antibody binds to a viral oncoprotein homolog having an Accession Number selected from the group consisting of: NM_{—}005564.1, AI760277_RC, AW592266_RC, AA927480_RC, AI356412_RC, NM_{—}005402.1, NM_{—}005402.1, NM_{—}002908.1, NM_{—}002467.1, M19720, NM_{—}002466.1 and NM_{—}000104.2.

In an alternative preferred embodiment, the present invention provides a method of diagnosing a pancreatic cancer in a human or animal subject being tested said method comprising contacting a biological sample from said subject being tested with an antibody for a time and under conditions sufficient for an antigen-antibody complex to form and then detecting the complex wherein an enhanced level of the antigen-antibody complex for the subject being tested compared to the amount of the antigen-antibody complex formed for a control subject not having pancreatic cancer indicates that the subject being tested has a pancreatic cancer, and wherein said antibody binds to an S100 calcium binding protein having an Accession Number selected from the group consisting of: NM_{—}005980.1, NM_{—}005978.2, NM_{—}014624.2, NM_{—}005620.1, NM_{—}002966.1, NM_{—}002961.2 and NM_{—}021039.1.

In an alternative preferred embodiment, the present invention provides a method of diagnosing a pancreatic cancer in a human or animal subject being tested said method comprising contacting a biological sample from said subject being tested with an antibody for a time and under conditions sufficient for an antigen-antibody complex to form and then detecting the complex wherein a modified level of the antigen-antibody complex for the subject being tested compared to the amount of the antigen-antibody complex formed for a control subject not having pancreatic cancer indicates that the subject being tested has a pancreatic cancer, and wherein said antibody binds to a homeobox protein having an Accession Number selected from the group consisting of: NM_{—}018952.1, NM_{—}002145.1, AK000445.1, S49765.1 and NM_{—}002144.1.

Preferably, the amount of the antigen-antibody complex for the subject being tested is enhanced compared to the amount of the antigen-antibody complex formed for a control subject not having pancreatic cancer and the homeobox protein is a homeo box B2 (HOXB2) protein exemplified herein by SEQ ID NO: 12 or a homolog thereof.

In an alternative preferred embodiment, the present invention provides a method of diagnosing a pancreatic cancer in a human or animal subject being tested said method comprising contacting a biological sample from said subject being tested with an antibody for a time and under conditions sufficient for an antigen-antibody complex to form and then detecting the complex wherein a modified level of the antigen-antibody complex for the subject being tested compared to the amount of the antigen-antibody complex formed for a control subject not having pancreatic cancer indicates that the subject being tested has a pancreatic cancer, and wherein said antibody binds to a zinc finger protein having an Accession Number selected from the group consisting of: AL567808_RC, NM_{—}006299.1, NM_{—}007150.1, AU150728_RC, NM_{—}003428.1, NM_{—}020657.1, AA121673_RC, NM_{—}006526.1, NM_{—}015871.1, AI493587_RC, NM_{—}006006.1 and NM_{—}006963.1.

In an alternative preferred embodiment, the present invention provides a method of diagnosing a pancreatic cancer in a human or animal subject being tested said method comprising contacting a biological sample from said subject being tested with an antibody for a time and under conditions sufficient for an antigen-antibody complex to form and then detecting the complex wherein a modified level of the antigen-antibody complex for the subject being tested compared to the amount of the antigen-antibody complex formed for a control subject not having pancreatic cancer indicates that the subject being tested has a pancreatic cancer, and wherein said antibody binds to a heat shock protein having an Accession Number selected from the group consisting of: NM_{—}004353.1, NM_{—}005346.2, NM_{—}005345.3, R01140_RC, BG403660, BE256479, AB034951.1, NM_{—}016292.1 and AI393937.

In one embodiment an elevated, enhanced or increased level of expression of the antigen-antibody complex is detected.

In an alternative preferred embodiment, a reduced level of a diagnostic marker is indicative of pancreatic cancer.

In a further related embodiment, the present invention provides a method of detecting a pancreatic cancer-associated antibody in a biological sample the method comprising contacting the biological sample with a polypeptide encoded by a polynucleotide that selectively hybridizes to a sequence at least 80% identical to a sequence as shown in Tables 3 or 4, wherein the polypeptide specifically binds to the pancreatic cancer-associated antibody.

The sample is preferably prepared on a solid matrix e.g., a histology slide or protein chip or antibody chip or tissue chip. Alternatively, the sample can be solubilized e.g., to produce an extract for immunoassay purposes.

Preferably, the subject method further comprises obtaining the sample from a subject. Preferably, the sample has been obtained previously from a subject.

In accordance with any one or more of the above methods, the biological sample can be contacted with a plurality of Nucleic acids, polypeptides or antibodies. Accordingly, a further aspect of the present invention provides multiplex assays or multianalyte tests for diagnosing pancreatic cancer in a human or animal subject. Such multiplex assays or multi-analyte tests are preferably antigen-based or nucleic acid based assays. Antibody-based assay methods are not to be excluded.

In a preferred embodiment, the present invention provides a nucleic acid-based multiplex assay for diagnosing a pancreatic cancer. In one embodiment, the invention provides a method of diagnosing pancreatic cancer, said method comprising contacting a biological sample from said subject being tested with at least two a nucleic acid probes for a time and under conditions sufficient for hybridization to occur and then detecting the hybridization wherein a modified level of hybridization for the subject being tested compared to the hybridization for a control subject not having pancreatic cancer indicates that the subject being tested has a pancreatic cancer, and wherein one nucleic acid probe comprises a nucleotide sequence selected from the group consisting of:
(i) a sequence comprising at least about 20 contiguous nucleotides from SEQ ID NO: 11;
(ii) a sequence that hybridizes under at least low stringency hybridization conditions to at least about 20 contiguous nucleotides from SEQ ID NO: 11;
(iii) a sequence that is at least about 80% identical to SEQ ID NO: 11;
(iv) a sequence that encodes the amino acid sequence set forth in SEQ ID NO: 12; and
(v) a sequence that is complementary to any one of the sequences set forth in (i) or (ii) or (iii) or (iv)
   and wherein the hybridization for the sequence set forth in any one of (i) to (v) is enhanced for the subject being tested compared to the hybridization for a sample from a control subject not having pancreatic cancer.

Preferably, another probe comprises a nucleotide sequence selected from the group consisting of:
(i) a sequence comprising at least about 20 contiguous nucleotides from a sequence selected from the group consisting of: SEQ ID NO: 1, SEQ ID NO: 3, SEQ ID NO: 5, SEQ ID NO: 7 and SEQ ID NO: 9;
(ii) a sequence that hybridizes under at least low stringency hybridization conditions to at least about 20 contiguous nucleotides from a sequence selected from the group consisting of: SEQ ID NO: 1, SEQ ID NO: 3, SEQ ID NO: 5, SEQ ID NO: 7 and SEQ ID NO:9;
(iii) a sequence that is at least about 80% identical to a sequence selected from the group consisting of: SEQ ID NO: 1, SEQ ID NO: 3, SEQ ID NO: 5, SEQ ID NO: 7 and SEQ ID NO: 9;
(iv) a sequence that encodes an amino acid sequence selected from the group consisting of: SEQ ID NO: 2, SEQ ID NO: 4, SEQ ID NO: 6, SEQ ID NO: 8 and SEQ ID NO: 10; and
(v) a sequence that is complementary to any one of the sequences set forth in (i) or (ii) or (iii) or (iv).

More preferably, the level of hybridization for the other probe is also enhanced for the subject being tested is enhanced compared to the hybridization for a sample from a control subject not having pancreatic cancer and

In an alternative preferred embodiment, the present invention provides an antibody-based multiplex assay or multi-analyte test for diagnosing a pancreatic cancer. In one embodiment, the invention provides a method of diagnosing a pancreatic cancer, said method comprising contacting a biological sample from said subject being tested with at least two antibodies for a time and under conditions sufficient for antigen-antibody complexes to form and then detecting the complexes wherein a modified level of the antigen-antibody complexes for the subject being tested compared to the amount of the antigen-antibody complexes formed for a control subject not having pancreatic cancer indicates that the subject being tested has a pancreatic cancer, and wherein one antibody binds to a HOX B2 polypeptide comprising the amino acid sequence set forth in SEQ ID NO: 12 and wherein the level of antigen-antibody complex formed using the antibody that binds to HOX B2 is enhanced for the subject being tested compared to the sample from a control subject not having pancreatic cancer.

Preferably, another antibody binds to a polypeptide comprising an amino acid sequence selected from the group consisting of: SEQ ID NO: 2, SEQ ID NO: 4, SEQ ID NO: 6, SEQ ID NO: 8 and SEQ ID NO: 10. More preferably, the level of antigen-antibody complex formed using the antibody that binds to any one of SEQ ID Nos: 2 or 4 or 6 or 8 or 10 is enhanced for the subject being tested compared to the sample from a control subject not having pancreatic cancer.

The sample is preferably prepared on a solid matrix e.g., a histology slide or protein chip or antibody chip or nucleic acid chip or tissue chip. Alternatively, the sample can be solubilized e.g., to produce an extract for hybridization or immunoassay purposes.

Preferably, the subject method further comprises obtaining the sample from a subject. Preferably, the sample has been obtained previously from a subject.

A further aspect of the present invention provides methods for determining the likelihood of a subject having pancreatic cancer surviving in the short-medium term, and for determining the suitability of a subject having pancreatic cancer for surgical resection therapy.

More particularly, the inventors sought to determine whether any correlation exists between the expression of any particular gene in a subject having pancreatic cancer and the survival, or likelihood for survival, of the subject during the medium to long term (i.e. in the period between about 1-2 years from primary diagnosis, or longer) compared to the short term survival (i.e., in the period up to about 6 months to 1 year from primary diagnosis). As exemplified herein, the present inventors have determined that elevated expression of the homeobox protein B2 (HOX B2) is correlated with a poor prognosis of survival into the medium-long term, and that normal or low or reduced levels of HOX B2 expression, optionally coupled with surgical resection therapy, are correlated to an improved likelihood for survival into the medium or long term. The data provided herein further suggest that a subject having a level of HOX B2 expression that is not elevated compared to the level in a sample from a healthy or normal control subject has an enhanced likelihood of surviving into the medium or long term, or enhanced life expectancy into the medium or long term following surgical resection, compared to a subject having an elevated HOX B2 expression level.

Accordingly, one embodiment of the present invention provides a method of determining the likelihood that a subject having a pancreatic cancer will survive, said method comprising performing a nucleic acid-based assay supra to thereby determine the level of nucleic acid encoding a HOX B2 protein wherein an elevated level of said nucleic acid encoding a HOX B2 protein compared to the level in a comparable sample from a healthy or normal subject indicates that the subject is unlikely to survive into the medium or short term.

In a related embodiment, the present invention provides a method of determining the likelihood that a subject having a pancreatic cancer will survive, said method comprising performing a nucleic acid-based assay supra to thereby determine the level of nucleic acid encoding a HOX B2 protein wherein a normal level of said nucleic acid encoding a HOX B2 protein compared to the level in a comparable sample from a healthy or normal subject indicates that the subject is likely to survive into the medium or short term.

In a preferred embodiment, the present invention provides a method of determining the likelihood that a subject having a pancreatic cancer will survive, said method comprising contacting a biological sample from said subject being tested with a nucleic acid probe for a time and under conditions sufficient for hybridization to occur and then detecting the hybridization wherein an elevated level of hybridization of the probe for the subject being tested compared to the hybridization obtained for a control subject not having pancreatic cancer indicates that the subject being tested has a poor prognosis for survival e.g., into the medium or long term, and wherein said nucleic acid probe comprises a sequence selected from the group consisting of:
(i) a sequence comprising at least about 20 contiguous nucleotides from SEQ ID NO: 11;
(ii) a sequence that hybridizes under at least low stringency hybridization conditions to at least about 20 contiguous nucleotides from SEQ ID NO: 11;
(iii) a sequence that is at least about 80% identical to SEQ ID NO: 11;
(iv) a sequence that encodes the amino acid sequence set forth in SEQ ID NO: 12; and
(v) a sequence that is complementary to any one of the sequences set forth in (i) or (ii) or (iii) or (iv).

In a related embodiment, the present invention provides a method of determining the likelihood that a subject having a pancreatic cancer will survive, said method comprising contacting a biological sample from said subject being tested with a nucleic acid probe for a time and under conditions sufficient for hybridization to occur and then detecting the hybridization wherein a level of hybridization of the probe for the subject being tested that is similar to the hybridization obtained for a control subject not having pancreatic cancer indicates that the subject being tested has a good prognosis for survival e.g., into the medium or long term, and wherein said nucleic acid probe comprises a sequence selected from the group consisting of:
(i) a sequence comprising at least about 20 contiguous nucleotides from SEQ ID NO: 11;
(ii) a sequence that hybridizes under at least low stringency hybridization conditions to at least about 20 contiguous nucleotides from SEQ ID NO: 11;
(iii) a sequence that is at least about 80% identical to SEQ ID NO: 11;
(iv) a sequence that encodes the amino acid sequence set forth in SEQ ID NO: 12; and
(v) a sequence that is complementary to any one of the sequences set forth in (i) or (ii) or (iii) or (iv).

In a further embodiment, the present invention provides a method of determining the suitability of a subject having a pancreatic cancer for surgical resection therapy, said method comprising contacting a biological sample from said subject being tested with a nucleic acid probe for a time and under conditions sufficient for hybridization to occur and then detecting the hybridization wherein am elevated level of hybridization of the probe for the subject being tested compared to the hybridization obtained for a control subject not having pancreatic cancer indicates that the subject being tested is unsuitable for surgical resection therapy, and wherein said nucleic acid probe comprises a sequence selected from the group consisting of:
(i) a sequence comprising at least about 20 contiguous nucleotides from SEQ ID NO: 11;
(ii) a sequence that hybridizes under at least low stringency hybridization conditions to at least about 20 contiguous nucleotides from SEQ ID NO: 11;
(iii) a sequence that is at least about 80% identical to SEQ ID NO: 11;
(iv) a sequence that encodes the amino acid sequence set forth in SEQ ID NO: 12; and
(v) a sequence that is complementary to any one of the sequences set forth in (i) or (ii) or (iii) or (iv).

In a related embodiment, the present invention provides a method of determining the suitability of a subject having a pancreatic cancer for surgical resection therapy, said method comprising contacting a biological sample from said subject being tested with a nucleic acid probe for a time and under conditions sufficient for hybridization to occur and then detecting the hybridization wherein a level of hybridization of the probe for the subject being tested that is similar to the hybridization obtained for a control subject not having pancreatic cancer indicates that the subject being tested is suitable for surgical resection therapy, and wherein said nucleic acid probe comprises a sequence selected from the group consisting of:
(i) a sequence comprising at least about 20 contiguous nucleotides from SEQ ID NO: 11;
(ii) a sequence that hybridizes under at least low stringency hybridization conditions to at least about 20 contiguous nucleotides from SEQ ID NO: 11;
(iii) a sequence that is at least about 80% identical to SEQ ID NO: 11;
(iv) a sequence that encodes the amino acid sequence set forth in SEQ ID NO: 12; and
(v) a sequence that is complementary to any one of the sequences set forth in (i) or (ii) or (iii) or (iv).

In a further embodiment, the present invention provides a method of determining the likelihood that a subject having a pancreatic cancer will survive, said method comprising contacting a biological sample from said subject being tested with an antibody for a time and under conditions sufficient for an antigen-antibody complex to form and then detecting the complex wherein an enhanced level of the antigen-antibody complex for the subject being tested compared to the amount of the antigen-antibody complex formed for a control subject not having pancreatic cancer indicates that the subject being tested has a poor prognosis for survival e.g., into the medium or long term, and wherein said antibody binds to a polypeptide comprising an amino acid sequence comprising at least about 10 contiguous amino acid residues of a sequence having at least about 80% identity to the sequence set forth in SEQ ID NO: 12.

In a related embodiment, the present invention provides a method of determining the likelihood that a subject having a pancreatic cancer will survive, said method comprising contacting a biological sample from said subject being tested with an antibody for a time and under conditions sufficient for an antigen-antibody complex to form and then detecting the complex wherein a similar level of the antigen-antibody complex for the subject being tested compared to the amount of the antigen-antibody complex formed for a control subject not having pancreatic cancer indicates that the subject being tested has a good prognosis for survival e.g., into the medium or long term, and wherein said antibody binds to a polypeptide comprising an amino acid sequence comprising at least about 10 contiguous amino acid residues of a sequence having at least about 80% identity to the sequence set forth in SEQ ID NO: 12.

In a further embodiment, the present invention provides a method of determining the suitability of a subject having a pancreatic cancer for surgical resection therapy, said method comprising contacting a biological sample from said subject being tested with an antibody for a time and under conditions sufficient for an antigen-antibody complex to form and then detecting the complex wherein an enhanced level of the antigen-antibody complex for the subject being tested compared to the amount of the antigen-antibody complex formed for a control subject not having pancreatic cancer indicates that the subject being tested is unsuitable for surgical resection therapy, and wherein said antibody binds to a polypeptide comprising an amino acid sequence comprising at least about 10 contiguous amino acid residues of a sequence having at least about 80% identity to the sequence set forth in SEQ ID NO: 12.

In a related embodiment, the present invention provides a method of determining the suitability of a subject having a pancreatic cancer for surgical resection therapy, said method comprising contacting a biological sample from said subject being tested with an antibody for a time and under conditions sufficient for an antigen-antibody complex to form and then detecting the complex wherein a similar level of the antigen-antibody complex for the subject being tested compared to the amount of the antigen-antibody complex formed for a control subject not having pancreatic cancer indicates that the subject being tested is suitable for surgical resection therapy, and wherein said antibody binds to a polypeptide comprising an amino acid sequence comprising at least about 10 contiguous amino acid residues of a sequence having at least about 80% identity to the sequence set forth in SEQ ID NO: 12.

The multi-analyte assays described supra are also adaptable to the prognostic assays described in the preceding paragraphs without undue experimentation. Accordingly, one embodiment of the present invention provides a nucleic acid-based multiplex prognostic assay for determining the likelihood of survival from a pancreatic cancer or determining the suitability of a subject having a pancreatic cancer for surgical resection therapy. In accordance with this embodiment, the invention provides a method for determining the likelihood that a subject having pancreatic cancer will survive or from a pancreatic cancer or the suitability of said subject for surgical resection, said method comprising contacting a biological sample from said subject being tested with at least two a nucleic acid probes for a time and under conditions sufficient for hybridization to occur and then detecting the hybridization wherein a modified level of hybridization for the subject being tested compared to the hybridization for a control subject not having pancreatic cancer indicates that the subject being tested has a poor prognosis or survival and/or is a poor candidate for surgical resection therapy, and wherein one nucleic acid probe comprises a nucleotide sequence selected from the group consisting of:
(i) a sequence comprising at least about 20 contiguous nucleotides from SEQ ID NO: 11;
(ii) a sequence that hybridizes under at least low stringency hybridization conditions to at least about 20 contiguous nucleotides from SEQ ID NO: 11;
(iii) a sequence that is at least about 80% identical to SEQ ID NO: 11;
(iv) a sequence that encodes the amino acid sequence set forth in SEQ ID NO: 12; and
(v) a sequence that is complementary to any one of the sequences set forth in (i) or (ii) or (iii) or (iv)
   and wherein the hybridization for the sequence set forth in any one of (i) to (v) is enhanced for the subject being tested compared to the hybridization for a sample from a control subject not having pancreatic cancer.

In a related embodiment, the present invention provides a method for determining the likelihood that a subject having pancreatic cancer will survive from a pancreatic cancer or the suitability of said subject for surgical resection, said method comprising contacting a biological sample from said subject being tested with at least two a nucleic acid probes for a time and under conditions sufficient for hybridization to occur and then detecting the hybridization wherein a level of hybridization for the subject being tested that is similar to the hybridization for a control subject not having pancreatic cancer indicates that the subject being tested has a good prognosis or survival and/or is a suitable candidate for surgical resection therapy, and wherein one nucleic acid probe comprises a nucleotide sequence selected from the group consisting of:
(i) a sequence comprising at least about 20 contiguous nucleotides from SEQ ID NO: 11;
(ii) a sequence that hybridizes under at least low stringency hybridization conditions to at least about 20 contiguous nucleotides from SEQ ID NO: 11;
(iii) a sequence that is at least about 80% identical to SEQ ID NO: 11;
(iv) a sequence that encodes the amino acid sequence set forth in SEQ ID NO: 12; and
(v) a sequence that is complementary to any one of the sequences set forth in (i) or (ii) or (iii) or (iv).

In an alternative preferred embodiment, the present invention provides an antibody-based multiplex assay or multi-analyte test for determining the likelihood of survival from a pancreatic cancer or suitability for surgical resection. In one embodiment, the invention provides a method for determining the likelihood that a subject having pancreatic cancer will survive from a pancreatic cancer or the suitability of said subject for surgical resection, said method comprising contacting a biological sample from said subject being tested with at least two antibodies for a time and under conditions sufficient for antigen-antibody complexes to form and then detecting the complexes wherein a modified level of the antigen-antibody complexes for the subject being tested compared to the amount of the antigen-antibody complexes formed for a control subject not having pancreatic cancer indicates that the subject being tested has a poor prognosis or survival and/or is a poor candidate for surgical resection therapy, and wherein one antibody binds to a HOX B2 polypeptide comprising the amino acid sequence set forth in SEQ ID NO: 12 and wherein the level of antigen-antibody complex formed using the antibody that binds to HOX B2 is enhanced for the subject being tested compared to the sample from a control subject not having pancreatic cancer.

In a related embodiment, the invention provides a method for determining the likelihood that a subject having pancreatic cancer will survive from a pancreatic cancer or the suitability of said subject for surgical resection, said method comprising contacting a biological sample from said subject being tested with at least two antibodies for a time and under conditions sufficient for antigen-antibody complexes to form and then detecting the complexes wherein a level of the antigen-antibody complexes for the subject being tested that is similar to the amount of the antigen-antibody complexes formed for a control subject not having pancreatic cancer indicates that the subject being tested has a good prognosis or survival and/or is a suitable candidate for surgical resection therapy, and wherein one antibody binds to a HOX B2 polypeptide comprising the amino acid sequence set forth in SEQ ID NO: 12

In performing the various diagnostic and prognostic assays of the present invention, it is within the scope of the invention to use a wide variety of biological samples and the invention is not to be limited by the source or nature of the biological sample. In one embodiment, the biological sample is from a patient undergoing a therapeutic regimen to treat pancreatic cancer. In an alternative preferred embodiment, the biological sample is from a patient suspected of having pancreatic cancer.

The sample is preferably prepared on a solid matrix e.g., a histology slide or protein chip or antibody chip or nucleic acid chip or tissue chip. Alternatively, the sample can be solubilized e.g., to produce an extract for hybridization or immunoassay purposes.

Preferably, the subject method further comprises obtaining the sample from a subject. Preferably, the sample has been obtained previously from a subject.

A further aspect of the present invention provides a method of monitoring the efficacy of a therapeutic treatment of pancreatic cancer, the method comprising:

(i) providing a biological sample from a patient undergoing the therapeutic treatment; and
(ii) determining the level of a pancreatic cancer-associated transcript in the biological sample by contacting the biological sample with a polynucleotide that selectively hybridizes to a sequence having at least about 80% identity to a sequence as shown in any one of Tables 3 or 4, thereby monitoring the efficacy of the therapy.

Preferably the method further comprises comparing the level of the pancreatic cancer-associated transcript to a level of the pancreatic cancer-associated transcript in a biological sample from the patient prior to, or earlier in, the therapeutic treatment.

In a related embodiment, the present invention provides a method of monitoring the efficacy of a therapeutic treatment of pancreatic cancer, the method comprising:

(i) providing a biological sample from a patient undergoing the therapeutic treatment; and
(ii) determining the level of a pancreatic cancer-associated antibody in the biological sample by contacting the biological sample with a polypeptide encoded by a polynucleotide that selectively hybridizes to a sequence at least 80% identical to a sequence as shown in Tables 3 or 4, wherein the polypeptide specifically binds to the pancreatic cancer-associated antibody, thereby monitoring the efficacy of the therapy.

Preferably the method further comprises comparing the level of the pancreatic cancer-associated antibody to a level of the pancreatic cancer-associated antibody in a biological sample from the patient prior to, or earlier in, the therapeutic treatment.

In a further related embodiment, the present invention provides a method of monitoring the efficacy of a therapeutic treatment of pancreatic cancer, the method comprising:

(i) providing a biological sample from a patient undergoing the therapeutic treatment; and
(ii) determining the level of a pancreatic cancer-associated polypeptide in the biological sample by contacting the biological sample with an antibody, wherein the antibody specifically binds to a polypeptide encoded by a polynucleotide that selectively hybridizes to a sequence at least 80% identical to a sequence as shown in Tables 3 or 4, thereby monitoring the efficacy of the therapy.

Preferably the method further comprises comparing the level of the pancreatic cancer-associated polypeptide to a level of the pancreatic cancer-associated polypeptide in a biological sample from the patient prior to, or earlier in, the therapeutic treatment.

A further aspect of the present invention provides a process for monitoring the efficacy of treatment of a cancer in a subject comprising performing the diagnostic method supra on a sample from a subject suffering from the cancer wherein treatment commenced before the time when the sample was taken and wherein a reduced level of expression relative to the level of expression in a healthy or normal subject indicates that the subject has responded to treatment.

In a related embodiment, the present invention provides a process for monitoring the efficacy of treatment of a cancer in a subject comprising performing the diagnostic method supra on a sample from a subject suffering from the cancer wherein treatment commenced before the time when the sample was taken and wherein a similar or enhanced level of expression relative to the level of expression in a healthy or normal subject indicates that the subject has not responded to treatment.

In a further embodiment, the present invention provides a process for monitoring the efficacy of treatment of a cancer in a subject comprising performing the diagnostic method supra on samples from a subject suffering from the cancer taken at least two different time points wherein treatment commenced at or following the first of said time points and wherein a reduced level of expression at a later time point indicates that the subject has responded to treatment.

In a related embodiment, the present invention provides a process for monitoring the efficacy of treatment of a cancer in a subject comprising performing the diagnostic method supra on samples from a subject suffering from the cancer taken at least two different time points wherein treatment commenced at or following the first of said time points and wherein a similar or enhanced level of expression at a later time point indicates that the subject has not responded to treatment.

The results of the diagnostic/prophylactic assays described herein are of particular use in designing and/or recommending effective or alternative therapeutic regimes for subjects suffering from cancer, based upon a primary diagnosis or assay result obtained following a primary diagnosis e.g., during primary treatment. Included within such recommendations are recommendations following surgical resection or chemotherapy or radiotherapy.

### BRIEF DESCRIPTION OF THE DRAWINGS

FIG. 1a is a graphical representation of a Kaplan-Meier survival curve showing the correlation between survival and level of HOX B2 nuclear expression for a cohort of 128 patients suffering from pancreatic cancer.

FIG. 1b is a graphical representation of a Kaplan-Meier survival curve showing the effect of surgical resection therapy on survival outcome for a cohort of 128 patients suffering from pancreatic cancer.

FIG. 1c is a graphical representation of a Kaplan-Meier survival curve showing the effect of stage of pancreatic cancer (Stage I/II vs stage III/IV) on survival for a cohort of 128 patients suffering from pancreatic cancer.

FIG. 1d is a graphical representation of a Kaplan-Meier survival curve showing the effect of degree of differentiation of pancreatic cancer (well/moderate differentiation vs poor differentiation) on survival for a cohort of 128 patients suffering from pancreatic cancer.

FIG. 1e is a graphical representation of a Kaplan-Meier survival curve showing the effect of enhanced HOX B2 expression on the outcome of surgical resection for a cohort of 48 patients suffering from pancreatic cancer.

FIG. 1f is a graphical representation of a Kaplan-Meier survival curve showing the effect of normal levels of HOX B2 expression on the outcome of surgical resection for a cohort of 80 patients suffering from pancreatic cancer.

FIG. 1g is a graphical representation of a Kaplan-Meier survival curve showing stratification of HOX B2 expression with outcome of surgical resection for a cohort of 128 patients suffering from pancreatic cancer.

FIG. 2a is a copy of a photographic representation showing HOX B2 protein expression in ovarian stromal tissue from a normal/healthy control subject. Data indicate negative staining (i.e., expression is not enhanced).

FIG. 2b is a copy of a photographic representation showing HOX B2 protein expression in a breast carcinoma. Data indicated positive staining (i.e., expression is enhanced).

FIG. 2c is a copy of a photographic representation showing HOX B2 protein expression in a precursor pancreatic cancer lesion. Data indicate negative staining (i.e., expression is not enhanced).

FIG. 2d is a copy of a photographic representation showing HOX B2 protein expression in a pancreatic cancer tissue section. Data indicate heterogeneous nuclear staining (i.e., expression is enhanced).

FIG. 2e is a copy of a photographic representation showing HOX B2 protein expression in a pancreatic cancer tissue section. Data indicate homogeneous nuclear staining (i.e., expression is enhanced).

FIG. 2f is a copy of a photographic representation showing HOX B2 protein expression in a pancreative cancer tissue section. Data indicate intense homogeneous nuclear staining (i.e., expression is enhanced).

FIG. 3a is a graphical representation of a Kaplan-Meier survival curve showing the correlation between survival and level of HOX B2 nuclear expression for a cohort of 76 patients suffering from pancreatic cancer that underwent surgical resection.

FIG. 3b is a graphical representation of a Kaplan Meier survival curve showing the correlation between survival and margin status for a cohort of 76 patients suffering from pancreatic cancer that underwent surgical resection.

FIG. 3c is a graphical representation of a Kaplan-Meier survival curve showing the correlation between survival and tumor size for a cohort of 76 patients suffering from pancreatic cancer that underwent surgical resection.

FIG. 3d is a graphical representation of a Kaplan-Meier survival curve showing the correlation between survival and lymph node status for a cohort of 76 patients suffering from pancreatic cancer that underwent surgical resection.

FIG. 3e is a graphical representation of a Kaplan-Meier survival curve showing the correlation between survival and degree of tumor differentiation for a cohort of 76 patients suffering from pancreatic cancer that underwent surgical resection.

### DETAILED DESCRIPTION OF THE PREFERRED EMBODIMENTS

### Pancreatic Cancer-Associated Sequences

Pancreatic cancer-associated sequences can include both nucleic acid (i.e., “pancreatic cancer-associated genes”) and protein (i.e., “pancreatic cancer-associated proteins”).

As used herein, the term “pancreatic cancer-associated protein” shall be taken to mean any protein that has an expression pattern correlated to a pancreatic cancer, the recurrence of a pancreatic cancer or the survival of a subject suffering from pancreatic cancer.

Similarly, the term “pancreatic cancer-associated gene” shall be taken to mean any nucleic acid encoding a pancreatic cancer-associated protein or nucleic acid having an expression profile that is correlated to a pancreatic cancer, the recurrence of a pancreatic cancer or the survival of a subject suffering from pancreatic cancer.

As will be appreciated by those in the art and is more fully outlined below, pancreatic cancer-associated genes are useful in a variety of applications, including diagnostic applications, which will detect naturally occurring nucleic acids, as well as screening applications; e.g., biochips comprising nucleic acid probes or PCR microtitre plates with selected probes to the pancreatic cancer sequences are generated.

For identifying pancreatic cancer-associated sequences, the pancreatic cancer screen typically includes comparing genes identified in different tissues, e.g., normal and cancerous tissues, or tumour tissue samples from patients who have metastatic disease vs. non metastatic tissue. Other suitable tissue comparisons include comparing pancreatic cancer samples with metastatic cancer samples from other cancers, such as lung, breast, gastrointestinal cancers, pancreatic, etc. Samples of different stages of pancreatic cancer, e.g., survivor tissue, drug resistant states, and tissue undergoing metastasis, are applied to biochips comprising nucleic acid probes. The samples are first microdissected, if applicable, and treated as is known in the art for the preparation of mRNA. Suitable biochips are commercially available, e.g. from Affymetrix. Gene expression profiles as described herein are generated and the data analyzed.

In one embodiment, the genes showing changes in expression as between normal and disease states are compared to genes expressed in other normal tissues, preferably normal pancreatic, but also including, and not limited to lung, heart, brain, liver, breast, kidney, muscle, colon, small intestine, large intestine, spleen, bone and placenta. In a preferred embodiment, those genes identified during the pancreatic cancer screen that are expressed in any significant amount in other tissues are removed from the profile, although in some embodiments, this is not necessary.

In a preferred embodiment, pancreatic cancer-associated sequences are those that are up-regulated in pancreatic cancer relative to a suitable control sample i.e., the expression of these genes is modified (up-regulated or down-regulated) in pancreatic cancer tissue as compared to non-cancerous tissue (see Table 3).

“Up-regulation” as used herein means at least about a two-fold change, preferably at least about a three fold change, with at least about five-fold or higher being preferred.

“Down-regulation” as used herein often means a level of expression that is less than that in the healthy/normal control subject (see Table 4). Preferably, the level of expression is less than about 50% (i.e. 0.5) of the level observed for a healthy or normal control subject. More preferably, the expression is reduced to a level that is about 30% or 20% or 10% or less of the level observed for a healthy or normal control sample.

### Detection of Pancreatic Cancer Sequences for Diagnostic/Prognostic Applications

In one aspect, the RNA expression levels of genes are determined for different cellular states in the pancreatic cancer phenotype. Expression levels of genes in normal tissue (i.e., not undergoing pancreatic cancer) and in pancreatic cancer tissue (and in some cases, for varying severities of pancreatic cancer that relate to prognosis, as outlined below) are evaluated to provide expression profiles. An expression profile of a particular cell state or point of development is essentially a “fingerprint” of the state. While two states may have any particular gene similarly expressed, the evaluation of a number of genes simultaneously allows the generation of a gene expression profile that is reflective of the state of the cell. By comparing expression profiles of cells in different states, information regarding which genes are important (including both up- and down-regulation of genes) in each of these states is obtained. Then, diagnosis are performed or confirmed to determine whether a tissue sample has the gene expression profile of normal or cancerous tissue. This will provide for molecular diagnosis of related conditions.

“Differential expression,” or grammatical equivalents as used herein, refers to qualitative or quantitative differences in the temporal and/or cellular gene expression patterns within and among cells and tissue. Thus, a differentially expressed gene can qualitatively have its expression altered, including an activation or inactivation, in, e.g., normal versus pancreatic cancer tissue. Genes are turned on or turned off in a particular state, relative to another state thus permitting comparison of two or more states. A qualitatively regulated gene will exhibit an expression pattern within a state or cell type which is detectable by standard techniques. Some genes will be expressed in one state or cell type, but not in both. Alternatively, the difference in expression are quantitative, e.g., in that expression is increased or decreased; i.e., gene expression is either upregulated, resulting in an increased amount of transcript, or downregulated, resulting in a decreased amount of transcript. The degree to which expression differs need only be large enough to quantify via standard characterization techniques as outlined below, such as by use of Affymetrix GeneChip™ expression arrays, Lockhart, Nature Biotechnology 14:1675-1680 (1996), hereby expressly incorporated by reference. Other techniques include, but are not limited to, quantitative reverse transcriptase PCR, northern analysis and RNase protection. As outlined above, preferably the change in expression (i.e., upregulation or downregulation) is at least about 50%, more preferably at least about 100%, more preferably at least about 150%, more preferably at least about 200%, with from 300 to at least 1000% being especially preferred.

Evaluation are at the gene transcript, or the protein level. The amount of gene expression are monitored using nucleic acid probes to the DNA or RNA equivalent of the gene transcript, and the quantification of gene expression levels, or, alternatively, the final gene product itself (protein) are monitored, e.g., with antibodies to the pancreatic cancer-associated protein and standard immunoassays (ELISAs, etc.) or other techniques, including mass spectroscopy assays, 2D gel electrophoresis assays, etc. Proteins corresponding to pancreatic cancer genes, i.e., those identified as being correlated to a pancreatic cancer phenotype, are evaluated in a pancreatic cancer diagnostic test.

In a preferred embodiment, gene expression monitoring is performed on a plurality of genes. Multiple protein expression monitoring are performed as well. Similarly, these assays are performed on an individual basis as well.

In this embodiment, the pancreatic cancer nucleic acid probes are attached to biochips as outlined herein for the detection and quantification of pancreatic cancer sequences in a particular cell. The assays are further described below in the example. PCR techniques are used to provide greater sensitivity.

In a preferred embodiment nucleic acids encoding the pancreatic cancer-associated protein are detected. Although DNA or RNA encoding the pancreatic cancer-associated protein are detected, of particular interest are methods wherein an mRNA encoding a pancreatic cancer-associated protein is detected. Probes to detect mRNA are a nucleotide/deoxynucleotide probe that is complementary to and hybridizes with the mRNA and includes, but is not limited to, oligonucleotides, cDNA or RNA. Probes also should contain a detectable label, as defined herein. In one method the mRNA is detected after immobilizing the nucleic acid to be examined on a solid support such as nylon membranes and hybridizing the probe with the sample. Following washing to remove the non-specifically bound probe, the label is detected. In another method detection of the mRNA is performed in situ. In this method permeabilized cells or tissue samples are contacted with a detectably labeled nucleic acid probe for sufficient time to allow the probe to hybridize with the target mRNA. Following washing to remove the non-specifically bound probe, the label is detected. For example a digoxygenin labeled riboprobe (RNA probe) that is complementary to the mRNA encoding a pancreatic cancer-associated protein is detected by binding the digoxygenin with an anti-digoxygenin secondary antibody and developed with nitro blue tetrazolium and 5-bromo-4-chloro-3indoyl phosphate.

In a preferred embodiment, various proteins from several classes of proteins as described herein by reference to Tables 3-25 are used in diagnostic assays. The pancreatic cancer-associated proteins, antibodies, nucleic acids, modified proteins and cells containing pancreatic cancer sequences are used in diagnostic assays. This are performed on an individual gene or corresponding polypeptide level. In a preferred embodiment, the expression profiles are used, preferably in conjunction with high throughput screening techniques to allow monitoring for expression profile genes and/or corresponding polypeptides.

As described and defined herein, pancreatic cancer-associated proteins, including intracellular, transmembrane or secreted proteins, find use as markers of pancreatic cancer. Detection of these proteins in putative pancreatic cancer tissue allows for detection or diagnosis of pancreatic cancer. In one embodiment, antibodies are used to detect pancreatic cancer-associated proteins. A preferred method separates proteins from a sample by electrophoresis on a gel (typically a denaturing and reducing protein gel, but are another type of gel, including isoelectric focusing gels and the like). Following separation of proteins, the pancreatic cancer-associated protein is detected, e.g., by immunoblotting with antibodies raised against the pancreatic cancer-associated protein. Methods of immunoblotting are well known to those of ordinary skill in the art.

In another preferred method, antibodies to the pancreatic cancer-associated protein find use in in situ imaging techniques, e.g., in histology (e.g., Methods in Cell Biology: Antibodies in Cell Biology, volume 37 (Asai, ed. 1993)). In this method cells are contacted with from one to many antibodies to the pancreatic cancer-associated protein(s). Following washing to remove non-specific antibody binding, the presence of the antibody or antibodies is detected. In one embodiment the antibody is detected by incubating with a secondary antibody that contains a detectable label. In another method the primary antibody to the pancreatic cancer-associated proteins) contains a detectable label, e.g. an enzyme marker that can act on a substrate. In another preferred embodiment each one of multiple primary antibodies contains a distinct and detectable label. This method finds particular use in simultaneous screening for a plurality of pancreatic cancer-associated proteins. As will be appreciated by one of ordinary skill in the art, many other histological imaging techniques are also provided by the invention.

In a preferred embodiment the label is detected in a fluorometer which has the ability to detect and distinguish emissions of different wavelengths. In addition, a fluorescence activated cell sorter (FACS) are used in the method. In another preferred embodiment, antibodies find use in diagnosing pancreatic cancer from blood, serum, plasma, stool, and other samples. Such samples, therefore, are useful as samples to be probed or tested for the presence of pancreatic cancer-associated proteins. Antibodies are used to detect a pancreatic cancer-associated protein by previously described immunoassay techniques including ELISA, immunoblotting (western blotting), immunoprecipitation, BIACORE technology and the like. Conversely, the presence of antibodies may indicate an immune response against an endogenous pancreatic cancer-associated protein.

In a preferred embodiment, in situ hybridization of labeled pancreatic cancer nucleic acid probes to tissue arrays is done. For example, arrays of tissue samples, including pancreatic cancer tissue and/or normal tissue, are made. In situ hybridization (see, e.g., Ausubel, supra) is then performed. When comparing the fingerprints between an individual and a standard, the skilled artisan can make a diagnosis, a prognosis, or a prediction based on the findings. It is further understood that the genes which indicate the diagnosis may differ from those which indicate the prognosis and molecular profiling of the condition of the cells may lead to distinctions between responsive or refractory conditions or are predictive of outcomes.

In a preferred embodiment, the pancreatic cancer-associated proteins, antibodies, nucleic acids, modified proteins and cells containing pancreatic cancer sequences are used in prognosis assays. As above, gene expression profiles are generated that correlate to pancreatic cancer, in terms of long term prognosis. Again, this are done on either a protein or gene level, with the use of genes being preferred. As above, pancreatic cancer probes are attached to biochips for the detection and quantification of pancreatic cancer sequences in a tissue or patient. The assays proceed as outlined above for diagnosis. PCR method may provide more sensitive and accurate quantification.

### Characteristics of Pancreatic Cancer-Associated Proteins and Genes Encoding Same

Pancreatic cancer-associated proteins of the present invention are classified as membrane proteins (Table 5), extracellular proteins (Table 6), proteins of the TGF-β signalling pathway (Table 7), WNT signalling pathway proteins (Table 8), proteins of nucleotide metabolism (Table 9), proteins involved in smooth muscle contraction (Table 10), mitochondrial proteins (Table 11), collagens or proteins of collagen synthesis or fibrillins (Table 12), inflammatory response pathway proteins (Table 13), endoplasmic reticulum (ER) proteins (Table 14), apoptotic proteins (Table 15), G1/S phase cell cycle control proteins (Table 16), matrix metalloproteinases (Table 17), proteins involved in retinoic acid signal transduction (Table 18), calcium channel proteins (Table 19), cathepsin proteins (Table 20), viral oncoprotein homologs (Table 21), S100 calcium binding proteins (Table 22), homeobox proteins (Table 23), zinc finger proteins (Table 24) and heat shock proteins (Table 25), amongst others.

In one embodiment, the pancreatic cancer-associated protein is an intracellular protein. Intracellular proteins are found in the cytoplasm and/or in the nucleus. Intracellular proteins are involved in all aspects of cellular function and replication (including, e.g., signaling pathways); aberrant expression of such proteins often results in unregulated or disregulated cellular processes (see, e.g., Molecular Biology of the Cell (Alberts, ed., 3rd ed., 1994). For example, many intracellular proteins have enzymatic activity such as protein kinase activity, protein phosphatase activity, protease activity, nucleotide cyclase activity, polymerase activity and the like. Intracellular proteins also serve as docking proteins that are involved in organizing complexes of proteins, or targeting proteins to various subcellular localizations, and are involved in maintaining the structural integrity of organelles.

An increasingly appreciated concept in characterising proteins is the presence in the proteins of one or more motifs for which defined functions have been attributed. In addition to the highly conserved sequences found in the enzymatic domain of proteins, highly conserved sequences have been identified in proteins that are involved in protein-protein interaction. For example, Src-identity-2 (SH2) domains bind tyrosine-phosphorylated targets in a sequence dependent manner. PTB domains, which are distinct from SH2 domains, also bind tyrosine phosphorylated targets. SH3 domains bind to proline-rich targets. In addition, PH domains, tetratricopeptide repeats and WD domains to name only a few, have been shown to mediate protein-protein interactions. Some of these may also be involved in binding to phospholipids or other second messengers. As will be appreciated by one of ordinary skill in the art, these motifs are identified on the basis of primary sequence; thus, an analysis of the sequence of proteins may provide insight into both the enzymatic potential of the molecule and/or molecules with which the protein may associate. One useful database is Pfam (protein families), which is a large collection of multiple sequence alignments and hidden Markov models covering many common protein domains. Versions are available via the internet from Washington University in St. Louis, the Sanger Center in England, and the Karolinska Institute in Sweden (see, e.g., Bateman et al., 2000, Nuc. Acids Res. 28: 263-266; Sonnhammer et al., 1997, Proteins 28: 405-420; Bateman et al., 1999, Nuc. Acids Res. 27:260-262; and Sonnhammer et al., 1998, Nuc. Acids Res. 26: 320-322.

In another embodiment, the pancreatic cancer sequences are transmembrane proteins. Transmembrane proteins are molecules that span a phospholipid bilayer of a cell. They may have an intracellular domain, an extracellular domain, or both. The intracellular domains of such proteins may have a number of functions including those already described for intracellular proteins. For example, the intracellular domain may have enzymatic activity and/or may serve as a binding site for additional proteins. Frequently the intracellular domain of transmembrane proteins serves both roles. For example certain receptor tyrosine kinases have both protein kinase activity and SH2 domains. In addition, autophosphorylation of tyrosines on the receptor molecule itself, creates binding sites for additional SH2 domain containing proteins.

Transmembrane proteins may contain from one to many transmembrane domains. For example, receptor tyrosine kinases, certain cytokine receptors, receptor guanylyl cyclases and receptor serine/threonine protein kinases contain a single transmembrane domain. However, various other proteins including channels and adenylyl cyclases contain numerous transmembrane domains. Many important cell surface receptors such as G protein coupled receptors (GPCRs) are classified as “seven transmembrane domain” proteins, as they contain 7 membrane spanning regions. Characteristics of transmembrane domains include approximately 20 consecutive hydrophobic amino acids that are followed by charged amino acids. Therefore, upon analysis of the amino acid sequence of a particular protein, the localization and number of transmembrane domains within the protein are predicted (see, e.g. PSORT web site http://psort.nibb.ac.jp/). Important transmembrane protein receptors include, but are not limited to the insulin receptor, insulin-like growth factor receptor, human growth hormone receptor, glucose transporters, transferrin receptor, epidermal growth factor receptor, low density lipoprotein receptor, epidermal growth factor receptor, leptin receptor, interleukin receptors, e.g. IL-1 receptor, IL-2 receptor.

The extracellular domains of transmembrane proteins are diverse; however, conserved motifs are found repeatedly among various extracellular domains. Conserved structure and/or functions have been ascribed to different extracellular motifs. Many extracellular domains are involved in binding to other molecules. In one aspect, extracellular domains are found on receptors. Factors that bind the receptor domain include circulating ligands, which are peptides, proteins, or small molecules such as adenosine and the like. For example, growth factors such as EGF, FGF and PDGF are circulating growth factors that bind to their cognate receptors to initiate a variety of cellular responses. Other factors include cytokines, mitogenic factors, neurotrophic factors and the like. Extracellular domains also bind to cell-associated molecules.

In this respect, they mediate cell-cell interactions., Cell-associated ligands are tethered to the cell, e.g., via a glycosylphosphatidylinositol (GPI) anchor, or may themselves be transmembrane proteins. Extracellular domains also associate with the extracellular matrix and contribute to the maintenance of the cell structure.

Pancreatic cancer-associated proteins that are transmembrane are particularly preferred in the present invention as they are readily accessible targets for immunotherapeutics, as are described herein. In addition, as outlined below, transmembrane proteins are also useful in imaging modalities. Antibodies are used to label such readily accessible proteins in situ. Alternatively, antibodies can also label intracellular proteins, in which case samples are typically permeablized to provide access to intracellular proteins.

It will also be appreciated by those in the art that a transmembrane protein are made soluble by removing transmembrane sequences, e.g., through recombinant methods. Furthermore, transmembrane proteins that have been made soluble are made to be secreted through recombinant means by adding an appropriate signal sequence.

In another embodiment, the pancreatic cancer-associated proteins are secreted proteins; the secretion of which are either constitutive or regulated. These proteins have a signal peptide or signal sequence that targets the molecule to the secretory pathway. Secreted proteins are involved in numerous physiological events; by virtue of their circulating nature, they serve to transmit signals to various other cell types. The secreted protein may function in an autocrine manner (acting on the cell that secreted the factor), a paracrine manner (acting on cells in close proximity to the cell that secreted the factor) or an endocrine manner (acting on cells at a distance). Thus secreted molecules find use in modulating or altering numerous aspects of physiology. Pancreatic cancer-associated proteins that are secreted proteins are particularly preferred in the present invention as they are suitable targets for diagnostic markers in non-invasive tests, e.g., for screening blood, plasma, serum, ascites, stool, or urine samples.

It will be understood by the skilled artisan that extracellular proteins are also suitable targets for diagnostic markers in non-invasive tests.

### Mammalian Subjects

The present invention provides nucleic acid and protein sequences that are differentially expressed in pancreatic cancer, herein termed “pancreatic cancer sequences.” As outlined below, pancreatic cancer sequences include those that are up-regulated (i.e., expressed at a higher level) in pancreatic cancer, as well as those that are down-regulated (i.e., expressed at a lower level). In a preferred embodiment, the pancreatic cancer sequences are from humans; however, as will be appreciated by those in the art, pancreatic cancer sequences from other organisms are useful in animal models of disease and drug evaluation; thus, other pancreatic cancer sequences are provided, from vertebrates, including mammals, including rodents (rats, mice, hamsters, guinea pigs, etc.), primates, farm animals (including sheep, goats, pigs, cows, horses, etc.) and pets, e.g., (dogs, cats, etc.).

### Assay Control Samples

It will be apparent from the preceding discussion that many of the diagnostic methods provided by the present invention involve a degree of quantification to determine, on the one hand, the over-expression or reduced-expression of a diagnostic/prognostic marker in tissue that is suspected of comprising a cancer cell. Such quantification can be readily provided by the inclusion of appropriate control samples in the assays described below, derived from healthy or normal individuals. Alternatively, if internal controls are not included in each assay conducted, the control may be derived from an established data set that has been generated from healthy or normal individuals.

In the present context, the term “healthy individual” shall be taken to mean an individual who is known not to suffer from pancreatic cancer, such knowledge being derived from clinical data on the individual, including, but not limited to, a different cancer assay to that described herein. As the present invention is particularly useful for the early detection of pancreatic cancer, it is preferred that the healthy individual is asymptomatic with respect to the early symptoms associated with pancreatic cancer. Although early detection using well-known procedures is difficult, reduced urinary frequency, rectal pressure, and abdominal bloating and swelling, are associated with the disease in its early stages, and, as a consequence, healthy individuals should not have any of these clinical symptoms. Clearly, subjects suffering from later symptoms associated with pancreatic cancer, such as, for example, metastases in the omentum, abdominal fluid, lymph nodes, lung, liver, brain, or bone, and subjects suffering from spinal cord compression, elevated calcium level, chronic pain, or pleural effusion, should also be avoided from the “healthy individual” data set.

The term “normal individual” shall be taken to mean an individual having a normal level of expression of a cancer-associate gene or cancer-associated protein in a particular sample derived from said individual. As will be known to those skilled in the art, data obtained from a sufficiently large sample of the population will normalize, allowing the generation of a data set for determining the average level of a particular parameter. Accordingly, the level of expression of a cancer-associate gene or cancer-associated protein can be determined for any population of individuals, and for any sample derived from said individual, for subsequent comparison to levels determined for a sample being assayed. Where such normalized data sets are relied upon, internal controls are preferably included in each assay conducted to control for variation.

In one embodiment, the present invention provides a method for detecting a pancreatic cancer cell in a subject, said method comprising:
(i) determining the level of mRNA encoding a pancreatic cancer-associated protein expressed in a test sample from said subject; and
(ii) comparing the level of mRNA determined at (i) to the level of mRNA encoding a pancreatic cancer-associated protein expressed in a comparable sample from a healthy or normal individual,
   wherein a level of mRNA at (i) that is modified in the test sample relative to the comparable sample from the normal or healthy individual is indicative of the presence of a pancreatic cancer cell in said subject.

Alternatively, or in addition, the control may comprise a cancer-associated sequence that is known to be expressed at a particular level in a pancreatic cancer, eg., TIMP1 (Gen Bank Accession No. X03124) or COL1A2 (GenBank Accession No. X55525).

### Biological Samples

Preferred biological samples in which the assays of the invention are performed include bodily fluids, pancreatic tissue and cells, and those tissues known to comprise cancer cells arising from a metastasis of a pancreatic cancer, such as, for example, in carcinomas of the ovary lung, prostate, breast, colon, placenta, or omentum, and in cells of brain anaplastic oligodendrogliomas.

Bodily fluids shall be taken to include urine, ascites, whole blood, serum, peripheral blood mononuclear cells (PBMC), or buffy coat fraction.

In the present context, the term “cancer cell” includes any biological specimen or sample comprising a cancer cell irrespective of its degree of isolation or purity, such as, for example, tissues, organs, cell lines, bodily fluids, or histology specimens that comprise a cell in the early stages of transformation or having been transformed.

As the present invention is particularly useful for the early detection and prognosis of cancer in the short term or in the medium-to-long term, the definition of “cancer cell” is not to be limited by the stage of a cancer in the subject from which said cancer cell is derived (ie. whether or not the patient is in remission or undergoing disease recurrence or whether or not the cancer is a primary tumor or the consequence of metastases). Nor is the term “cancer cell” to be limited by the stage of the cell cycle of said cancer cell.

Preferably, the sample comprises pancreatic tissue, prostate tissue, kidney tissue, uterine tissue, placenta, a cervical specimen, omentum, rectal tissue, brain tissue, bone tissue, lung tissue, lymphatic tissue, urine, semen, blood, abdominal fluid, serum, or faeces, or a cell preparation or nucleic acid preparation derived therefrom. More preferably, the sample comprises serum or abdominal fluid, or a tissue selected from the group consisting of: pancreas, lymph, lung, liver, brain, placenta, brain, omentum, and prostate. Even more preferably, the sample comprises serum or abdominal fluid, pancreas, or lymph node tissue. The sample can be prepared on a solid matrix for histological analyses, or alternatively, in a suitable solution such as, for example, an extraction buffer or suspension buffer, and the present invention clearly extends to the testing of biological solutions thus prepared.

### Polynucleotide Probes and Amplification Primers

Polynucleotide probes are derived from or comprise the nucleic acid sequences whose nucleotide sequences are provided by reference to the public database accession numbers given in any one of Tables 3-25 and sequences homologues thereto as well as variants, derivatives and fragments thereof.

Whilst the probes may comprise double-stranded or single-stranded nucleic acid, single-stranded probes are preferred because they do not require melting prior to use in hybridizations. On the other hand, longer probes are also preferred because they can be used at higher hybridization stringency than shorter probes and may produce lower background hybridization than shorter probes.

So far as shorter probes are concerned, single-stranded, chemically-synthesized oligonucleotide probes are particularly preferred by the present invention. To reduce the noise associated with the use of such probes during hybridization, the nucleotide sequence of the probe is carefully selected to maximize the Tm at which hybridizations can be performed, reduce non-specific hybridization, and to reduce self-hybridization. Such considerations may be particularly important for applications involving high throughput screening using microarray technology. In general, this means that the nucleotide sequence of an oligonucleotide probe is selected such that it is unique to the target RNA or protein-encoding sequence, has a low propensity to form secondary structure, low self-complementary, and is not highly A/T-rich.

The only requirement for the probes is that they cross-hybridize to nucleic acid encoding the target diagnostic protein or the complementary nucleotide sequence thereto and are sufficiently unique in sequence to generate high signal:noise ratios under specified hybridization conditions. As will be known to those skilled in the art, long nucleic acid probes are preferred because they tend to generate higher signal:noise ratios than shorter probes and/or the duplexes formed between longer molecules have higher melting temperatures (i.e. Tm values) than duplexes involving short probes. Accordingly, full-length DNA or RNA probes are contemplated by the present invention, as are specific probes comprising the sequence of the 3′-untranslated region or complementary thereto.

In a particularly preferred embodiment, the nucleotide sequence of an oligonucleotide probe has no detectable nucleotide sequence identity to a nucleotide sequence in a BLAST search (Altschul et al., J. Mol. Biol. 215, 403-410, 1990) or other database search, other than a sequence selected from the group consisting of: (a) a sequence encoding a polypeptide listed in any one of Tables 3-25; (b) the 5′-untranslated region of a sequence encoding a polypeptide listed in any one of Tables 3-25; (c) a 3′-untranslated region of a sequence encoding a polypeptide listed in any one of Tables 3-25; and (d) an exon region of a sequence encoding a polypeptide listed in any one of Tables 3-25.

Additionally, the self-complementarity of a nucleotide sequence can be determined by aligning the sequence with its reverse complement, wherein detectable regions of identity are indicative of potential self-complementarity. As will be known to those skilled in the art, such sequences may not necessarily form secondary structures during hybridization reaction, and, as a consequence, successfully identify a target nucleotide sequence. It is also known to those skilled in the art that, even where a sequence does form secondary structures during hybridization reactions, reaction conditions can be modified to reduce the adverse consequences of such structure formation. Accordingly, a potential for self-complementarity should not necessarily exclude a particular candidate oligonucleotide from selection. In cases where it is difficult to determine nucleotide sequences having no potential self-complementarity, the uniqueness of the sequence should outweigh a consideration of its potential for secondary structure formation.

Recommended pre-requisites for selecting oligonucleotide probes, particularly with respect to probes suitable for microarray technology, are described in detail by Lockhart et al., “Expression monitoring by hybridization to high-density oligonucleotide arrays”, Nature Biotech. 14, 1675-1680, 1996.

The nucleic acid probe may comprise a nucleotide sequence that is within the coding strand of a gene listed in any one of Tables 3-25. Such “sense” probes are useful for detecting RNA by amplification procedures, such as, for example, polymerase chain reaction (PCR), and more preferably, quantitative PCR or reverse transcription polymerase chain reaction (RT-PCR). Alternatively, “sense” probes may be expressed to produce polypeptides or immunologically active derivatives thereof that are useful for detecting the expressed protein in samples.

The nucleotide sequences referred to in Tables 3-25 and homologues thereof encode polypeptides. It will be understood by a skilled person that numerous different Nucleic acids can encode the same polypeptide as a result of the degeneracy of the genetic code. In addition, it is to be understood that skilled persons may, using routine techniques, make nucleotide substitutions that do not affect the polypeptide sequence encoded by the Nucleic acids of the invention to reflect the codon usage of any particular host organism in which the polypeptides of the invention are to be expressed.

Nucleic acids may comprise DNA or RNA. They are single-stranded or double-stranded. They may also be nucleic acids which include within them synthetic or modified nucleotides.

A number of different types of modification to nucleic acids are known in the art. These include methylphosphonate and phosphorothioate backbones, addition of acridine or polylysine chains at the 3′ and/or 5′ ends of the molecule. For the purposes of the present invention, it is to be understood that the nucleic acids described herein are modified by any method available in the art. Such modifications are carried out in order to enhance the in vivo activity or half-life of the diagnostic/prognostic nucleic acids in use.

The terms “variant” or “derivative” in relation to the nucleotide sequences of the present invention include any substitution of, variation of, modification of, replacement of, deletion of or addition of one (or more) nucleic acid from or to the sequence provided that the resultant nucleotide sequence codes for a polypeptide having biological activity, preferably having substantially the same activity as the polypeptide sequences presented in the sequence listings.

With respect to sequence identity, preferably there is at least 75%, more preferably at least 85%, more preferably at least 90% identity to a sequence shown in Tables 1-3 herein over a region of at least 20, preferably at least 25 or 30, for instance at least 40, 60, 100, 500, 1000 or more contiguous nucleotides. More preferably there is at least 95%, more preferably at least 98%, identity. In one embodiment, homologues are naturally occurring sequences, such as orthologues, tissue-specific isoforms and allelic variants.

Identity comparisons are conducted by eye, or more usually, with the aid of readily available sequence comparison programs. These commercially available computer programs can calculate % identity between two or more sequences.

Percentages (%) identity are calculated over contiguous sequences, i.e. one sequence is aligned with the other sequence and each nucleotide in one sequence directly compared with the corresponding nucleotide in the other sequence, one base at a time. This is called an “ungapped” alignment. Typically, such ungapped alignments are performed only over a relatively short number of bases (for example less than 50 contiguous nucleotides).

Although this is a very simple and consistent method, it fails to take into consideration that, for example, in an otherwise identical pair of sequences, one insertion or deletion will cause the following nucleotides to be put out of alignment, thus potentially resulting in a large reduction in % identity when a global alignment is performed. Consequently, most sequence comparison methods are designed to produce optimal alignments that take into consideration possible insertions and deletions without penalising unduly the overall identity score. This is achieved by inserting “gaps” in the sequence alignment to try to maximise local identity.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons.

In determining whether or not two amino acid sequences fall within the stated defined percentage identity limits, those skilled in the art will be aware that it is necessary to conduct a side-by-side comparison of amino acid sequences. In such comparisons or alignments, differences will arise in the positioning of non-identical amino acid residues depending upon the algorithm used to perform the alignment. In the present context, references to percentage identities and similarities between two or more amino acid sequences shall be taken to refer to the number of identical and similar residues respectively, between said sequences as determined using any standard algorithm known to those skilled in the art. In particular, amino acid identities and similarities are calculated using the GAP program of the Computer Genetics Group, Inc., University Research Park, Madison, Wis., United States of America (Devereaux et al, Nucl. Acids Res. 12, 387-395, 1984), which utilizes the algorithm of Needleman and Wunsch J. Mol. Biol. 48, 443-453, 1970, or alternatively, the CLUSTAL W algorithm of Thompson et al., Nucl. Acids Res. 22, 4673-4680, 1994, for multiple alignments, to maximize the number of identical/similar amino acids and to minimize the number and/or length of sequence gaps in the alignment.

A suitable computer program for carrying out such an alignment is the GCG Wisconsin Bestfit package (University of Wisconsin, U.S.A.; Devereux et al., 1984, Nucleic Acids Research 12:387). The default scoring matrix has a match value of 10 for each identical nucleotide and −9 for each mismatch. The default gap creation penalty is −50 and the default gap extension penalty is −3 for each nucleotide.

Examples of other software than can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However it is preferred to use the GCG Bestfit program.

Once the software has produced an optimal alignment, it is possible to calculate % identity, preferably % sequence identity. The software typically does this as part of the sequence comparison and generates a numerical result.

A preferred sequence comparison program is the GCG Wisconsin Bestfit program described above.

The present invention also encompasses the use of nucleotide sequences that are capable of hybridizing selectively to the sequences presented herein, or any variant, fragment or derivative thereof, or to the complement of any of the above. Nucleotide sequences are preferably at least 15 nucleotides in length, more preferably at least 20, 30, 40 or 50 nucleotides in length.

The term “hybridization” as used herein shall include “the process by which a strand of nucleic acid joins with a complementary strand through base pairing” as well as the process of amplification as carried out in polymerase chain reaction technologies.

Nucleic acids capable of selectively hybridizing to the nucleotide sequences presented herein, or to their complement, will be generally at least 70%, preferably at least 80 or 90% and more preferably at least 95% or 98% homologous to the corresponding nucleotide sequences referred to in Tables 3-25 over a region of at least 20, preferably at least 25 or 30, for instance at least 40, 60, 100, 500, 1000 or more contiguous nucleotides.

The term “selectively hybridizable” means that the polynucleotide used as a probe is used under conditions where a target polynucleotide is found to hybridize to the probe at a level significantly above background. The background hybridization may occur because of other Nucleic acids present, for example, in the cDNA or genomic DNA library being screening. In this event, background implies a level of signal generated by interaction between the probe and a non-specific DNA member of the library which is less than 10 fold, preferably less than 100 fold as intense as the specific interaction observed with the target DNA. The intensity of interaction are measured, for example, by radiolabelling the probe, e.g. with ³²P.

Hybridization conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego Calif.), and confer a defined “stringency” as explained below.

For the purposes of defining the level of stringency, a high stringency hybridization is achieved using a hybridization buffer and/or a wash solution comprising the following:

(i) a salt concentration that is equivalent to 0.1×SSC-0.2×SSC buffer or lower salt concentration;
(ii) a detergent concentration equivalent to 0.1% (w/v) SDS or higher; and
(iii) an incubation temperature of 55° C. or higher.

Conditions for specifically hybridizing nucleic acid, and conditions for washing to remove non-specific hybridizing nucleic acid, are well understood by those skilled in the art. For the purposes of further clarification only, reference to the parameters affecting hybridization between nucleic acid molecules is found in Ausubel et al. (Current Protocols in Molecular Biology, Wiley Interscience, ISBN 047150338, 1992), which is herein incorporated by reference.

Maximum stringency typically occurs at about Tm-5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridization are used to identify or detect identical polynucleotide sequences while an intermediate (or low) stringency hybridization are used to identify or detect similar or related polynucleotide sequences.

In a preferred embodiment, the present invention encompasses the use of nucleotide sequences that can hybridize to a stated nucleotide sequence under stringent conditions (e.g. 65° C. and 0.1×SSC {1×SSC=0.15 M NaCl, 0.015 M Na₃Citrate pH 7.0}).

Where the diagnostic/prognostic polynucleotide is double-stranded, both strands of the duplex, either individually or in combination, are encompassed by the present invention. Where the polynucleotide is single-stranded, it is to be understood that the complementary sequence of that polynucleotide is also included within the scope of the present invention.

Nucleic acids which are not 100% homologous to the sequences of the present invention but are useful in performing the diagnostic and/or prognostic assays of the invention by virtue of their ability to selectively hybridize to the target gene transcript, or to encode an immunologically cross-reactive protein to the target protein, are obtained in a number of ways, such as, for example by probing DNA libraries made from a range of individuals, for example individuals from different populations. In particular, given that that changes in the expression of diagnostic/prognostic cancer-associated genes correlate with pancreatic cancer, characterisation of variant sequences in individuals suffering from pancreatic cancer is used to identify variations in the sequences of pancreatic-cancer associated genes (and proteins) that are predictive of and/or causative of pancreatic cancer.

Accordingly the present invention also encompasses the use of a variant sequence of a marker disclosed herein that is associated with pancreatic cancer.

In addition, other viral, bacterial or cellular homologues particularly cellular homologues found in mammalian cells (e.g. rat, mouse, bovine and primate cells), are obtained and such homologues and fragments thereof in general will be capable of selectively hybridizing to the sequences shown in Tables 3-25 or the Sequence Listing. Such sequences are obtained by probing cDNA libraries made from or genomic DNA libraries from other animal species, and probing such libraries with probes comprising all or part of the sequences specifically referred to in Tables 3-25 or the Sequence Listing under conditions of medium to high stringency.

Variants and strain/species homologues may also be obtained using degenerate PCR which will use primers designed to target sequences within the variants and homologues encoding conserved amino acid sequences within the sequences of the present invention. Conserved sequences are predicted, for example, by aligning the amino acid sequences from several variants/homologues. Sequence alignments are performed using computer software known in the art. For example the GCG Wisconsin PileUp program is widely used.

Primers used in degenerate PCR will contain one or more degenerate positions and will be used at stringency conditions lower than those used for cloning sequences with single sequence primers against known sequences.

Alternatively, such nucleic acids are obtained by site-directed mutagenesis of characterised sequences, such as the sequences referred to in Tables 3-25 or the Sequence Listing. This are useful where for example silent codon changes are required to sequences to optimise codon preferences for a particular host cell in which the polynucleotide sequences are being expressed or maintained.

Nucleic acids comprising a diagnostic/prognostic cancer-associated gene are used to produce a primer by standard derivatization means, e.g. a PCR primer, a primer for an alternative amplification reaction. In accordance with this embodiment, a probe is genreally labelled with a detectable label by conventional means using radioactive or non-radioactive labels. Such primers, probes and other fragments will be at least 15, preferably at least 20, for example at least 25, 30 or 40 nucleotides in length. Preferred fragments are less than 5000, 2000, 1000, 500 or 200 nucleotides in length.

Nucleic acids such as a DNA probes or riboprobes according to the invention are produced by recombinant or synthetic means, including cloning by standard techniques.

In general, primers will be produced by synthetic means, involving a step wise manufacture of the desired nucleic acid sequence one nucleotide at a time. Techniques for accomplishing this using automated techniques are readily available in the art.

Longer nucleic acid probes will generally be produced using recombinant means, for example using PCR (polymerase chain reaction) cloning techniques. This will involve making a pair of primers (e.g. of about 15 to 30 nucleotides) flanking a region of the sequence which it is desired to clone, bringing the primers into contact with mRNA or cDNA obtained from an animal or human cell, performing a polymerase chain reaction under conditions which bring about amplification of the desired region, isolating the amplified fragment (e.g. by purifying the reaction mixture on an agarose gel) and recovering the amplified DNA. The primers are designed to contain suitable restriction enzyme recognition sites so that the amplified DNA are cloned into a suitable cloning vector

Polynucleotide probes or primers preferably carry a detectable label. Suitable labels include radioisotopes such as ³²P or ³⁵S, enzyme labels, or other protein labels such as biotin. Such labels are added to nucleic acids or primers and are detected using by techniques known in the art.

Polynucleotide probes or primers, labeled or unlabeled, are used by those skilled in the art in nucleic acid-based tests for detecting or sequencing a diagnostic/prognostic cancer-associated gene.

Methods for probe synthesis by enzymic means generally comprises elongating, in the presence of suitable reagents, a primer complementary to a protion of the target DNA or RNA. Suitable reagents include a DNA polymerase enzyme, the deoxynucleotides dATP, dCTP, dGTP and dTTP, a buffer and ATP.

The probes/primers may conveniently be packaged in the form of a test kit in a suitable container. In such kits the probe are bound to a solid support where the assay format for which the kit is designed requires such binding. The kit may also contain suitable reagents for treating the sample to be probed, hybridizing the probe to nucleic acid in the sample, control reagents, instructions, and the like.

Preferably, a kit of the invention comprises primers/probes suitable for selectively detecting a plurality of sequences, more preferably for selectively detecting a plurality of sequences that are listed in one or more of Tables 3-25. Similarly, a kit of the invention preferably comprises primers suitable for selectively detecting a plurality of sequences referred to in any one of Tables 3-25.

### Nucleic Acid-Based Assay Formats

Nucleic acid-based tests for detecting a pancreatic cancer cell generally comprise bringing a biological sample containing DNA or RNA into contact with a probe comprising a polynucleotide probe or primer under at least low stringency hybridization conditions and detecting any duplex formed between the probe/primer and nucleic acid in the sample. Such detection are achieved using techniques such as PCR or by immobilising the probe on a solid support, removing nucleic acid in the sample which is not hybridized to the probe, and then detecting nucleic acid which has hybridized to the probe. Alternatively, the sample nucleic acid are immobilised on a solid support, and the amount of probe bound to such a support are detected. Suitable assay methods of this and other formats are found in for example WO89/03891 and WO90/13667.

As discussed in detail below, the status of expression of a cancer-associated gene in patient samples may be analyzed by a variety protocols that are well known in the art including in situ hybridization, northern blotting techniques, RT-PCR analysis (such as, for example, performed on laser capture microdissected samples), and microarray technology, such as, for example, using tissue microarrays probed with nucleic acid probes, or nucleic acid microarrays (ie. RNA microarrays or amplified DNA microarrays) microarrays probed with nucleic acid probes. All such assay formats are encompassed by the present invention.

For high throughput screening of large numbers of samples, such as, for example, public health screening of subjects, particularly human subjects, having a higher risk of developing cancer, microarray technology is a preferred assay format.

In accordance with such high throughput formats, techniques for producing immobilised arrays of DNA molecules have been described in the art. Generally, most prior art methods describe how to synthesise single-stranded nucleic acid molecule arrays, using for example masking techniques to build up various permutations of sequences at the various discrete positions on the solid substrate. U.S. Pat. No. 5,837,832, the contents of which are incorporated herein by reference, describes an improved method for producing DNA arrays immobilised to silicon substrates based on very large scale integration technology. In particular, U.S. Pat. No. 5,837,832 describes a strategy called “tiling” to synthesize specific sets of probes at spatially-defined locations on a substrate which are used to produced the immobilised DNA arrays. U.S. Pat. No. 5,837,832 also provides references for earlier techniques that may also be used.

Thus DNA are synthesised in situ on the surface of the substrate. However, DNA may also be printed directly onto the substrate using for example robotic devices equipped with either pins or piezo electric devices.

The plurality of polynucleotide sequences are typically immobilised onto or in discrete regions of a solid substrate. The substrate are porous to allow immobilisation within the substrate or substantially non-porous, in which case the library sequences are typically immobilised on the surface of the substrate. The solid substrate are made of any material to which polypeptides can bind, either directly or indirectly. Examples of suitable solid substrates include flat glass, silicon wafers, mica, ceramics and organic polymers such as plastics, including polystyrene and polymethacrylate. It may also be possible to use semi-permeable membranes such as nitrocellulose or nylon membranes, which are widely available. The semi-permeable membranes are mounted on a more robust solid surface such as glass. The surfaces may optionally be coated with a layer of metal, such as gold, platinum or other transition metal. A particular example of a suitable solid substrate is the commercially available BIACore™ chip (Pharmacia Biosensors).

Preferably, the solid substrate is generally a material having a rigid or semi-rigid surface. In preferred embodiments, at least one surface of the substrate will be substantially flat, although in some embodiments it are desirable to physically separate synthesis regions for different polymers with, for example, raised regions or etched trenches. It is also preferred that the solid substrate is suitable for the high density application of DNA sequences in discrete areas of typically from 50 to 100 μm, giving a density of 10000 to 40000 cm².

The solid substrate is conveniently divided up into sections. This are achieved by techniques such as photoetching, or by the application of hydrophobic inks, for example teflon-based inks (Cel-line, USA).

Discrete positions, in which each different member of the array is located may have any convenient shape, e.g., circular, rectangular, elliptical, wedge-shaped, etc.

Attachment of the polynucleotide sequences to the substrate are by covalent or non-covalent means. A plurality of polynucleotide sequences are attached to the substrate via a layer of molecules to which the sequences bind. For example, the sequences are labelled with biotin and the substrate coated with avidin and/or streptavidin. A convenient feature of using biotinylated sequences is that the efficiency of coupling to the solid substrate are determined easily. Since the library sequences may bind only poorly to some solid substrates, it is often necessary to provide a chemical interface between the solid substrate (such as in the case of glass) and the sequences. Examples of suitable chemical interfaces include hexaethylene glycol. Another example is the use of polylysine coated glass, the polylysine then being chemically modified using standard procedures to introduce an affinity ligand. Other methods for attaching molecules to the surfaces of solid substrate by the use of coupling agents are known in the art, see for example WO98/49557.

The complete DNA array is typically read at the same time by charged coupled device (CCD) camera or confocal imaging system. Alternatively, the DNA array are placed for detection in a suitable apparatus that can move in an x-y direction, such as a plate reader. In this way, the change in characteristics for each discrete position are measured automatically by computer controlled movement of the array to place each discrete element in turn in line with the detection means.

The detection means are capable of interrogating each position in the library array optically or electrically. Examples of suitable detection means include CCD cameras or confocal imaging systems.

In a preferred embodiment, the level of expression of the cancer-associated gene in the test sample is determined by hybridizing a probe/primer to RNA in the test sample under at least low stringency hybridization conditions and detecting the hybridization using a detection means.

Similarly, the level of mRNA in the comparable sample from the healthy or normal individual is preferably determined by hybridizing a probe/primer to RNA in said comparable sample under at least low stringency hybridization conditions and detecting the hybridization using a detection means.

For the purposes of defining the level of stringency to be used in these diagnostic assays, a low stringency is defined herein as being a hybridization and/or a wash carried out in 6×SSC buffer, 0.1% (w/v) SDS at 28° C., or equivalent conditions. A moderate stringency is defined herein as being a hybridization and/or washing carried out in 2×SSC buffer, 0.1% (w/v) SDS at a temperature in the range 45° C. to 65° C., or equivalent conditions. A high stringency is defined herein as being a hybridization and/or wash carried out in 0.1×SSC buffer, 0.1% (w/v) SDS, or lower salt concentration, and at a temperature of at least 65° C., or equivalent conditions. Reference herein to a particular level of stringency encompasses equivalent conditions using wash/hybridization solutions other than SSC known to those skilled in the art.

Generally, the stringency is increased by reducing the concentration of SSC buffer, and/or increasing the concentration of SDS and/or increasing the temperature of the hybridization and/or wash. Those skilled in the art will be aware that the conditions for hybridization and/or wash may vary depending upon the nature of the hybridization matrix used to support the sample RNA, or the type of hybridization probe used.

In general, the sample or the probe is immobilized on a solid matrix or surface (e.g., nitrocellulose). For high throughput screening, the sample or probe will generally comprise an array of nucleic acids on glass or other solid matrix, such as, for example, as described in WO 96/17958. Techniques for producing high density arrays are described, for example, by Fodor et al., Science 767-773, 1991, and in U.S. Pat. No. 5,143,854. Typical protocols for other assay formats can be found, for example in Current Protocols In Molecular Biology, Unit 2 (Northern Blotting), Unit 4 (Southern Blotting), and Unit 18 (PCR Analysis), Frederick M. Ausubul et al. (ed)., 1995.

The detection means according to this aspect of the invention may be any nucleic acid-based detection means such as, for example, nucleic acid hybridization or amplification reaction (eg. PCR), a nucleic acid sequence-based amplification (NASBA) system, inverse polymerase chain reaction (iPCR), in situ polymerase chain reaction, or reverse transcription polymerase chain reaction (RT-PCR), amongst others.

The probe can be labelled with a reporter molecule capable of producing an identifiable signal (e.g., a radioisotope such as ³²P or ³⁵S, or a fluorescent or biotinylated molecule). According to this embodiment, those skilled in the art will be aware that the detection of said reporter molecule provides for identification of the probe and that, following the hybridization reaction, the detection of the corresponding nucleotide sequences in the sample is facilitated. Additional probes can be used to confirm the assay results obtained using a single probe.

Wherein the detection means is an amplification reaction such as, for example, a polymerase chain reaction or a nucleic acid sequence-based amplification (NASBA) system or a variant thereof, one or more nucleic acid probes molecules of at least about 20 contiguous nucleotides in length is hybridized to mRNA encoding a cancer-associated protein, or alternatively, hybridized to cDNA or cRNA produced from said mRNA, and nucleic acid copies of the template are enzymically-amplified.

Those skilled in the art will be aware that there must be a sufficiently high percentage of nucleotide sequence identity between the probes and the RNA sequences in the sample template molecule for hybridization to occur. As stated previously, the stringency conditions can be selected to promote hybridization.

In one format, PCR provides for the hybridization of non-complementary probes to different strands of a double-stranded nucleic acid template molecule (ie. a DNA/RNA, RNA/RNA or DNA/DNA template), such that the hybridized probes are positioned to facilitate the 5′- to 3′ synthesis of nucleic acid in the intervening region, under the control of a thermostable DNA polymerase enzyme. In accordance with this embodiment, one sense probe and one antisense probe as described herein would be used to amplify DNA from the hybrid RNA/DNA template or cDNA.

In the present context, the cDNA would generally be produced by reverse transcription of mRNA present in the sample being tested (ie. RT-PCR). RT-PCR is particularly useful when it is desirable to determine expression of a cancer-associated gene. It is also known to those skilled in the art to use mRNA/DNA hybrid molecules as a template for such amplification reactions, and, as a consequence, first strand cDNA synthesis is all that is required to be performed prior to the amplification reaction.

Variations of the embodiments described herein are described in detail by McPherson et al., PCR: A Practical Approach. (series eds, D. Rickwood and B. D. Hames), IRL Press Limited, Oxford. pp 1-253, 1991.

The amplification reaction detection means described supra can be further coupled to a classical hybridization reaction detection means to further enhance sensitivity and specificity of the inventive method, such as by hybridizing the amplified DNA with a probe which is different from any of the probes used in the amplification reaction.

Similarly, the hybridization reaction detection means described supra can be further coupled to a second hybridization step employing a probe which is different from the probe used in the first hybridization reaction.

The comparison to be performed in accordance with the present invention may be a visual comparison of the signal generated by the probe, or alternatively, a comparison of data integrated from the signal, such as, for example, data that have been corrected or normalized to allow for variation between samples. Such comparisons can be readily performed by those skilled in the art.

### Polypeptides

Pancreatic cancer-associated polypeptides are encoded by pancreatic cancer-associated genes. It will be understood that such polypeptides include those polypeptide and fragments thereof that are homologous to the polypeptides encoded by the nucleotide sequences referred to in Tables 3-25, which are obtained from any source, for example related viral/bacterial proteins, cellular homologues and synthetic peptides, as well as variants or derivatives thereof.

Thus, the present invention encompasses the use of variants, homologues or derivatives of the cancer-associated proteins described in the accompanying Tables. In one embodiment, homologues are naturally occurring sequences, such as orthologues, tissue-specific isoforms and allelic variants.

In the context of the present invention, a homologous sequence is taken to include an amino acid sequence which is at least 60, 70, 80 or 90% identical, preferably at least 95 or 98% identical at the amino acid level over at least 20, 40, 60 or 80 amino acids with a sequence encoded by a nucleotide sequence referred to in any one of Tables 3-25. In particular, identity should typically be considered with respect to those regions of the sequence known to be essential for specific biological functions rather than non-essential neighbouring sequences.

Although amino acid identity can also be considered in terms of similarity (i.e. amino acid residues having similar chemical properties/functions), in the context of the present invention it is preferred to express identity in terms of sequence identity.

Identity comparisons are carried out as described above for nucleotide sequences with the appropriate modifications for amino acid sequences. For example when using the GCG Wisconsin Bestfit package (see below) the default gap penalty for amino acid sequences is −12 for a gap and −4 for each extension.

It should also be noted that where computer algorithms are used to align amino acid sequences, although the final % identity are measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). It is preferred to use the public default values for the GCG package, or in the case of other software, the default matrix, such as BLOSUM62.

The terms “variant” or “derivative” in relation to the amino acid sequences of the present invention includes any substitution of, variation of, modification of, replacement of, deletion of or addition of one (or more) amino acids from or to the sequence providing the resultant amino acid sequence preferably has biological activity, preferably having at least 25 to 50% of the activity as the polypeptides referred to in the sequence listings, more preferably at least substantially the same activity. Particular details of biological activity for each polypeptide are given in Tables 3-25.

Thus, the polypeptides referred to in Tables 3-25 and homologues thereof, are modified for use in the present invention. Typically, modifications are made that maintain the activity of the sequence. Thus, in one embodiment, amino acid substitutions are made, for example from 1, 2 or 3 to 10, 20 or 30 substitutions provided that the modified sequence retains at least about 25 to 50% of, or substantially the same activity. However, in an alternative preferred embodiment, modifications to the amino acid sequences of a cancer-associated protein are made intentionally to reduce the biological activity of the polypeptide. For example truncated polypeptides that remain capable of binding to target molecules but lack functional effector domains are useful as inhibitors of the biological activity of the full length molecule.

In general, preferably less than 20%, 10% or 5% of the amino acid residues of a variant or derivative are altered as compared with the corresponding region of the polypeptides referred to in Tables 3-25.

Amino acid substitutions may include the use of non-naturally occurring analogues, for example, to increase blood plasma half-life of a therapeutically administered polypeptide.

Conservative substitutions are made, for example according to the Table below. Amino acids in the same block in the second column and preferably in the same line in the third column are substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

Cancer-associated proteins also include fragments of the above mentioned full length polypeptides and variants thereof, including fragments of the sequences referred to in Tables 3-25 and homologues thereof. Preferred fragments include those which include an epitope. Suitable fragments will be at least about 6 or 8, e.g. at least 10, 12, 15 or 20 amino acids in length. They may also be less than 200, 100 or 50 amino acids in length. Polypeptide fragments may contain one or more (e.g. 2, 3, 5, or 10) substitutions, deletions or insertions, including conserved substitutions. Where substitutions, deletion and/or insertions have been made, for example by means of recombinant technology, preferably less than 20%, 10% or 5% of the amino acid residues of a protein referred to in Tables 3-25 or depicted in the Sequence Listing are altered.

Pancreatic cancer-associated proteins are preferably in a substantially isolated form. It will be understood that the protein are mixed with carriers or diluents which will not interfere with the intended purpose of the protein and still be regarded as substantially isolated. A pancreatic cancer-associated protein of the invention may also be in a substantially purified form, in which case it will generally comprise the protein in a preparation in which more than 90%, e.g. 95%, 98% or 99% pure as determined by SDS/PAGE or other art-recognized means for assessing protein purity.

### Protein Production

For producing full-length polypeptides or immunologically active derivatives thereof by recombinant means e.g., for antibody production, a protein-encoding region comprising at least about 15 contiguous nucleotides of the protein-encoding region of a nucleic acid referred to in any one of Tables 3-25 is placed in operable connection with a promoter or other regulatory sequence capable of regulating expression in a cell-free system or cellular system.

Reference herein to a “promoter” is to be taken in its broadest context and includes the transcriptional regulatory sequences of a classical genomic gene, including the TATA box which is required for accurate transcription initiation, with or without a CCAAT box sequence and additional regulatory elements (i.e., upstream activating sequences, enhancers and silencers) which alter gene expression in response to developmental and/or external stimuli, or in a tissue-specific manner. In the present context, the term “promoter” is also used to describe a recombinant, synthetic or fusion molecule, or derivative which confers, activates or enhances the expression of a nucleic acid molecule to which it is operably connected, and which encodes the polypeptide or peptide fragment. Preferred promoters can contain additional copies of one or more specific regulatory elements to further enhance expression and/or to alter the spatial expression and/or temporal expression of the said nucleic acid molecule.

Placing a nucleic acid molecule under the regulatory control of, i.e., “in operable connection with”, a promoter sequence means positioning said molecule such that expression is controlled by the promoter sequence. Promoters are generally positioned 5′ (upstream) to the coding sequence that they control. To construct heterologous promoter/structural gene combinations, it is generally preferred to position the promoter at a distance from the gene transcription start site that is approximately the same as the distance between that promoter and the gene it controls in its natural setting, i.e., the gene from which the promoter is derived. Furthermore, the regulatory elements comprising a promoter are usually positioned within 2 kb of the start site of transcription of the gene. As is known in the art, some variation in this distance can be accommodated without loss of promoter function. Similarly, the preferred positioning of a regulatory sequence element with respect to a heterologous gene to be placed under its control is defined by the positioning of the element in its natural setting, i.e., the genes from which it is derived. Again, as is known in the art, some variation in this distance can also occur.

The prerequisite for producing intact polypeptides and peptides in bacteria such as E. coli is the use of a strong promoter with an effective ribosome binding site. Typical promoters suitable for expression in bacterial cells such as E. coli include, but are not limited to, the lacz promoter, temperature-sensitive λ_{L} or λ_{R} promoters, T7 promoter or the IPTG-inducible tac promoter. A number of other vector systems for expressing the nucleic acid molecule of the invention in E. coil are well-known in the art and are described, for example, in Ausubel et al (In: Current Protocols in Molecular Biology. Wiley Interscience, ISBN 047150338, 1987) or Sambrook et al (In: Molecular cloning. A laboratory manual, second edition, Cold Spring Harbor Laboratory, Cold Spring Harbor, N.Y., 1989). Numerous plasmids with suitable promoter sequences for expression in bacteria and efficient ribosome binding sites have been described, such as for example, pKC30 (λ_{L}: Shimatake and Rosenberg, Nature 292, 128, 1981); pKK173-3 (tac. Amann and Brosius, Gene 40, 183, 1985), pET-3 (T7: Studier and Moffat, J. Mol. Biol. 189, 113, 1986); the pBAD/TOPO or pBAD/Thio-TOPO series of vectors containing an arabinose-inducible promoter (Invitrogen, Carlsbad, Calif.), the latter of which is designed to also produce fusion proteins with thioredoxin to enhance solubility of the expressed protein; the pFLEX series of expression vectors (Pfizer Inc., CT, USA); or the pOE series of expression vectors (Qiagen, CA), amongst others.

Typical promoters suitable for expression in viruses of eukaryotic cells and eukaryotic cells include the SV40 late promoter, SV40 early promoter and cytomegalovirus (CMV) promoter, CMV IE (cytomegalovirus immediate early) promoter amongst others. Preferred vectors for expression in mammalian cells (eg. 293, COS, CHO, 293T cells) include, but are not limited to, the pcDNA vector suite supplied by Invitrogen, in particular pcDNA 3.1 myc-His-tag comprising the CMV promoter and encoding a C-terminal 6×His and MYC tag; and the retrovirus vector pSRαtkneo (Muller et al., Mol. Cell. Biol., 11, 1785, 1991). The vector pcDNA 3.1 myc-His (Invitrogen) is particularly preferred for expressing a secreted form of a protein in 293T cells, wherein the expressed peptide or protein can be purified free of conspecific proteins, using standard affinity techniques that employ a Nickel column to bind the protein via the His tag.

A wide range of additional host/vector systems suitable for expressing polypeptides or immunological derivatives thereof are available publicly, and described, for example, in Sambrook et al (In: Molecular cloning. A laboratory manual, second edition, Cold Spring Harbor Laboratory, Cold Spring Harbor, N.Y., 1989).

Means for introducing the isolated nucleic acid molecule or a gene construct comprising same into a cell for expression are well-known to those skilled in the art. The technique used for a given organism depends on the known successful techniques. Means for introducing recombinant DNA into animal cells include microinjection, transfection mediated by DEAE-dextran, transfection mediated by liposomes such as by using lipofectamine (Gibco, Md., USA) and/or cellfectin (Gibco, Md., USA), PEG-mediated DNA uptake, electroporation and microparticle bombardment such as by using DNA-coated tungsten or gold particles (Agracetus Inc., WI, USA) amongst others.

For producing mutants, nucleotide insertion derivatives of the protein-encoding region are produced by making 5′ and 3′ terminal fusions, or by making intra-sequence insertions of single or multiple nucleotides or nucleotide analogues. Insertion nucleotide sequence variants are produced by introducing one or more nucleotides or nucleotide analogues into a predetermined site in the nucleotide sequence of said sequence, although random insertion is also possible with suitable screening of the resulting product being performed. Deletion variants are produced by removing one or more nucleotides from the nucleotide sequence. Substitutional nucleotide variants are produced by substituting at least one nucleotide in the sequence with a different nucleotide or a nucleotide analogue in its place, with the immunologically active derivative encoded therefor having an identical amino acid sequence, or only a limited number of amino acid modifications that do not alter its antigenicity compared to the base peptide or its ability to bind antibodies prepared against the base peptide. Such mutant derivatives will preferably have at least 80% identity with the base amino acid sequence from which they are derived.

Preferred immunologically active derivatives of a full-length polypeptide encoded by a gene referred to in any one of Tables 3-25 will comprise at least about 5-10 contiguous amino acids of the full-length amino acid sequence, more preferably at least about 10-20 contiguous amino acids in length, and even more preferably 20-30 contiguous amino acids in length.

For the purposes of producing derivatives using standard peptide synthesis techniques, such as, for example, Fmoc chemistry, a length not exceeding about 30-50 amino acids in length is preferred, as longer peptides are difficult to produce at high efficiency. Longer peptide fragments are readily achieved using recombinant DNA techniques wherein the peptide is expressed in a cell-free or cellular expression system comprising nucleic acid encoding the desired peptide fragment.

It will be apparent to the skilled artisan that any sufficiently antigenic region of at least about 5-10 amino acid residues can be used to prepare antibodies that bind generally to the polypeptides listed in Tables 3-25 or in the Sequence Listing.

An expressed protein or synthetic peptide is preferably produced as a recombinant fusion protein, such as for example, to aid in extraction and purification. To produce a fusion polypeptide, the open reading frames are covalently linked in the same reading frame, such as, for example, using standard cloning procedures as described by Ausubel et al. (Current Protocols in Molecular Biology, Wiley Interscience, ISBN 047150338, 1992), and expressed under control of a promoter. Examples of fusion protein partners include glutathione-S-transferase (GST), FLAG, hexahistidine, GAL4 (DNA binding and/or transcriptional activation domains) and β-galactosidase. It may also be convenient to include a proteolytic cleavage site between the fusion protein partner and the protein sequence of interest to allow removal of fusion protein sequences. Preferably the fusion protein will not hinder the immune function of the target protein.

In a particularly preferred embodiment, polypeptides are produced substantially free of conspecific proteins. Such purity can be assessed by standard procedures, such as, for example, SDS/polyacrylamide gel electrophoresis, 2-dimensional gene electrophoresis, chromatography, amino acid composition analysis, or amino acid sequence analysis.

To produce isolated polypeptides or fragments, eg., for antibody production, standard protein purification techniques may be employed. For example, gel filtration, ion exchange chromatography, reverse phase chromatography, or affinity chromatography, or a combination of any one or more said procedures, may be used. High pressure and low pressure procedures can also be employed, such as, for example, FPLC, or HPLC. To isolate the full-length proteins or peptide fragments comprising more than about 50-100 amino acids in length, it is particularly preferred to express the polypeptide in a suitable cellular expression system in combination with a suitable affinity tag, such as a 6×His tag, and to purify the polypeptide using an affinity step that bonds it via the tag (supra). Optionally, the tag may then be cleaved from the expressed polypeptide.

Alternatively, for short immunologically active derivatives of a full-length polypeptide, preferably those peptide fragments comprising less than about 50 amino acids in length, chemical synthesis techniques are conveniently used. As will be known to those skilled in the art, such techniques may also produce contaminating peptides that are shorter than the desired peptide, in which case the desired peptide is conveniently purified using reverse phase and/or ion exchange chromatography procedures at high pressure (ie. HPLC or FPLC).

### Antibodies

The invention also provides monoclonal or polyclonal antibodies that bind specifically to polypeptides of the invention or fragments thereof. Thus, the present invention further provides a process for the production of monoclonal or polyclonal antibodies to polypeptides of the invention.

The phrase “binds specifically” to a polypeptide means that the binding of the antibody to the protein or peptide is determinative of the presence of the protein, in a heterogeneous population of proteins and other biologics. Thus, under designated immunoassay conditions, the specified antibodies bind to a particular protein at least two times the background and more typically more than 10 to 100 times background. Typically, antibodies of the invention bind to a protein of interest with a Kd of at least about 0.1 mM, more usually at least about 1 TM, preferably at least about 0.1 TM, and most preferably at least, 0.01 TM.

Reference herein to antibody or antibodies includes whole polyclonal and monoclonal antibodies, and parts thereof, either alone or conjugated with other moieties. Antibody parts include Fab and F(ab)₂ fragments and single chain antibodies. The antibodies may be made in vivo in suitable laboratory animals, or, in the case of engineered antibodies (Single Chain Antibodies or SCABS, etc) using recombinant DNA techniques in vitro.

In accordance with this aspect of the invention, the antibodies may be produced for the purposes of immunizing the subject, in which case high titer or neutralizing antibodies that bind to a B cell epitope will be especially preferred. Suitable subjects for immunization will, of course, depend upon the immunizing antigen or antigenic B cell epitope. It is contemplated that the present invention will be broadly applicable to the immunization of a wide range of animals, such as, for example, farm animals (e.g. horses, cattle, sheep, pigs, goats, chickens, ducks, turkeys, and the like), laboratory animals (e.g. rats, mice, guinea pigs, rabbits), domestic animals (cats, dogs, birds and the like), feral or wild exotic animals (e.g. possums, cats, pigs, buffalo, wild dogs and the like) and humans.

Alternatively, the antibodies may be for commercial or diagnostic purposes, in which case the subject to whom the diagnostic/prognostic protein or immunogenic fragment or epitope thereof is administered will most likely be a laboratory or farm animal. A wide range of animal species are used for the production of antisera. Typically the animal used for production of antisera is a rabbit, a mouse, rat, hamster, guinea pig, goat, sheep, pig, dog, horse, or chicken. Because of the relatively large blood volume of rabbits, a rabbit is a preferred choice for production of polyclonal antibodies. However, as will be known to those skilled in the art, larger amounts of immunogen are required to obtain high antibodies from large animals as opposed to smaller animals such as mice. In such cases, it will be desirable to isolate the antibody from the immunized animal.

Preferably, the antibody is a high titer antibody. By “high titer” means a sufficiently high titer to be suitable for use in diagnostic or therapeutic applications. As will be known in the art, there is some variation in what might be considered “high titer”. For most applications a titer of at least about 10³-10⁴ is preferred. More preferably, the antibody titer will be in the range from about 10⁴ to about 10⁵, even more preferably in the range from about 10⁵ to about 10⁶.

More preferably, in the case of B cell epitopes from pathogens, viruses or bacteria, the antibody is a neutralizing antibody (i.e. it is capable of neutralizing the infectivity of the organism from which the B cell epitope is derived).

To generate antibodies, the diagnostic/prognostic protein or immunogenic fragment or epitope thereof, optionally formulated with any suitable or desired carrier, adjuvant, BRM, or pharmaceutically acceptable excipient, is conveniently administered in the form of an injectable composition. Injection may be intranasal, intramuscular, sub-cutaneous, intravenous, intradermal, intraperitoneal, or by other known route. For intravenous injection, it is desirable to include one or more fluid and nutrient replenishers. Means for preparing and characterizing antibodies are well known in the art. (See, e.g., ANTIBODIES: A LABORATORY MANUAL, Cold Spring Harbor Laboratory, 1988, incorporated herein by reference).

The efficacy of the diagnostic/prognostic protein or immunogenic fragment or epitope thereof in producing an antibody is established by injecting an animal, for example, a mouse, rat, rabbit, guinea pig, dog, horse, cow, goat or pig, with a formulation comprising the diagnostic/prognostic protein or immunogenic fragment or epitope thereof, and then monitoring the immune response to the B cell epitope, as described in the Examples. Both primary and secondary immune responses are monitored. The antibody titer is determined using any conventional immunoassay, such as, for example, ELISA, or radio immunoassay.

The production of polyclonal antibodies may be monitored by sampling blood of the immunized animal at various points following immunization. A second, booster injection, may be given, if required to achieve a desired antibody titer. The process of boosting and titering is repeated until a suitable titer is achieved. When a desired level of immunogenicity is obtained, the immunized animal is bled and the serum isolated and stored, and/or the animal is used to generate monoclonal antibodies (Mabs).

For the production of monoclonal antibodies (Mabs) any one of a number of well-known techniques may be used, such as, for example, the procedure exemplified in U.S. Pat. No. 4,196,265, incorporated herein by reference.

For example, a suitable animal will be immunized with an effective amount of the diagnostic/prognostic protein or immunogenic fragment or epitope thereof under conditions sufficient to stimulate antibody producing cells. Rodents such as mice and rats are preferred animals, however, the use of rabbit, sheep, or frog cells is also possible. The use of rats may provide certain advantages, but mice are preferred, with the BALB/c mouse being most preferred as the most routinely used animal and one that generally gives a higher percentage of stable fusions.

Following immunization, somatic cells with the potential for producing antibodies, specifically B lymphocytes (B cells), are selected for use in the MAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of animal with the highest antibody titer removed. Spleen lymphocytes are obtained by homogenizing the spleen with a syringe. Typically, a spleen from an immunized mouse contains approximately 5×10⁷ to 2×10⁸ lymphocytes.

The B cells from the immunized animal are then fused with cells of an immortal myeloma cell, generally derived from the same species as the animal that was immunized with the diagnostic/prognostic protein or immunogenic fragment or epitope thereof. Myeloma cell lines suited for use in hybridoma-producing fusion procedures preferably are non-antibody-producing, have high fusion efficiency and enzyme deficiencies that render them incapable of growing in certain selective media which support the growth of only the desired fused cells, or hybridomas. Any one of a number of myeloma cells may be used and these are known to those of skill in the art (e.g. murine P3-X63/Ag8, X63-Ag8.653, NS1/1.Ag 4 1, Sp210-Ag14, FO, NSO/U, MPC-11, MPC11-X45-GTG 1.7 and S194/5XXO; or rat R210.RCY3, Y3-Ag 1.2.3, IR983F and 4B210; and U-266, GM1500-GRG2, LICR-LON-HMy2 and UC729-6). A preferred murine myeloma cell is the NS-1 myeloma cell line (also termed P3-NS-1-Ag4-1), which is readily available from the NIGMS Human Genetic Mutant Cell Repository under Accession No. GM3573. Alternatively, a murine myeloma SP2/0 non-producer cell line that is 8-azaguanine-resistant is used.

To generate hybrids of antibody-producing spleen or lymph node cells and myeloma cells, somatic cells are mixed with myeloma cells in a proportion between about 20:1 to about 1:1, respectively, in the presence of an agent or agents (chemical or electrical) that promote the fusion of cell membranes. Fusion methods using Sendai virus have been described by Kohler and Milstein, Nature 256, 495-497, 1975; and Kohler and Milstein, Eur. J. Immunol. 6, 511-519, 1976. Methods using polyethylene glycol (PEG), such as 37% (v/v) PEG, are described in detail by Gefter et al., Somatic Cell Genet. 3, 231-236, 1977. The use of electrically induced fusion methods is also appropriate.

Hybrids are amplified by culture in a selective medium comprising an agent that blocks the de novo synthesis of nucleotides in the tissue culture media. Exemplary and preferred agents are aminopterin, methotrexate and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT, because only those hybridomas capable of operating nucleotide salvage pathways are able to survive in HAT medium, whereas myeloma cells are defective in key enzymes of the salvage pathway, (e.g., hypoxanthine phosphoribosyl transferase or HPRT), and they cannot survive. B cells can operate this salvage pathway, but they have a limited life span in culture and generally die within about two weeks. Accordingly, the only cells that can survive in the selective media are those hybrids formed from myeloma and B cells.

The amplified hybridomas are subjected to a functional selection for antibody specificity and/or titer, such as, for example, by immunoassay (e.g. radioimmunoassay, enzyme immunoassay, cytotoxicity assay, plaque assay, dot immunobinding assay, and the like).

The selected hybridomas are serially diluted and cloned into individual antibody-producing cell lines, which clones can then be propagated indefinitely to provide MAbs. The cell lines may be exploited for MAb production in two basic ways. A sample of the hybridoma is injected, usually in the peritoneal cavity, into a histocompatible animal of the type that was used to provide the somatic and myeloma cells for the original fusion. The injected animal develops tumors secreting the specific monoclonal antibody produced by the fused cell hybrid. The body fluids of the animal, such as serum or ascites fluid, can then be tapped to provide MAbs in high concentration. The individual cell lines could also be cultured in vitro, where the MAbs are naturally secreted into the culture medium from which they are readily obtained in high concentrations. MAbs produced by either means may be further purified, if desired, using filtration, centrifugation and various chromatographic methods such as HPLC or affinity chromatography.

Monoclonal antibodies of the present invention also include anti-idiotypic antibodies produced by methods well-known in the art. Monoclonal antibodies according to the present invention also may be monoclonal heteroconjugates, (i.e., hybrids of two or more antibody molecules). In another embodiment, monoclonal antibodies according to the invention are chimeric monoclonal antibodies. In one approach, the chimeric monoclonal antibody is engineered by cloning recombinant DNA containing the promoter, leader, and variable-region sequences from a mouse anti-PSA producing cell and the constant-region exons from a human antibody gene. The antibody encoded by such a recombinant gene is a mouse-human chimera. Its antibody specificity is determined by the variable region derived from mouse sequences. Its isotype, which is determined by the constant region, is derived from human DNA.

In another embodiment, the monoclonal antibody according to the present invention is a “humanized” monoclonal antibody, produced by any one of a number of techniques well-known in the art. That is, mouse complementary determining regions (“CDRs”) are transferred from heavy and light V-chains of the mouse Ig into a human V-domain, followed by the replacement of some human residues in the framework regions of their murine counterparts. “Humanized” monoclonal antibodies in accordance with this invention are especially suitable for use in vivo in diagnostic and therapeutic methods.

As stated above, the monoclonal antibodies and fragments thereof according to this invention are multiplied according to in vitro and in vivo methods well-known in the art. Multiplication in vitro is carried out in suitable culture media such as Dulbecco's modified Eagle medium or RPMI 1640 medium, optionally replenished by a mammalian serum such as fetal calf serum or trace elements and growth-sustaining supplements, e.g., feeder cells, such as normal mouse peritoneal exudate cells, spleen cells, bone marrow macrophages or the like. In vitro production provides relatively pure antibody preparations and allows scale-up to give large amounts of the desired antibodies. Techniques for large scale hybridoma cultivation under tissue culture conditions are known in the art and include homogenous suspension culture, (e.g., in an airlift reactor or in a continuous stirrer reactor or immobilized or entrapped cell culture).

Large amounts of the monoclonal antibody of the present invention also may be obtained by multiplying hybridoma cells in vivo. Cell clones are injected into mammals which are histocompatible with the parent cells, (e.g., syngeneic mice, to cause growth of antibody-producing tumors. Optionally, the animals are primed with a hydrocarbon, especially oils such as Pristane (tetramethylpentadecane) prior to injection.

In accordance with the present invention, fragments of the monoclonal antibody of the invention are obtained from monoclonal antibodies produced as described above, by methods which include digestion with enzymes such as pepsin or papain and/or cleavage of disulfide bonds by chemical reduction. Alternatively, monoclonal antibody fragments encompassed by the present invention are synthesized using an automated peptide synthesizer, or they may be produced manually using techniques well known in the art.

The monoclonal conjugates of the present invention are prepared by methods known in the art, e.g., by reacting a monoclonal antibody prepared as described above with, for instance, an enzyme in the presence of a coupling agent such as glutaraldehyde or periodate. Conjugates with fluorescein markers are prepared in the presence of these coupling agents, or by reaction with an isothiocyanate. Conjugates with metal chelates are similarly produced. Other moieties to which antibodies may be conjugated include radionuclides such as, for example, ³H, ¹²⁵I, ³²P, ³⁵S, ¹⁴C, ⁵¹Cr, ³⁶Cl, ⁵⁷Co, ⁵⁸Co, ⁵⁹Fe, ⁷⁵Se, and ¹⁵²Eu.

Radioactively labeled monoclonal antibodies of the present invention are produced according to well-known methods in the art. For instance, monoclonal antibodies are iodinated by contact with sodium or potassium iodide and a chemical oxidizing agent such as sodium hypochlorite, or an enzymatic oxidizing agent, such as lactoperoxidase. Monoclonal antibodies according to the invention may be labeled with technetium⁹⁹ by ligand exchange process, for example, by reducing pertechnetate with stannous solution, chelating the reduced technetium onto a Sephadex column and applying the antibody to this column or by direct labeling techniques, (e.g., by incubating pertechnate, a reducing agent such as SNCl₂, a buffer solution such as sodium-potassium phthalate solution, and the antibody).

Any immunoassay may be used to monitor antibody production by the diagnostic/prognostic protein or immunogenic fragment or epitope thereof. Immunoassays, in their most simple and direct sense, are binding assays. Certain preferred immunoassays are the various types of enzyme linked immunosorbent assays (ELISAs) and radioimmunoassays (RIA) known in the art. Immunohistochemical detection using tissue sections is also particularly useful. However, it will be readily appreciated that detection is not limited to such techniques, and Western blotting, dot blotting, FACS analyses, and the like may also be used.

Most preferably, the assay will be capable of generating quantitative results.

For example, antibodies are tested in simple competition assays. A known antibody preparation that binds to the B cell epitope and the test antibody are incubated with an antigen composition comprising the B cell epitope, preferably in the context of the native antigen. “Antigen composition” as used herein means any composition that contains some version of the B cell epitope in an accessible form. Antigen-coated wells of an ELISA plate are particularly preferred. In one embodiment, one would pre-mix the known antibodies with varying amounts of the test antibodies (e.g., 1:1, 1:10 and 1:100) for a period of time prior to applying to the antigen composition. If one of the known antibodies is labeled, direct detection of the label bound to the antigen is possible; comparison to an unmixed sample assay will determine competition by the test antibody and, hence, cross-reactivity. Alternatively, using secondary antibodies specific for either the known or test antibody, one will be able to determine competition.

An antibody that binds to the antigen composition will be able to effectively compete for binding of the known antibody and thus will significantly reduce binding of the latter. The reactivity of the known antibodies in the absence of any test antibody is the control. A significant reduction in reactivity in the presence of a test antibody is indicative of a test antibody that binds to the B cell epitope (i.e., it cross-reacts with the known antibody).

In one exemplary ELISA, the antibodies against the diagnostic/prognostic protein or immunogenic fragment or B cell epitope are immobilized onto a selected surface exhibiting protein affinity, such as a well in a polystyrene microtiter plate. Then, a composition containing a peptide comprising the B cell epitope is added to the wells. After binding and washing to remove non-specifically bound immune complexes, the bound epitope may be detected. Detection is generally achieved by the addition of a second antibody that is known to bind to the B cell epitope and is linked to a detectable label. This type of ELISA is a simple “sandwich ELISA”. Detection may also be achieved by the addition of said second antibody, followed by the addition of a third antibody that has binding affinity for the second antibody, with the third antibody being linked to a detectable label.

Antibodies of the invention may be bound to a solid support and/or packaged into kits in a suitable container along with suitable reagents, controls, instructions and the like.

### Immunoassay Formats

In one embodiment, a cancer-associated protein or an immunogenic fragment or epitope thereof is detected in a patient sample, wherein the level of the protein or immunogenic fragment or epitope in the sample is indicative of pancreatic cancer or disease recurrence or an indicator of poor survival. Preferably, the method comprises contacting a biological sample derived from the subject with an antibody capable of binding to a cancer-associated protein or an immunogenic fragment or epitope thereof, and detecting the formation of an antigen-antibody complex.

In another embodiment, an antibody against a cancer-associated protein or epitope thereof is detected in a patient sample, wherein the level of the antibody in the sample is indicative of pancreatic cancer or disease recurrence or an indicator of poor survival. Preferably, the method comprises contacting a biological sample derived from the subject with a cancer-associated protein or an antigenic fragment eg., a B cell epitope or other immunogenic fragment thereof, and detecting the formation of an antigen-antibody complex.

The diagnostic assays of the invention are useful for determining the progression of pancreatic cancer or a metastasis thereof in a subject. In accordance with these prognostic applications of the invention, the level of a cancer-associated protein or an immunogenic fragment or epitope thereof in a biological sample is correlated with the disease state eg., as determined by clinical symptoms or biochemical tests.

Accordingly, a further embodiment of the invention provides a method for detecting a pancreatic cancer cell in a subject, said method comprising:
(i) determining the level of a pancreatic cancer-associate protein in a test sample from said subject; and
(ii) comparing the level determined at (i) to the level of said pancreatic cancer-associated protein in a comparable sample from a healthy or normal individual,
   wherein a level of said pancreatic cancer-associate protein at (i) that is modified in the test sample relative to the comparable sample from the normal or healthy individual is indicative of the presence of a pancreatic cancer cell in said subject.

In one embodiment of the diagnostic/prognostic methods described herein, the biological sample is obtained previously from the subject. In accordance with such an embodiment, the prognostic or diagnostic method is performed ex vivo.

In yet another embodiment, the subject diagnostic/prognostic methods further comprise processing the sample from the subject to produce a derivative or extract that comprises the analyte.

Preferred detection systems contemplated herein include any known assay for detecting proteins or antibodies in a biological sample isolated from a human subject, such as, for example, SDS/PAGE, isoelectric focussing, 2-dimensional gel electrophoresis comprising SDS/PAGE and isoelectric focussing, an immunoassay, a detection based system using an antibody or non-antibody ligand of the protein, such as, for example, a small molecule (e.g. a chemical compound, agonist, antagonist, allosteric modulator, competitive inhibitor, or non-competitive inhibitor, of the protein). In accordance with these embodiments, the antibody or small molecule may be used in any standard solid phase or solution phase assay format amenable to the detection of proteins. Optical or fluorescent detection, such as, for example, using mass spectrometry, MALDI-TOF, biosensor technology, evanescent fiber optics, or fluorescence resonance energy transfer, is clearly encompassed by the present invention. Assay systems suitable for use in high throughput screening of mass samples, particularly a high throughput spectroscopy resonance method (e.g. MALDI-TOF, electrospray MS or nano-electrospray MS), are particularly contemplated.

Immunoassay formats are particularly preferred, eg., selected from the group consisting of, an immunoblot, a Western blot, a dot blot, an enzyme linked immunosorbent assay (ELISA), radioimmunoassay (RIA), enzyme immunoassay. Modified immunoassays utilizing fluorescence resonance energy transfer (FRET), isotope-coded affinity tags (ICAT), matrix-assisted laser desorption/ionization time of flight (MALDI-TOF), electrospray ionization (ESI), biosensor technology, evanescent fiber-optics technology or protein chip technology are also useful.

Preferably, the assay is a semi-quantitative assay or quantitative assay.

Standard solid phase ELISA formats are particularly useful in determining the concentration of a protein or antibody from a variety of patient samples.

In one form such as an assay involves immobilising a biological sample comprising antibodies against the cancer-associated protein or epitope, or alternatively a pancreatic cancer-associated protein or an immunogenic fragment thereof, onto a solid matrix, such as, for example a polystyrene or polycarbonate microwell or dipstick, a membrane, or a glass support (e.g. a glass slide).

In the case of an antigen-based assay, an antibody that specifically binds a pancreatic cancer-associated protein is brought into direct contact with the immobilised biological sample, and forms a direct bond with any of its target protein present in said sample. For an antibody-based assay, an immobilized pancreatic cancer-associated protein or an immunogenic fragment or epitope thereof is contacted with the sample. The added antibody or protein in solution is generally labelled with a detectable reporter molecule, such as for example, a fluorescent label (e.g. FITC or Texas Red) or an enzyme (e.g. horseradish peroxidase (HRP)), alkaline phosphatase (AP) or β-galactosidase. Alternatively, or in addition, a second labelled antibody can be used that binds to the first antibody or to the isolated/recombinant antigen. Following washing to remove any unbound antibody or antigen, as appropriate, the label is detected either directly, in the case of a fluorescent label, or through the addition of a substrate, such as for example hydrogen peroxide, TMB, or toluidine, or 5-bromo-4-chloro-3-indol-beta-D-galaotopyranoside (x-gal).

Such ELISA based systems are particularly suitable for quantification of the amount of a protein or antibody in a sample, such as, for example, by calibrating the detection system against known amounts of a standard.

In another form, an ELISA consists of immobilizing an antibody that specifically binds a pancreatic cancer-associated protein on a solid matrix, such as, for example, a membrane, a polystyrene or polycarbonate microwell, a polystyrene or polycarbonate dipstick or a glass support. A patient sample is then brought into physical relation with said antibody, and the antigen in the sample is bound or ‘captured’. The bound protein can then be detected using a labelled antibody. For example if the protein is captured from a human sample, an anti-human antibody is used to detect the captured protein. Alternatively, a third labelled antibody can be used that binds the second (detecting) antibody.

It will be apparent to the skilled person that the assay formats described herein are amenable to high throughput formats, such as, for example automation of screening processes, or a microarray format as described in Mendoza et al, Biotechniques 27(4): 778-788, 1999. Furthermore, variations of the above described assay will be apparent to those skilled in the art, such as, for example, a competitive ELISA.

Alternatively, the presence of antibodies against the cancer-associate protein, or alternatively an oarian cancer-associated protein or an immunogenic fragment thereof, is detected using a radioimmunoassay (RIA). The basic principle of the assay is the use of a radiolabelled antibody or antigen to detect antibody antigen interactions. For example, an antibody that specifically binds to a pancreatic cancer-associated protein can be bound to a solid support and a biological sample brought into direct contact with said antibody. To detect the bound antigen, an isolated and/or recombinant form of the antigen is radiolabelled is brought into contact with the same antibody. Following washing the amount of bound radioactivity is detected. As any antigen in the biological sample inhibits binding of the radiolabelled antigen the amount of radioactivity detected is inversely proportional to the amount of antigen in the sample. Such an assay may be quantitated by using a standard curve using increasing known concentrations of the isolated antigen.

As will be apparent to the skilled artisan, such an assay may be modified to use any reporter molecule, such as, for example, an enzyme or a fluorescent molecule, in place of a radioactive label.

Western blotting is also useful for detecting a pancreatic cancer-associated protein or an immunogenic fragment thereof. In such an assay protein from a biological sample is separated using sodium dodecyl sulphate (SDS) polyacrylamide gel electrophoresis (SDS-PAGE) using techniques well known in the art and described in, for example, Scopes (In: Protein Purification: Principles and Practice, Third Edition, Springer Verlag, 1994). Separated proteins are then transferred to a solid support, such as, for example, a membrane or more specifically PVDF membrane, using methods well known in the art, for example, electrotransfer. This membrane may then be blocked and probed with a labelled antibody or ligand that specifically binds a pancreatic cancer-associated protein. Alternatively, a labelled secondary, or even tertiary, antibody or ligand can be used to detect the binding of a specific primary antibody.

High-throughput methods for detecting the presence or absence of antibodies, or alternatively pancreatic cancer-associated protein or an immunogenic fragment thereof are particularly preferred.

In one embodiment, MALDI-TOF is used for the rapid identification of a protein. Accordingly, there is no need to detect the proteins of interest using an antibody or ligand that specifically binds to the protein of interest. Rather, proteins from a biological sample are separated using gel electrophoresis using methods well known in the art and those proteins at approximately the correct molecular weight and/or isoelectric point are analysed using MALDI-TOF to determine the presence or absence of a protein of interest.

Alternatively, MALDI or ESI or a combination of approaches is used to determine the concentration of a particular protein in a biological sample, such as, for example sputum. Such proteins are preferably well characterised previously with regard to parameters such as molecular weight and isoelectric point.

Biosensor devices generally employ an electrode surface in combination with current or impedance measuring elements to be integrated into a device in combination with the assay substrate (such as that described in U.S. Pat. No. 5,567,301). An antibody or ligand that specifically binds to a protein of interest is preferably incorporated onto the surface of a biosensor device and a biological sample isolated from a patient (for example sputum that has been solubilised using the methods described herein) contacted to said device. A change in the detected current or impedance by the biosensor device indicates protein binding to said antibody or ligand. Some forms of biosensors known in the art also rely on surface plasmon resonance to detect protein interactions, whereby a change in the surface plasmon resonance surface of reflection is indicative of a protein binding to a ligand or antibody (U.S. Pat. Nos. 5,485,277 and 5,492,840).

Biosensors are of particular use in high throughput analysis due to the ease of adapting such systems to micro- or nano-scales. Furthermore, such systems are conveniently adapted to incorporate several detection reagents, allowing for multiplexing of diagnostic reagents in a single biosensor unit. This permits the simultaneous detection of several epitopes in a small amount of body fluids.

Evanescent biosensors are also preferred as they do not require the pretreatment of a biological sample prior to detection of a protein of interest. An evanescent biosensor generally relies upon light of a predetermined wavelength interacting with a fluorescent molecule, such as for example, a fluorescent antibody attached near the probe's surface, to emit fluorescence at a different wavelength upon binding of the diagnostic protein to the antibody or ligand.

To produce protein chips, the proteins, peptides, polypeptides, antibodies or ligands that are able to bind specific antibodies or proteins of interest are bound to a solid support such as for example glass, polycarbonate, polytetrafluoroethylene, polystyrene, silicon oxide, metal or silicon nitride. This immobilization is either direct (e.g. by covalent linkage, such as, for example, Schiff's base formation, disulfide linkage, or amide or urea bond formation) or indirect. Methods of generating a protein chip are known in the art and are described in for example U.S. Patent Application No. 20020136821, 20020192654, 20020102617 and U.S. Pat. No. 6,391,625. In order to bind a protein to a solid support it is often necessary to treat the solid support so as to create chemically reactive groups on the surface, such as, for example, with an aldehyde-containing silane reagent. Alternatively, an antibody or ligand may be captured on a microfabricated polyacrylamide gel pad and accelerated into the gel using microelectrophoresis as described in, Arenkov et al. Anal. Biochem. 278:123-131, 2000.

A protein chip is preferably generated such that several proteins, ligands or antibodies are arrayed on said chip. This format permits the simultaneous screening for the presence of several proteins in a sample.

Alternatively, a protein chip may comprise only one protein, ligand or antibody, and be used to screen one or more patient samples for the presence of one polypeptide of interest. Such a chip may also be used to simultaneously screen an array of patient samples for a polypeptide of interest.

Preferably, a sample to be analysed using a protein chip is attached to a reporter molecule, such as, for example, a fluorescent molecule, a radioactive molecule, an enzyme, or an antibody that is detectable using methods well known in the art. Accordingly, by contacting a protein chip with a labelled sample and subsequent washing to remove any unbound proteins the presence of a bound protein is detected using methods well known in the art, such as, for example using a DNA microarray reader.

Alternatively, biomolecular interaction analysis-mass spectrometry (BIA-MS) is used to rapidly detect and characterise a protein present in complex biological samples at the low- to sub-fmole level (Nelson et al. Electrophoresis 21: 1155-1163, 2000). One technique useful in the analysis of a protein chip is surface enhanced laser desorption/ionization-time of flight-mass spectrometry (SELDI-TOF-MS) technology to characterise a protein bound to the protein chip. Alternatively, the protein chip is analysed using ESI as described in U.S. Patent Application 20020139751.

As will be apparent to the skilled artisan, protein chips are particularly amenable to multiplexing of detection reagents. Accordingly, several antibodies or ligands each able to specifically bind a different peptide or protein may be bound to different regions of said protein chip. Analysis of a biological sample using said chip then permits the detecting of multiple proteins of interest, or multiple B cell epitopes of the pancreatic cancer-associated protein. Multiplexing of diagnostic and prognostic markers is particularly contemplated in the present invention.

In a further embodiment, the samples are analysed using ICAT, essentially as described in US Patent Application No. 20020076739. This system relies upon the labelling of a protein sample from one source (i.e. a healthy individual) with a reagent and the labelling of a protein sample from another source (i.e. a tuberculosis patient) with a second reagent that is chemically identical to the first reagent, but differs in mass due to isotope composition. It is preferable that the first and second reagents also comprise a biotin molecule. Equal concentrations of the two samples are then mixed, and peptides recovered by avidin affinity chromatography. Samples are then analysed using mass spectrometry. Any difference in peak heights between the heavy and light peptide ions directly correlates with a difference in protein abundance in a biological sample. The identity of such proteins may then be determined using a method well known in the art, such as, for example MALDI-TOF, or ESI.

As will be apparent to those skilled in the art a diagnostic or prognostic assay described herein may be a multiplexed assay. As used herein the term “multiplex”, shall be understood not only to mean the detection of two or more diagnostic or prognostic markers in a single sample simultaneously, but also to encompass consecutive detection of two or more diagnostic or prognostic markers in a single sample, simultaneous detection of two or more diagnostic or prognostic markers in distinct but matched samples, and consecutive detection of two or more diagnostic or prognostic markers in distinct but matched samples. As used herein the term “matched samples” shall be understood to mean two or more samples derived from the same initial biological sample, or two or more biological samples isolated at the same point in time.

Accordingly, a multiplexed assay may comprise an assay that detects several antibodies and/or epitopes in the same reaction and simultaneously, or alternatively, it may detect other one or more antigens/antibodies in addition to one or more antibodies and/or epitopes. As will be apparent to the skilled artisan, if such an assay is antibody or ligand based, both of these antibodies must function under the same conditions.

### Diagnostic Assay Kits

A further aspect of the present invention provides a kit for detecting a pancreactic cancer cell in a biological sample. In one embodiment, the kit comprises:
(i) one or more isolated antibodies that bind to a pancreatic cancer-associated protein or an immunogenic fragment or epitope thereof; and
(ii) means for detecting the formation of an antigen-antibody complex.

In an alternative embodiment, the kit comprises:
(i) an isolated or recombinant pancreatic cancer-associated protein or an immunogenic fragment or epitope thereof; and
(ii) means for detecting the formation of an antigen-antibody complex.

Optionally, the kit further comprises means for the detection of the binding of an antibody, fragment thereof or a ligand to a pancreatic cancer-associated protein. Such means include a reporter molecule such as, for example, an enzyme (such as horseradish peroxidase or alkaline phosphatase), a substrate, a cofactor, an inhibitor, a dye, a radionucleotide, a luminescent group, a fluorescent group, biotin or a colloidal particle, such as colloidal gold or selenium. Preferably such a reporter molecule is directly linked to the antibody or ligand.

In yet another embodiment, a kit may additionally comprise a reference sample. Such a reference sample.

In another embodiment, a reference sample comprises a peptide that is detected by an antibody or a ligand. Preferably, the peptide is of known concentration. Such a peptide is of particular use as a standard. Accordingly various known concentrations of such a peptide may be detected using a prognostic or diagnostic assay described herein.

In yet another embodiment, a kit comprises means for protein isolation (Scopes (In: Protein Purification: Principles and Practice, Third Edition, Springer Verlag, 1994).

### Bioinformatics

The ability to identify genes that are over or under expressed in pancreatic cancer can additionally provide high-resolution, high-sensitivity datasets which are used in the areas of diagnostics, therapeutics, drug development, pharmacogenetics, protein structure, biosensor development, and other related areas. For example, the expression profiles are used in diagnostic or prognostic evaluation of patients with pancreatic cancer. Or as another example, subcellular toxicological information are generated to better direct drug structure and activity correlation (see Anderson, Pharmaceutical Proteomics: Targets, Mechanism, and Function, paper presented at the IBC Proteomics conference, Coronado, Calif. (Jun. 11-12, 1998)).

Subcellular toxicological information can also be utilized in a biological sensor device to predict the likely toxicological effect of chemical exposures and likely tolerable exposure thresholds (see U.S. Pat. No. 5,811,231). Similar advantages accrue from datasets relevant to other biomolecules and bioactive agents (e.g., nucleic acids, saccharides, lipids, drugs, and the like).

Thus, in another embodiment, the present invention provides a database that includes at least one set of assay data. The data contained in the database is acquired, e.g., using array analysis either singly or in a library format. The database are in substantially any form in which data are maintained and transmitted, but is preferably an electronic database. The electronic database of the invention are maintained on any electronic device allowing for the storage of and access to the database, such as a personal computer, but is preferably distributed on a wide area network, such as the World Wide Web.

The focus of the present section on databases that include peptide sequence data is for clarity of illustration only. It will be apparent to those of skill in the art that similar databases are assembled for any assay data acquired using an assay of the invention.

The compositions and methods for identifying and/or quantitating the relative and/or absolute abundance of a variety of molecular and macromolecular species from a biological sample undergoing pancreatic cancer, i.e., the identification of pancreatic cancer-associated sequences described herein, provide an abundance of information, which are correlated with pathological conditions, predisposition to disease, drug testing, therapeutic monitoring, gene-disease causal linkages, identification of correlates of immunity and physiological status, among others. Although the data generated from the assays of the invention is suited for manual review and analysis, in a preferred embodiment, prior data processing using high-speed computers is utilized.

An array of methods for indexing and retrieving biomolecular information is known in the art. For example, U.S. Pat. Nos. 6,023,659 and 5,966,712 disclose a relational database system for storing biomolecular sequence information in a manner that allows sequences to be catalogued and searched according to one or more protein function hierarchies. U.S. Pat. No. 5,953,727 discloses a relational database having sequence records containing information in a format that allows a collection of partial-length DNA sequences to be catalogued and searched according to association with one or more sequencing projects for obtaining full-length sequences from the collection of partial length sequences. U.S. Pat. No. 5,706,498 discloses a gene database retrieval system for making a retrieval of a gene sequence similar to a sequence data item in a gene database based on the degree of similarity between a key sequence and a target sequence. U.S. Pat. No. 5,538,897 discloses a method using mass spectroscopy fragmentation patterns of peptides to identify amino acid sequences in computer databases by comparison of predicted mass spectra with experimentally-derived mass spectra using a closeness-of-fit measure. U.S. Pat. No. 5,926,818 discloses a multi-dimensional database comprising a functionality for multi-dimensional data analysis described as on-line analytical processing (OLAP), which entails the consolidation of projected and actual data according to more than one consolidation path or dimension. U.S. Pat. No. 5,295,261 reports a hybrid database structure in which the fields of each database record are divided into two classes, navigational and informational data, with navigational fields stored in a hierarchical topological map which are viewed as a tree structure or as the merger of two or more such tree structures.

See also Mount et al., Bioinformatics (2001); Biological Sequence Analysis: Probabilistic Models of Proteins and Nucleic Acids (Durbin et al., eds., 1999); Bioiraformatics: A Practical Guide to the Analysis of Genes and Proteins (Baxevanis & Oeullette eds., 1998)); Rashidi & Buehler, Bioinformatics: Basic Applications in Biological Science and Medicine (1999); Introduction to Computational Molecular Biology (Setubal et al., eds 1997); Bioinformatics: Methods and Protocols (Misener & Krawetz, eds, 2000); Bioinformatics: Sequence, Structure, and Databanks: A Practical Approach (Higgins & Taylor, eds., 2000); Brown, Bioinfor7natics: A Biologist's Guide to Biocomputing and the Internet (2001); Han & Kamber, Data Mining: Concepts and Techniques (2000); and Waterman, Introduction to Computational Biology: Maps, Sequences, and Genomes (1995).

The present invention provides a computer database comprising a computer and software for storing in computer-retrievable form assay data records cross-tabulated, e.g., with data specifying the source of the target-containing sample from which each sequence specificity record was obtained.

In an exemplary embodiment, at least one of the sources of target-containing sample is from a control tissue sample known to be free of pathological disorders. In a variation, at least one of the sources is a known pathological tissue specimen, e.g., a neoplastic lesion or another tissue specimen to be analyzed for prostate cancer. In another variation, the assay records cross-tabulate one or more of the following parameters for each target species in a sample: (1) a unique identification code, which can include, e.g., a target molecular structure and/or characteristic separation coordinate (e.g., electrophoretic coordinates); (2) sample source; and (3) absolute and/or relative quantity of the target species present in the sample.

The invention also provides for the storage and retrieval of a collection of target data in a computer data storage apparatus, which can include magnetic disks, optical disks, magneto-optical disks, DRAM, SRAM, SGRAM, SDRAM, RDRAM, DDR RAM, magnetic bubble memory devices, and other data storage devices, including CPU registers and on-CPU data storage arrays. Typically, the target data records are stored as a bit pattern in an array of magnetic domains on a magnetizable medium or as an array of charge states or transistor gate states, such as an array of cells in a DRAM device (e.g., each cell comprised of a transistor and a charge storage area, which are on the transistor). In one embodiment, the invention provides such storage devices, and computer systems built therewith, comprising a bit pattern encoding a protein expression fingerprint record comprising unique identifiers for at least 10 target data records cross-tabulated with target source.

When the target is a peptide or nucleic acid, the invention preferably provides a method for identifying related peptide or nucleic acid sequences, comprising performing a computerised comparison between a peptide or nucleic acid sequence assay record stored in or retrieved from a computer storage device or database and at least one other sequence. The comparison can include a sequence analysis or comparison algorithm or computer program embodiment thereof (e.g., BLAST, FASTA, TFASTA, GAP, BESTFIT—see above) and/or the comparison are of the relative amount of a peptide or nucleic acid sequence in a pool of sequences determined from a polypeptide or nucleic acid sample of a specimen.

The invention also preferably provides a magnetic disk, such as an IBM-compatible (DOS, Windows, Windows95/0.98/2000, Windows NT, OS/2) or other format (e.g., Linux, SunOS, Solaris, AIX, SCO Unix, VMS, MV, Macintosh, etc.) floppy diskette or hard (fixed, Winchester) disk drive, comprising a bit pattern encoding data from an assay of the invention in a file format suitable for retrieval and processing in a computerized sequence analysis, comparison, or relative quantitation method.

The invention also provides a network, comprising a plurality of computing devices linked via a data link, such as an Ethernet cable (coax or IOBaseT), telephone line, ISDN line, wireless network, optical fiber, or other suitable signal transmission medium, whereby at least one network device (e.g., computer, disk array, etc.) comprises a pattern of magnetic domains (e.g., magnetic disk) and/or charge domains (e.g., an array of DRAM cells) composing a bit pattern encoding data acquired from an assay of the invention.

The invention also provides a method for transmitting assay data that includes generating an electronic signal on an electronic communications device, such as a modem, ISDN terminal adapter, DSL, cable modem, ATM switch, or the like, wherein the signal includes (in native or encrypted format) a bit pattern encoding data from an assay or a database comprising a plurality of assay results obtained by the method of the invention.

In a preferred embodiment, the invention provides a computer system for comparing a query target to a database containing an array of data structures, such as an assay result obtained by the method of the invention, and ranking database targets based on the degree of identity and gap weight to the target data. A central processor is preferably initialized to load and execute the computer program for alignment and/or comparison of the assay results. Data for a query target is entered into the central processor via an I/O device. Execution of the computer program results in the central processor retrieving the assay data from the data file, which comprises a binary description of an assay result.

The target data or record and the computer program are transferred to secondary memory, which is typically random access memory (e.g., DRAM, SRAM, SGRAM, or SDRAM). Targets are ranked according to the degree of correspondence between a selected assay characteristic (e.g., binding to a selected affinity moiety) and the same characteristic of the query target and results are output via an I/O device. For example, a central processor are a conventional computer (e.g., Intel Pentium, PowerPC, Alpha, PA-8000, SPARC, MIPS 4400, MIPS 10000, VAX, etc.); a program are a commercial or public domain molecular biology software package (e.g., UWGCG Sequence Analysis Software, Darwin); a data file are an optical or magnetic disk, a data server, a memory device (e.g., DRAM, SRAM, SGRAM, SDRAM, EPROM, bubble memory, flash memory, etc.); an I/O device are a terminal comprising a video display and a keyboard, a modem, an ISDN terminal adapter, an Ethernet port, a punched card reader, a magnetic strip reader, or other suitable I/O device.

The invention also preferably provides the use of a computer system, such as that described above, which comprises: (1) a computer; (2) a stored bit pattern encoding a collection of peptide sequence specificity records obtained by the methods of the invention, which are stored in the computer; (3) a comparison target, such as a query target; and (4) a program for alignment and comparison, typically with rank-ordering of comparison results on the basis of computed similarity values.

### Therapeutic Peptides

In accordance with this embodiment, pancreatic cancer-associated proteins of the present invention are administered therapeutically to patients for a time and under conditions sufficient to ameliorate the growth of a tumor in the subject or to prevent tumor recurrence.

It is preferred to use peptides that do not consisting solely of naturally-occurring amino acids but which have been modified, for example to reduce immunogenicity, to increase circulatory half-life in the body of the patient, to enhance bioavailability and/or to enhance efficacy and/or specificity.

A number of approaches have been used to modify peptides for therapeutic application. One approach is to link the peptides or proteins to a variety of polymers, such as polyethylene glycol (PEG) and polypropylene glycol (PPG)—see for example U.S. Pat. Nos. 5,091,176, 5,214,131 and U.S. Pat. No. 5,264,209.

Replacement of naturally-occurring amino acids with a variety of uncoded or modified amino acids such as D-amino acids and N-methyl amino acids may also be used to modify peptides

Another approach is to use bifunctional crosslinkers, such as N-succinimidyl 3-(2 pyridyldithio) propionate, succinimidyl 6-[3-(2 pyridyldithio) propionamido]hexanoate, and sulfosuccinimidyl 6-[3-(2 pyridyldithio) propionamido]hexanoate (see U.S. Pat. No. 5,580,853).

It are desirable to use derivatives of the pancreatic cancer-associated proteins of the invention which are conformationally constrained. Conformational constraint refers to the stability and preferred conformation of the three-dimensional shape assumed by a peptide. Conformational constraints include local constraints, involving restricting the conformational mobility of a single residue in a peptide; regional constraints, involving restricting the conformational mobility of a group of residues, which residues may form some secondary structural unit; and global constraints, involving the entire peptide structure.

The active conformation of the peptide are stabilized by a covalent modification, such as cyclization or by incorporation of gamma-lactam or other types of bridges. For example, side chains are cyclized to the backbone so as create a L-gamma-lactam moiety on each side of the interaction site. See, generally, Hruby et al., “Applications of Synthetic Peptides,” in Synthetic Peptides: A User's Guide: 259-345 (W.H. Freeman & Co. 1992). Cyclization also are achieved, for example, by formation of cystine bridges, coupling of amino and carboxy terminal groups of respective terminal amino acids, or coupling of the amino group of a Lys residue or a related homolog with a carboxy group of Asp, Glu or a related homolog. Coupling of the .alpha-amino group of a polypeptide with the epsilon-amino group of a lysine residue, using iodoacetic anhydride, are also undertaken. See Wood and Wetzel, 1992, Int'l J. Peptide Protein Res. 39: 533-39.

Another approach described in U.S. Pat. No. 5,891,418 is to include a metal-ion complexing backbone in the peptide structure. Typically, the preferred metal-peptide backbone is based on the requisite number of particular coordinating groups required by the coordination sphere of a given complexing metal ion. In general, most of the metal ions that may prove useful have a coordination number of four to six. The nature of the coordinating groups in the peptide chain includes nitrogen atoms with amine, amide, imidazole, or guanidino functionalities; sulfur atoms of thiols or disulfides; and oxygen atoms of hydroxy, phenolic, carbonyl, or carboxyl functionalities. In addition, the peptide chain or individual amino acids are chemically altered to include a coordinating group, such as for example oxime, hydrazino, sulfhydryl, phosphate, cyano, pyridino, piperidino, or morpholino. The peptide construct are either linear or cyclic, however a linear construct is typically preferred. One example of a small linear peptide is Gly-Gly-Gly-Gly which has four nitrogens (an N₄ complexation system) in the back bone that can complex to a metal ion with a coordination number of four.

A further technique for improving the properties of therapeutic peptides is to use non-peptide peptidomimetics. A wide variety of useful techniques are used to elucidating the precise structure of a peptide. These techniques include amino acid sequencing, x-ray crystallography, mass spectroscopy, nuclear magnetic resonance spectroscopy, computer-assisted molecular modeling, peptide mapping, and combinations thereof. Structural analysis of a peptide generally provides a large body of data which comprise the amino acid sequence of the peptide as well as the three-dimensional positioning of its atomic components. From this information, non-peptide peptidomimetics are designed that have the required chemical functionalities for therapeutic activity but are more stable, for example less susceptible to biological degradation. An example of this approach is provided in U.S. Pat. No. 5,811,512.

Techniques for chemically synthesising therapeutic peptides of the invention are described in the above references and also reviewed by Borgia and Fields, 2000, TibTech 18: 243-251 and described in detail in the references contained therein.

### Assays for Therapeutic Compounds

The pancreatic cancer proteins, nucleic acids, and antibodies as described herein are used in drug screening assays to identify candidate compounds for use in treating pancreatic cancer. The pancreatic cancer-associated proteins, antibodies, nucleic acids, modified proteins and cells containing pancreatic cancer sequences are used in drug screening assays or by evaluating the effect of drug candidates on a “gene expression profile” or expression profile of polypeptides. In a preferred embodiment, the expression profiles are used, preferably in conjunction with high throughput screening techniques to allow monitoring for expression profile genes after treatment with a candidate agent (e.g., Zlokarnik, et al., 1998, Science 279: 84-88); Heid, 1996, Genome Res 6: 986-94).

In a preferred embodiment, the pancreatic cancer-associated proteins, antibodies, nucleic acids, modified proteins and cells containing the native or modified pancreatic cancer-associated proteins are used in screening assays. That is, the present invention provides methods for screening for compounds/agents which modulate the pancreatic cancer phenotype or an identified physiological function of a pancreatic cancer-associated protein. As above, this are done on an individual gene level or by evaluating the effect of drug candidates on a “gene expression profile”. In a preferred embodiment, the expression profiles are used, preferably in conjunction with high throughput screening techniques to allow monitoring for expression profile genes after treatment with a candidate agent, see Zlokarnik, supra.

Having identified the differentially expressed genes herein, a variety of assays are executed. In a preferred embodiment, assays are run on an individual gene or protein level. That is, having identified a particular gene as up regulated in pancreatic cancer, test compounds are screened for the ability to modulate gene expression or for binding to the pancreatic cancer-associated protein. “Modulation” thus includes both an increase and a decrease in gene expression. The preferred amount of modulation will depend on the original change of the gene expression in normal versus tissue undergoing pancreatic cancer, with changes of at least 10%, preferably 50%, more preferably 100-300%, and in some embodiments 300-1000% or greater. Thus, if a gene exhibits a 4-fold increase in pancreatic cancer tissue compared to normal tissue, a decrease of about four-fold is often desired; similarly, a 10-fold decrease in pancreatic cancer tissue compared to normal tissue often provides a target value of a 10-fold increase in expression to be induced by the test compound.

The amount of gene expression are monitored using nucleic acid probes and the quantification of gene expression levels, or, alternatively, the gene product itself are monitored, e.g., through the use of antibodies to the pancreatic cancer-associated protein and standard immunoassays. Proteomics and separation techniques may also allow quantification of expression.

In a preferred embodiment, gene expression or protein monitoring of a number of entities, i.e., an expression profile, is monitored simultaneously. Such profiles will typically involve a plurality of those entities described herein.

In this embodiment, the pancreatic cancer nucleic acid probes are attached to biochips as outlined herein for the detection and quantification of pancreatic cancer sequences in a particular cell. Alternatively, PCR are used. Thus, a series are used with dispensed primers in desired wells. A PCR reaction can then be performed and analyzed for each well.

Expression monitoring are performed to identify compounds that modify the expression of one or more pancreatic cancer-associated sequences, e.g., a polynucleotide sequence set out in Tables 3-25. In a preferred embodiment, a test modulator is added to the cells prior to analysis. Moreover, screens are also provided to identify agents that modulate pancreatic cancer, modulate pancreatic cancer-associated proteins, bind to a pancreatic cancer-associated protein, or interfere with the binding of a pancreatic cancer-associated protein and an antibody or other binding partner.

The term “test compound” or “drug candidate” or “modulator” or grammatical equivalents as used herein describes any molecule, e.g., protein, oligopeptide, small organic molecule, polysaccharide, polynucleotide, etc., to be tested for the capacity to directly or indirectly alter the pancreatic cancer phenotype or the expression of a pancreatic cancer sequence, e.g., a nucleic acid or protein sequence. In preferred embodiments, modulators alter expression profiles, or expression profile nucleic acids or proteins provided herein. In one embodiment, the modulator suppresses a pancreatic cancer phenotype, e.g. to a normal tissue fingerprint. In another embodiment, a modulator induced a pancreatic cancer phenotype. Generally, a plurality of assay mixtures are run in parallel with different agent concentrations to obtain a differential response to the various concentrations. Typically, one of these concentrations serves as a negative control, i.e., at zero concentration or below the level of detection.

Drug candidates encompass numerous chemical classes, though typically they are organic molecules, preferably small organic compounds having a molecular weight of more than 100 and less than about 2,500 daltons. Preferred small molecules are less than 2000, or less than 1500 or less than 1000 or less than 500 Daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Particularly preferred are peptides.

In one aspect, a modulator will neutralize the effect of a pancreatic cancer-associated protein. By “neutralize” is meant that activity of a protein is inhibited or blocked and the consequent effect on the cell.

In certain embodiments, combinatorial libraries of potential modulators will be screened for an ability to bind to a pancreatic cancer polypeptide or to modulate activity. Conventionally, new chemical entities with useful properties are generated by identifying a chemical compound (called a “lead compound”) with some desirable property or activity, e.g., inhibiting activity, creating variants of the lead compound, and evaluating the property and activity of those variant compounds. Often, high throughput screening (HTS) methods are employed for such an analysis.

In one preferred embodiment, high throughput screening methods involve providing a library containing a large number of potential therapeutic compounds (candidate compounds). Such “combinatorial chemical libraries” are then screened in one or more assays to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis by combining a number of chemical “building blocks” such as reagents. For example, a linear combinatorial chemical library, such as a polypeptide (e.g., mutein) library, is formed by combining a set of chemical building blocks called amino acids in every possible way for a given compound length (i.e., the number of amino acids in a polypeptide compound). Millions of chemical compounds are synthesized through such combinatorial mixing of chemical building blocks (Gallop et al., 1994, J. Med. Chem. 37(9):1233-1251).

Preparation and screening of combinatorial chemical libraries is well known to those of skill in the art. Such combinatorial chemical libraries include, but are not limited to, peptide libraries, peptoids, encoded peptides, random bio-oligomers, nonpeptidal peptidomimetics, analogous organic syntheses of small compound libraries, nucleic acid libraries, peptide nucleic acid libraries, antibody libraries, carbohydrate libraries and small organic molecule libraries.

The assays to identify modulators are amenable to high throughput screening. Preferred assays thus detect enhancement or inhibition of pancreatic cancer gene transcription, inhibition or enhancement of polypeptide expression, and inhibition or enhancement of polypeptide activity.

High throughput assays for the presence, absence, quantification, or other properties of particular nucleic acids or protein products are well known to those of skill in the art. Similarly, binding assays and reporter gene assays are similarly well known. Thus, e.g., U.S. Pat. No. 5,559,410 discloses high throughput screening methods for proteins, U.S. Pat. No. 5,585,639 discloses high throughput screening methods for nucleic acid binding (i.e., in arrays), while U.S. Pat. Nos. 5,576,220 and 5,541,061 disclose high throughput methods of screening for ligand/antibody binding.

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures, including all samlsle and reagent pipetting, liquid dispensing, timed incubations, and final readings of the microplate in detectors) appropriate for the assay. These configurable systems provide high throughput and rapid start up as well as a high degree of flexibility and customization. The manufacturers of such systems provide detailed protocols for various high throughput systems. Thus, e.g., Zymark Corp. provides technical bulletins describing screening systems for detecting the modulation of gene transcription, ligand binding, and the like.

In one embodiment, modulators are proteins, often naturally occurring proteins or fragments of naturally occurring proteins. Thus, e.g., cellular extracts containing proteins, or random or directed digests of proteinaceous cellular extracts, are used. In this way libraries of proteins are made for screening in the methods of the invention. Particularly preferred in this embodiment are libraries of bacterial, fungal, viral, and mammalian proteins, with the latter being preferred, and human proteins being especially preferred. Particularly useful test compound will be directed to the class of proteins to which the target belongs, e.g., substrates for enzymes or ligands and receptors.

In a preferred embodiment, modulators are peptides of from about 5 to about 30 amino acids, with from about 5 to about 20 amino acids being preferred, and from about 7 to about 15 being particularly preferred. The peptides are digests of naturally occurring proteins as is outlined above, random peptides, or “biased” random peptides. By “randomized” or grammatical equivalents herein is meant that each nucleic acid and peptide consists of essentially random nucleotides and amino acids, respectively. Since generally these random peptides (or nucleic acids, discussed below) are chemically synthesized, they may incorporate any nucleotide or amino acid at any position. The synthetic process are designed to generate randomized proteins or nucleic acids, to allow the formation of all or most of the possible combinations over the length of the sequence, thus forming a library of randomized candidate bioactive proteinaceous agents.

In one embodiment, the library is fully randomized, with no sequence preferences or constants at any position. In a preferred embodiment, the library is biased. That is, some positions within the sequence are either held constant, or are selected from a limited number of possibilities. For example, in a preferred embodiment, the nucleotides or amino acid residues are randomized within a defined class, e.g., of hydrophobic amino acids, hydrophilic residues, sterically biased (either small or large) residues, towards the creation of nucleic acid binding domains, the creation of cysteines, for cross-linking, prolines for SH-3 domains, serines, threonines, tyrosines or histidines for phosphorylation sites, etc., or to purines, etc.

Modulators of pancreatic cancer can also be nucleic acids, as defined below. As described above generally for proteins, nucleic acid modulating agents are naturally occurring nucleic acids, random nucleic acids, or “biased” random nucleic acids. For example, digests of procaryotic or eucaryotic genomes are used as is outlined above for proteins.

In certain embodiments, the activity of a pancreatic cancer-associated protein is down-regulated, or entirely inhibited, by the use of antisense polynucleotide, i.e., a nucleic acid complementary to, and which can preferably hybridize specifically to, a coding mRNA nucleic acid sequence, e.g., a pancreatic cancer-associated protein mRNA, or a subsequence thereof. Binding of the antisense polynucleotide to the mRNA reduces the translation and/or stability of the mRNA.

In the context of this invention, antisense nucleic acids can comprise naturally-occurring nucleotides, or synthetic species formed from naturally-occurring subunits or their close homologs. Antisense Nucleic acids may also have altered sugar moieties or inter-sugar linkages. Exemplary among these are the phosphorothioate and other sulfur containing species which are known for use in the art. Analogs are comprehended by this invention so long as they function effectively to hybridize with the pancreatic cancer-associated protein mRNA. See, e.g., Isis Pharmaceuticals, Carlsbad, Calif.; Sequitor, Inc., Natick, Mass.

Such antisense nucleic acids can readily be synthesized using recombinant means, or are synthesized in vitro. Equipment for such synthesis is sold by several vendors, including Applied Biosystems. The preparation of other oligonucleotides such as phosphorothioates and alkylated derivatives is also well known to those of skill in the art.

Antisense molecules as used herein include antisense or sense oligonucleotides. Sense oligonucleotides can, e.g., be employed to block transcription by binding to the anti-sense strand. The antisense and sense oligonucleotide comprise a single-stranded nucleic acid sequence (either RNA or DNA) capable of binding to target mRNA (sense) or DNA (antisense) sequences for pancreatic cancer molecules. Antisense or sense oligonucleotides, according to the present invention, comprise a fragment generally at least about 14 nucleotides, preferably from about 14 to 30 nucleotides. The ability to derive an antisense or a sense oligonucleotide, based upon a cDNA sequence encoding a given protein is described in, e.g., Stein & Cohen (Cancer Res. 48:2659 (1988 and van der Krol et al. (BioTechniques 6:958 (1988)).

In addition to antisense nucleic acids, ribozymes are used to target and inhibit transcription of pancreatic cancer-associated nucleotide sequences. A ribozyme is an RNA molecule that catalytically cleaves other RNA molecules. Different kinds of ribozymes have been described, including group I ribozymes, hammerhead ribozymes, hairpin ribozymes, RNase P, and axhead ribozymes (see, e.g., Castanotto et al., Adv. in Pharmacology 25: 289-317 (1994) for a general review of the properties of different 5 ribozymes).

Methods of preparing ribozymes are well known to those of skill in the art (see, e.g., WO 94/26877; Ojwang et al., Proc. Natl. Acad. Sci. USA 90:6340-6344 (1993); Yamada et al., Human Gene Therapy 1:39-45 (1994); Leavitt et al., Proc. Natl. Acad. Sci. USA 92:699-703 (1995); Leavitt et al., Human Gene Therapy 5:1151-120 (1994); and Yamada et al., Virology 205: 121-126 (1994)).

Polynucleotide modulators of pancreatic cancer are introduced into a cell containing the target nucleotide sequence by formation of a conjugate with a ligand binding molecule, as described in WO 91/04753. Suitable ligand binding molecules include, but are not limited to, cell surface receptors, growth factors, other cytokines, or other ligands that bind to cell surface receptors. Preferably, conjugation of the ligand binding molecule does not substantially interfere with the ability of the ligand binding molecule to bind to its corresponding molecule or receptor, or block entry of the sense or antisense oligonucleotide or its conjugated version into the cell. Alternatively, a polynucleotide modulator of pancreatic cancer are introduced into a cell containing the target nucleic acid sequence, e.g., by formation of an polynucleotide-lipid complex, as described in WO 90/10448. It is understood that the use of antisense molecules or knock out and knock in models may also be used in screening assays as discussed above, in addition to methods of treatment.

As noted above, gene expression monitoring is conveniently used to test candidate modulators (e.g., protein, nucleic acid or small molecule). After the candidate agent has been added and the cells allowed to incubate for some period of time, the sample containing a target sequence to be analyzed is added to the biochip. If required, the target sequence is prepared using known techniques. For example, the sample are treated to lyse the cells, using known lysis buffers, electroporation, etc., with purification and/or amplification such as PCR performed as appropriate. For example, an in vitro transcription with labels covalently attached to the nucleotides is performed. Generally, the nucleic acids are labeled with biotin-FITC or PE, or with cy3 or cy5.

In a preferred embodiment, the target sequence is labeled with, e.g., a fluorescent, a chemiluminescent, a chemical, or a radioactive signal, to provide a means of detecting the target sequence's specific binding to a probe. The label also are an enzyme, such as, alkaline phosphatase or horseradish peroxidase, which when provided with an appropriate substrate produces a product that are detected. Alternatively, the label are a labeled compound or small molecule, such as an enzyme inhibitor, that binds but is not catalyzed or altered by the enzyme. The label also are a moiety or compound, such as, an epitope tag or biotin which specifically binds to streptavidin. For the example of biotin, the streptavidin is labeled as described above, thereby, providing a detectable signal for the bound target sequence. Unbound labeled streptavidin is typically removed prior to analysis.

As will be appreciated by those in the art, these assays are direct hybridization assays or can comprise “sandwich assays”, which include the use of multiple probes, as is generally outlined in U.S. Pat. Nos. 5,681,702, 5,597,909, 5,545,730, 5,594,117, 5,591,584, 5,571,670, 5,580,731, 5,571,670, 5,591,584, 5,624,802, 5,635,352, 5,594,118, 5,359,100, 5,124,246 and 5,681,697, all of which are hereby incorporated by reference. In this embodiment, in general, the target nucleic acid is prepared as outlined above, and then added to the biochip comprising a plurality of nucleic acid probes, under conditions that allow the formation of a hybridization complex.

A variety of hybridization conditions are used in the present invention, including high, moderate and low stringency conditions as outlined above. The assays are generally run under stringency conditions which allows formation of the label probe hybridization complex only in the presence of target. Stringency are controlled by altering a step parameter that is a thermodynamic variable, including, but not limited to, temperature, formamide concentration, salt concentration, chaotropic salt concentration pH, organic solvent concentration, etc.

These parameters may also be used to control non-specific binding, as is generally outlined in U.S. Pat. No. 5,681,697. Thus it are desirable to perform certain steps at higher stringency conditions to reduce non-specific binding.

The reactions outlined herein are accomplished in a variety of ways. Components of the reaction are added simultaneously, or sequentially, in different orders, with preferred embodiments outlined below. In addition, the reaction may include a variety of other reagents. These include salts, buffers, neutral proteins, e.g. albumin, detergents, etc. which are used to facilitate optimal hybridization and detection, and/or reduce non-specific or background interactions. Reagents that otherwise improve the efficiency of the assay, such as protease inhibitors, nuclease inhibitors, anti-microbial agents, etc., may also be used as appropriate, depending on the sample preparation methods and purity of the target.

The assay data are analyzed to determine the expression levels, and changes in expression levels as between states, of individual genes, forming a gene expression profile.

Screens are performed to identify modulators of the pancreatic cancer phenotype. In one embodiment, screening is performed to identify modulators that can induce or suppress a particular expression profile, thus preferably generating the associated phenotype. In another embodiment, e.g., for diagnostic applications, having identified differentially expressed genes important in a particular state, screens are performed to identify modulators that alter expression of individual genes. In an another embodiment, screening is performed to identify modulators that alter a biological function of the expression product of a differentially expressed gene. Again, having identified the importance of a gene in a particular state, screens are performed to identify agents that bind and/or modulate the biological activity of the gene product.

In addition screens are done for genes that are induced in response to a candidate agent. After identifying a modulator based upon its ability to suppress a pancreatic cancer expression pattern leading to a normal expression pattern, or to modulate a single pancreatic cancer gene expression profile so as to mimic the expression of the gene from normal tissue, a screen as described above are performed to identify genes that are specifically modulated in response to the agent. Comparing expression profiles between normal tissue and agent treated pancreatic cancer tissue reveals genes that are not expressed in normal tissue or pancreatic cancer tissue, but are expressed in agent treated tissue. These agent-specific sequences are identified and used by methods described herein for pancreatic cancer genes or proteins. In particular these sequences and the proteins they encode find use in marking or identifying agent treated cells. In addition, antibodies are raised against the agent induced proteins and used to target novel therapeutics to the treated pancreatic cancer tissue sample.

Thus, in one embodiment, a test compound is administered to a population of pancreatic cancer cells, that have an associated pancreatic cancer expression profile. By “administration” or “contacting” herein is meant that the candidate agent is added to the cells in such a manner as to allow the agent to act upon the cell, whether by uptake and intracellular action, or by action at the cell surface. In some embodiments, nucleic acid encoding a proteinaceous candidate agent (i.e., a peptide) are put into a viral construct such as an adenoviral or retroviral construct, and added to the cell, such that expression of the peptide agent is accomplished. Regulatable gene administration systems can also be used.

Once the test compound has been administered to the cells, the cells are washed if desired and are allowed to incubate under preferably physiological conditions for some period of time. The cells are then harvested and a new gene expression profile is generated, as outlined herein.

Thus, e.g., pancreatic cancer tissue are screened for agents that modulate, e.g., induce or suppress the pancreatic cancer phenotype. A change in at least one gene, preferably many, of the expression profile indicates that the agent has an effect on pancreatic cancer activity. By defining such a signature for the pancreatic cancer phenotype, screens for new drugs that alter the phenotype are devised. With this approach, the drug target need not be known and need not be represented in the original expression screening platform, nor does the level of transcript for the target protein need to change.

In a preferred embodiment, as outlined above, screens are done on individual genes and gene products (proteins). That is, having identified a particular differentially expressed gene as important in a particular state, screening of modulators of either the expression of the gene or the gene product itself are done. The gene products of differentially expressed genes are sometimes referred to herein as “pancreatic cancer-associated proteins” or a “pancreatic cancer modulatory protein”. The pancreatic cancer modulatory protein are a fragment, or alternatively, be the full length protein to the fragment encoded by the nucleic acids referred to in Tables 1-3. Preferably, the pancreatic cancer modulatory protein is a fragment. In a preferred embodiment, the pancreatic cancer amino acid sequence which is used to determine sequence identity or similarity is encoded by a nucleic acid referred to in Tables 1-3. In another embodiment, the sequences are naturally occurring allelic variants of a protein encoded by a nucleic acid referred to in Tables 1-3. In another embodiment, the sequences are sequence variants as further described herein.

Preferably, the pancreatic cancer modulatory protein is a fragment of approximately 14 to 24 amino acids long. More preferably the fragment is a soluble fragment. Preferably, the fragment includes a non-transmembrane region. In a preferred embodiment, the fragment has an N-terminal Cys to aid in solubility. In one embodiment, the C-terminus of the fragment is kept as a free acid and the N-terminus is a free amine to aid in coupling, i.e., to cysteine.

In one embodiment the pancreatic cancer-associated proteins are conjugated to an immunogenic agent as discussed herein. In one embodiment the pancreatic cancer-associated protein is conjugated to BSA.

Measurements of pancreatic cancer polypeptide activity, or of pancreatic cancer or the pancreatic cancer phenotype are performed using a variety of assays. For example, the effects of the test compounds upon the function of the pancreatic cancer polypeptides are measured by examining parameters described above. A suitable physiological change that affects activity are used to assess the influence of a test compound on the polypeptides of this invention. When the functional consequences are determined using intact cells or animals, one can also measure a variety of effects such as, in the case of pancreatic cancer associated with tumours, tumour growth, tumour metastasis, neovascularization, hormone release, transcriptional changes to both known and uncharacterized genetic markers (e.g., northern blots), changes in cell metabolism such as cell growth or pH changes, and changes in intracellular second messengers such as cGMP. In tire assays of the invention, mammalia pancreatic cancer polypeptide is typically used, e.g., mouse, preferably human.

Assays to identify compounds with modulating activity are performed in vitro. For example, a pancreatic cancer polypeptide is first contacted with a potential modulator and incubated for a suitable amount of time, e.g., from 0.5 to 48 hours. In one embodiment, the pancreatic cancer polypeptide levels are determined in vitro by measuring the level of protein or mRNA. The level of protein is measured using immunoassays such as western blotting, ELISA and the like with an antibody that selectively binds to the pancreatic cancer polypeptide or a fragment thereof. For measurement of mRNA, amplification, e.g., using PCR, LCR, or hybridization assays, e.g., northern hybridization, RNAse protection, dot blotting, are preferred. The level of protein or mRNA is detected using directly or indirectly labeled detection agents, e.g., fluorescently or radioactively labeled nucleic acids, radioactively or enzymatically labeled antibodies, and the like, as described herein.

Alternatively, a reporter gene system are devised using the pancreatic cancer-associated protein promoter operably linked to a reporter gene such as luciferase, green fluorescent protein, CAT, or (beta-gal. The reporter construct is typically transfected into a cell. After treatment with a potential modulator, the amount of reporter gene transcription, translation, or activity is measured according to standard techniques known to those of skill in the art.

In a preferred embodiment, as outlined above, screens are done on individual genes and gene products (proteins). That is, having identified a particular differentially expressed gene as important in a particular state, screening of modulators of the expression of the gene or the gene product itself are done. The gene products of differentially expressed genes are sometimes referred to herein as “pancreatic cancer-associated proteins.” The pancreatic cancer-associated protein are a fragment, or alternatively, be the full length protein to a fragment shown herein.

In one embodiment, screening for modulators of expression of specific genes is performed. Typically, the expression of only one or a few genes are evaluated. In another embodiment, screens are designed to first find compounds that bind to differentially expressed proteins. These compounds are then evaluated for the ability to modulate differentially expressed activity. Moreover, once initial candidate compounds are identified, variants are further screened to better evaluate structure activity relationships.

In a preferred embodiment, binding assays are done. In general, purified or isolated gene product is used; that is, the gene products of one or more differentially expressed nucleic acids are made. For example, antibodies are generated to the protein gene products, and standard immunoassays are run to determine the amount of protein present. Alternatively, cells comprising the pancreatic cancer-associated proteins are used in the assays.

Thus, in a preferred embodiment, the methods comprise combining a pancreatic cancer-associated protein and a candidate compound, and determining the binding of the compound to the pancreatic cancer-associated protein. Preferred embodiments utilize the huma pancreatic cancer-associated protein, although other mammalian proteins may also be used, e.g. for the development of animal models of human disease. In some embodiments, as outlined herein, variant or derivative pancreatic cancer-associated proteins are used.

Generally, in a preferred embodiment of the methods herein, the pancreatic cancer-associated protein or the candidate agent is non-diffusably bound to an insoluble support having isolated sample receiving areas (e.g. a microtiter plate, an array, etc.). The insoluble supports are made of any composition to which the compositions are bound, is readily separated from soluble material, and is otherwise compatible with the overall method of screening. The surface of such supports are solid or porous and of any convenient shape. Examples of suitable insoluble supports include microtiter plates, arrays, membranes and beads. These are typically made of glass, plastic (e.g., polystyrene), polysaccharides, nylon or nitrocellulose, Teflon™, etc. microtitre plates and arrays are especially convenient because a large number of assays are carried out simultaneously, using small amounts of reagents and samples. The particular manner of binding of the composition is not crucial so long as it is compatible with the reagents and overall methods of the invention, maintains the activity of the composition and is nondiffusable. Preferred methods of binding include the use of antibodies (which do not sterically block either the ligand binding site or activation sequence when the protein is bound to the support), direct binding to “sticky” or ionic supports, chemical crosslinking, the synthesis of the protein or agent on the surface, etc. Following binding of the protein or agent, excess unbound material is removed by washing. The sample receiving areas may then be blocked through incubation with bovine serum albumin (BSA), casein or other innocuous protein or other moiety.

In a preferred embodiment, the pancreatic cancer-associated protein is bound to the support, and a test compound is added to the assay. Alternatively, the candidate agent is bound to the support and the pancreatic cancer-associated protein is added. Novel binding agents include specific antibodies, non-natural binding agents identified in screens of chemical libraries, peptide analogs, etc. Of particular interest are screening assays for agents that have a low toxicity for human cells. A wide variety of assays are used for this purpose, including labeled in vitro protein-protein binding assays, electrophoretic mobility shift assays, immunoassays for protein binding, functional assays (phosphorylation assays, etc.) and the like.

The determination of the binding of the test modulating compound to the pancreatic cancer-associated protein are done in a number of ways. In a preferred embodiment, the compound is labeled, and binding determined directly, e.g., by attaching all or a portion of the pancreatic cancer-associated protein to a solid support, adding a labeled candidate agent (e.g., a fluorescent label), washing off excess reagent, and determining whether the label is present on the solid support. Various blocking and washing steps are utilized as appropriate.

In some embodiments, only one of the components is labeled, e.g., the proteins (or proteinaceous candidate compounds) are labeled. Alternatively, more than one component are labeled with different labels, e.g., ¹²⁵I for the proteins and a fluorophor for the compound. Proximity reagents, e.g., quenching or energy transfer reagents are also useful.

In one embodiment, the binding of the test compound is determined by competitive binding assay. The competitor is a binding moiety known to bind to the target molecule (i.e., a pancreatic cancer-associated protein), such as an antibody, peptide, binding partner, ligand, etc. Under certain circumstances, there are competitive binding between the compound and the binding moiety, with the binding moiety displacing the compound. In one embodiment, the test compound is labeled. Either the compound, or the competitor, or both, is added first to the protein for a time sufficient to allow binding, if present. Incubations are performed at a temperature which facilitates optimal activity, typically between 4 and 40° C. Incubation periods are typically optimized, e.g., to facilitate rapid high throughput screening. Typically between 0.1 and 1 hour will be sufficient. Excess reagent is generally removed or washed away. The second component is then added, and the presence or absence of the labeled component is followed, to indicate binding.

In a preferred embodiment, the competitor is added first, followed by the test compound. Displacement of the competitor is an indication that the test compound is binding to the pancreatic cancer-associated protein and thus is capable of binding to, and potentially modulating, the activity of the pancreatic cancer-associated protein. In this embodiment, either component are labeled. Thus, e.g., if the competitor is labeled, the presence of label in the wash solution indicates displacement by the agent. Alternatively, if the test compound is labeled, the presence of the label on the support indicates displacement.

In an alternative preferred embodiment, the test compound is added first, with incubation and washing, followed by the competitor. The absence of binding by the competitor may indicate that the test compound is bound to the pancreatic cancer-associated protein with a higher affinity. Thus, if the test compound is labeled, the presence of the label on the support, coupled with a lack of competitor binding, may indicate that the test compound is capable of binding to the pancreatic cancer-associated protein.

In a preferred embodiment, the methods comprise differential screening to identity agents that are capable of modulating the activity of the pancreatic cancer-associated proteins. In this embodiment, the methods comprise combining a pancreatic cancer-associated protein and a competitor in a first sample. A second sample comprises a test compound, a pancreatic cancer-associated protein, and a competitor. The binding of the competitor is determined for both samples, and a change, or difference in binding between the two samples indicates the presence of an agent capable of binding to the pancreatic cancer-associated protein and potentially modulating its activity. That is, if the binding of the competitor is different in the second sample relative to the first sample, the agent is capable of binding to the pancreatic cancer-associated protein.

Alternatively, differential screening is used to identify drug candidates that bind to the native pancreatic cancer-associated protein, but cannot bind to modified pancreatic cancer-associated proteins. The structure of the pancreatic cancer-associated protein are modeled, and used in rational drug design to synthesize agents that interact with that site. Drug candidates that affect the activity of a pancreatic cancer-associated protein are also identified by screening drugs for the ability to either enhance or reduce the activity of the protein.

Positive controls and negative controls are used in the assays. Preferably control and test samples are performed in at least triplicate to obtain statistically significant results. Incubation of all samples is for a time sufficient for the binding of the agent to the protein. Following incubation, samples are washed free of non-specifically bound material and the amount of bound, generally labeled agent determined. For example, where a radiolabel is employed, the samples are counted in a scintillation counter to determine the amount of bound compound.

A variety of other reagents are included in the screening assays. These include reagents like salts, neutral proteins, e.g. albumin, detergents, etc. which are used to facilitate optimal protein-protein binding and/or reduce non-specific or background interactions. Also reagents that otherwise improve the efficiency of the assay, such as protease inhibitors, nuclease inhibitors, anti-microbial agents, etc., are used. The mixture of components are added in an order that provides for the requisite binding.

In a preferred embodiment, the invention provides methods for screening for a compound capable of modulating the activity of a pancreatic cancer-associated protein. The methods comprise adding a test compound, as defined above, to a cell comprising pancreatic cancer-associated proteins. Preferred cell types include almost any cell. The cells contain a recombinant nucleic acid that encodes a pancreatic cancer-associated protein. In a preferred embodiment, a library of candidate agents are tested on a plurality of cells.

In one aspect, the assays are evaluated in the presence or absence or previous or subsequent exposure of physiological signals, e.g. hormones, antibodies, peptides, antigens, cytokines, growth factors, action potentials, pharmacological agents including chemotherapeutics, radiation, carcinogenics, or other cells (i.e. cell-cell contacts). In another example, the determinations are determined at different stages of the cell cycle process.

In this way, compounds that modulate pancreatic cancer agents are identified. Compounds with pharmacological activity are able to enhance or interfere with the activity of the pancreatic cancer-associated protein. Once identified, similar structures are evaluated to identify critical structural feature of the compound.

In one embodiment, a method of inhibiting pancreatic cancer cell division is provided. The method comprises administration of a pancreatic cancer inhibitor. In another embodiment, a method of inhibiting pancreatic cancer is provided. The method comprises administration of a pancreatic cancer inhibitor. In a further embodiment, methods of treating cells or individuals with pancreatic cancer are provided. The method comprises administration of a pancreatic cancer inhibitor.

In one embodiment, a pancreatic cancer inhibitor is an antibody as discussed above. In another embodiment, the pancreatic cancer inhibitor is an antisense molecule.

A variety of cell growth, proliferation, and metastasis assays are known to those of skill in the art, as described below.

### Soft Agar Growth or Colony Formation in Suspension

Normal cells require a solid substrate to attach and grow. When the cells are transformed, they lose this phenotype and grow detached from the substrate. For example, transformed cells can grow in stirred suspension culture or suspended in semi-solid media, such as semi-solid or soft agar. The transformed cells, when transfected with tumour suppressor genes, regenerate normal phenotype and require a solid substrate to attach and grow. Soft agar growth or colony formation in suspension assays are used to identify modulators of pancreatic cancer sequences, which when expressed in host cells, inhibit abnormal cellular proliferation and transformation. A therapeutic compound would reduce or eliminate the host cells' ability to grow in stirred suspension culture or suspended in semisolid media, such as semi-solid or soft.

Techniques for soft agar growth or colony formation in suspension assays are described in Freshney, Culture of Animal Cells a Manual of Basic Technique (3rd ed., 1994), herein incorporated by reference. See also, the methods section of Garkavtsev et al. (1996), supra, herein incorporated by reference.

### Contact Inhibition and Density Limitation of Growth

Normal cells typically grow in a flat and organized pattern in a petri dish until they touch other cells. When the cells touch one another, they are contact inhibited and stop growing. When cells are transformed, however, the cells are not contact inhibited and continue to grow to high densities in disorganized foci. Thus, the transformed cells grow to a higher saturation density than normal cells. This are detected morphologically by the formation of a disoriented monolayer of cells or rounded cells in foci within the regular pattern of normal surrounding cells. Alternatively, labeling index with (³H)-thymidine at saturation density are used to measure density limitation of growth. See Freshney (1994), supra. The transformed cells, when transfected with tumour suppressor genes, regenerate a normal phenotype and become contact inhibited and would grow to a lower density.

In this assay, labeling index with (³H)-thymidine at saturation density is a preferred method of measuring density limitation of growth. Transformed host cells are transfected with a pancreatic cancer-associated sequence and are grown for 24 hours at saturation density in non-limiting medium conditions. The percentage of cells labeling with (³H)-thymidine is determined autoradiographically. See, Freshney (1994), supra.

### Growth Factor or Serum Dependence

Transformed cells have a lower serum dependence than their normal counterparts (see, e.g., Temin, J. Natl. Cancer Insti. 37:167-175 (1966); Eagle et al., J. Exp. Med. 131:836-879 (1970)); Freshney, supra. This is in part due to release of various growth factors by the transformed cells. Growth factor or serum dependence of transformed host cells are compared with that of control. Tumor specific markers levels Tumor cells release an increased amount of certain factors (hereinafter “tumour specific markers”) than their normal counterparts. For example, plasminogen activator (PA) is released from human glioma at a higher level than from normal brain cells (see, e.g., Gullino, Angiogenesis, tumour vascularization, and potential interference with tumour growth. in Biological Responses in Cancer, pp. 178-184 (Mihich (ed.) 1985)). Similarly, Tumor angiogenesis factor (TAF) is released at a higher level in tumour cells than their normal counterparts. See, e.g., Folkman, Angiogenesis and Cancer, Sem Cancer Biol. (1992)). Various techniques which measure the release of these factors are described in Freshney (1994), supra. Also, see, Unkless et al., J. Biol. Chem. 249:4295-4305 (1974); Strickland & Beers, J. Biol. Chem. 251:5694-5702 (1976); Whur et al., Br. J. Cancer 42:305 312 (1980); Gullino, Angiogenesis, tumour vascularization, and potential interference with tumour growth. in Biological Responses in Cancer, pp. 178-184 (Mihich (ed.) 1985); Freshney Anticancer Res. 5:111-130 (1985).

Invasiveness into Matrigel

The degree of invasiveness into Matrigel- or some other extracellular matrix constituent are used as an assay to identify compounds that modulate pancreatic cancer-associated sequences. Tumor cells exhibit a good correlation between malignancy and invasiveness of cells into Matrigel or some other extracellular matrix constituent. In this assay, tumourigenic cells are typically used as host cells. Expression of a tumour suppressor gene in these host cells would decrease invasiveness of the host cells.

Techniques described in Freshney (1994), supra, are used. Briefly, the level of invasion of host cells are measured by using filters coated with Matrigel or some other extracellular matrix constituent. Penetration into the gel, or through to the distal side of the filter, is rated as invasiveness, and rated histologically by number of cells and distance moved, or by prelabeling the cells with 125 1 and counting the radioactivity on the distal side of the filter or bottom of the dish. See, e.g., Freshney (1984), supra.

### Tumor Growth In Vivo

Effects of pancreatic cancer-associated sequences on cell growth are tested in transgenic or immune-suppressed mice. Knock-out transgenic mice are made, in which the pancreatic cancer gene is disrupted or in which a pancreatic cancer gene is inserted. Knock-out transgenic mice are made by insertion of a marker gene or other heterologous gene into the endogenous pancreatic cancer gene site in the mouse genome via homologous recombination. Such mice can also be made by substituting the endogenous pancreatic cancer gene with a mutated version of the pancreatic cancer gene, or by mutating the endogenous pancreatic cancer gene, e.g., by exposure to carcinogens.

A DNA construct is introduced into the nuclei of embryonic stem cells. Cells containing the newly engineered genetic lesion are injected into a host mouse embryo, which is re-implanted into a recipient female. Some of these embryos develop into chimeric mice that possess germ cells partially derived from the mutant cell line. Therefore, by breeding the chimeric mice it is possible to obtain a new line of mice containing the introduced genetic lesion (see, e.g., Capecchi et al., Science 244:1288 (1989)). Chimeric targeted mice are derived according to Hogan et al., Manipulating the Mouse Embryo: A Laboratory Manual, Cold Spring Harbor Laboratory (1988) and Teratocarcinomas and Embryonic Stem Cells: A Practical Approach, Robertson, ed., IRL Press, Washington, D.C., (1987).

Alternatively, various immune-suppressed or immune-deficient host animals are used. For example, genetically athymic “nude” mouse (see, e.g., Giovanella et al., J. Natl. Cancer Inst. 52:921 (1974)), a SCID mouse, a thymectomized mouse, or an irradiated mouse (see, e.g., Bradley et al., Br. J. Cancer 38:263 (1978); Selby et al., Br. J. Cancer 41:52 (1980)) are used as a host. Transplantable tumour cells (typically about 10⁶ cells) injected into isogenic hosts will produce invasive tumours in a high proportions of cases, while normal cells of similar origin will not. In hosts which developed invasive tumours, cells expressing a pancreatic cancer-associated sequences are injected subcutaneously. After a suitable length of time, preferably 4 to 8 weeks, tumour growth is measured (e.g. by volume or by its two largest dimensions) and compared to the control. Tumours that have a statistically significant reduction (using, e.g. Student's T test) are said to have inhibited growth.

### Administration

Therapeutic reagents of the invention are administered to patients, therapeutically. Typically, such proteins/Nucleic acids and substances may preferably be combined with various components to produce compositions of the invention. Preferably the compositions are combined with a pharmaceutically acceptable carrier or diluent to produce a pharmaceutical composition (which are for human or animal use). Suitable carriers and diluents include isotonic saline solutions, for example phosphate-buffered saline. The composition of the invention are administered by direct injection. The composition are formulated for parenteral, intramuscular, intravenous, subcutaneous, intraocular, oral, vaginal or transdermal administration. Typically, each protein are administered at a dose of from 0.01 to 30 mg/kg body weight, preferably from 0.1 to 10 mg/kg, more preferably from 0.1 to 1 mg/kg body weight.

Nucleic acids/vectors encoding polypeptide components for use in modulating the activity of the pancreatic cancer-associated proteins/Nucleic acids are administered directly as a naked nucleic acid construct. When the Nucleic acids/vectors are administered as a naked nucleic acid, the amount of nucleic acid administered may typically be in the range of from 1 μg to 10 mg, preferably from 100 μg to 1 mg.

Uptake of naked nucleic acid constructs by mammalian cells is enhanced by several known transfection techniques for example those including the use of transfection agents. Example of these agents include cationic agents (for example calcium phosphate and DEAE-dextran) and lipofectants (for example Lipofectam™ and Transfectam™). Typically, nucleic acid constructs are mixed with the transfection agent to produce a composition.

Preferably the polynucleotide or vector of the invention is combined with a pharmaceutically acceptable carrier or diluent to produce a pharmaceutical composition. Suitable carriers and diluents include isotonic saline solutions, for example phosphate-buffered saline. The composition are formulated for parenteral, intramuscular, intravenous, subcutaneous, oral, intraocular or transdermal administration.

The pharmaceutical compositions are administered in a range of unit dosage forms depending on the method of administration. For example, unit dosage forms suitable for oral administration include, powder, tablets, pills, capsules and lozenges. Orally administered dosage forms will typically be formulated to protect the active ingredient from digestion and may therefore be complexed with appropriate carrier molecules and/or packaged in an appropriately resistant carrier. Suitable carrier molecules and packaging materials/barrier materials are known in the art.

The compositions of the invention are administered for therapeutic or prophylatic treatments. In therapeutic applications, compositions are administered to a patient suffering from a disease (e.g. pancreatic cancer) in an amount sufficient to cure or at least partially ameliorate the disease and its complications. An amount adequate to accomplish this is defined as a “therapeutically effective dose”. An amount of the composition that is capable of preventing or slowing the development of cancer in a patient is referred to as a “prophylactically effective dose”.

The routes of administration and dosages described are intended only as a guide since a skilled practitioner will be able to determine readily the optimum route of administration and dosage for any particular patient and condition.

The present invention is further described with reference to the accompanying drawings and the following non-limiting examples.

### EXAMPLE 1

### Gene Expression Profiling to Identify Differentially-Expressed Genes in Pancreatic Cancer

### RNA Preparation and Transcript Profiling:

Total RNA was isolated from 12 pancreatic cancer specimens and 6 matched macroscopically and microscopically normal appearing pancreas from the resected specimens. Biotinylated cRNA for Affymetrix Genechip hybridization was prepared through a single round of reverse transcription with Superscript II (Life Technologies, Maryland) followed by second strand synthesis to create double stranded cDNA. After purification the cDNA was transcribed using a T7 polymerase (Enzo Technologies, New York, N.Y.) and purified (Baugh L R, Hill M, Brown E L, Hunter C P. Quantitative analysis of mRNA amplification by in vitro transcription. Nucleic Acids Research 2001; 29:e29). Hybridization cocktails were prepared as per Affymetrix protocol (Affymetrix, Santa Clara, Calif., USA) and quality assured on Affymetrix Test3 arrays, prior to hybridization to Affymetrix HG-U133A and B oligonucleotide microarrays.

### Analysis:

A relational database was constructed using FileMaker Pro 5.5 (FileMaker, Inc., San Francisco, Calif.) to facilitate multiple queries of data obtained from the above experiments. The database incorporated absolute signal strength of each oligonucleotide on the genechip for each specimen, with mathematical algorithms and statistical analyses generated using the Affymetrix Data Mining Tool Software (MAS 5.0; Affymetrix Inc. San Francisco, Calif.), which included t-test and Mann-Whitney U test data. In addition, Genbank, Unigene, Locuslink, OMIM, SwissProt and PubMed identification strings were linked to Affymetrix propriety probeset identification strings (http://www.affymetrix.com/analysis/download_center.affx), which allowed for the incorporation of our data with hierarchical clustering analyses using dchip software of the World Wide Web URL biostat.harvard.edu/complab/dchip.

GenMAPP software (http://www.GenMAPP.org/) was used to incorporate transcript profile data into maps of known pathways. This enabled the rapid construction of interactive molecular pathway maps which presented transcript profile comparisons between experimental groups for molecules within a given pathway or family of interest.

### Results

### Transcript Profiling Data Analysis:

Gene expression profiles of 12 pancreatic cancer and 5 normal pancreata were generated using Affymetrix high density oligonucletide microarrays, comprising 44,929 probe sets which interrogated about 33,000 substantiated genes. Initially a global analysis of differential gene expression in the pancreatic cancer samples compared with the normal pancreatic tissue, was performed utilizing a relational transcript profile database to validate that the data demonstrated differential expression of gene transcripts based on current knowledge of the cellular and genetic composition of the normal pancreas and pancreatic cancer (Logsdon et al., Cancer Res. 63, 2649-2657, 2003; Argani et al., Cancer Res 61, 4320-4324, 2001; Kuwada et al., Int J Oncol 22, 765-771, 2003) Filtering of the 22,283 genes interrogated by the Affymetrix Genechip HG-U133A oligonucleotide array identified 218 unique genes that exhibited the largest mean differential expression between normal pancreatic tissue and pancreatic cancer.

Hierachical clustering of these 218 gene expression profiles identified exocrine-specific genes (e.g. elastase, lipase); genes involved in the development of fibrous tissue e.g. collagen; markers of ductal epithelium expressed at high levels in pancreatic cancer, e.g. keratin 19; immune response related genes, e.g. interleukin-8; and both genes upregulated in pancreatic cancer, e.g. prostate stem cell antigen. Many genes identified were not previously identified to be associated with pancreatic cancer. These findings validate the technique as a reliable representation of the relative amplitudes of differential mRNA expression.

Novel genes with potential to be relevant to pancreatic cancer were next identified by querying the entire 44,929 probesets interrogated by the Affymetrix Genechip HG-U133A and B oligonucleotide arrays for genes differentially expressed between pancreatic cancer and normal pancreata. Specific criteria were a fold change of <0.5 or >2.0 between all cancer and all normal specimens, and a P value on paired t test and Mann-Whitney U test of <0.05. We identified 954 genes as overexpressed >2-fold between pancreatic cancer and normal pancreata, and of these 269 (28%) genes demonstrated >5-fold differential expression levels between pancreatic cancer and normal pancreata. Eight hundred and thirty three genes were identified as underexpressed (<0.5-fold change) between pancreatic cancer and normal pancreata), of which 75 genes showed a <0.2-fold change in expression levels between pancreatic cancer and normal pancreata.

While previous studies have limited their analyses to identifying single genes differentially expressed in pancreatic cancer, we employed a strategy that utilized GenMAPP software to identify molecular pathways not previously identified in the development and progression of pancreatic cancer in which a significant proportion of the genes identified as under- or over-expressed (Tables 3 and 4) were altered.

Using this approach, we identified a number of known molecular pathways that showed dysregulated expression in specific genes (Tables 5-25), including genes within the WNT and TGF-β signalling pathways.

Of particular interest, a significant number of components of the HOX family of transcriptional factors were upregulated in pancreatic cancer.

### EXAMPLE 2

### Overexpression of HOXB2 is an Intermediate Event in the Development of Pancreatic Intraepithelial Neoplasia and is Associated with a Poor Prognosis in Pancreatic Cancer

### Materials and Methods.

### Patient Cohort:

The inventors identified a cohort of 128 patients with the diagnosis of pancreatic adenocarcinoma that underwent pancreatic resection or biopsy between January 1972 and November 2001 from Westmead Hospital, Concord Hospital, The Royal Prince Alfred Hospital and The St. Vincent's Hospital Campus in Sydney, Australia. This cohort represents a subset of a previously described group of 348 patients (Biankin et al., J Clin Oncol 2002; In Press). Ethical approval for data and tissue collection was granted by the ethics committees of each hospital. Archival formalin-fixed, paraffin-embedded tissue from all the 128 pancreata that were resected or biopsied were used to construct seven pancreatic cancer tissue arrays, which contained up to 55×1.6 mm cores per slide. Conventional sections of 18 cases of normal pancreas from areas distal to the pancreatic cancer were used to assess gene expression in benign ductal epithelial cells. In addition conventional sections of 8 cases of pancreatic tissue containing tissue adjacent to pancreatic cancer were used to assess gene expression in pancreatic intraepithelial neoplasia (PanIN) the precursor lesion of pancreatic cancer.

For this cohort, the average age at diagnosis was 63.8 years (median 66.5, range 34-86, Table 26). Of the 128 patients for whom tissue was available, 76 were from pancreatic resections, 46 intraoperative incision biopsies and 6 post mortem specimens. Median follow-up for the cohort was 7.7 months (range 0 to 117 months). Eight patients were alive at the census date (Sep. 21, 2002). Median survival and disease-specific survival was 7.6 months. For the resected group of 76 patients, 37 (47%) had lymph node metastasis (Table 26). The mean tumor size was 31 mm. Resection margins were microscopically free of tumor in 40 (51%). Poorly differentiated tumors occurred in 25 patients (33%). Median follow-up was 11.0 months with a median disease-specific survival of 10.1 months, 1-year survival of 48.6% and 5-year survival of 1%. The 30-day mortality for resection was 2 (4%). The only patients still living in the cohort underwent resection.

Ethics approval was obtained from the same 4 teaching hospitals in Sydney for the acquisition of fresh pancreatic tissue from pancreatectomy specimens. Multiple samples of approximately 500 mg were excised from 12 resected pancreata, snap frozen in liquid nitrogen and stored at minus 80° C., prior to RNA extraction.

### Immunohistochemistry:

Pancreatic tissue microarrays were dewaxed and rehydrated before unmasking in target retrieval solution (EDTA and citrate, DAKO Corporation, Carpenteria, Calif.) in a microwave for 30 min. Using a DAKO autostainer, endogenous peroxidase activity was quenched in 3% hydrogen peroxide in methanol, followed by avidin/biotin and serum free protein blocks (DAKO Corporation, Carpenteria, Calif.). Sections were incubated for 30 min with 1:200 anti-HOXB2 (M19) antibody (Santa Cruz Biotechnology, Santa Cruz, Calif.). A streptavidin-biotin peroxidase detection system was used according to the manufacturer's instructions (LSAB label+link kit; DAKO Corporation, Carpenteria, Calif.) with 3,3′-diaminobenzidine as a substrate. Counterstaining was performed with Mayer's hematoxylin.

### Immunohistochemical Scoring:

Up to two separate samples of pancreas were examined per patient. Staining was assessed by two independent observers blinded to patient outcome (D.S. and J.G.K.). Standardization of scoring was achieved by comparison of scores between observers, and by conferencing, where any discrepancies were resolved by consensus. Scores were given as a percentage of nuclei staining positive within the representative area of the tissue microarray core and the absolute intensity of nuclear staining on a scale of 0 to 3 (0 representing no staining 1, representing heterogenous nuclear staining, 2, representing homogenous nuclear staining and 3 representing intense homogenous nuclear staining). The criteria to achieve a positive score was: HOX B2 nuclear intensity being >1 in >20% of nuclei.

### Statistical Analysis:

Kaplan-Meier survival models for univariate analysis and the Cox proportional hazards model for multivariate analysis were derived using Statview 5.0 Software (Abacus Systems, Berkeley, Calif.). A p value of <0.05 was accepted as statistically significant. Those factors that were prognostic on univariate analysis were then assessed in a multivariable model to identify factors that were independently prognostic and those that were the result of confounding. This analysis was performed sequentially on all patients who had available tissue (n=128) and on a subgroup of patients who underwent operative resection (n=76).

### Results

### HOX B2 Expression and Analysis:

One of the HOX genes demonstrating significant increased expression in pancreatic cancer compared with normal pancreata was the gene for HOX B2 (Genbank reference sequence NM_{—}002145, UniGene Cluster Hs.290432). The data show a 6.4-fold increase in the mean HOXB2 level in pancreatic cancer (393 average intensity units) with respect to normal tissue expression (59 average intensity units). Investigation of HOX B2 expression in pancreatic cancer identified a series of genes that encode overexpressed cell surface molecules and are thus therapeutic targets as well as transcription factors, protein tyrosine phosphatases and protein kinases, and genes involved in cell proliferation and cell adhesion.

While HOXB2 expression was identified in 32 of 52 (61.5%) unresected tumors examined, HOXB2 nuclear expression was observed in only 16 of 76 (21%) of the resected pancreata (χ² p<0.0001), suggesting that HOXB2 overexpression may be associated with surgical non-resectability. To investigate this further, we examined the association of HOXB2 protein expression with outcome, in all patients and in those patients who had undergone resection only.

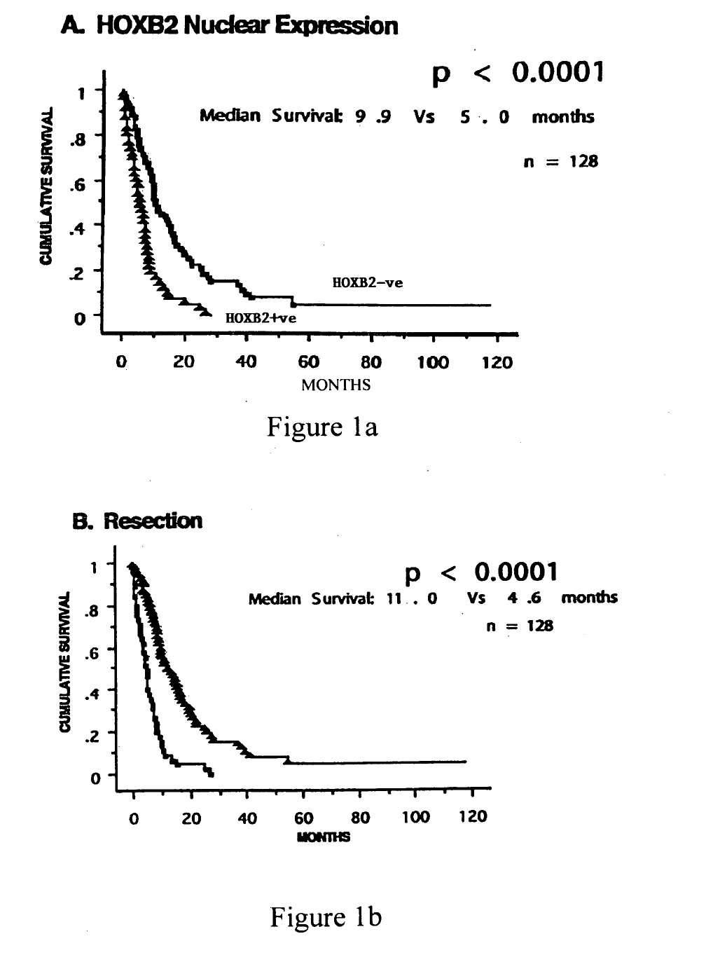

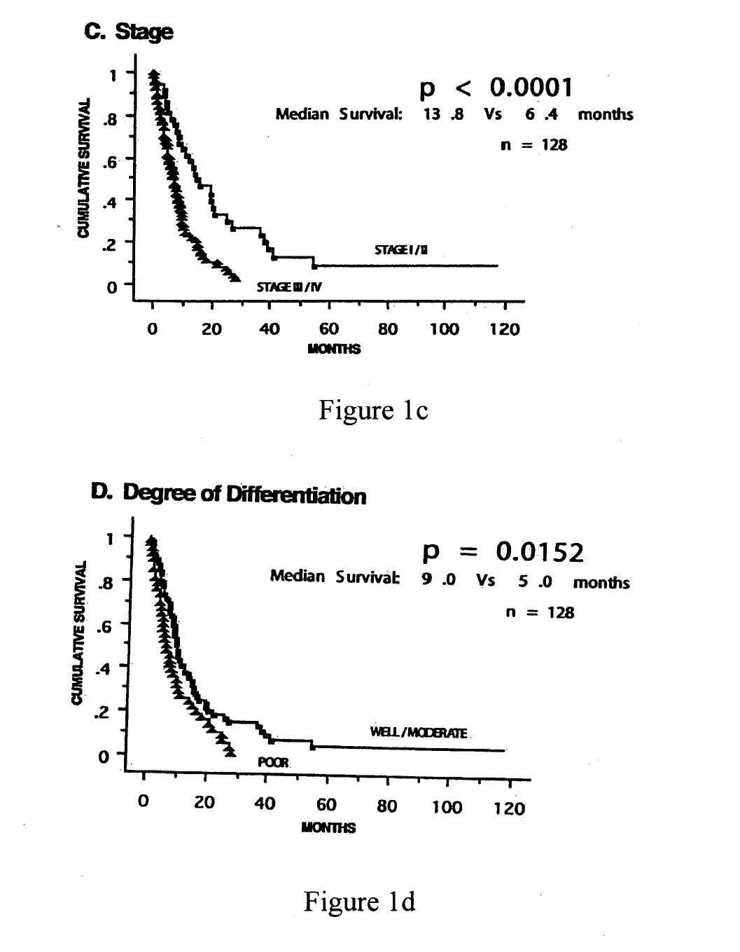

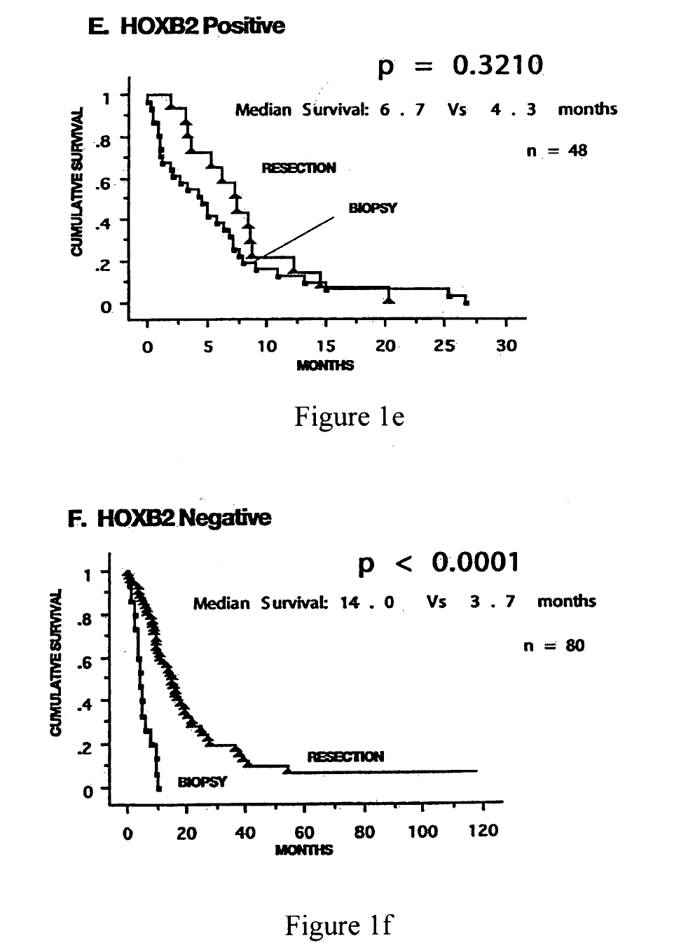

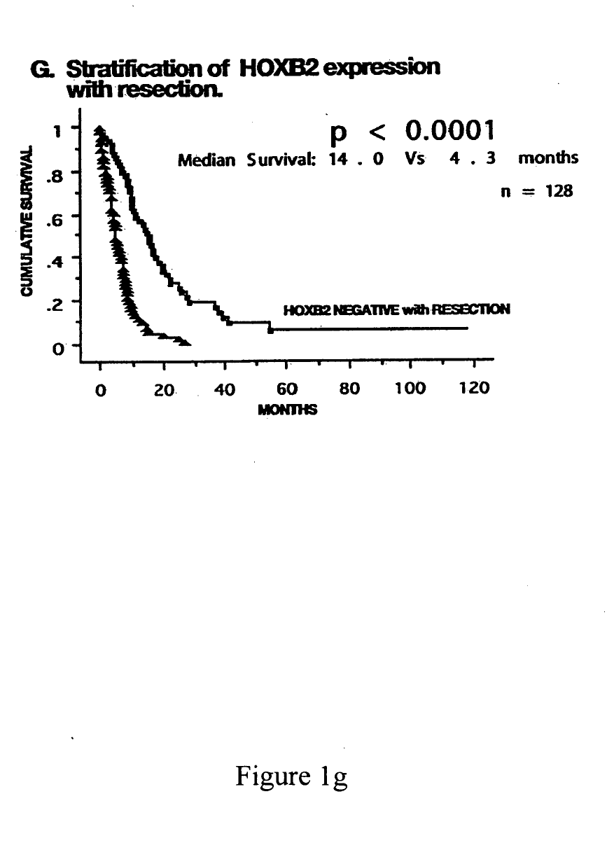

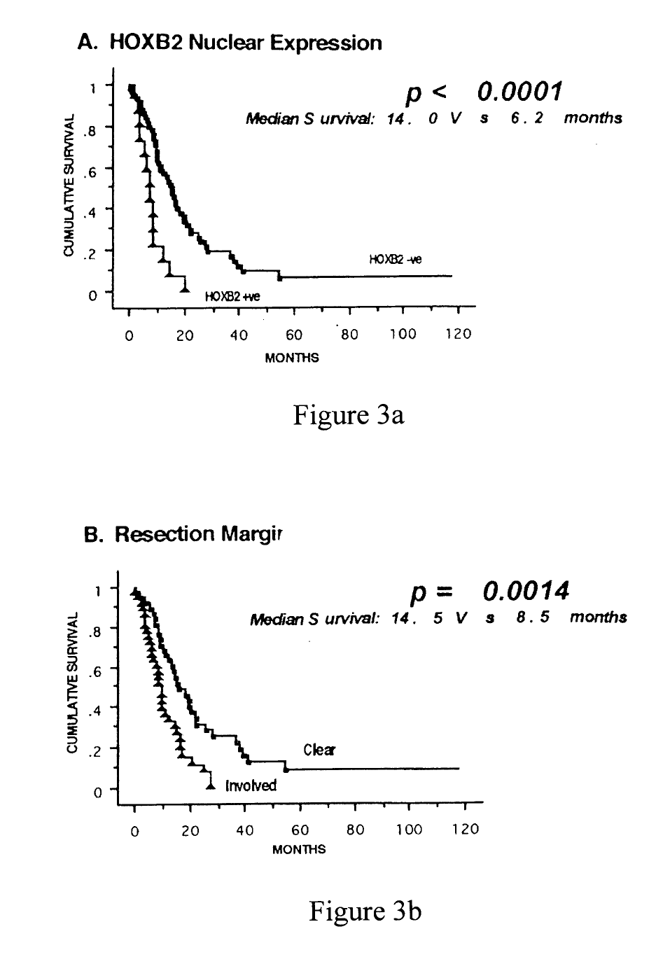

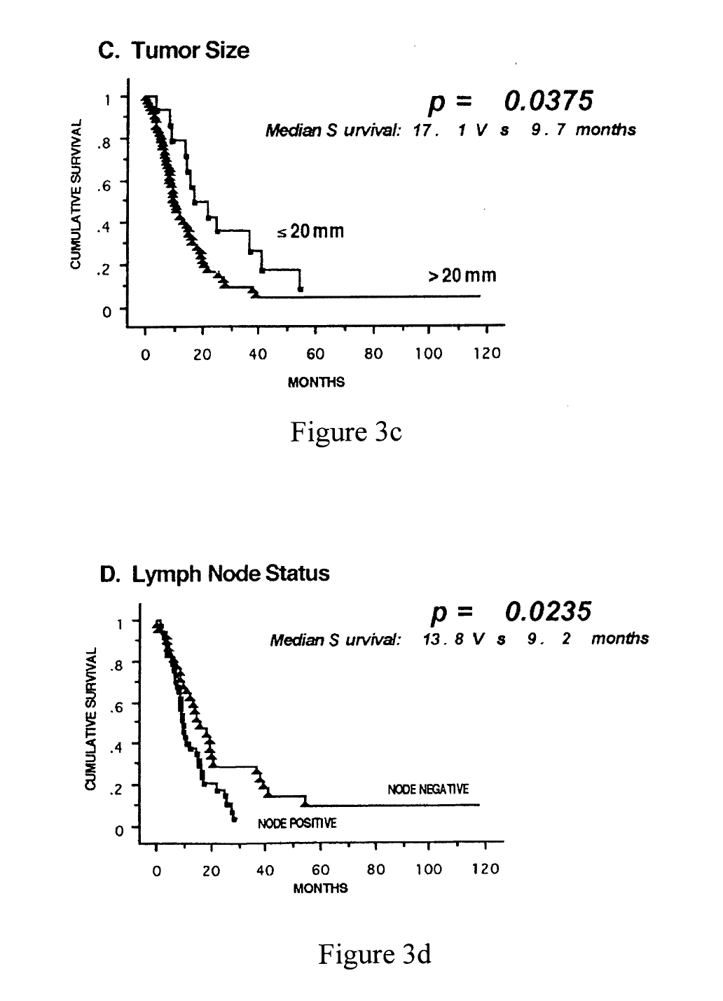

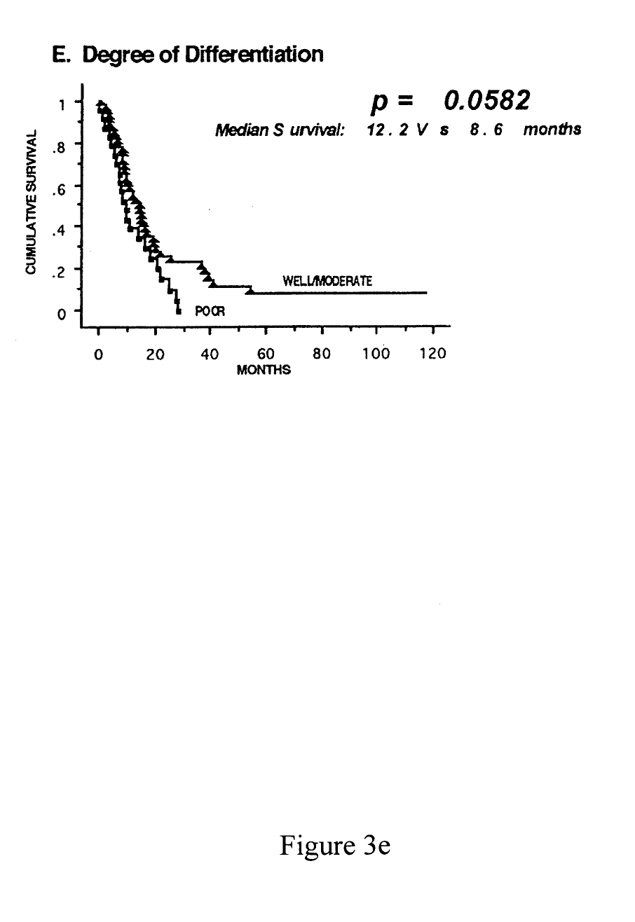

HOXB2 expression in the whole cohort was associated with a poor outcome (median survival 5 months and 9.9 months logrank, p<0.0001: FIG. 1a). In addition operative resection, low tumor stage and well-differentiated tumors were associated with significantly improved survival using Kaplan-Meier analysis (FIG. 1b-d). However, multivariate analysis identified resection and stage as the only independent prognostic factors when modelled together with degree of differentiation and HOXB2 status, in the whole cohort (Table 27). Operative resection did not benefit those patients whose tumors expressed HOXB2 (logrank p=0.32 FIG. 3E), but was beneficial to those patients who did not express HOXB2 (median survival advantage of 10.3 months, logrank p<0.0001 FIG. 1f). Survival for patients with tumors that were HOXB2 negative and who underwent resection was significantly longer than survival in all other groups (14 months versus 4.3 months; logrank p=0.04 FIG. 1g). Hence in this cohort lack of HOXB2 expression co-segregated with operative resectability. Importantly, only those who were HOXB2 negative benefited from operative resection.

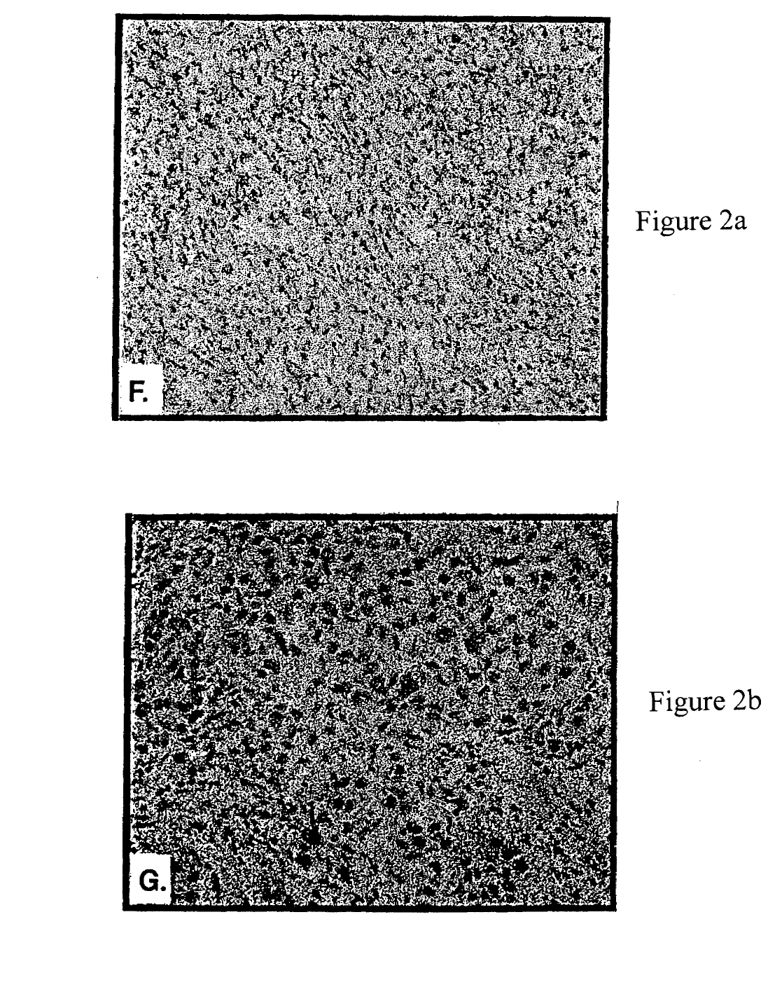

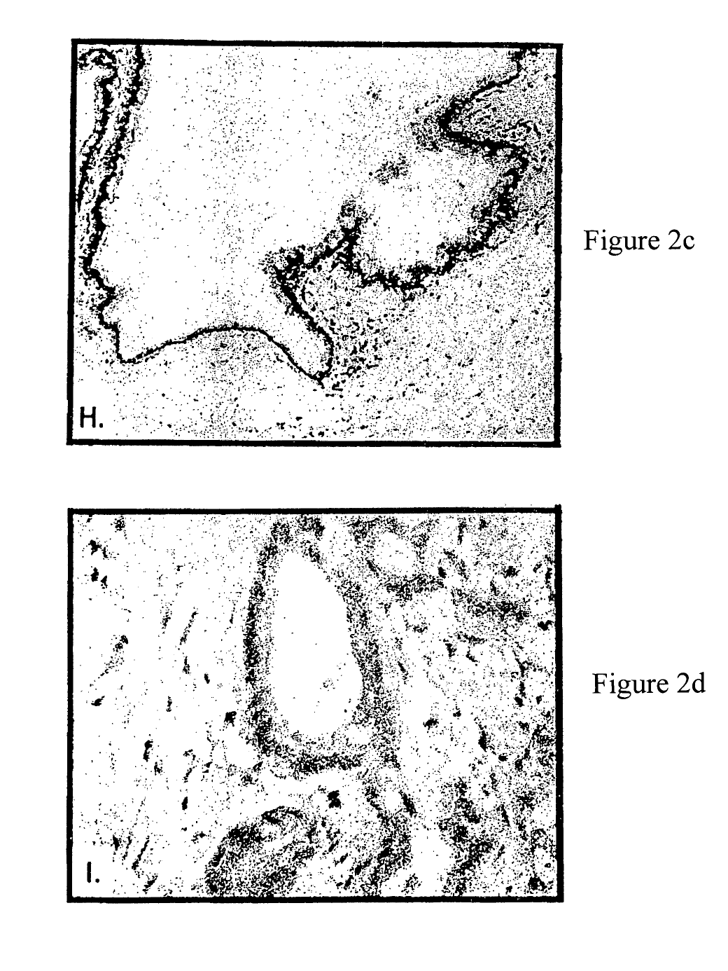

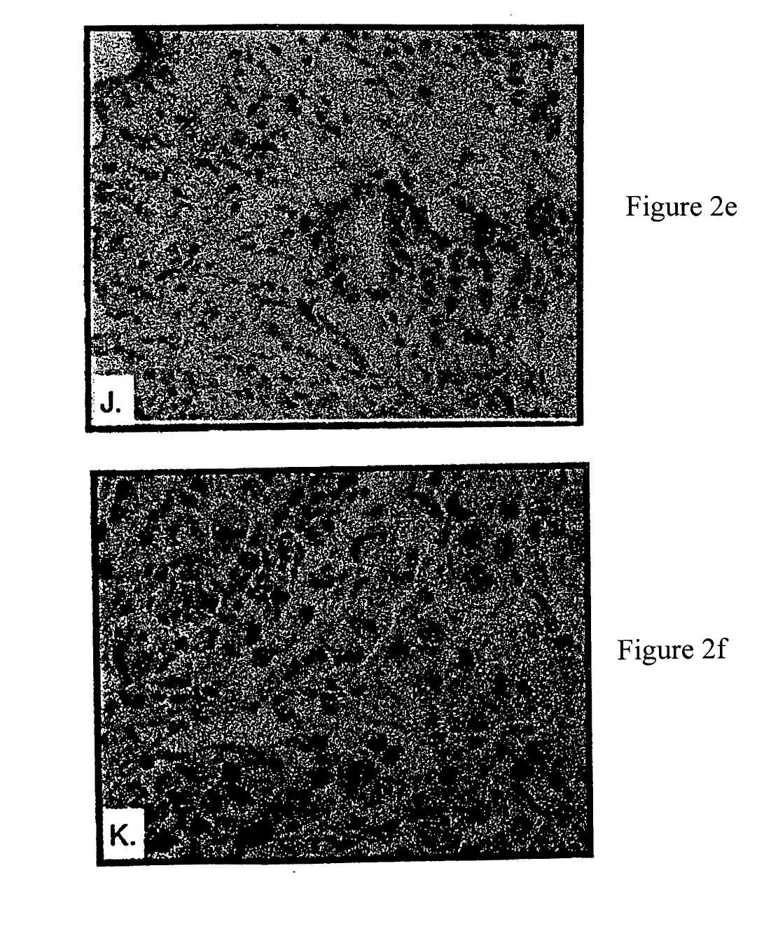

Survival analysis of the resected cohort identified decreased survival associated with HOXB2 nuclear expression (median survival disadvantage of 7.3 months, logrank p<0.001; FIG. 2a). Kaplan Meier analyses identified margin status, tumor size ≦20 mm and lymph node involvement as being associated with a survival advantage. Degree of differentiation conferred no survival advantage (FIGS. 2b-e). HOXB2 expression and involved surgical margins were independent prognostic factors when modelled against all combinations of; involved surgical margin, lymph node involvement, and tumor size in the subgroup of patients who underwent surgical resection (Table 28).

The expression of HOXB2 at the cellular level in benign, dysplastic and malignant pancreatic tissue was analysed by IHC (FIGS. 3a-f). HOXB2 overexpression defined as homogeneous nuclear staining was identified in 48 (37.5%) of 128 tumors. When HOXB2 expression was present within the tumor more than 80% of the nuclei were stained.

HOX B2 expression was identified in only 2 of 18 (11%) normal pancreata examined thereby validating the transcript profile data (χ² p=0.027). Of interest when we examined HOXB2 nuclear expression in the precursor lesions of pancreatic cancer (PanIN), staining was noted in 1 of 14 (7%) PanIN1a lesions, 3 of 13 (23%) Pan IN 1b lesions, 3 of 5 (60%) PanIN 2 lesions and 1 of 2 (50%) PanIN 3 lesions, suggesting increased HOXB2 nuclear staining in the intermediate and advanced precursor lesions

### Discussion.

Pancreatic cancer is thought to develop through a series of premalignant duct lesions termed pancreatic intraepithelial neoplasia (PanIN). Normal duct epithelium develops into PanIN-1A, to PanIN-1B then to PanIN-2 each differentiated by increasing ductal papillary hyperplasia and nuclear atypia (nuclear stratification and pleomorphism, mitoses and visible nucleoli). PanIN-3, demonstrates severe atypia and has in the past been called carcinoma in situ and is likely to progress to invasive carcinoma (Hruban et al., Am J Pathol 156, 1821-1825, 2000). The more advanced PanIN lesions (PanIN-2 and PanIN-3) exhibited increased HOXB2 nuclear expression. HOXB2 expression was increased in pancreatic cancer compared to normal pancreatic ducts and was increased during the intermediate and late stages of the known progression model for pancreatic cancer.

HOXB2 expression within pancreatic cancer in our cohort was associated with a poor outcome, with this association being maintained in the subset of patients who underwent resection. Multivariate analysis identified HOXB2 expression as an independent predictor of survival in the subgroup of patients that underwent pancreatic resection. Although HOXB2 expression was not identified as an independent predictor of survival in the whole cohort, lack of HOXB2 expression combined with surgical resection conferred a significant survival advantage. Because all known prognostic indicators in pancreatic cancer, such as tumor size, resection margins, and lymph node status can only be determined post resection, HOXB2 expression has utility as a prognostic indicator in pancreatic cancer, with the advantage that it can be assessed using biopsy techniques that are currently used as part of the preoperative assessment of a patient with pancreatic cancer, utilising available endoscopic and laparoscopic techniques.

Although pancreatic resection offers the best chance of cure and disease palliation in patients with pancreatic cancer, it is a procedure, which carries significant morbidity and mortality. The development of a reliable preoperative assessment of HOXB2 status is an important addition to a physician's limited diagnostic armamentarium in this disease and may be used, together with current clinico-pathological parameters of disease progression, to determine a patient's suitability for operative resection.

TABLE 3
Genes that are up-regulated in subjects having pancreatic cancer

| Fold | Affymetrix | GenBANK | GenBANK | Unigene | |
|---|---|---|---|---|---|
| Change | Code | Accession No. | Gene Symbol | cluster | Annotation |
| 152.4 | 204351_at | NM_005980.1 | S100P | Hs.2962 | S100 calcium-binding protein P |
| 64.2 | 205476_at | NM_004591.1 | SCYA20 | Hs.75498 | small inducible cytokine subfamily A (Cys-Cys), |
| | | | | | member 20 |
| 45.5 | 214974_x_at | AK026546.1 | | Hs.287716 | cDNA: FLJ22893 fis, clone KAT04792 |
| 40.5 | 37892_at | | | | Cluster Human alpha-1 type XI collagen |
| | | | | | (COL11A1) |
| 38.2 | 205044_at | NM_014211.1 | GABRP | Hs.70725 | GABA-A receptor |
| 36.5 | 201884_at | NM_004363.1 | CEACAM5 | Hs.220529 | carcinoembryonic antigen-related cell adhesion |
| | | | | | molecule 5 (CEACAM5) |
| 35 | 210809_s_at | D13665.1 | osf-2 | Hs.136348 | osteoblast specific factor 2 |
| 29.3 | 203256_at | NM_001793.1 | CDH3 | Hs.2877 | P-cadherin (placental) (CDH3) |
| 26.3 | 203108_at | NM_003979.2 | RAI3 | Hs.194691 | GPCR (retinoic acid induced 3) |
| 26 | 33323_r_at | | | | Cluster Incl. X57348: H. sapiens mRNA (clone |
| | | | | | 9112) |
| 25.5 | 211959_at | AW007532_RC | | Hs.103391 | insulin-like growth factor binding protein 5 |
| | | | | | (IGFBP5) |
| 24.6 | 217109_at | AJ242547.1 | MUC4 | Hs.198267 | MUC4 apomucin, tracheobronchial |
| 24.5 | 210511_s_at | M13436.1 | INHBA | Hs.727 | ovarian beta-A inhibin |
| 22.7 | 204268_at | NM_005978.2 | S100A2 | Hs.38991 | S100 calcium-binding protein A2 |
| 22.1 | 218960_at | NM_016425.1 | TMPRSS4 | Hs.63325 | transmembrane protease, serine 4 (TMPRSS4) |
| 21.8 | 223586_at | AF256215.1 | | Hs.222024 | 1. cycle-like factor CLIF 2. transcription factor |
| | | | | | BMAL2 |
| 21.6 | 204855_at | NM_002639.1 | SERPINB5 | Hs.55279 | clade B (ovalbumin), member 5 |
| 21.3 | 202404_s_at | NM_000089.1 | COL1A2 | Hs.179573 | collagen, type I, alpha 2 (COL1A2) |
| 20.8 | 211161_s_at | AF130082.1 | | Hs.119571 | collagen, type III, alpha 1 (Ehlers-Danlos |
| | | | | | syndrome type IV, autosomal dominant) |
| 20.8 | 212444_at | AA156240_RC | | Hs.288660 | cDNA: FLJ22182 fis, clone HRC00953 |
| 20 | 209360_s_at | D43968.1 | AML1 | Hs.129914 | acute myeloid leukemia 1 |
| 19.4 | 210095_s_at | M31159.1 | IGFBP1 | Hs.77326 | Insulin-like growth factor-binding protein 3 |
| 19.1 | 202465_at | NM_002593.2 | PCOLCE | Hs.202097 | procollagen C-endopeptidase enhancer |
| | | | | | (PCOLCE) |
| 18.7 | 205234_at | NM_004696.1 | SLC16A4 | Hs.23590 | solute carrier family 16, member 4 (SLC16A4) |
| 18.2 | 211719_x_at | BC005858.1 | | | Unknown (protein for MGC: 3255) |
| 17.9 | 33322_i_at | | | | Cluster Incl. X57348: H. sapiens mRNA (clone |
| | | | | | 9112) |
| 17.6 | 217755_at | NM_016185.1 | HN1 | Hs.109706 | hematological and neurological expressed 1 |
| | | | | | (HN1) |
| 17.4 | 211657_at | M18728.1 | NCA; NCA; NCA | | nonspecific crossreacting antigen |
| 17 | 37020_at | | | | C-reactive protein |
| 16.5 | 206392_s_at | NM_002888.1 | RARRES1 | Hs.82547 | retinoic acid receptor responder (tazarotene |
| | | | | | induced) 1 (RARRES1) |
| 16.4 | 203878_s_at | NM_005940.2 | MMP11 | Hs.155324 | MMP11 |
| 16.3 | 204320_at | NM_001854.1 | COL11A1 | Hs.82772 | collagen, type XI, alpha 1 (COL11A1) |
| 15.9 | 219463_at | NM_012261.1 | HS1119D91 | Hs.22920 | similar to S68401 (cattle) glucose induced gene |
| | | | | | (chromosome 20 open reading frame 103) |
| 15.5 | 205009_at | NM_003225.1 | TFF1 | Hs.1406 | TFF1 |
| 15.4 | 203757_s_at | BC005008.1 | | Hs.73848 | carcinoembryonic antigen-related cell adhesion |
| | | | | | molecule 6 |
| 15.2 | 217728_at | NM_014624.2 | S100A6 | Hs.275243 | S100 calcium-binding protein A6 (calcyclin) |
| | | | | | (S100A6) |
| 14.8 | 212464_s_at | X02761.1 | | Hs.287820 | fibronectin precursor |
| 14.5 | 227183_at | AI417267_RC | | Hs.84630 | EST |
| 14.4 | 205319_at | NM_005672.1 | PSCA | Hs.20166 | prostate stem cell antigen (PSCA) |
| 14.4 | 205366_s_at | NM_018952.1 | HOXB6 | Hs.98428 | homeo box B6 (HOXB6) |
| 14.2 | 203234_at | NM_003364.1 | UP | Hs.77573 | uridine phosphorylase (UP) |
| 14.1 | 204619_s_at | BF590263_RC | | Hs.81800 | chondroitin sulfate proteoglycan 2 (versican) |
| | | | | | (CSPG2) |
| 13.8 | 203789_s_at | NM_006379.1 | SEMA3C | Hs.171921 | sema domain, secreted, (semaphorin) 3C |
| | | | | | (SEMA3C) |
| 13.6 | 216834_at | S59049.1 | BL34 | Hs.75256 | regulator of G-protein signalling 1 |
| 13.6 | 204698_at | NM_002201.2 | ISG20 | Hs.183487 | interferon stimulated gene |
| 13.5 | 211924_s_at | AY029180.1 | SUPAR | | soluble urokinase plasminogen activator |
| | | | | | receptor precursor (SUPAR) |
| 13.5 | 212531_at | NM_005564.1 | LCN2 | Hs.204238 | lipocalin 2 (oncogene 24p3) |
| 13.5 | 212354_at | BE500977_RC | | Hs.70823 | KIAA1077 protein |
| 13.2 | 221731_x_at | BF218922 | | Hs.81800 | chondroitin sulfate proteoglycan 2 (versican) |
| | | | | | (CSPG2) |
| 13.1 | 221729_at | AL575735_RC | | Hs.82985 | collagen, type V, alpha 2 |
| 12.9 | 204439_at | NM_006820.1 | GS3686 | Hs.75470 | hypothetical protein, expressed in osteoblast |
| | | | | | (GS3686) |
| 12.8 | 205713_s_at | NM_000095.1 | COMP | Hs.1584 | cartilage oligomeric matrix protein |
| | | | | | (pseudoachondroplasia, epiphyseal dysplasia 1, |
| | | | | | multiple) (COMP), |
| 12.6 | 205927_s_at | NM_001910.1 | CTSE | Hs.1355 | cathepsin E (CTSE) |
| 12.6 | 211597_s_at | AB059408.1 | | | mRNA, complete cds, clone: SMAP31-12. |
| 12.5 | 202859_x_at | NM_000584.1 | IL8 | Hs.624 | interleukin 8 (IL8) |
| 12.3 | 225328_at | N21643_RC | | Hs.6630 | cDNA FLJ13329 fis, clone OVARC1001795 |
| 12.2 | 212353_at | AI479175_RC | | Hs.70823 | KIAA1077 protein |
| 12.1 | 212489_at | AI983428_RC | | Hs.146428 | collagen, type V, alpha 1 |
| 12.1 | 209792_s_at | BC002710.1 | | Hs.69423 | kallikrein 10 |
| 12 | 210495_x_at | AF130095.1 | | Hs.287820 | fibronectin 1 |
| 12 | 209373_at | BC003179.1 | | Hs.185055 | BENE protein |
| 11.7 | 211430_s_at | M87789.1 | | Hs.300697 | immunoglobulin heavy constant gamma 3 (G3m |
| | | | | | marker) |
| 11.4 | 209803_s_at | AF001294.1 | IPL | Hs.154036 | tumor suppressing subtransferable candidate 3 |
| 11.3 | 206023_at | NM_006681.1 | NMU | Hs.2841 | neuromedin U (NMU) |
| 11.3 | 217428_s_at | X98568 | | Hs.179729 | collagen, type X, alpha 1 (Schmid metaphyseal |
| | | | | | chondrodysplasia) |
| 11.2 | 218644_at | NM_016445.1 | PLEK2 | Hs.39957 | pleckstrin 2 (mouse) homolog (PLEK2) |
| 11 | 216442_x_at | AK026737.1 | | Hs.287820 | fibronectin (FN precursor) |
| 10.9 | 202497_x_at | AI631159_RC | | Hs.7594 | solute carrier family 2 (facilitated glucose |
| | | | | | transporter), member 3 |
| 10.6 | 218468_s_at | AF154054.1 | DRM | Hs.40098 | cysteine knot superfamily 1, BMP antagonist 1 |
| 10.3 | 219404_at | NM_024526.1 | FLJ21522 | Hs.5366 | hypothetical protein FLJ21522 |
| 10.1 | 202153_s_at | NM_016553.1 | DKFZp547L134 | Hs.9877 | hypothetical protein (DKFZp547L134) |
| 10.1 | 212768_s_at | AL390736 | | Hs.273321 | GW112 protein with two isoforms (GW112 and |
| | | | | | KIAA4294) |
| 10 | 205860_x_at | NM_004476.1 | FOLH1 | Hs.1915 | folate hydrolase (prostate-specific membrane |
| | | | | | antigen) 1 (FOLH1) |
| 9.9 | 202267_at | NM_005562.1 | LAMC2 | Hs.54451 | laminin, gamma 2, isoform a precursor |
| 9.8 | 201467_s_at | AI039874_RC | | Hs.80706 | cytochrome b-5 reductase ? (NADPH |
| | | | | | Dehydrogenase Quinone - Ug) |
| 9.6 | 203083_at | NM_003247.1 | THBS2 | Hs.108623 | thrombospondin 2 (THBS2) |
| 9.6 | 204885_s_at | NM_005823.2 | MSLN | Hs.155981 | mesothelin (MSLN), transcript variant 1 |
| 9.6 | 201645_at | NM_002160.1 | HXB | Hs.289114 | hexabrachion (tenascin C, cytotactin) |
| 9.5 | 207172_s_at | NM_001797.1 | CDH11 | Hs.75929 | cadherin 11, type 2, OB-cadherin (osteoblast) |
| | | | | | (CDH11) |
| 9.5 | 207714_s_at | NM_004353.1 | SERPINH1 | Hs.241579 | clade H (heat shock protein 47), member 1 |
| | | | | | (SERPINH1) |
| 9.3 | 206560_s_at | NM_006533.1 | MIA | Hs.279651 | melanoma inhibitory activity (MIA) |
| 9.3 | 221558_s_at | AF288571.1 | LEF1 | Hs.44865 | lymphoid enhancer factor-1 (LEF1) |
| 9 | 203726_s_at | NM_000227.1 | LAMA3 | Hs.83450 | laminin alpha 3 subunit precursor |
| 9 | 219478_at | NM_021197.1 | WFDC1 | Hs.36688 | WAP four-disulfide core domain 1 (WFDC1) |
| 9 | 202311_s_at | AI743621_RC | | Hs.172928 | collagen, type I |
| 9 | 202307_s_at | NM_000593.2 | ABCB2 | Hs.158164 | ATP-binding cassette, sub-family B, member 2 |
| 8.9 | 40472_at | | | | Cluster Incl. AF007155: Homo sapiens clone |
| | | | | | 23763 unknown mRNA |
| 8.8 | 205997_at | NM_021778.1 | ADAM28 | Hs.174030 | disintegrin and metalloproteinase domain 28 |
| | | | | | (ADAM28) |
| 8.8 | 212992_at | AI935123_RC | | Hs.57548 | EST |
| 8.8 | 205157_s_at | NM_000422.1 | KRT17 | Hs.2785 | keratin 17 (KRT17) |
| 8.7 | 204933_s_at | NM_002546.1 | TNFRSF11B | Hs.81791 | tumor necrosis factor receptor superfamily, |
| | | | | | member 11b (osteoprotegerin) (TNFRSF11B) |
| 8.6 | 209728_at | BC005312.1 | | Hs.318720 | major histocompatibility complex, class II, DR |
| | | | | | beta 4 |
| 8.6 | | NM_000104.2 | | | Cytochrome P450, subfamily I (dioxin-inducible) |
| 8.6 | 217875_s_at | NM_020182.1 | TMEPAI | Hs.83883 | transmembrane, prostate androgen induced |
| | | | | | RNA |
| 8.6 | 212702_s_at | N45111_RC | | Hs.330988 | Similar to Bicaudal D (Drosophila) homolog 1 |
| 8.5 | 209955_s_at | U76833.1 | | Hs.418 | integral membrane serine protease 2, fibroblast |
| | | | | | activation protein, alpha |
| 8.4 | 203153_at | NM_001548.1 | IFIT1 | Hs.20315 | interferon-induced protein with tetratricopeptide |
| | | | | | repeats 1 (IFIT1) |
| 8.3 | 202555_s_at | NM_005965.1 | MYLK | Hs.211582 | myosin, light polypeptide kinase (MYLK) |
| 8.1 | 212236_x_at | Z19574 | | Hs.2785 | keratin 17 |
| 8.1 | 218469_at | NM_013372.1 | CKTSF1B1 | Hs.40098 | cysteine knot superfamily 1, BMP antagonist 1 |
| 8.1 | 202450_s_at | NM_000396.1 | CTSK | Hs.83942 | cathepsin K (pycnodysostosis) (CTSK) |
| 8.1 | 201474_s_at | NM_002204.1 | ITGA3 | Hs.265829 | integrin, alpha 3 (antigen CD49C, alpha 3 |
| | | | | | subunit of VLA-3 receptor) (ITGA3) |
| 8.1 | 214476_at | NM_005423.1 | TFF2 | Hs.2979 | trefoil factor 2 (spasmolytic protein 1) (TFF2) |
| 7.9 | 213988_s_at | BE971383 | | Hs.28491 | spermidinespermine N1-acetyltransferase |
| 7.8 | 204620_s_at | NM_004385.1 | CSPG2 | Hs.81800 | chondroitin sulfate proteoglycan 2 (versican) |
| | | | | | (CSPG2) |
| 7.6 | 201798_s_at | NM_013451.1 | FER1L3 | Hs.234680 | fer-1 (C. elegans)-like 3 (myoferlin) (FER1L3) |
| 7.5 | 220066_at | NM_022162.1 | NOD2 | Hs.135201 | NOD2 protein NOD2 |
| 7.4 | 200665_s_at | NM_003118.1 | SPARC | Hs.111779 | secreted protein, acidic, cysteine-rich |
| | | | | | (osteonectin) (SPARC) |
| 7.4 | 222872_x_at | AU157541_RC | | Hs.118183 | hypothetical protein FLJ22833 |
| 7.3 | 201621_at | NM_005380.1 | NBL1 | Hs.76307 | neuroblastoma, suppression of tumorigenicity 1 |
| | | | | | (NBL1) |
| 7.2 | 218308_at | NM_006342.1 | TACC3 | Hs.104019 | transforming, acidic coiled-coil containing protein |
| | | | | | 3 (TACC3) |
| 7.2 | 226047_at | N66571_RC | | Hs.26243 | Homo sapiens cDNA FLJ11177 fis, clone |
| | | | | | PLACE1007402 |
| 7.2 | 224469_s_at | BC006173.1 | | | Unknown (protein for MGC: 13251) |
| 7.1 | 201058_s_at | NM_006097.1 | MYRL2 | Hs.9615 | myosin regulatory light chain 2, smooth muscle |
| | | | | | isoform (MYRL2) |
| 7 | 217764_s_at | AF183421.1 | | Hs.223025 | small GTP-binding protein rab22b |
| 7 | 213975_s_at | AV711904 | | Hs.277431 | Homo sapiens cDNA: FLJ23356 fis, clone |
| | | | | | HEP14919 |
| 7 | 206391_at | NM_002888.1 | RARRES1 | Hs.82547 | retinoic acid receptor responder (tazarotene |
| | | | | | induced) 1 (RARRES1) |
| 6.9 | 218376_s_at | NM_022765.1 | FLJ11937 | Hs.33476 | hypothetical protein FLJ11937 |
| 6.9 | | NM_005620.1 | S100A11 | | S100 calcium-binding protein A11 (calgizzarin) |
| 6.9 | 212344_at | AW043713_RC | | Hs.70823 | KIAA1077 protein |
| 6.8 | 209596_at | AF245505.1 | | Hs.72157 | adlican |
| 6.8 | 208131_s_at | NM_000961.1 | PTGIS | | prostaglandin I2 (prostacyclin) synthase (PTGIS) |
| 6.8 | 209016_s_at | BC002700.1 | | Hs.23881 | keratin 7 |
| 6.8 | 204415_at | NM_022873.1 | G1P3 | Hs.265827 | interferon, alpha-inducible protein (clone IFI-6- |
| | | | | | 16) (G1P3), transcript variant 3 |
| 6.7 | 209900_s_at | AL162079.1 | DKFZp762B2310 | Hs.75231 | solute carrier family 16 (monocarboxylic acid |
| | | | | | transporters) |
| 6.7 | 205453_at | NM_002145.1 | HOXB2 | Hs.2733 | homeo box B2 (HOXB2) |
| 6.7 | 218051_s_at | NM_022908.1 | FLJ12442 | Hs.84753 | hypothetical protein FLJ12442 |
| 6.7 | 206994_at | NM_001899.1 | CST4 | Hs.56319 | cystatin S |
| 6.7 | 204052_s_at | NM_003014.2 | SFRP4 | Hs.105700 | sFRP4 |
| 6.7 | 231175_at | N48613_RC | | Hs.12431 | EST |
| 6.6 | 203821_at | NM_001945.1 | DTR | Hs.799 | diphtheria toxin receptor (heparin-binding |
| | | | | | epidermal growth factor-like growth factor) |
| | | | | | (DTR) |
| 6.6 | 205499_at | NM_014467.1 | SRPUL | Hs.126782 | sushi-repeat protein (SRPUL) |
| 6.6 | 208727_s_at | BC002711.1 | | Hs.146409 | cell division cycle 42 (GTP-binding protein, |
| | | | | | 25 kD) |
| 6.5 | | AF311912.1 | SRFP2 | | Secreted frizzled-related protein 2 (sFRP2) |
| 6.5 | 201655_s_at | M85289.1 | HSPG2 | Hs.211573 | heparan sulfate proteoglycan (HSPG2) |
| 6.5 | 206662_at | NM_002064.1 | GLRX | Hs.28988 | glutaredoxin (thioltransferase) |
| 6.5 | 225544_at | AI806338_RC | | Hs.267182 | T-box 3 (ulnar mammary syndrome) |
| 6.4 | 219014_at | NM_016619.1 | LOC51316 | Hs.107139 | hypothetical protein LOC51316 |
| 6.4 | 203510_at | BG170541 | | Hs.285754 | met proto-oncogene (hepatocyte growth factor |
| | | | | | receptor) |
| 6.3 | 238063_at | AA806283_RC | | Hs.120219 | EST |
| 6.2 | 213338_at | BF062629_RC | | Hs.35861 | DKFZP586E1621 protein |
| 6.2 | 202411_at | NM_005532.1 | IFI27 | Hs.278613 | interferon, alpha-inducible protein 27 |
| 6.2 | 217238_s_at | AK026411.1 | | Hs.234234 | KAIA0811, highly similar to HUMALDB Human |
| | | | | | aldolase B mRNA. |
| 6.1 | 201860_s_at | NM_000930.1 | PLAT | Hs.274404 | plasminogen activator, tissue (PLAT) |
| 6.1 | 213125_at | AW007573_RC | | Hs.43658 | hypothetical protein DKFZp586L151.1 |
| 6.1 | 212667_at | AL575922_RC | | Hs.111779 | secreted protein, acidic, cysteine-rich |
| | | | | | (osteonectin) |
| 6.0 | | NM_004995.2 | MMP14 | | Matrix metalloproteinase 14 (membrane |
| | | | | | inserted) |
| 6.0 | 210143_at | AF196478.1 | ANX14 | Hs.188401 | annexin 14 (ANX14) |
| 5.9 | 201666_at | NM_003254.1 | TIMP1 | Hs.5831 | tissue inhibitor of metalloproteinase 1 (erythroid |
| | | | | | potentiating activity, collagenase inhibitor) |
| | | | | | (TIMP1) |
| 5.9 | 200872_at | NM_002966.1 | S100A10 | Hs.119301 | S100 calcium-binding protein A10 (annexin II |
| | | | | | ligand, calpactin I, light polypeptide (p11)) |
| | | | | | (S100A10) |
| 5.9 | 219229_at | NM_013272.2 | SLC21A11 | Hs.14805 | solute carrier family 21 (organic anion |
| | | | | | transporter), member 11 (SLC21A11) |
| 5.9 | 202766_s_at | NM_000138.1 | FBN1 | Hs.750 | fibrillin 1 (Marfan syndrome) (FBN1) |
| 5.7 | 213230_at | AI422335_RC | | Hs.78358 | paraneoplastic antigen |
| 5.7 | 215235_at | AL110273.1 | DKFZp564P0562 | Hs.77196 | spectrin, alpha, non-erythrocytic 1 (alpha-f |
| 5.6 | 216942_s_at | D28586.1 | | Hs.75626 | lymphocyte function-associated antigen 3 |
| 5.6 | 201792_at | NM_001129.2 | AEBP1 | Hs.118397 | AE-binding protein 1 (AEBP1) |
| 5.5 | 201141_at | NM_002510.1 | GPNMB | Hs.82226 | glycoprotein (transmembrane) nmb (GPNMB) |
| 5.5 | 215077_at | AU144167_RC | | Hs.297909 | highly similar to PROCOLLAGEN ALPHA 1(III) |
| | | | | | CHAIN PRECURSOR |
| 5.5 | 215633_x_at | AV713720 | | Hs.306434 | LST-1N protein |
| 5.5 | | AI760277_RC | | | v-raf murine sarcoma 3611 viral oncogene |
| | | | | | homolog 1 |
| 5.5 | 212647_at | NM_006270.1 | RRAS | Hs.9651 | RAS viral (r-ras) oncogene homolog |
| 5.5 | 225618_at | AI769587_RC | | Hs.180958 | EST |
| 5.4 | 217762_s_at | BE789881 | | Hs.223025 | RAB31, member RAS oncogene family |
| 5.4 | 215125_s_at | AV691323 | | Hs.2056 | UDP glycosyltransferase 1 family, polypeptide |
| | | | | | A9 |
| 5.3 | 204137_at | NM_003272.1 | TM7SF1 | Hs.15791 | transmembrane 7 superfamily member 1 |
| | | | | | (upregulated in kidney) (TM7SF1) |
| 5.3 | 212687_at | AL110164.1 | | Hs.193700 | Homo sapiens mRNA; cDNA DKFZp586I0324 |
| | | | | | (from clone DKFZp586I0324) |
| 5.3 | 209969_s_at | BC002704.1 | | Hs.21486 | Similar to signal transducer and activator of |
| | | | | | transcription 1, 91 kD, clone MGC: 3493 |
| 5.3 | 229883_at | AI524330_RC | | Hs.99389 | EST |
| 5.1 | 201012_at | NM_000700.1 | ANXA1 | Hs.78225 | annexin I |
| 5 | 204068_at | NM_006281.1 | STK3 | Hs.166684 | serine/threonine kinase 3 (STE20 homolog, |
| | | | | | yeast) |
| 5 | 221756_at | AL540260_RC | | Hs.26670 | Human PAC clone RP3-515N1 from 22q11.2-q22 |
| 5 | 203490_at | NM_001421.1 | ELF4 | Hs.151139 | E74-like factor 4 (ets domain transcription factor) |
| | | | | | (ELF4) |
| 5 | 235521_at | AW137982_RC | | Hs.222446 | EST |
| 5 | 229391_s_at | AV734646 | | Hs.54277 | DNA segment on chromosome X (unique) 9928 |
| | | | | | expressed sequence |
| 4.9 | 206323_x_at | NM_002547.1 | OPHN1 | Hs.128824 | oligophrenin 1, Rho-GTPase activating protein |
| 4.8 | 200923_at | NM_005567.2 | LGALS3BP | Hs.79339 | lectin, galactoside-binding, soluble, 3 binding |
| | | | | | protein (galectin 6 binding protein) (LGALS3BP) |
| 4.7 | | AL567808_RC | KOX16 | | Zinc finger protein 23 (KOX 16) |
| 4.7 | | NM_005346.2 | Hsp 1B | | Heat shock 70 kD protein 1B |
| 4.7 | | NM_005771.1 | | | Retinol dehydrogenase homolog |
| 4.7 | | NM_001237.1 | CYCA2 | | Cyclin A2 |
| 4.6 | 210861_s_at | AF143679.1 | LIBC | Hs.194678 | WNT1 inducible signaling pathway protein 3 |
| 4.6 | 201697_s_at | NM_001379.1 | DNMT1 | Hs.77462 | DNA (cytosine-5-)-methyltransferase 1 |
| 4.6 | 212873_at | BE349017_RC | | Hs.196914 | minor histocompatibility antigen HA-1 |
| 4.5 | 37005_at | | | | Cluster Incl. D28124: Human mRNA for unknown |
| | | | | | product |
| 4.5 | 237680_at | AI821585_RC | | Hs.181895 | EST |
| 4.4 | 213275_x_at | W47179_RC | | Hs.297939 | cathepsin B |
| 4.4 | 200974_at | NM_001613.1 | ACTA2 | Hs.195851 | actin, alpha 2, smooth muscle, aorta (ACTA2) |
| 4.4 | 201431_s_at | NM_001387.1 | DPYSL3 | Hs.74566 | dihydropyrimidinase-like 3 |
| 4.4 | 229504_at | AI810826_RC | | Hs.61539 | EST |
| 4.4 | 229390_at | AV734646 | | Hs.54277 | DNA segment on chromosome X (unique) 9928 |
| | | | | | expressed sequence |
| 4.3 | | NM_004530.1 | MMP2 | | Matrix metalloproteinase 2 (gelatinase A, 72 kD |
| | | | | | gelatinase, 72 kD type IV collagenase) |
| 4.3 | | NM_005345.3 | Hsp 1A | | Heat shock 70 kD protein 1A |
| 4.3 | 207850_at | NM_002090.1 | GRO3 | Hs.89690 | GRO3 oncogene (GRO3) |
| 4.3 | 218880_at | N36408_RC | | Hs.325364 | hypothetical protein FLJ23306 |
| 4.3 | 202196_s_at | NM_013253.1 | DKK3 | Hs.4909 | dickkopf (Xenopus laevis) homolog 3 (DKK3) |
| 4.3 | 205715_at | NM_004334.1 | BST1 | Hs.169998 | bone marrow stromal cell antigen 1 (BST1) |
| 4.3 | 212077_at | AL583520 | | Hs.182183 | caldesmon, 3 UTR |
| 4.3 | 224937_at | BF311866 | | Hs.300591 | KIAA1436 protein |
| 4.3 | | NM_002961.2 | S100A4 | | S100 calcium-binding protein A4 |
| 4.2 | | NM_012420.1 | | | Retinoic acid and interferon-inducible protein (58 kD) |
| 4.2 | | AF219624.1 | MMP28 | | Matrix metalloproteinase 28 |
| 4.2 | | L37882.1 | | | frizzled (Drosophila) homolog 2 |
| 4.2 | 218224_at | NM_006029.2 | PNMA1 | Hs.194709 | paraneoplastic antigen MA1 |
| 4.2 | 204908_s_at | NM_005178.1 | BCL3 | Hs.31210 | B-cell CLLlymphoma 3 |
| 4.2 | 201746_at | NM_000546.2 | TP53 | Hs.1846 | Tumor protein p53 (Li-Fraumeni syndrome) |
| 4.1 | 211012_s_at | BC000080.1 | | Hs.89633 | promyelocytic leukemia |
| 4.1 | 221653_x_at | BC004395.1 | | Hs.241412 | Similar to apolipoprotein L, clone MGC: 10978 |
| 4.1 | 214022_s_at | AA749101_RC | | Hs.146360 | interferon induced transmembrane protein 1 (9-27) |
| 4.1 | 218847_at | NM_006548.1 | IMP-2 | Hs.30299 | IGF-II mRNA-binding protein 2 (IMP-2) |
| 4 | 209651_at | BC001830.1 | | Hs.25511 | Similar to transforming growth factor beta |
| | | | | | 1induced transcript 1 |
| 4 | | AW592266_RC | | | v-myb avain myeloblastosis viral oncogene |
| | | | | | homolog-like 1 |
| 4 | | AI246687_RC | | | Cathepsin C |
| 4 | 218638_s_at | NM_012445.1 | SPON2 | Hs.288126 | spondin 2, extracellular matrix protein |
| 4 | 211964_at | X05610.1 | | Hs.75617 | collagen, type IV, alpha 2 |
| 4 | 201328_at | AL575509_RC | | Hs.85146 | avian erythroblastosis virus E26 oncogene |
| | | | | | homolog 2 |
| 4 | 204057_at | AI073984_RC | | Hs.14453 | interferon consensus sequence binding protein 1 |
| 4 | 209879_at | AI741056_RC | | Hs.79283 | selectin P ligand |
| 4 | 219033_at | NM_024615.1 | FLJ21308 | Hs.29977 | hypothetical protein FLJ21308 |
| 4 | 226364_at | AU145049_RC | | Hs.38489 | EST |
| 3.9 | 212887_at | AI753659_RC | | Hs.321403 | DKFZp564O2363 |
| 3.9 | | AA927480_RC | | | v-ski avian sarcoma viral oncogene homolog |
| 3.9 | | NM_003674.1 | CDK10 | | Cyclin-dependent kinase (CDC2-like) 10 |
| 3.9 | 242907_at | BF509371_RC | | Hs.160628 | EST |
| 3.8 | 48531_at | | | | Cluster Incl. AA522816: ni40e12.s1 Homo |
| | | | | | sapiens cDNA, 3 end |
| 3.8 | | NM_006299.1 | | | Zinc finger protein 193 |
| 3.8 | 211067_s_at | BC006454.1 | | | growth arrest-specific 7 |
| 3.8 | 209118_s_at | AF141347.1 | | Hs.272897 | tubulin, alpha 3 |
| 3.8 | 213798_s_at | AA806142_RC | | Hs.104125 | adenylyl cyclase-associated protein |
| 3.8 | 208636_at | AI082078_RC | | Hs.119000 | actinin, alpha 1 |
| 3.8 | 209530_at | U07139.1 | | Hs.250712 | calcium channel, voltage-dependent, beta 3 |
| | | | | | subunit |
| 3.8 | 202625_at | AI356412_RC | | Hs.80887 | Yamaguchi sarcoma viral related oncogene |
| | | | | | homolog |
| 3.8 | 200750_s_at | AF054183.1 | | Hs.10842 | RAN small monomeric GTPase |
| 3.8 | 202949_s_at | NM_001450.1 | FHL2 | Hs.8302 | four and a half LIM domains 2 |
| 3.8 | 208851_s_at | AL161958.1 | DKFZp761B15121 | Hs.125359 | Thy-1 cell surface antigen /FL = BC005175.1 |
| 3.8 | 202748_at | NM_004120.2 | GBP2 | Hs.171862 | guanylate binding protein 2, interferon-inducible |
| | | | | | (GBP2) |
| 3.7 | 214684_at | X63381.1 | RSRFC4 | Hs.182280 | serum response factor-related protein, RSRFC4. |
| | | | | | MADS box transcription enhancer factor 2, |
| 3.7 | 213603_s_at | BE138888_RC | | Hs.301175 | HSPC022 protein |
| 3.7 | 228345_at | AI745136_RC | | Hs.34656 | EST |
| 3.6 | 201458_s_at | NM_004725.1 | BUB3 | Hs.40323 | BUB3 budding uninhibited by benzimidazoles 3 |
| | | | | | homolog (yeast) |
| 3.5 | 213923_at | AW005535_RC | | Hs.155218 | E1B-55 kDa-associated protein 5 |
| 3.5 | 203044_at | NM_014918.1 | KIAA0990 | Hs.110488 | KIAA0990 protein (KIAA0990) |
| 3.5 | 213646_x_at | BE300252_RC | | Hs.240615 | hypothetical protein FLJ13556 similar to N-myc |
| | | | | | downstream regulated 3 |
| 3.5 | 228245_s_at | AW594320_RC | | Hs.110080 | Weakly similar to S13495 pregnancy zone |
| | | | | | protein |
| 3.5 | 227326_at | BE966768_RC | | Hs.11924 | Weakly similar to ALU1_HUMAN ALU |
| | | | | | SUBFAMILY J SEQUENCE |
| 3.5 | 226944_at | AW518728_RC | | Hs.60440 | Weakly similar to serin protease with IGF- |
| | | | | | binding motif |
| 3.4 | 203455_s_at | NM_002970.1 | SAT | Hs.28491 | spermidinespermine N1-acetyltransferase (SAT) |
| 3.4 | 203554_x_at | NM_004219.2 | PTTG1 | Hs.252587 | pituitary tumor-transforming 1 |
| 3.4 | 214247_s_at | AU148057_RC | | Hs.278503 | EST regulated in glioma |
| 3.4 | 203476_at | NM_006670.1 | 5T4 | Hs.82128 | 5T4 oncofetal trophoblast glycoprotein |
| 3.4 | 202820_at | NM_001621.2 | AHR | Hs.170087 | aryl hydrocarbon receptor (AHR) |
| 3.4 | 201601_x_at | NM_003641.1 | IFITM1 | Hs.146360 | interferon induced transmembrane protein 1 (9-27) |
| | | | | | (IFITM1) |
| 3.4 | 239345_at | AI671566_RC | | Hs.200313 | EST |
| 3.4 | 227036_at | N66622_RC | | Hs.29263 | hypothetical protein FLJ11896 |
| 3.3 | 213857_s_at | BG230614_RC | | Hs.82685 | CD47 antigen (Rh-related antigen, integrin- |
| | | | | | associated signal transducer) |
| 3.3 | 213017_at | AL534702_RC | | Hs.13377 | EST |
| 3.3 | 213537_at | AI128225_RC | | Hs.914 | major histocompatibility complex, class II, DP |
| | | | | | alpha 1 |
| 3.3 | 217118_s_at | AK025608.1 | | Hs.13255 | KIAA0930 protein |
| 3.3 | 202968_s_at | Y09216.1 | Dyrk2 | Hs.173135 | 1. protein kinase, Dyrk2. 2. dual-specificity |
| | | | | | tyrosine-(Y)-phosphorylation regulated kinase 2 |
| 3.3 | 41220_at | | | | MLL septin-like fusion* |
| 3.3 | 213199_at | AL080220.1 | DKFZp586P0123 | Hs.6285 | (DKFZP586P0123) protein |
| 3.3 | 208896_at | X98743.1 | | Hs.100555 | DEADH (Asp-Glu-Ala-AspHis) box polypeptide |
| | | | | | 18 (Myc-regulated) |
| 3.3 | 226435_at | AU145309_RC | | Hs.301152 | DKFZp434F053 |
| 3.3 | 228280_at | AI188445_RC | | Hs.152618 | EST |
| 3.3 | | NM_003882.1 | | | WNT1 inducible signalling pathway protein 1 |
| 3.3 | 230110_at | AV713773 | | Hs.210792 | Weakly similar to ALU8_HUMAN ALU |
| | | | | | SUBFAMILY SX SEQUENCE |
| 3.2 | | NM_000784.1 | | | Cytochrome P450, subfamily XXVIIA |
| 3.2 | | AK000445.1 | HOX C9 | | Homeo box C9 |
| 3.2 | 212632_at | N32035_RC | | Hs.8906 | Homo sapiens clone 24889 mRNA sequence |
| 3.2 | 201090_x_at | NM_006082.1 | K-ALPHA-1 | Hs.278242 | tubulin, alpha, ubiquitous |
| 3.2 | 211758_x_at | BC005968.1 | | | ATP binding protein associated with cell |
| | | | | | differentiation, clone MGC: 14620 |
| 3.2 | 210629_x_at | AF000425.1 | LST1 | Hs.88411 | lymphocyte antigen 117 |
| 3.1 | 212012_at | BF342851 | | Hs.118893 | Melanoma associated gene |
| 3.1 | 203414_at | NM_012329.1 | MMD | Hs.79889 | monocyte to macrophage differentiation- |
| | | | | | associated (MMD) |
| 3.1 | | NM_005402.1 | | | v-ral simian leukemia viral oncogene homolog A |
| | | | | | (ras related) |
| 3.1 | 213102_at | Z78330 | | Hs.10927 | hypothetical protein EUROIMAGE1875335 |
| 3.1 | 206414_s_at | NM_003887.1 | DDEF2 | Hs.12802 | Homo sapiens development and differentiation |
| | | | | | enhancing factor 2 (DDEF2) |
| 3.1 | 225364_at | BE222274_RC | | Hs.250824 | Homo sapiens cDNA: FLJ23435 fis, clone |
| | | | | | HRC12631 |
| 3.1 | 223501_at | AW151360_RC | | Hs.270737 | tumor necrosis factor (ligand) superfamily, |
| | | | | | member 13b |
| 3.1 | 225188_at | AA194149_RC | | Hs.42656 | KIAA1681 protein |
| 3 | 219700_at | NM_020405.1 | TEM7 | Hs.125036 | tumor endothelial marker 7 precursor |
| 3 | 200697_at | NM_000188.1 | HK1 | Hs.118625 | hexokinase 1 |
| 3 | 34726_at | | | | Cluster includes Human voltage-gated calcium |
| | | | | | channel beta subunit |
| 3 | 208540_x_at | NM_021039.1 | S100A14 | Hs.247697 | S100 calcium-binding protein A14 (calgizzarin) |
| | | | | | (S100A14), |
| 3 | | R01140_RC | Hsp1 alpha | | Heat shock 90 kD protein 1, alpha |
| 3 | 206219_s_at | NM_005428.2 | VAV1 | Hs.116237 | vav 1 oncogene (VAV1) |
| 3 | 209370_s_at | BE502377_RC | | Hs.167679 | SH3-domain binding protein 2 |
| 3 | 219878_s_at | NM_015995.1 | KLF13 | Hs.7104 | Homo sapiens Kruppel-like factor 13 (KLF13), |
| 3 | 226837_at | BE967019_RC | | Hs.94133 | EST |
| 3 | 223095_at | BC004995.1 | | Hs.209614 | Unknown (protein for MGC: 4415) |
| 3 | 227386_s_at | N63821_RC | | Hs.268024 | cDNA DKFZp434C184 |
| 3 | 223738_s_at | AL136705.1 | DKFZp566B1524 | Hs.23363 | hypothetical protein FLJ10983 |
| 3 | 223361_at | AF116682.1 | | Hs.238205 | hypothetical protein PRO2013 |
| 3 | 239269_at | AW449577_RC | | Hs.200577 | EST |
| 3 | | NM_007150.1 | | | Zinc finger protein 185 (LIM domain) |
| 3 | | AU150728_RC | | | Zinc finger protein 267 |
| 2.9 | | AI806984_RC | | | Retinoic acid receptor, alpha |
| 2.9 | | U91903.1 | | | frizzled-related protein |
| 2.9 | 201776_s_at | AK001487.1 | | Hs.62515 | KIAA0494 gene product |
| 2.9 | 212837_at | D63877.1 | KIAA0157 | Hs.82324 | KIAA0157 protein |
| 2.9 | 205542_at | NM_012449.1 | STEAP | Hs.61635 | six transmembrane epithelial antigen of the |
| | | | | | prostate (STEAP) |
| 2.9 | 201042_at | AL031651 | | Hs.8265 | transglutaminase 2 (C polypeptide, protein- |
| | | | | | glutamine-gamma-glutamyltransferase) |
| 2.9 | 225665_at | AI129320_RC | | Hs.16930 | EST |
| 2.8 | 211058_x_at | BC006379.1 | | | tubulin alpha 1 |
| 2.8 | 202720_at | NM_015641.1 | DKFZP586B2022 | Hs.165986 | testin (DKFZP586B2022) |
| 2.8 | 205051_s_at | NM_000222.1 | KIT | Hs.81665 | v-kit Hardy-Zuckerman 4 feline sarcoma viral |
| | | | | | oncogene homolog |
| 2.8 | 209939_x_at | AF005775.1 | clarp | Hs.195175 | CASP8 and FADD-like apoptosis regulator |
| 2.8 | | NM_003428.1 | HPF2 | | Zinc finger protein 84 (HPF2) |
| 2.8 | 219039_at | NM_017789.1 | FLJ20369 | Hs.7188 | hypothetical protein FLJ20369 (FLJ20369), |
| 2.8 | 223463_at | AF161486.1 | | Hs.94769 | RAB23, member RAS oncogene family |
| 2.7 | | BE407516 | CYCB1 | | Cyclin B1 |
| 2.7 | 214853_s_at | AI091079_RC | | Hs.81972 | SHC (Src homology 2 domain containing) |
| | | | | | transforming protein 1 |
| 2.7 | | AA121673_RC | | | Zinc finger protein 281 |
| 2.7 | 203706_s_at | NM_003507.1 | FZD7 | Hs.173859 | frizzled homolog 7 (Drosophila) |
| 2.7 | 213513_x_at | BG034239 | | Hs.252280 | Rho guanine nucleotide exchange factor (GEF) 1 |
| 2.7 | 208690_s_at | BC000915.1 | | Hs.75807 | Similar to LIM protein, clone UG PDZ and LIM |
| | | | | | domain 1 (elfin) |
| 2.7 | 216973_s_at | S49765.1 | homeobox gene | Hs.819 | homeo box B7 (homeobox gene) |
| 2.7 | 213168_at | AU145005_RC | | Hs.44450 | Sp3 transcription factor |
| 2.7 | 211072_x_at | BC006481.1 | | | tubulin alpha 1, |
| 2.7 | 227489_at | BE962027_RC | | Hs.169872 | EST |
| 2.6 | | R78668_RC | CDKI 1C, P57^{KIP2} | | Cyclin-dependent kinase inhibitor 1C (p57^{KIP2}) |
| 2.6 | | NM_030775.1 | WNT5b | | WNT5b protein |
| 2.6 | | BG403660 | | | Heat shock 105 kD protein |
| 2.6 | | NM_020657.1 | | | Zinc finger protein 304 |
| 2.6 | 213261_at | AA035414_RC | | Hs.16950 | KIAA0342 gene product |
| 2.6 | 201061_s_at | M81635.1 | stomatin peptide | Hs.160483 | erythrocyte membrane protein (stomatin peptide) |
| 2.6 | 209762_x_at | AF280094.1 | | Hs.38125 | 1. transcriptional coactivator Sp110b 2. |
| | | | | | interferon-induced protein 75 |
| 2.6 | 204122_at | NM_003332.1 | TYROBP | Hs.9963 | TYRO protein tyrosine kinase binding protein |
| | | | | | (TYROBP) |
| 2.6 | 202202_s_at | NM_002290.2 | LAMA4 | Hs.78672 | laminin, alpha 4 (LAMA4) |
| 2.6 | 203343_at | NM_003359.1 | UGDH | Hs.28309 | UDP-glucose dehydrogenase (UGDH) |
| 2.6 | 203370_s_at | NM_005451.2 | ENIGMA | Hs.102948 | enigma (LIM domain protein) |
| 2.6 | 209250_at | BC000961.2 | | Hs.185973 | degenerative spermatocyte (homolog |
| | | | | | Drosophila; lipid desaturase), |
| 2.6 | 226914_at | AU158936_RC | | Hs.234174 | Homo sapiens cDNA FLJ13819 fis, clone |
| | | | | | THYRO1000452 |
| 2.6 | 226474_at | AA005023_RC | | Hs.10888 | Homo sapiens cDNA: FLJ21559 fis, clone |
| | | | | | COL06406 |
| 2.6 | 223082_at | AF230904.1 | CIN85 | Hs.153260 | c-Cbl-interacting protein (CIN85) |
| 2.6 | 238327_at | AI962367_RC | | Hs.289039 | Moderately similar to S72487 11 orf3 5 of PD- |
| | | | | | ECGFTP |
| 2.5 | 202565_s_at | NM_003174.2 | SVIL | Hs.154567 | supervillin (SVIL), transcript variant 1, |
| 2.5 | 202377_at | AW026535_RC | | Hs.23581 | leptin receptor gene-related protein |
| 2.5 | 201999_s_at | NM_006519.1 | TCTEL1 | Hs.266940 | t-complex-associated-testis-expressed 1-like 1 |
| | | | | | (TCTEL1) |
| 2.5 | 203312_x_at | NM_001663.2 | ARF6 | Hs.89474 | ADP-ribosylation factor 6 (ARF6) |
| 2.5 | | NM_006526.1 | | | Zinc finger protein 217 |
| 2.5 | 239577_at | AV699781_RC | | Hs.54245 | EST |
| 2.5 | 225366_at | AI652855_RC | | Hs.23363 | hypothetical protein FLJ10983 |
| 2.4 | 205962_at | NM_002577.1 | PAK2 | Hs.30692 | p21 (CDKN1A)-activated kinase 2 (PAK2) |
| 2.4 | 201930_at | NM_005915.2 | MCM6 | Hs.155462 | minichromosome maintenance deficient (mis5, |
| | | | | | S. pombe) 6 (MCM6) |
| 2.4 | 35820_at | | | | Cluster includes GM2 activator protein |
| 2.4 | 202526_at | U44378.1 | DPC4 | Hs.75862 | Human homozygous deletion target in |
| | | | | | pancreatic carcinoma (DPC4) |
| 2.4 | 218627_at | NM_018370.1 | FLJ11259 | Hs.184465 | hypothetical protein FLJ11259 (FLJ11259) |
| 2.4 | 209787_s_at | BC001282.1 | | Hs.236774 | high-mobility group (nonhistone chromosomal) |
| | | | | | protein 17-like 3, |
| 2.4 | 228299_at | AV707142 | | Hs.188757 | Homo sapiens, clone MGC: 5564, mRNA, |
| | | | | | complete cds |
| 2.4 | 226632_at | AL513673_RC | | Hs.95120 | EST |
| 2.3 | 207988_s_at | NM_005731.1 | ARPC2 | Hs.83583 | actin related protein 23 complex, subunit 2 (34 kD) |
| | | | | | (ARPC2) |
| 2.3 | | AK024855.1 | | | Cathepsin S |
| 2.3 | 202165_at | BF966540_RC | | Hs.267819 | phosphatase 1, regulatory (inhibitor) subunit 2 |
| 2.3 | 219151_s_at | NM_007081.1 | RABL2B | Hs.145409 | RAB, member of RAS oncogene family-like 2B |
| 2.3 | 230236_at | AL045590_RC | | Hs.180197 | EST |
| 2.3 | 226878_at | AL581873_RC | | Hs.11135 | major histocompatibility complex, class II, DN |
| | | | | | alpha |
| 2.3 | 241353_s_at | AW471181_RC | | Hs.160874 | EST |
| 2.2 | 220177_s_at | NM_024022.1 | TMPRSS3 | Hs.298241 | Transmembrane protease, serine 3 (TMPRSS3) |
| 2.2 | 207039_at | NM_000077.1 | CDKN2A | Hs.1174 | p16INK4A |
| 2.2 | 208374_s_at | NM_006135.1 | CAPZA1 | Hs.184270 | capping protein (actin filament) muscle Z-line, |
| | | | | | alpha 1 (CAPZA1) |
| 2.2 | 209332_s_at | BC003525.1 | | Hs.42712 | MAX protein |
| 2.2 | 211762_s_at | BC005978.1 | | | karyopherin alpha 2 (RAG cohort 1, importin |
| | | | | | alpha 1) |
| 2.2 | 238025_at | AA706818_RC | | Hs.119878 | EST |
| 2.2 | 225917_at | AA766897_RC | | Hs.122444 | EST |
| 2.2 | 225889_at | BF475280_RC | | Hs.285833 | Homo sapiens cDNA: FLJ22135 fis, clone |
| | | | | | HEP20858 |
| 2.1 | 209974_s_at | AF047473.1 | BUB3 | Hs.40323 | testis mitotic checkpoint BUB3 (BUB3) |
| 2.1 | 211063_s_at | BC006403.1 | | | NCK adaptor protein 1 |
| 2.1 | 218053_at | NM_017892.1 | FLJ20585 | Hs.107213 | hypothetical protein FLJ20585 |
| 2.1 | 218669_at | NM_021183.1 | LOC57826 | Hs.225979 | hypothetical protein similar to small G proteins, |
| | | | | | especially RAP-2A (LOC57826), |
| 1.9 | 209348_s_at | AF055376.1 | c-maf | Hs.30250 | |
| 1.8 | 217993_s_at | NM_013283.1 | MAT2B | Hs.54642 | methionine adenosyltransferase II, beta |
| | | | | | (MAT2B). |
| 1.7 | 201533_at | NM_001904.1 | CTNNB1 | Hs.171271 | Beta-Catenin (CTNNB1) |
| 1.7 | 212203_x_at | BF338947 | | Hs.182241 | interferon induced transmembrane protein 3 (1-8 |
| | | | | | U) |
| 1.7 | 210621_s_at | M23612.1 | GAP | Hs.758 | GTPase-activating protein (GAP) |
| 1.5 | 201833_at | NM_001527.1 | HDAC2 | Hs.3352 | histone deacetylase 2 |

TABLE 4
Genes that are down-regulated in subjects having pancreatic cancer

| Fold | Affymetrix | GenBANK | GenBANK | Unigene | |
|---|---|---|---|---|---|
| Change | Code | Accession No. | Gene Symbol | cluster | Annotations |
| 1 | 216836_s_at | X03363.1 | | Hs.323910 | c-erb-B-2 |
| 1 | 208711_s_at | BC000076.1 | | Hs.82932 | cyclin D1 (PRAD1: parathyroid adenomatosis 1) |
| 0.7 | 202284_s_at | NM_000389.1 | CDKN1A | Hs.179665 | p21 |
| 0.5 | 215721_at | X58397.1 | immunoglobulin | Hs.81220 | CLL-12 transcript of unrearranged immunoglobulin |
| | | | heavy chain | | V(H)5 gene. |
| 0.5 | 210960_at | M76446.1 | | Hs.557 | alpha-A1-adrenergic receptor |
| 0.5 | 220815_at | NM_013266.1 | VR22 | Hs.257051 | alpha catenin-like protein |
| 0.4 | | NM_015871.1 | | | Zinc finger protein. |
| 0.4 | | R92925_RC | | | Mitochondrial solute carrier protein |
| 0.4 | | NM_004294.1 | | | Mitochondrial translational release factor 1 |
| 0.4 | | T67741_RC | | | Cytochrome P450, subfamily IIA |
| 0.4 | | NM_001914.1 | | | Cytochrome b-5 |
| 0.4 | | W45551_RC | MMP24 | | Matrix metalloproteinase 24 (membrane inserted) |
| 0.4 | | NM_002908.1 | | | v-rel avian retculoendotheliosis viral oncogene |
| | | | | | homolog |
| 0.4 | | NM_002144.1 | HOX B1 | | Homeo box B1 |
| 0.4 | | BE256479 | | | Heat shock 60 kD protein 1 (chaperonin) |
| 0.4 | | AB034951.1 | | | Heat shock 70 kD protein 8 |
| 0.4 | | NM_016292.1 | | | Heat shock protein 75 |
| 0.4 | | AI393937 | | | Heat shock transcription factor 1 |
| 0.4 | | NM_003793.2 | | | Cathepsin F |
| 0.4 | 218264_at | NM_016567.1 | TOK-1 | Hs.279862 | p21 binding protein (TOK-1) |
| 0.4 | 215414_at | AI524687_RC | | Hs.57969 | phenylalanine-tRNA synthetase |
| 0.4 | 211832_s_at | AF201370.1 | | Hs.170027 | 1. MDM2-a1 2. mouse double minute 2, human |
| | | | | | homolog of; p53-binding protein |
| 0.4 | 220843_s_at | NM_014156.1 | DKFZP564O0463 | Hs.273344 | DKFZP564O0463 protein (DKFZP564O0463) |
| 0.3 | 201827_at | AF113019.1 | | Hs.250581 | matrix associated, actin dependent regulator of |
| | | | | | chromatin, subfamily d, member 2 |
| 0.3 | | NM_002467.1 | | | v-myc avian myelocytomatosis viral oncogene |
| | | | | | homolog |
| 0.3 | | M19720 | | | v-myc avian myelocytomatosis viral oncogene |
| | | | | | homolog 1, lung carcinoma derived |
| 0.3 | | AI493587_RC | | | Zinc finger protein 106 |
| 0.3 | | NM_006006.1 | | | Zinc finger protein 145 |
| 0.3 | | NM_006963.1 | KOX15 | | Zinc finger protein 22 (KOX15) |
| 0.3 | | NM_001335.1 | | | Cathepsin W |
| 0.3 | 205360_at | AI718295_RC | | Hs.91161 | prefoldin 4 |
| 0.3 | 211541_s_at | U52373.1 | mnb | Hs.75842 | serinethreonine kinase MNB (mnb) |
| 0.3 | 205309_at | NM_014474.1 | ASML3B | Hs.123659 | acid sphingomyelinase-like phosphodiesterase |
| | | | | | (ASML3B) |
| 0.3 | 203904_x_at | NM_002231.2 | KAI1 | Hs.323949 | kangai 1 |
| 0.3 | 216906_at | U20428.1 | | Hs.56937 | suppression of tumorigenicity 14 (colon carcinoma, |
| | | | | | matriptase, epithin) |
| 0.2 | 211513_s_at | AF172449.1 | | Hs.67896 | opioid growth factor receptor |
| 0.2 | 210060_at | M36476.1 | PDEG | Hs.1857 | cGMP phosphodiesterase gamma-subunit |
| 0.2 | 211141_s_at | AF180474.1 | NOT3 | Hs.108300 | CCR4-NOT transcription complex, subunit 3 |
| 0.2 | | NM_002466.1 | | | v-myb avian myeloblastosis viral oncogene |
| | | | | | homolog-like 2 |
| 0.2 | 218922_s_at | NM_024552.1 | FLJ12089 | Hs.11896 | hypothetical protein FLJ12089 |
| 0.2 | 215251_at | AA595276_RC | | Hs.243804 | Homo sapiens cDNA FLJ13800 fis, clone |
| | | | | | THYRO1000156 |
| 0.1 | 202063_s_at | AB020335.1 | TSA305 | Hs.181300 | sal-1 (suppressor of lin-12, C. elegans)-like |
| 0.1 | 210401_at | U45448.1 | | Hs.41735 | purinergic receptor P2X, ligand-gated ion channel, 1 |
| 0.1 | 221295_at | NM_001279.1 | CIDEA | Hs.249129 | cell death-inducing DFFA-like effector a (CIDEA) |
| 0.1 | 220012_at | NM_019891.1 | ERO1-L(BETA) | Hs.150763 | endoplasmic reticulum oxidoreductin 1-Lbeta |
| | | | | | (ERO1-L(BETA)) |
| 0.1 | 210339_s_at | BC005196.1 | | Hs.181350 | kallikrein 2, prostatic |
| 0.1 | 208377_s_at | NM_005183.1 | CACNA1F | Hs.139263 | calcium channel, voltage-dependent, alpha 1F |
| | | | | | subunit (CACNA1F) |
| 0.1 | 221868_at | AB032981.1 | KIAA1155 | Hs.102657 | KIAA1155 protein |
| 0.1 | | BC000069.1 | RARRES2 | | retinoic acid receptor responder (tazarotene |
| | | | | | induced) 2 |
| 0.1 | 204394_at | NM_003627.1 | POV1 | Hs.18910 | prostate cancer overexpressed gene 1 (POV1) |
| 0 | 207412_x_at | NM_001808.1 | CELL | Hs.169271 | carboxyl ester lipase-like (bile salt-stimulated lipase- |
| | | | | | like) (CELL) |
| 0 | 206681_x_at | NM_001502.1 | GP2 | Hs.53985 | glycoprotein 2 (zymogen granule membrane) (GP2) |
| 0 | 206784_at | NM_001169.1 | AQP8 | Hs.176658 | aquaporin 8 (AQP8) |
| 0 | 208473_s_at | NM_016295.1 | LOC51724 | Hs.274493 | pancreatic zymogen granule membrane associated |
| | | | | | protein GP2 beta form (LOC51724) |
| 0 | 205164_at | NM_014291.1 | GCAT | Hs.54609 | glycine C-acetyltransferase (2-amino-3-ketobutyrate |
| | | | | | coenzyme A ligase) (GCAT) |
| 0 | 201785_at | NM_002933.1 | RNASE1 | Hs.78224 | ribonuclease; RNase A family, 1 (pancreatic) |
| | | | | | (RNASE1) |
| 0 | 214377_s_at | BF508685_RC | | Hs.150601 | chymotrypsin-like |
| 0 | 221259_s_at | NM_031276.1 | TEX11 | | testis expressed sequence 11 (TEX11) |
| 0 | 206694_at | NM_006229.1 | PNLIPRP1 | Hs.73923 | pancreatic lipase-related protein 1 (PNLIPRP1) |

TABLE 5
Genes encoding membrane proteins that are diagnostic of pancreatic cancer

| | GenBank | Unigene | Gene | | |
|---|---|---|---|---|---|
| Affy Code | Accession | Accession | Symbol | Unigene Descriptor | Fold Change |
| 201884_at | NM_004363.1 | Hs.220529 | CEACAM5 | carcinoembryonic antigen-related cell adhesion molecule 5 | 36.5 |
| 203108_at | NM_003979.2 | Hs.194691 | RAI3 | retinoic acid induced 3 | 26.3 |
| 205234_at | NM_004696.1 | Hs.23590 | SLC16A4 | solute carrier family 16 (monocarboxylic acid transporters), | 18.7 |
| | | | | member 4 | |
| 206392_s_at | NM_002888.1 | Hs.82547 | RARRES1 | retinoic acid receptor responder (tazarotene induced) 1 | 16.5 |
| 203757_s_at | BC005008.1 | Hs.73848 | | carcinoembryonic antigen-related cell adhesion molecule 6 (non- | 15.4 |
| | | | | specific cross reacting antigen) | |
| 205319_at | NM_005672.1 | Hs.20166 | PSCA | prostate stem cell antigen | 14.4 |
| 216834_at | S59049.1 | Hs.75256 | BL34 | regulator of G-protein signalling 1 | 13.6 |
| 202497_x_at | AI631159_RC | Hs.7594 | | solute carrier family 2 (facilitated glucose transporter), member 3 | 10.9 |
| 205860_x_at | NM_004476.1 | Hs.1915 | FOLH1 | folate hydrolase (prostate-specific membrane antigen) 1 | 10 |
| 203726_s_at | NM_000227.1 | Hs.83450 | LAMA3 | laminin, alpha 3 (nicein (150 kD), kalinin (165 kD), BM600 | 9 |
| | | | | (150 kD), epilegrin) | |
| 202307_s_at | NM_000593.2 | Hs.158164 | ABCB2 | ATP-binding cassette, sub-family B (MDRTAP), member 2 | 9 |
| 201798_s_at | NM_013451.1 | Hs.234680 | FER1L3 | fer-1 (C. elegans)-like 3 (myoferlin) | 7.6 |
| 206391_at | NM_002888.1 | Hs.82547 | RARRES1 | retinoic acid receptor responder (tazarotene induced) 1 | 7 |
| 209900_s_at | AL162079.1 | Hs.75231 | DKFZp762B2310 | solute carrier family 16 (monocarboxylic acid transporters), | 6.7 |
| | | | | member 1 | |
| 203821_at | NM_001945.1 | Hs.799 | DTR | diphtheria toxin receptor (heparin-binding epidermal growth | 6.6 |
| | | | | factor-like growth factor) | |
| 201655_s_at | M85289.1 | Hs.211573 | HSPG2 | heparan sulfate proteoglycan 2 (perlecan) | 6.5 |
| 203510_at | BG170541 | Hs.285754 | | met proto-oncogene (hepatocyte growth factor receptor) | 6.4 |
| 201141_at | NM_002510.1 | Hs.82226 | GPNMB | glycoprotein (transmembrane) nmb | 5.5 |
| 215633_x_at | AV713720 | Hs.306434 | | Homo sapiens mRNA for LST-1N protein | 5.5 |
| 204137_at | NM_003272.1 | Hs.15791 | TM7SF1 | transmembrane 7 superfamily member 1 (upregulated in kidney) | 5.3 |
| 205715_at | NM_004334.1 | Hs.169998 | BST1 | bone marrow stromal cell antigen 1 | 4.3 |
| 209879_at | AI741056_RC | Hs.79283 | | selectin P ligand | 4 |
| 209530_at | U07139.1 | Hs.250712 | | calcium channel, voltage-dependent, beta 3 subunit | 3.8 |
| 202625_at | AI356412_RC | Hs.80887 | | v-yes-1 Yamaguchi sarcoma viral related oncogene homolog | 3.8 |
| 208851_s_at | AL161958.1 | Hs.125359 | DKFZp761B15121 | Thy-1 cell surface antigen | 3.8 |
| 203476_at | NM_006670.1 | Hs.82128 | 5T4 | 5T4 oncofetal trophoblast glycoprotein | 3.4 |
| 201601_x_at | NM_003641.1 | Hs.146360 | IFITM1 | interferon induced transmembrane protein 1 (9-27) | 3.4 |
| 210629_x_at | AF000425.1 | Hs.88411 | LST1 | lymphocyte antigen 117 | 3.2 |
| 203414_at | NM_012329.1 | Hs.79889 | MMD | monocyte to macrophage differentiation-associated | 3.1 |
| 223501_at | AW151360_RC | Hs.270737 | | tumor necrosis factor (ligand) superfamily, member 13b | 3.1 |
| 34726_at | | | | calcium channel, voltage-dependent, beta 3 subunit | 3 |
| 205542_at | NM_012449.1 | Hs.61635 | STEAP | six transmembrane epithelial antigen of the prostate | 2.9 |
| 203706_s_at | NM_003507.1 | Hs.173859 | FZD7 | frizzled (Drosophila) homolog 7 | 2.7 |
| 201061_s_at | M81635.1 | Hs.160483 | stomatin peptide | erythrocyte membrane protein band 7.2 (stomatin) | 2.6 |
| 204122_at | NM_003332.1 | Hs.9963 | TYROBP | TYRO protein tyrosine kinase binding protein | 2.6 |
| 209250_at | BC000961.2 | Hs.185973 | | degenerative spermatocyte (homolog Drosophila; lipid | 2.6 |
| | | | | desaturase) | |
| 202565_s_at | NM_003174.2 | Hs.154567 | SVIL | supervillin | 2.5 |
| 203312_x_at | NM_001663.2 | Hs.89474 | ARF6 | ADP-ribosylation factor 6 | 2.5 |
| 201533_at | NM_001904.1 | Hs.171271 | CTNNB1 | catenin (cadherin-associated protein), beta 1 (88 kD) | 1.7 |
| 210960_at | M76446.1 | Hs.557 | | adrenergic, alpha-1D-, receptor | 0.5 |
| 203904_x_at | NM_002231.2 | Hs.323949 | KAI1 | kangai 1 (suppression of tumorigenicity 6, prostate; CD82 | 0.3 |
| | | | | antigen (R2 leukocyte antigen, antigen detected by monoclonal | |
| | | | | and antibody IA4)) | |
| 210401_at | U45448.1 | Hs.41735 | | purinergic receptor P2X, ligand-gated ion channel, 1 | 0.1 |
| 206681_x_at | NM_001502.1 | Hs.53985 | GP2 | glycoprotein 2 (zymogen granule membrane) | 0 |
| 206784_at | NM_001169.1 | Hs.176658 | AQP8 | aquaporin 8 | 0 |
| 208473_s_at | NM_016295.1 | Hs.274493 | LOC51724 | pancreatic zymogen granule membrane associated protein GP2 | 0 |
| | | | | beta form | |

TABLE 6
Genes encoding extracellular proteins that are diagnostic of pancreatic cancer

| | GenBank | Unigene | Gene | | |
|---|---|---|---|---|---|
| Affy Code | Accession | Accession | Symbol | Unigene Descriptor | Fold Change |
| 205476_at | NM_004591.1 | Hs.75498 | SCYA20 | small inducible cytokine subfamily A (Cys-Cys), | 64.2 |
| | | | | member 20 | |
| 210511_s_at | M13436.1 | Hs.727 | INHBA | inhibin, beta A (activin A, activin AB alpha | 24.5 |
| | | | | polypeptide) | |
| 210095_s_at | M31159.1 | Hs.77326 | IGFBP1 | insulin-like growth factor binding protein 3 | 19.4 |
| 203878_s_at | NM_005940.2 | Hs.155324 | MMP11 | matrix metalloproteinase 11 (stromelysin 3) | 16.4 |
| 212464_s_at | X02761.1 | Hs.287820 | | fibronectin 1 | 14.8 |
| 204619_s_at | BF590263_RC | Hs.81800 | | chondroitin sulfate proteoglycan 2 (versican) | 14.1 |
| 221731_x_at | BF218922 | Hs.81800 | | chondroitin sulfate proteoglycan 2 (versican) | 13.2 |
| 205713_s_at | NM_000095.1 | Hs.1584 | COMP | cartilage oligomeric matrix protein | 12.8 |
| | | | | (pseudoachondroplasia, epiphyseal dysplasia 1, | |
| | | | | multiple) | |
| 202859_x_at | NM_000584.1 | Hs.624 | IL8 | interleukin 8 | 12.5 |
| 209792_s_at | BC002710.1 | Hs.69423 | | kallikrein 10 | 12.1 |
| 218468_s_at | AF154054.1 | Hs.40098 | DRM | cysteine knot superfamily 1, BMP antagonist 1 | 10.6 |
| 203083_at | NM_003247.1 | Hs.108623 | THBS2 | thrombospondin 2 | 9.6 |
| 201645_at | NM_002160.1 | Hs.289114 | HXB | hexabrachion (tenascin C, cytotactin) | 9.6 |
| 206560_s_at | NM_006533.1 | Hs.279651 | MIA | melanoma inhibitory activity | 9.3 |
| 204933_s_at | NM_002546.1 | Hs.81791 | TNFRSF11B | tumor necrosis factor receptor superfamily, member | 8.7 |
| | | | | 11b (osteoprotegerin) | |
| 218469_at | NM_013372.1 | Hs.40098 | CKTSF1B1 | cysteine knot superfamily 1, BMP antagonist 1 | 8.1 |
| 204620_s_at | NM_004385.1 | Hs.81800 | CSPG2 | chondroitin sulfate proteoglycan 2 (versican) | 7.8 |
| 200665_s_at | NM_003118.1 | Hs.111779 | SPARC | secreted protein, acidic, cysteine-rich (osteonectin) | 7.4 |
| 204052_s_at | NM_003014.2 | Hs.105700 | SFRP4 | secreted frizzled-related protein 4 | 6.7 |
| 203821_at | NM_001945.1 | Hs.799 | DTR | Diphtheria toxin receptor (heparin-binding epidermal | 6.6 |
| | | | | growth factor-like growth factor) | |
| 201655_s_at | M85289.1 | Hs.211573 | HSPG2 | heparan sulfate proteoglycan 2 (perlecan) | 6.5 |
| 202766_s_at | NM_000138.1 | Hs.750 | FBN1 | fibrillin 1 (Marfan syndrome) | 5.9 |
| 200923_at | NM_005567.2 | Hs.79339 | LGALS3BP | lectin, galactoside-binding, soluble, 3 binding protein | 4.8 |
| | | | | (galectin 6 binding protein) | |
| 207850_at | NM_002090.1 | Hs.89690 | GRO3 | GRO3 oncogene | 4.3 |
| 202196_s_at | NM_013253.1 | Hs.4909 | DKK3 | dickkopf (Xenopus laevis) homolog 3 | 4.3 |
| 218638_s_at | NM_012445.1 | Hs.288126 | SPON2 | spondin 2, extracellular matrix protein | 4 |
| 201785_at | NM_002933.1 | Hs.78224 | RNASE1 | ribonuclease, RNase A family, 1 (pancreatic) | 0 |
| 214377_s_at | BF508685_RC | Hs.150601 | | chymotrypsin-like | 0 |
| 206694_at | NM_006229.1 | Hs.73923 | PNLIPRP1 | pancreatic lipase-related protein 1 | 0 |

TABLE 7
Genes encoding proteins of the TGF-beta signalling pathway that are diagnostic of pancreatic cancer

| | GenBank | Unigene | Gene | | |
|---|---|---|---|---|---|
| Affy Code | Accession | Accession | Symbol | Unigene Descriptor | Fold Change |
| 210511_s_at | M13436.1 | Hs.727 | INHBA | inhibin, beta A (activin A, activin AB alpha | 24.5 |
| | | | | polypeptide) | |
| 221558_s_at | AF288571.1 | Hs.44865 | LEF1 | lymphoid enhancer binding factor-1 | 9.3 |
| 209969_s_at | BC002704.1 | Hs.21486 | | signal transducer and activator of | 5.3 |
| | | | | transcription 1, 91 kD | |
| 202526_at | U44378.1 | Hs.75862 | DPC4 | MAD (mothers against decapentaplegic, | 2.4 |
| | | | | Drosophila) homolog 4 | |
| 201533_at | NM_001904.1 | Hs.171271 | CTNNB1 | catenin (cadherin-associated protein), beta 1 | 1.7 |
| | | | | (88 kD) | |

TABLE 8
Genes encoding proteins of the WNT signalling pathway that are diagnostic of pancreatic cancer

| | GenBank | Unigene | Gene | | |
|---|---|---|---|---|---|
| Affy Code | Accession | Accession | Symbol | Unigene Descriptor | Fold Change |
| 204052_s_at | NM_003014.2 | Hs.105700 | SFRP4 | secreted frizzled-related protein 4 | 6.7 |
| | AF311912.1 | | SRFP2 | Secreted frizzled-related protein 2 (sFRP2) | 6.5 |
| 210861_s_at | AF143679.1 | Hs.194678 | LIBC | WNT1 inducible signaling pathway protein 3 | 4.6 |
| 202196_s_at | NM_013253.1 | Hs.4909 | DKK3 | dickkopf (Xenopus laevis) homolog 3 | 4.3 |
| | L37882.1 | | | frizzled (Drosophila) homolog 2 | 4.2 |
| | NM_003882.1 | | | WNT1 inducible signalling pathway protein 1 | 3.3 |
| | U91903.1 | | | frizzled-related protein | 2.9 |
| 203706_s_at | NM_003507.1 | Hs.173859 | FZD7 | frizzled (Drosophila) homolog 7 | 2.7 |
| | NM_030775.1 | | WNT5b | WNT5b protein | 2.6 |
| 201533_at | NM_001904.1 | Hs.171271 | CTNNB1 | catenin (cadherin-associated protein), beta 1 (88 kD) | 1.7 |
| 220815_at | NM_013266.1 | Hs.257051 | VR22 | alpha-catenin-like protein | 0.5 |

TABLE 9
Genes encoding proteins of nucleotide metabolism that are diagnostic of pancreatic cancer

| | GenBank | Unigene | Gene | | Fold |
|---|---|---|---|---|---|
| Affy Code | Accession | Accession | Symbol | Unigene Descriptor | Change |
| 213988_s_at | BE971383 | Hs.28491 | | spermidinespermine N1-acetyltransferase | 7.9 |
| 203455_s_at | NM_002970.1 | Hs.28491 | SAT | spermidinespermine N1-acetyltransferase | 3.4 |

TABLE 10
Genes encoding proteins involved in smooth muscle contraction that are diagnostic of pancreatic cancer

| | GenBank | Unigene | Gene | | |
|---|---|---|---|---|---|
| Affy Code | Accession | Accession | Symbol | Unigene Descriptor | Fold Change |
| 202555_s_at | NM_005965.1 | Hs.211582 | MYLK | myosin, light polypeptide kinase | 8.3 |
| 201058_s_at | NM_006097.1 | Hs.9615 | MYRL2 | myosin regulatory light chain 2, smooth muscle | 7.1 |
| | | | | isoform | |
| 200974_at | NM_001613.1 | Hs.195851 | ACTA2 | actin, alpha 2, smooth muscle, aorta | 4.4 |
| 208636_at | AI082078_RC | Hs.119000 | | actinin, alpha 1 | 3.8 |

TABLE 11
Genes encoding mitochondrial proteins that are diagnostic of pancreatic cancer

| | | Unigene | Gene | | Fold |
|---|---|---|---|---|---|
| Affy Code | GenBank Accession | Accession | Symbol | Unigene Descriptor | Change |
| | NM_000104.2 | | | Cytochrome P450, subfamily I (dioxin-inducible) | 8.6 |
| 206662_at | NM_002064.1 | Hs.28988 | GLRX | glutaredoxin (thioltransferase) | 6.5 |
| | NM_000784.1 | | | Cytochrome P450, subfamily XXVIIA | 3.2 |
| 203343_at | NM_003359.1 | Hs.28309 | UGDH | UDP-glucose dehydrogenase | 2.6 |
| | R92925_RC | | | Mitochondrial solute carrier protein | 0.4 |
| | NM_004294.1 | | | Mitochondrial translational release factor 1 | 0.4 |
| | T67741_RC | | | Cytochrome P450, subfamily IIA | 0.4 |
| | NM_001914.1 | | | Cytochrome b-5 | 0.4 |

TABLE 12
Genes encoding collagens or proteins involved in collagen synthesis that are diagnostic of pancreatic cancer

| | GenBank | Unigene | Gene | | |
|---|---|---|---|---|---|
| Affy Code | Accession | Accession | Symbol | Unigene Descriptor | Fold Change |
| 202404_s_at | NM_000089.1 | Hs.179573 | COL1A2 | collagen, type I, alpha 2 | 21.3 |
| 202465_at | NM_002593.2 | Hs.202097 | PCOLCE | Procollagen C-endopeptidase enhancer | 19.1 |
| 204320_at | NM_001854.1 | Hs.82772 | COL11A1 | collagen, type XI, alpha 1 | 16.3 |
| 221729_at | AL575735_RC | Hs.82985 | | collagen, type V, alpha 2 | 13.1 |
| 212489_at | AI983428_RC | Hs.146428 | | collagen, type V, alpha 1 | 12.1 |
| 202766_s_at | NM_000138.1 | Hs.750 | FBN1 | fibrillin 1 (Marfan syndrome) | 5.9 |
| 202311_s_at | AI743621_RC | Hs.172928 | | collagen, type I, alpha 1 | 9 |
| 215077_at | AU144167_RC | Hs.297909 | | Homo sapiens cDNA FLJ11428 fis, clone | 5.5 |
| | | | | HEMBA1001071, highly similar to PROCOLLAGEN | |
| | | | | ALPHA 1(III) CHAIN PRECURSOR | |
| 211964_at | X05610.1 | Hs.75617 | | collagen, type IV, alpha 2 | 4 |

TABLE 13
Genes encoding inflammatory response pathway proteins that are diagnostic of pancreatic cancer

| | GenBank | Unigene | Gene | | |
|---|---|---|---|---|---|
| Affy Code | Accession | Accession | Symbol | Unigene Descriptor | Fold Change |
| 202404_s_at | NM_000089.1 | Hs.179573 | COL1A2 | collagen, type I, alpha 2 | 21.3 |
| 211719_x_at | BC005858.1 | | | fibronectin 1 | 18.2 |
| 212464_s_at | X02761.1 | Hs.287820 | | fibronectin 1 | 14.8 |
| 216442_x_at | AK026737.1 | Hs.287820 | | fibronectin 1 | 11 |
| 202267_at | NM_005562.1 | Hs.54451 | LAMC2 | laminin, gamma 2 (nicein (100 kD), kalinin (105 kD), | 9.9 |
| | | | | BM600 (100 kD), Herlitz junctional epidermolysis | |
| | | | | bullosa)) | |
| 202311_s_at | AI743621_RC | Hs.172928 | | collagen, type I, alpha 1 | 9 |
| 215077_at | AU144167_RC | Hs.297909 | | Homo sapiens cDNA FLJ11428 fis, clone | 5.5 |
| | | | | HEMBA1001071, highly similar to PROCOLLAGEN | |
| | | | | ALPHA 1(III) CHAIN PRECURSOR | |

TABLE 14
Genes encoding endoplasmic reticulum (ER) proteins that are diagnostic of pancreatic cancer

| | GenBank | Unigene | Gene | | |
|---|---|---|---|---|---|
| Affy Code | Accession | Accession | Symbol | Unigene Descriptor | Fold Change |
| 207714_s_at | NM_004353.1 | Hs.241579 | SERPINH1 | serine (or cysteine) proteinase inhibitor, clade H | 9.5 |
| | | | | (heat shock protein 47), member 1 | |
| 208131_s_at | NM_000961.1 | | PTGIS | | 6.8 |
| 215125_s_at | AV691323 | Hs.2056 | | UDP glycosyltransferase 1 family, polypeptide A9 | 5.4 |
| 212887_at | AI753659_RC | Hs.321403 | | Homo sapiens mRNA; cDNA DKFZp564O2363 | 3.9 |
| | | | | (from clone DKFZp564O2363) | |
| 209250_at | BC000961.2 | Hs.185973 | | degenerative spermatocyte (homolog Drosophila; | 2.6 |
| | | | | lipid desaturase) | |

TABLE 15
Genes involved in apoptosis that are diagnostic of pancreatic cancer

| | GenBank | Unigene | Gene | | Fold |
|---|---|---|---|---|---|
| Affy Code | Accession | Accession | Symbol | Unigene Descriptor | Change |
| 201746_at | NM_000546.2 | Hs.1846 | TP53 | Tumor protein p53 (Li-Fraumeni syndrome) | 4.2 |
| 211832_s_at | AF201370.1 | Hs.170027 | | Mouse double minute 2, human homolog of; p53-binding | 0.4 |
| | | | | protein | |

TABLE 16
Genes encoding G1/S phase cell cycle control proteins that are diagnostic of pancreatic cancer

| | GenBank | Unigene | Gene | | Fold |
|---|---|---|---|---|---|
| Affy Code | Accession | Accession | Symbol | Unigene Descriptor | Change |
| | NM_001237.1 | | CYCA2 | Cyclin A2 | 4.7 |
| 201746_at | NM_000546.2 | Hs.1846 | TP53 | Tumor protein p53 (Li-Fraumeni syndrome) | 4.2 |
| | NM_003674.1 | | CDK10 | Cyclin-dependent kinase (CDC2-like) 10 | 3.9 |
| | BE407516 | | CYCB1 | Cyclin B1 | 2.7 |
| | R78668_RC | | CDKI 1C, P57^{KIP2} | Cyclin-dependent kinase inhibitor 1C (p57^{KIP2}) | 2.6 |
| 207039_at | NM_000077.1 | Hs.1174 | CDKN2A | Cyclin-dependent kinase inhibitor 2A (melanoma, p16, | 2.2 |
| | | | | inhibits CDK4) | |
| 208711_s_at | BC000076.1 | Hs.82932 | | cyclin D1 (PRAD1: parathyroid adenomatosis 1) | 1.0 |
| 202284_s_at | NM_000389.1 | Hs.179665 | CDKN1A | Cyclin-dependent kinase inhibitor 1A (p21, Cip1) | 0.7 |

TABLE 17
Genes encoding matrix metalloproteinases that are diagnostic of pancreatic cancer

| | GenBank | Unigene | Gene | | Fold |
|---|---|---|---|---|---|
| Affy Code | Accession | Accession | Symbol | Unigene Descriptor | Change |
| 203878_s_at | NM_005940.2 | Hs.155324 | MMP11 | Matrix metalloproteinase 11 (stromelysin 3) | 16.4 |
| | NM_004995.2 | | MMP14 | Matrix metalloproteinase 14 (membrane inserted) | 6.0 |
| 201666_at | NM_003254.1 | Hs.5831 | TIMP1 | tissue inhibitor of metalloproteinase 1 (erythroid | 5.9 |
| | | | | potentiating activity, collagenase inhibitor) | |
| | NM_004530.1 | | MMP2 | Matrix metalloproteinase 2 (gelatinase A, 72 kD | 4.3 |
| | | | | gelatinase, 72 kD type IV collagenase) | |
| | AF219624.1 | | MMP28 | Matrix metalloproteinase 28 | 4.2 |
| | W45551_RC | | MMP24 | Matrix metalloproteinase 24 (membrane inserted) | 0.4 |

TABLE 18
Genes encoding proteins involved in retinoic acid signal transduction that are diagnostic of pancreatic cancer

| | GenBank | Unigene | | | Fold |
|---|---|---|---|---|---|
| Affy Code | Accession | Accession | Gene Symbol | Unigene Descriptor | Change |
| 203108_at | NM_003979.2 | Hs.194691 | RAI3 | retinoic acid induced 3 | 26.3 |
| 206392_s_at | NM_002888.1 | Hs.82547 | RARRES1 | retinoic acid receptor responder (tazarotene induced) 1 | 16.5 |
| 206391_at | NM_002888.1 | Hs.82547 | RARRES1 | retinoic acid receptor responder (tazarotene induced) 1 | 7 |
| | NM_005771.1 | | | Retinol dehydrogenase homolog | 4.7 |
| | NM_012420.1 | | | Retinoic acid and interferon-inducible protein (58 kD) | 4.2 |
| | AI806984_RC | | | Retinoic acid receptor, alpha | 2.9 |
| | BC000069.1 | | RARRES2 | retinoic acid receptor responder (tazarotene induced) 2 | 0.1 |

TABLE 19
Genes encoding calcium channel proteins that are diagnostic of pancreatic cancer

| | GenBank | Unigene | Gene | | Fold |
|---|---|---|---|---|---|
| Affy Code | Accession | Accession | Symbol | Unigene Descriptor | Change |
| 209530_at | U07139.1 | Hs.250712 | | calcium channel, voltage-dependent, beta 3 subunit | 3.8 |
| 34726_at | | | | calcium channel, voltage-dependent, beta 3 subunit | 3 |
| 208377_s_at | NM_005183.1 | Hs.139263 | CACNA1F | calcium channel, voltage-dependent, alpha 1F subunit | 0.1 |

TABLE 20
Genes encoding cathepsin proteins that are diagnostic of pancreatic cancer

| | GenBank | Unigene | Gene | | Fold |
|---|---|---|---|---|---|
| Affy Code | Accession | Accession | Symbol | Unigene Descriptor | Change |
| 205927_s_at | NM_001910.1 | Hs.1355 | CTSE | Cathepsin E (CTSE) | 12.6 |
| 202450_s_at | NM_000396.1 | Hs.83942 | CTSK | cathepsin K | 8.1 |
| | | | | (pycnodysostosis) (CTSK) | |
| 213275_x_at | W47179_RC | Hs.297939 | | cathepsin B | 4.4 |
| | AI246687_RC | | | Cathepsin C | 4.0 |
| | AK024855.1 | | | Cathepsin S | 2.3 |
| | NM_003793.2 | | | Cathepsin F | 0.4 |
| | NM_001335.1 | | | Cathepsin W | 0.3 |

TABLE 21
Genes encoding viral oncoprotein homologs that are diagnostic of pancreatic cancer

| | GenBank | Unigene | Gene | | Fold |
|---|---|---|---|---|---|
| Affy Code | Accession | Accession | Symbol | Unigene Descriptor | Change |
| 212531_at | NM_005564.1 | Hs.204238 | LCN2 | lipocalin 2 (oncogene 24p3) | 13.5 |
| | AI760277_RC | | | v-raf murine sarcoma 3611 viral oncogene homolog 1 | 5.5 |
| | AW592266_RC | | | v-myb avian myeloblastosis viral oncogene homolog-like 1 | 4.0 |
| | AA927480_RC | | | v-ski avian sarcoma viral oncogene homolog | 3.9 |
| 202625_at | AI356412_RC | Hs.80887 | | v-yes-1 Yamaguchi sarcoma viral related oncogene | 3.8 |
| | | | | homolog | |
| | NM_005402.1 | | | v-ral simian leukemia viral oncogene homolog A (ras | 3.1 |
| | | | | related) | |
| 205051_s_at | NM_000222.1 | Hs.81665 | KIT | v-kit | 2.8 |
| | NM_002908.1 | | | v-rel avian reticuloendotheliosis viral oncogene homolog | 0.4 |
| | NM_002467.1 | | | v-myc avian myelocytomatosis viral oncogene homolog | 0.3 |
| | M19720 | | | v-myc avian myelocytomatosis viral oncogene homolog | 0.3 |
| | | | | 1, lung carcinoma derived | |
| | NM_002466.1 | | | v-myb avian myeloblastosis viral oncogene homolog-like 2 | 0.2 |
| | NM_000104.2 | | | Cytochrome P450, subfamily I (dioxin-inducible) | |

TABLE 22
Genes encoding S100 calcium binding proteins that are diagnostic of pancreatic cancer

| | GenBank | Unigene | Gene | | Fold |
|---|---|---|---|---|---|
| Affy Code | Accession | Accession | Symbol | Unigene Descriptor | Change |
| 204351_at | NM_005980.1 | Hs.2962 | S100P | S100 calcium-binding protein P | 152.4 |
| 204268_at | NM_005978.2 | Hs.38991 | S100A2 | S100 calcium-binding protein A2 | 22.7 |
| 217728_at | NM_014624.2 | Hs.275243 | S100A6 | S100 calcium-binding protein A6 (calcyclin) (S100A6) | 15.2 |
| | NM_005620.1 | | S100A11 | S100 calcium-binding protein A11 (calgizzarin) | 6.9 |
| 200872_at | NM_002966.1 | Hs.119301 | S100A10 | S100 calcium-binding protein A10 (annexin II ligand, | 5.9 |
| | | | | calpactin I, light polypeptide (p11)) (S100A10) | |
| | NM_002961.2 | | S100A4 | S100 calcium-binding protein A4 | 4.3 |
| 208540_x_at | NM_021039.1 | Hs.247697 | S100A14 | S100 calcium-binding protein A14 (calgizzarin) | 3 |
| | | | | (S100A14), | |

TABLE 23
Genes encoding Homeobox proteins that are diagnostic of pancreatic cancer

| | GenBank | Unigene | Gene | | Fold |
|---|---|---|---|---|---|
| Affy Code | Accession | Accession | Symbol | Unigene Descriptor | Change |
| 205366_s_at | NM_018952.1 | Hs.98428 | HOXB6 | homeo box B6 (HOXB6) | 14.4 |
| 205453_at | NM_002145.1 | Hs.2733 | HOXB2 | homeo box B2 (HOXB2) | 6.7 |
| | AK000445.1 | | HOX C9 | Homeo box C9 | 3.2 |
| 216973_s_at | S49765.1 | Hs.819 | Homeobox | homeo box B7 (HOX B7 gene) | 2.7 |
| | | | gene | | |
| | NM_002144.1 | | HOX B1 | homeo box B1 | 0.4 |

TABLE 24
Genes encoding Zinc finger proteins that are diagnostic of pancreatic cancer

| | GenBank | Unigene | Gene | | Fold |
|---|---|---|---|---|---|
| Affy Code | Accession | Accession | Symbol | Unigene Descriptor | Change |
| | AL567808_RC | | KOX16 | Zinc finger protein 23 (KOX 16) | 4.7 |
| | NM_006299.1 | | | Zinc finger protein 193 | 3.8 |
| | NM_007150.1 | | | Zinc finger protein 185 (LIM domain) | 3.0 |
| | AU150728_RC | | | Zinc finger protein 267 | 3.0 |
| | NM_003428.1 | | HPF2 | Zinc finger protein 84 (HPF2) | 2.8 |
| | NM_020657.1 | | | Zinc finger protein 304 | 2.7 |
| | AA121673_RC | | | Zinc finger protein 281 | 2.6 |
| | NM_006526.1 | | | Zinc finger protein 217 | 2.5 |
| | NM_015871.1 | | | Zinc finger protein | 0.4 |
| | AI493587_RC | | | Zinc finger protein 106 | 0.3 |
| | NM_006006.1 | | | Zinc finger protein 145 | 0.3 |
| | NM_006963.1 | | KOX15 | Zinc finger protein 22 (KOX15) | 0.3 |

TABLE 25
Genes encoding heat shock proteins that are diagnostic of pancreatic cancer

| | GenBank | Unigene | Gene | | Fold |
|---|---|---|---|---|---|
| Affy Code | Accession | Accession | Symbol | Unigene Descriptor | Change |
| 207714_s_at | NM_004353.1 | SERPINH1 | Hs.241579 | clade H (heat shock protein 47), member 1 | 9.5 |
| | | | | (SERPINH1) | |
| | NM_005346.2 | | Hsp 1B | Heat shock 70 kD protein 1B | 4.7 |
| | NM_005345.3 | | Hsp 1A | Heat shock 70 kD protein 1A | 4.3 |
| | R01140_RC | | Hsp1 alpha | Heat shock 90 kD protein 1, alpha | 3.0 |
| | BG403660 | | | Heat shock 105 kD protein | 2.6 |
| | BE256479 | | | Heat shock 60 kD protein 1 (chaperonin) | 0.4 |
| | AB034951.1 | | | Heat shock 70 kD protein 8 | 0.4 |
| | NM_016292.1 | | | Heat shock protein 75 | 0.4 |
| | AI393937 | | | Heat shock transcription factor 1 | 0.4 |

TABLE 26
Clinicopathologic and outcome data for all patients within the cohort

| | Whole | Resected | | Median |
|---|---|---|---|---|
| | Cohort No. | Cohort No. | Grouping | survival |
| Parameter | (%) | (%) | No. | (Months) |
| Sex | | | | |
| Male | 72 (56) | 45 (59) | | |
| Female | 56 (44) | 31 (41) | | |
| Age (years) | | | | |
| Mean | 63.8 | 61 | | |
| Median | 66.5 | 65 | | |
| Range | 34-86 | 34-83 | | |
| Treatment | | | | |
| Resection | 76 (59) | | | 11 |
| Operative | 46 (36) | | | 4.6 |
| biopsy | | | | |
| No operative | 6 (5) | | | 0 |
| intervention | | | | |
| Year of | | | | |
| treatment | | | | |
| 1972-1989 | 27 (21) | 6 (8) | | 4.6 |
| | | | | 8.25 |
| 1990-2001 | 101 (79) | 70 (92) | | 8.7 |
| | | | | 12.2 |
| Outcome | | | | |
| Follow-up | 0-117 | | | |
| (months) | | | | |
| Median | 7.7 | | | |
| 30 day | | 2 (3) | | |
| mortality | | | | |
| Death from PC | 114 (92) | 63 (83) | | |
| Death from | 2 (1) | 2 (3) | | |
| other causes | | | | |
| Alive | 8 (5) | 8 (10) | | |
| Lost to follow- | 4 (1) | 3 (4) | | |
| up | | | | |
| Cancer stage | | | | |
| I | 27 (21) | | | |
| II | 13 (10) | | 40 (31) | 13.8 |
| III | 70 (55) | | | |
| IV | 17 (13) | | 87 (68) | 6.4 |
| Differentiation | | | | |
| Well | 11 (9) | 7 (9) | | |
| Moderate | 68 (53) | 44 (58) | 79 (62) | 9 |
| | | | 51 (57) | 12.2 |
| Poor | 48 (38) | 25 (33) | 48 (38) | 5 |
| | | | 25 (33) | 8.6 |
| Tumor size | | | | |
| <20 mm | | 61 (80) | | 17.1 |
| >20 mm | | 15 (20) | | 9.6 |
| Margins | | | | |
| Clear | | 40 (53) | | 14.5 |
| Involved | | 36 (47) | | 8.5 |
| Lymph node | | | | |
| status | | | | |
| Positive | | 39 (53) | | 9.2 |
| Negative | | 35 (47) | | 13.8 |

TABLE 27
Multivariate analysis of whole cohort

| Variable | Hazard's Ratio | 95% CI | P Value |
|---|---|---|---|
| A (n = 127) | | | |
| Stage I/II vs Stage III/IV | 2.21 | 1.37-3.57 | 0.0012 |
| Resection | 0.44 | 0.26-0.74 | 0.0019 |
| HOX B2 expression | 1.61 | 0.97-2.67 | 0.0659 |
| Differentiation | 1.31 | 0.88-1.96 | 0.1877 |
| B (n = 127) | | | |
| Resection | 0.33 | 0.22-0.51 | <0.0001 |
| Stage I/II vs Stage III/IV | 2.17 | 1.34-3.51 | 0.0016 |
| Differentiation | 1.28 | 0.85-1.91 | 0.2350 |
| C (n = 127) | | | |
| Resection | 0.33 | 0.22-0.50 | <0.0001 |
| Stage I/II vs Stage III/IV | 2.3 | 1.42-3.67 | 0.0007 |

TABLE 28
Multivariate analysis for clinicophathological parameters and
HOX B2 nuclear expression in resected pancreata

| Variable | Hazard's Ratio | 95% CI | P Value |
|---|---|---|---|
| A | | | |
| Hox B2 expression | 2.90 | 1.51-5.57 | 0.0014 |
| Margin involvement | 1.89 | 1.02-3.48 | 0.0428 |
| Lymph node involvement | 1.30 | 0.71-2.40 | 0.3981 |
| B | | | |
| Hox B2 expression | 2.82 | 1.48-5.40 | 0.0017 |
| Margin involvement | 2.04 | 1.17-3.53 | 0.0115 |
| Tumor Size >20 mm | 1.48 | 0.75-2.90 | 0.2567 |
| C | | | |
| Hox B2 expression | 2.69 | 1.39-5.20 | 0.0032 |
| Margin involvement | 1.75 | 0.94-3.25 | 0.0777 |
| Lymph node involvement | 1.34 | 0.73-2.46 | 0.3525 |
| Tumor Size >20 mm | 1.49 | 0.76-2.94 | 0.2474 |

1. A method of diagnosing pancreatic cancer in a subject comprising (i) contacting a biological sample of the subject being tested with a polynucleotide probe that selectively hybridizes to a sequence set forth in SEQ ID NO:15 and (ii) detecting the level of hybridization, wherein an enhanced level of hybridization for the subject detected in step (ii) compared to that obtained from a biological sample from a control subject not having pancreatic cancer indicates that the subject being tested has, or is susceptible to developing, pancreatic cancer.

2-28. (canceled)

29. The method of claim 1 wherein the polynucleotide probe comprises a sequence selected from the group consisting of:
(i) a sequence comprising at least about 20 contiguous nucleotides from the sequence set forth in SEQ ID NO:15;
(ii) a sequence that hybridizes to at least about 20 contiguous nucleotides from the sequence set forth in SEQ ID NO:15;
(iii) a sequence that is at least about 80% identical to the sequence set forth in SEQ ID NO:15;
(iv) a sequence that encodes the amino acid sequence set forth in SEQ ID NO:16; and
(v) a sequence that is complementary to any one of the sequences set forth in (i) or (ii) or (iii) or (iv).

30-31. (canceled)

32. The method according to claim 1, wherein step (ii) comprises performing a PCR reaction.

33. The method according to claim 1, wherein step (ii) comprises performing a nucleic acid hybridization.

34. The method according to claim 1, further comprising obtaining the biological sample from the subject.

35. The method according to claim 1, wherein the biological sample has been obtained previously from the subject.

36-92. (canceled)

93. A method of monitoring the efficacy of a therapeutic treatment of pancreatic cancer, the method comprising:
(i) providing a biological sample from a patient undergoing the therapeutic treatment; and
(ii) determining the level of a pancreatic cancer-associated transcript in the biological sample by contacting the biological sample with a polynucleotide that selectively hybridizes to a sequence set forth SEQ ID NO:15, thereby monitoring the efficacy of the therapy; and
(iii) comparing the level of the pancreatic cancer-associated transcript to a level of the pancreatic cancer-associated transcript in a biological sample from the patient prior to, or earlier in, the therapeutic treatment,
wherein a reduction in the level of the pancreatic-associated cancer over time is an indication of success of the therapeutic treatment.

94-106. (canceled)

107. The method according to claim 1, wherein the biological sample is contacted with at least one additional polynucleotide probe.

108. The method of claim 107 wherein the at least one additional polynucleotide probe comprises a nucleotide sequence selected from the group consisting of:
(i) a sequence comprising at least about 20 contiguous nucleotides from a sequence selected from the group consisting of: SEQ ID NO:1, SEQ ID NO:3, SEQ ID NO:5, SEQ ID NO:7 and SEQ ID NO:9;
(ii) a sequence that hybridizes to at least about 20 contiguous nucleotides from a sequence selected from the group consisting of: SEQ ID NO:1, SEQ ID NO:3, SEQ ID NO:5, SEQ ID NO:7 and SEQ ID NO:9;
(iii) a sequence that is at least about 80% identical to a sequence selected from the group consisting of: SEQ ID NO:1, SEQ ID NO:3, SEQ ID NO:5, SEQ ID NO:7 and SEQ ID NO:9;
(iv) a sequence that encodes an amino acid sequence selected from the group consisting of: SEQ ID NO:2, SEQ ID NO:4, SEQ ID NO:6, SEQ ID NO:8 and SEQ ID NO:10; and
(v) a sequence that is complementary to any one of the sequences set forth in (i) or (ii) or (iii) or (iv).

109. A method of determining the likelihood that a subject having a pancreatic cancer will survive comprising (i) contacting a biological sample from the subject with a polynucleotide probe that selectively hybridizes to a sequence set forth in SEQ ID NO:15 and (ii) detecting the level of hybridization of the polynucleotide probe in the biological sample, wherein an elevated level of hybridization of the probe for the subject compared to the hybridization obtained for a control subject not having pancreatic cancer indicates that the subject has a poor prognosis for survival.

110. The method of claim 109, wherein the biological sample is contacted with at least one additional polynucleotide probe which comprises a sequence selected from the group consisting of:
(i) a sequence comprising at least about 20 contiguous nucleotides from SEQ ID NO:15;
(ii) a sequence that hybridizes to at least about 20 contiguous nucleotides from SEQ ID NO:15;
(iii) a sequence that is at least about 80% identical to SEQ ID NO:15;
(iv) a sequence that encodes the amino acid sequence set forth in SEQ ID NO:16; and
(v) a sequence that is complementary to any one of the sequences set forth in (i) or (ii) or (iii) or (iv).

111. The method of claim 110, wherein another polynucleotide probe comprises a sequence selected from the group consisting of:
(i) a sequence comprising at least about 20 contiguous nucleotides from nucleic acid encoding an S100 calcium binding protein and having a sequence as shown in SEQ ID NO:13, SEQ ID NO:17, SEQ ID NO:19, SEQ ID NO:21, SEQ ID NO:23 or SEQ ID NO:25;
(ii) a sequence that hybridizes to at least about 20 contiguous nucleotides from nucleic acid encoding an S100 calcium binding protein and having a sequence as shown in SEQ ID NO:13, SEQ ID NO:17, SEQ ID NO:19, SEQ ID NO:21, SEQ ID NO:23 or SEQ ID NO:25;
(iii) a sequence that encodes an S100 calcium binding protein having a sequence as shown in SEQ ID NO:14, SEQ ID NO:18, SEQ ID NO:20, SEQ ID NO:22, SEQ ID NO:24 or SEQ ID NO:26; and
(iv) a sequence that is complementary to any one of the sequences set forth in (i) or (ii) or (iii).

112. The method of claim 109, wherein step (ii) comprises performing a PCR reaction.

113. A method of determining the suitability of a subject having a pancreatic cancer for surgical resection therapy, said method comprising (i) contacting a biological sample from said subject being tested with one or more polynucleotide probes for a time and under conditions sufficient for hybridization to occur and (ii) detecting the hybridization wherein an elevated level of hybridization of the probe for the subject being tested compared to the hybridization obtained for a control subject not having pancreatic cancer indicates that the subject being tested is unsuitable for surgical resection therapy, and wherein one polynucleotide probe comprises a sequence selected from the group consisting of:
(i) a sequence comprising at least about 20 contiguous nucleotides from SEQ ID NO:15;
(ii) a sequence that hybridizes to at least about 20 contiguous nucleotides from SEQ ID NO:15;
(iii) a sequence that is at least about 80% identical to SEQ ID NO:15;
(iv) a sequence that encodes the amino acid sequence set forth in SEQ ID NO:16; and
(v) a sequence that is complementary to any one of the sequences set forth in (i) or (ii) or (iii) or (iv).

114. The method of claim 113, wherein another polynucleotide probe comprises a sequence selected from the group consisting of:
(i) a sequence comprising at least about 20 contiguous nucleotides from nucleic acid encoding an S100 calcium binding protein and having a sequence as shown in SEQ ID NO:13, SEQ ID NO:17, SEQ ID NO:19, SEQ ID NO:21, SEQ ID NO:23 or SEQ ID NO:25;
(ii) a sequence that hybridizes to at least about 20 contiguous nucleotides from nucleic acid encoding an S100 calcium binding protein and having a sequence as shown in SEQ ID NO:13, SEQ ID NO:17, SEQ ID NO:19, SEQ ID NO:21, SEQ ID NO:23 or SEQ ID NO:25;
(iii) a sequence that encodes an S100 calcium binding protein having a sequence as shown in SEQ ID NO:14, SEQ ID NO:18, SEQ ID NO:20, SEQ ID NO:22, SEQ ID NO:24 or SEQ ID NO:26; and
(iv) a sequence that is complementary to any one of the sequences set forth in (i) or (ii) or (iii).

115. The method of claim 113, wherein step (ii) comprises performing a PCR reaction.

116. The method of claim 107 wherein the at least one additional polynucleotide probe comprises a nucleotide sequence selected from the group consisting of:
(i) a sequence comprising at least about 20 contiguous nucleotides from nucleic acid encoding an S100 calcium binding protein and having a sequence as shown in SEQ ID NO:13, SEQ ID NO:17, SEQ ID NO:19, SEQ ID NO:21, SEQ ID NO:23 or SEQ ID NO:25;
(ii) a sequence that hybridizes to at least about 20 contiguous nucleotides from nucleic acid encoding an S100 calcium binding protein and having a sequence as shown in SEQ ID NO:13, SEQ ID NO:17, SEQ ID NO:19, SEQ ID NO:21, SEQ ID NO:23 or SEQ ID NO:25;
(iii) a sequence that encodes an S100 calcium binding protein having a sequence as shown in SEQ ID NO:14, SEQ ID NO:18, SEQ ID NO:20, SEQ ID NO:22, SEQ ID NO:24 or SEQ ID NO:26; and
(iv) a sequence that is complementary to any one of the sequences set forth in (i) or (ii) or (iii).
